(19) United States
(12) Patent Application Publication
(10) Pub. No.: US 2014/0271549 A1
(43) Pub. Date: Sep. 18, 2014

(54) Use of Antibiotics to Enhance Treatment With Therapeutic Viruses

(71) Applicant: Aladar A. Szalay, Highland, CA (US)

(72) Inventor: Aladar A. Szalay, Highland, CA (US)

(21) Appl. No.: 13/999,616

(22) Filed: Mar. 11, 2014

Related U.S. Application Data
(60) Provisional application No. 61/852,133, filed on Mar. 15, 2013.

(51) Int. Cl. A61K 38/14; A61K 48/00; A61K 31/431; A61K 31/407; A61K 35/76; A61K 31/7036

(52) U.S. Cl. CPC A61K 38/14; A61K 35/768; A61K 31/7036; A61K 31/431; A61K 31/407; A61K 48/00
USPC 424/93.2

(57) ABSTRACT

Provided are methods for increasing the therapeutic efficacy of viral therapy by administering an antibiotic effective against commensal bacteria with the viral therapy. Included are methods for treating cancers, tumors and metastases by administering the virus and the antibiotic.

### RELATED APPLICATIONS

Benefit of priority is claimed to U.S. Provisional Application Ser. No. 61/852,133, filed Mar. 15, 2013, to Aladar A. Szalay, entitled “USE OF ANTIBIOTICS TO ENHANCE TREATMENT WITH THERAPEUTIC VIRUSES.” The subject matter of this application is incorporated by reference in its entirety.

### INCORPORATION BY REFERENCE OF SEQUENCE LISTING PROVIDED ON COMPACT DISCS

An electronic version on compact disc (CD-R) of the Sequence Listing is filed herewith in duplicate (labeled Copy 1 and Copy 2), the contents of which are incorporated by reference in their entirety. The computer-readable file on each of the aforementioned compact discs, created on Mar. 10, 2014, is identical, 5.27 MB in size, and titled 4826SEQ.001.txt.

### FIELD OF INVENTION

Provided are methods of increasing the therapeutic efficacy of viral therapy, such as oncolytic viral therapy and gene therapy by administration of an antibiotics, and methods of combination therapy in which antibiotics that inhibit gut bacteria are administered with therapeutic viruses. Combinations and kits for practicing the methods also are provided.

### BACKGROUND

Biological therapies, such as gene therapies, cell therapies and oncolytic viral therapies are viable treatment modalities. Treatment of cancers and other disorders with therapeutic viruses is an important therapeutic regimens. Increasing the effectiveness of such therapies would be advantageous. This and other needs are addressed herein.

### SUMMARY

Provided are methods for enhancing the effectiveness of a therapeutic virus by administering an antibiotic with, before, after or during treatment with the therapeutic virus. Corresponding methods of treatment also are provided.

The antibiotic is one that inhibits the growth of or kills commensal gut bacteria and thereby reduces the number of gut bacteria and is not an anti-cancer antibiotic. Administration of such antibiotics can be employed with any type of viral therapy, including, for example viral therapy to provide gene therapy and/or to treat cancers and tumors. Included among the methods are methods for treating cancers, tumors and/or metastases by administering a therapeutic virus for treatment of cancers, tumors or metastases; and administering an antibiotic that is effective against commensal gut bacteria, wherein the oncolytic therapeutic virus is effective for treating tumors. Also provided are compositions for use for increasing the effectiveness of therapeutic viral therapy for the treatment of tumors. The compositions contain an antibiotic that inhibits the growth of or kills commensal gut bacteria to thereby reduce the number of gut bacteria and is not an anti-cancer antibiotic.

For the methods and compositions for use, the therapeutic virus includes, for example viruses selected from among a retrovirus, adenovirus, lentivirus, herpes simplex virus, poxvirus and adeno-associated virus (AAV). The therapeutic viruses can be oncolytic viruses, such as, but not limited to Newcastle Disease virus, parvovirus, pox virus, such as vaccinia virus, measles virus, reovirus, vesicular stomatitis virus (VSV), oncolytic adenovirus, poliovirus, herpes virus and derivative and modified forms thereof. Exemplary vaccinia viruses include strains designated Western Reserve (WR), Copenhagen, Tashkent, Tian Tan, Lister, Wyeth, such as the viruses designated JX-294 or JX-594, IHD-J, and IHD-W, Brighton, Ankara, MVA, Dairen I, LIVP, LC16M8, LC16MO, LIVP and WR 65-16. All of the viruses can be modified to include and encode a therapeutic product and or a diagnostic or detectable product. Viruses, such as adenoviruses, that are not inherently oncolytic, can be modified to be oncolytic. Such viruses are well known in the art.

Vaccinia viruses include Lister strain vaccinia viruses, such as the LIVP strain virus. Exemplary of such viruses is the virus designated GLV-1h68 and derivatives and modified forms thereof, as well as clonal strains of an LIVP virus and a modified forms thereof containing nucleic acid encoding a heterologous gene product.

The genome of the LIVP virus and modified forms thereof can include a sequence of nucleotides set forth in SEQ ID NO:2, or a sequence of nucleotides that has at least 95% sequence identity to SEQ ID NO:2; or virus comprises a sequence of nucleotides set forth in any of SEQ ID NOS: 3-9, or a sequence of nucleotides that has at least 97% sequence identity to a sequence of nucleotides set forth in any of SEQ ID NOS: 3-9, or a sequence of nucleotides selected from among any of SEQ ID NOS:1 and 10-19, or a sequence of nucleotides that exhibits at least 99% sequence identity to any of SEQ ID NOS: 1 and 10-19.

Also exemplary of the viruses are clonal strains of LIVP, such as the viruses whose genomes contain a sequence of nucleotides selected from: a) nucleotides 2,256-180,095 of SEQ ID NO:3, nucleotides 11,243-182,721 of SEQ ID NO:4, nucleotides 6,264-181,390 of SEQ ID NO:5, nucleotides 7,044-181,820 of SEQ ID NO:6, nucleotides 6,674-181,409 of SEQ ID NO:7, nucleotides 6,716-181,367 of SEQ ID NO:8 or nucleotides 6,899-181,870 of SEQ ID NO:9; b) a sequence of nucleotides that has at least 97% sequence identity to a sequence of nucleotides 2,256-180,095 of SEQ ID NO:3, nucleotides 11,243-182,721 of SEQ ID NO:4, nucleotides 6,264-181,390 of SEQ ID NO:5, nucleotides 7,044-181,820 of SEQ ID NO:6, nucleotides 6,674-181,409 of SEQ ID NO:7, nucleotides 6,716-181,367 of SEQ ID NO:8 or nucleotides 6,899-181,870 of SEQ ID NO:9

Also contemplated are modified forms of any of the therapeutic viruses that include heterologous nucleic acid encoding therapeutic and/or diagnostic products as required. The nucleic acid encoding the heterologous gene product can be inserted into or in place of a non-essential gene or region in the genome of the virus. For example, the nucleic acid encoding the heterologous gene product is inserted into a vaccinia virus, such as LIVP, at the hemagglutinin (HA), thymidine kinase (TK), F14.5L, vaccinia growth factor (VGF), A35R, N1L, E2L/E3L, K1L/K2L, superoxide dismutase locus, 7.5K, C7-K1L, B13R+B14R, A26L or 14L gene loci in the genome of the virus.

Heterologous products include therapeutic proteins and diagnostic products, such as detectable products and products that produce a detectable signal, such as reporter gene products, such as a fluorescent protein, a bioluminescent protein, a receptor and an enzyme. For example, the fluorescent protein can be selected from among a green fluorescent protein, an enhanced green fluorescent protein, a blue fluorescent protein, a cyan fluorescent protein, a yellow fluorescent protein, a red fluorescent protein and a far-red fluorescent protein, such as the protein designated TurboFP635. Enzymes include, for example, a luciferase, β-glucuronidase, β-galactosidase, chloramphenicol acetyl tranferase (CAT), alkaline phosphatase, and horseradish peroxidase. Enzymes include, but are not limited to, enzymes that modify a substrate to produce a detectable product or signal or are detectable by antibodies, proteins that can bind a contrasting agent, genes for optical imaging or detection, genes for PET imaging and genes for MRI imaging. Receptors include those that bind to a detectable moiety or a ligand attached to a detectable moiety, including, but not limited to a radiolabel, a chromogen, or a fluorescent moiety. Therapeutic products can be selected from among, for example, an anticancer agent, an antimetastatic agent, an antiangiogenic agent, an immunomodulatory molecule, an antigen, a cell matrix degradative gene, genes for tissue regeneration and reprogramming human somatic cells to pluripotency.

The antibiotic is administered and/in the compositions in an amount that reduces or eliminates commensal gut bacteria or provided. The single dosage or daily dosage of antibiotic depends upon the particular antibiotic, but such dosages are well known. Exemplary single and daily dosages include, for example, an amount between at or about at least 1 mg and at or about at least 10 g; or between at or about at least 1 mg and at or about at least 1000 mg; or between at or about at 500 mg and at or about at least 5 g; or is or is at least about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975 or 1000 mg, 1.5, 2, 2.5, 3, 3.5, 4, 4.5, or 5 g.

For the methods, the antibiotic can be administered prior to, with, after, during or intermittently with virus. It can be administered once or a plurality of times. It can be administered with each viral therapy cycle or at other intervals. For example, the antibiotic can be administered at least, at about or at 1, 2, 3, 4, 5, 6, 7, 8, 9, 10, 11, 12, 13, 14, 15, 16, 17, 18, 19, 20, 21, 22, 23, 24, 36 or 48 or more hours prior to administration of the virus. When administered after administration of the virus, the antibiotic can be administered, for example, at about or at ¼, ½, 1, 2, 3, 4, 5, 6, 7, 8, 9, 10, 11, 12, 13, 14, 15, 16, 17, 18, 19, 20, 21, 22, 23, 24 or more hours, 2, 3, 4, 5, 6, 7, 8, 9, 10, 11, 12, 13, 14 or more days after administration of the virus.

Exemplary antibiotics for use in the methods and in the compositions include any antibiotic that reduces the number or amount of commensal gut bacteria. These include, but are not limited to, penicillins, penicillin combinations, cephalosporins, tetracyclines, β-lactam antibiotics, carbacephems, glycopeptides, aminoglycosides, ansamycins, macrolides, monobactams, nitrofurans, sulfonamides, lincosamides, lipopeptides, polypeptides, quinolones, drugs against mycobacteria, oxazolidinones, arsphenamine, chloramphenicol, fosfomycin, fusidic acid, metronidazole, tazobactam, mupirocin, platensimycin, quinupristin/dalfopristin, thiamphenicol, tigecycline, tinidazole and trimethoprim and mixtures thereof. The antibiotic can be selected from among penicillin, benzylpenicillin (penicillin G), procaine benzylpenicillin (procaine penicillin), benzathine benzylpenicillin (benzathine penicillin), phenoxymethylpenicillin (penicillin V), amoxicillin, ampicillin, azlocillin, carbenicillin, cloxacillin, dicloxacillin, flucloxacillin, mezlocillin, methicillin, nafcillin, oxacillin, temocillin, ticarcillin, amoxicillin/clavulanate, ampicillin/sulbactam, piperacillin/tazobactam, ticarcillin/clavulanate, demeclocycline, doxycycline, minocycline, oxytetracycline, tetracycline, cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefmetazole, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, cefaloram, cefaparole, cefcanel, cefedrolor, cefempidone, cefetrizole, cefivitril, cefmepidium, cefoxazole, cefrotil, cefsumide, ceftioxide, cefuracetime, ertapenem, doripenem, imipenem, imipenem/cilastatin, meropenem, panipenem/betamipron, biapenem, razupenem, tebipenem, loracarbef, teicoplanin, vancomycin, bleomycin, ramoplanin, decaplanin, telavancin, streptomycin, gentamicin, kanamycin, neomycin, netilmicin, tobramycin, spectinomycin, paromomycin, framycetin, ribostamycin, amikacin, arbekacin, bekanamycin, dibekacin, rhodostreptomycin, apramycin, hygromycin B, paromomycin sulfate, sisomicin, isepamicin, verdamicin, astromicin, geldanamycin, herbimycin, rifaximin, azithromycin, clarithromycin, dirithromycin, erythromycin, roxithromycin, telithromycin, carbomycin A, josamycin, kitasamycin, midecamycin, midecamycin acetate, oleandomycin, solithromycin, spiramycin, troleandomycin, tylosin, tylocine, ketolides such as telithromycin, cethromycin, solithromycin, spiramycin, ansamycin, oleandomycin, carbomycin, tylosin, aztreonam, furazolidone, nitrofurantoin, mafenide, sulfamethoxazole, sulfisomidine, sulfadiazine, silver sulfadiazine, sulfamethoxine, sulfamethizole, sulfanilamide, sulfasalazine, sulfisoxazole, trimethoprim-sulfamethoxazole, sulfonamidochrysoidine, sulfacetamide, sulfadoxine, dichlorphenamide, clindamycin, lincomycin, daptomycin, bacitracin, colistin, polymyxin B, moxifloxacin, ciprofloxacin, levofloxacin, cinoxacin, nalidixic acid, oxolinic acid, piromidic acid, pipemidic acid, rosoxacin, enoxacin, fleroxacin, lomefloxacin, nadifloxacin, norfloxacin, ofloxacin, pefloxacin, rufloxacin, balofloxacin, grepafloxacin, pazufloxacin, sparfloxacin, tosufloxacin, clinafloxacin, gatifloxacin, gemifloxacin, moxifloxacin, sitafloxacin, trovafloxacin, prulifloxacin, clofazimine, dapsone, capreomycin, cycloserine, ethambutol, ethionamide, isoniazid, pyrazinamide, rifampicin, rifabutin, rifapentine, streptomycin, linezolid, posizolid, radezolid, cycloserine, torezolid, arsphenamine, chloramphenicol, fosfomycin, fusidic acid, metronidazole, tazobactam, mupirocin, platensimycin, quinupristin/dalfopristin, thiamphenicol, tigecycline, tinidazole and trimethoprim and mixtures of any of the antibiotics. Particular antibiotics include penicillin, streptomycin, ampicillin, neomycin, metronidazole, vancomycin, tazobactam, meropenem, a mixture of penicillin and streptomycin, a mixture of ampicillin, neomycin, metronidazole and vancomycin, and a mixture of tazobactam, meropenem and vancomycin.

An antimycotic can be administered with the antibiotic or before or after the antibiotic or with the virus or before or after the virus. The antimycotic can be included in the compositions. Exemplary antimycotics include, but are not limited to, amphotericin B, candicidin, filipin, hamycin, natamycin, nystatin, rimocidin, imidazole antifungals, bifonazole, butoconazole, clotrimazole, econazole, fenticonazole, isoconazole, ketoconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfine, butenafine, naftifine, terbinafine, anidulafungin, caspofungin, micafungin, ciclopirox, flucytosine, 5-fluorocytosine, griseofulvin, haloprogin, polygodial, tolnaftate, undecylenic acid and crystal violet. The antimycotic can be administered as part of the methods for enhancing the viral therapy, and also to control and fungal infections consequent to antibiotic administration.

Viral dosages depend upon the virus, the regimen, the indication and also the subject and, if necessary can be empirically determined. Exemplary dosages include, for example, 1×10⁶ pfu to 1×10¹⁴ pfu, or an amount that is at least or at least about or is or is about 1×10⁶ pfu, 1×10⁷ pfu or 1×10⁸ pfu, 1×10⁹ pfu, 3×10⁹ pfu, 1×10¹⁰ pfu, 1×10¹¹ pfu, 1×10¹² pfu, 1×10¹³ pfu, or 1×10¹⁴ pfu.

Diseases and conditions whose treatment with viral therapy that is enhanced, includes any disease or condition treated by viral therapy, including tumors, cancers and metastases. These include solid tumors and disseminated tumors, CTCs, blood and cancers of other body fluids, such as leptomeningeal metastases (LM), which result from the spread of metastatic tumor cells to the cerebrospinal fluid (CSF) and leptomeninges, and of peritoneal carcinomatosis. Cancers and tumor include, for example, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma, epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia and Wilm's tumor. The therapies include combination therapies such as combining the viral treatment with an additional anti-cancer therapy, such as, but not limited to chemotherapeutic compounds, toxins, alkylating agents, nitrosoureas, anticancer antibiotics, antimetabolites, antimitotics, topoisomerase inhibitors, cytokines, growth factors, hormones, photosensitizing agents, radionuclides, signaling modulators, anticancer antibodies, anticancer oligopeptides, anticancer oligonucleotides, angiogenesis inhibitors or radiation therapy, or combinations thereof.

### DETAILED DESCRIPTION

### Outline

### A. DEFINITIONS

B. OVERVIEW

1. Gut Bacteria and Immune Response

2. Viral Therapy

3. Methods of Treatment with Antibiotics to Increase the Therapeutic Efficacy of

### Viral Therapy

### C. ANTIBIOTICS

Administrations and dosages

### D. VIRUSES

1. Exemplary Oncolytic Viruses
a. Poxviruses—Vaccinia Viruses
   i. Modified Vaccinia Viruses
b. Other Oncolytic Viruses

3. Modification of Viruses
a. Heterologous Nucleic Acid and Exemplary Modifications
   i. Diagnostic or reporter gene products
   ii. Therapeutic gene products
   iii. Antigens
   iv. Modifications to alter attenuation of the viruses
b. Control of heterologous gene expression
c. Methods for generating modified viruses

4. Methods of Producing Viruses
a. Host cells for propagation
b. Concentration determination
c. Storage methods
d. Preparation of virus

### E. METHODS OF TREATMENT WITH ANTIBIOTICS FOR INCREASING THE THERAPEUTIC EFFICACY OF VIRAL THERAPY

1. Therapeutic Methods

2. Pharmaceutical Compositions, Combinations and Kits
a. Pharmaceutical compositions
b. Combinations
c. Kits

3. Dosages and Administration
a. Steps prior to administering the virus
b. Mode of administration
c. Dosages and dosage regime
d. Combination Therapy
   i. Administering a plurality of viruses
   ii. Therapeutic Compounds
   iii. Immunotherapies and biological therapies
e. State of subject

4. Monitoring Oncolytic Viral Therapy
a. Monitoring viral gene expression
b. Monitoring tumor size
c. Monitoring antibody titer
d. Monitoring general health diagnostics
e. Monitoring coordinated with treatment

### F. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, an “antibiotic” refers to an agent used for elimination of bacteria, such as for treatment of infections therefrom. Antibiotics for use herein are not employed for treatment of cancer and are distinct from anti-cancer antibiotics. Exemplary antibiotics for use in the methods herein are those that eliminate gut bacteria, are not anti-cancer antibiotics and include, but are not limited to, penicillin, streptomycin, ampicillin, neomycin, metronidazole, vancomycin, tazobactam, meropenem, or mixtures thereof.

As used herein, anti-cancer antibiotics are antibiotics that have anti-tumor activity and are employed a therapeutic agents for treatment of cancers. Exemplary anti-cancer antibiotics include, but are not limited to, anthracyclines such as doxorubicin hydrochloride (adriamycin), idarubicin hydrochloride, daunorubicin hydrochloride, aclarubicin hydrochloride, epirubicin hydrochloride and pirarubicin hydrochloride, phleomycins such as phleomycin and peplomycin sulfate, mitomycins such as mitomycin C, actinomycins such as actinomycin D, zinostatin stimalamer and polypeptides such as neocarzinostatin.

As used herein, an anti-mycotic is are agents used for the treatment of fungal infections, including those that follow antibiotic treatment. Exemplary of an anti-mycotic is amphotericin B.

As used herein, a subject includes any organism, including an animal, for whom diagnosis, screening, monitoring or treatment is contemplated. Animals include mammals, such as, for example, primates, domesticated animals and livestock. An exemplary primate is a human. Subject include any animals, such as, such as a mammal, primate, human, domesticated animal or livestock, or other animal subject afflicted with a disease condition or for which a disease condition is to be determined or risk of a disease condition is to be determined.

As used herein, a patient refers to a human subject exhibiting symptoms of a disease or disorder.

As used herein, animals include any animal, such as, but are not limited to, primates, including humans, apes and monkeys; rodents, such as mice, rats, rabbits, and ferrets; fowl, such as chickens; ruminants, such as goats, cows, deer, and sheep; horses, pigs, dogs, cats, fish, and other animals. Non-human animals exclude humans as the contemplated animal.

As used herein, the term “suffering from disease” refers to a subject (e.g., a human) who is experiencing a particular disease. It is not intended that the methods provided be limited to any particular signs or symptoms, nor disease. Thus, it is intended that the methods provided encompass subjects that are experiencing any range of disease, from sub-clinical to full-blown disease, wherein the subject exhibits at least some of the indicia (e.g., signs and symptoms) associated with the particular disease.

As used herein, the term “subject diagnosed with a cancer” refers to a subject who has been tested and found to have cancerous cells. The cancer can be diagnosed using any suitable method, including but not limited to, biopsy, x-ray, MRI, PET, blood test, and any diagnostic methods described herein.

As used herein, a “metastatic cell” is a cell that has the potential for metastasis. Metastatic cells have the ability to metastasize from a first tumor in a subject and can colonize tissue at a different site in the subject to form a second tumor at the site.

As used herein, a “metastasis” refers to the spread of cancer from one part of the body to another. For example, in the metastatic process, malignant cells can spread from the site of the primary tumor in which the malignant cells arose and move into lymphatic and blood vessels, which transport the cells to normal tissues elsewhere in an organism where the cells continue to proliferate. A tumor formed by cells that have spread by metastasis is called a “metastatic tumor,” a “secondary tumor” or a “metastasis.”

As used herein, “tumorigenic cell,” is a cell that, when introduced into a suitable site in a subject, can form a tumor. The cell can be non-metastatic or metastatic.

As used herein, a “normal cell” or “non-tumor cell” are used interchangeably and refer to a cell that is not derived from a tumor.

As used herein, the term “cell” refers to the basic unit of structure and function of a living organism as is commonly understood in the biological sciences. A cell can be a unicellular organism that is self-sufficient and that can exist as a functional whole independently of other cells. A cell also can be one that, when not isolated from the environment in which it occurs in nature, is part of a multicellular organism made up of more than one type of cell. Such a cell, which can be thought of as a “non-organism” or “non-organismal” cell, generally is specialized in that it performs only a subset of the functions performed by the multicellular organism as whole. Thus, this type of cell is not a unicellular organism. Such a cell can be a prokaryotic or eukaryotic cell, including animal cells, such as mammalian cells, human cells and non-human animal cells or non-human mammalian cells. Animal cells include any cell of animal origin that can be found in an animal. Thus, animal cells include, for example, cells that make up the various organs, tissues and systems of an animal.

As used herein an “isolated cell” is a cell that exists in vitro and is separate from the organism from which it was originally derived.

As used herein, a “cell line” is a population of cells derived from a primary cell that is capable of stable growth in vitro for many generations. Cell lines are commonly referred to as “immortalized” cell lines to describe their ability to continuously propagate in vitro.

As used herein a “tumor cell line: is a population of cells that is initially derived from a tumor. Such cells typically have undergone some change in vivo such that they theoretically have indefinite growth in culture; unlike primary cells, which can be cultured only for a finite period of time. Such cells can form tumors after they are injected into susceptible animals.

As used herein, a “primary cell” is a cell that has been isolated from a subject.

As used herein, a “host cell” or “target cell” are used interchangeably to mean a cell that can be infected by a virus.

As used herein, the term “tissue” refers to a group, collection or aggregate of similar cells generally acting to perform a specific function within an organism.

As used herein, “virus” refers to any of a large group of infectious entities that cannot grow or replicate without a host cell. Viruses typically contain a protein coat surrounding an RNA or DNA core of genetic material, but no semipermeable membrane, and are capable of growth and multiplication only in living cells. Viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picornavirus, Sindbis virus, papillomavirus, parvovirus, reovirus, coxsackievirus, influenza virus, mumps virus, poliovirus, and semliki forest virus.

As used herein, oncolytic viruses refer to viruses that replicate selectively in tumor cells in tumorous subjects. Some oncolytic viruses can kill a tumor cell following infection of the tumor cell. For example, an oncolytic virus can cause death of the tumor cell by lysing the tumor cell or inducing cell death of the tumor cell.

As used herein the term “vaccinia virus” or “VACV” denotes a large, complex, enveloped virus belonging to the poxvirus family. It has a linear, double-stranded DNA genome approximately 190 kbp in length, and which encodes approximately 200 proteins. Vaccinia virus strains include, but are not limited to, strains of, derived from, or modified forms of Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health vaccinia virus strains.

As used herein, Lister Strain of the Institute of Viral Preparations (LIVP) or LIVP virus strain refers to a virus strain that is the attenuated Lister strain (ATCC Catalog No. VR-1549) that was produced by adaption to calf skin at the Institute of Viral Preparations, Moscow, Russia (Al'tshtein et al. (1985) Dokl. Akad. Nauk USSR 285:696-699). The LIVP strain can be obtained, for example, from the Institute of Viral Preparations, Moscow, Russia (see. e.g., Kutinova et al. (1995) Vaccine 13:487-493); the Microorganism Collection of FSRI SRC VB Vector (Kozlova et al. (2010) Environ. Sci. Technol. 44:5121-5126); or can be obtained from the Moscow Ivanovsky Institute of Virology (C0355 K0602; Agranovski et al. (2006) Atmospheric Environment 40:3924-3929). It also is well known to those of skill in the art; it was the vaccine strain used for vaccination in the USSR and throughout Asia and India. The strain is used by researchers and is well known (see e.g., Altshteyn et al. (1985) Dokl. Akad. Nauk USSR 285:696-699; Kutinova et al. (1994) Arch. Virol. 134:1-9; Kutinova et al. (1995) Vaccine 13:487-493; Shchelkunov et al. (1993) Virus Research 28:273-283; Sroller et al. (1998) Archives Virology 143:1311-1320; Zinoviev et al. (1994) Gene 147:209-214; and Chkheidze et al. (1993) FEBS 336:340-342). Among the LIVP strains is one that contains a genome having a sequence of nucleotides set forth in SEQ ID NO:2, or a sequence that is at least or at least about 99% identical to the sequence of nucleotides set forth in SEQ ID NO:2.

As used herein, an LIVP clonal strain or LIVP clonal isolate refers to a virus that is derived from the LIVP virus strain by plaque isolation, or other method in which a single clone is propagated, and that has a genome that is homogenous in sequence. Hence, an LIVP clonal strain includes a virus whose genome is present in a virus preparation propagated from LIVP. An LIVP clonal strain does not include a recombinant LIVP virus that is genetically engineered by recombinant means using recombinant DNA methods to introduce heterologous nucleic acid. In particular, an LIVP clonal strain has a genome that does not contain heterologous nucleic acid that contains an open reading frame encoding a heterologous protein. For example, an LIVP clonal strain has a genome that does not contain non-viral heterologous nucleic acid that contains an open reading frame encoding a non-viral heterologous protein. As described herein, however, it is understood that any of the LIVP clonal strains provided herein can be modified in its genome by recombinant means to generate a recombinant virus. For example, an LIVP clonal strain can be modified to generate a recombinant LIVP virus that contains insertion of nucleotides that contain an open reading frame encoding a heterologous protein.

As used herein, LIVP 1.1.1 is an LIVP clonal strain that has a genome having a sequence of nucleotides set forth in SEQ ID NO:3 or a genome having a sequence of nucleotides that has at least 97%, 98%, or 99% sequence identity to the sequence of nucleotides set forth in SEQ ID NO:3.

As used herein, LIVP 2.1.1 is an LIVP clonal strain that has a genome having a sequence of nucleotides set forth in SEQ ID NO:4, or a genome having a sequence of nucleotides that has at least 97%, 98%, or 99% sequence identity to the sequence of nucleotides set forth in SEQ ID NO:4.

As used herein, LIVP 4.1.1 is an LIVP clonal strain that has a genome having a sequence of nucleotides set forth in SEQ ID NO:5, or a genome having a sequence of nucleotides that has at least 97%, 98%, or 99% sequence identity to the sequence of nucleotides set forth in SEQ ID NO:5.

As used herein, LIVP 5.1.1 is an LIVP clonal strain that has a genome having a sequence of nucleotides set forth in SEQ ID NO:6, or a genome having a sequence of nucleotides that has at least 97%, 98%, or 99% sequence identity to the sequence of nucleotides set forth in SEQ ID NO:6.

As used herein, LIVP 6.1.1 is an LIVP clonal strain that has a genome having a sequence of nucleotides set forth in SEQ ID NO:7, or a genome having a sequence of nucleotides that has at least 97%, 98%, or 99% sequence identity to the sequence of nucleotides set forth in SEQ ID NO:7.

As used herein, LIVP 7.1.1 is an LIVP clonal strain that has a genome having a sequence of nucleotides set forth in SEQ ID NO:8, or a genome having a sequence of nucleotides that has at least 97%, 98%, or 99% sequence identity to the sequence of nucleotides set forth in SEQ ID NO:8.

As used herein, LIVP 8.1.1 is an LIVP clonal strain that has a genome having a sequence of nucleotides set forth in SEQ ID NO:9, or a genome having a sequence of nucleotides that has at least 97%, 98%, or 99% sequence identity to the sequence of nucleotides set forth in SEQ ID NO:9.

As used herein, the term “modified virus” refers to a virus that is altered compared to a parental strain of the virus. Typically modified viruses have one or more truncations, mutations, insertions or deletions in the genome of virus. A modified virus can have one or more endogenous viral genes modified and/or one or more intergenic regions modified. Exemplary modified viruses can have one or more heterologous nucleic acid sequences inserted into the genome of the virus. Modified viruses can contain one or more heterologous nucleic acid sequences in the form of a gene expression cassette for the expression of a heterologous gene.

As used herein, a modified LIVP virus strain refers to an LIVP virus that has a genome that is not contained in LIVP, but is a virus that is produced by modification of a genome of a strain derived from LIVP. Typically, the genome of the virus is modified by substitution (replacement), insertion (addition) or deletion (truncation) of nucleotides. Modifications can be made using any method known to one of skill in the art such as genetic engineering and recombinant DNA methods. Hence, a modified virus is a virus that is altered in its genome compared to the genome of a parental virus. Exemplary modified viruses have one or more heterologous nucleic acid sequences inserted into the genome of the virus. Typically, the heterologous nucleic acid contains an open reading frame encoding a heterologous protein. For example, modified viruses herein can contain one or more heterologous nucleic acid sequences in the form of a gene expression cassette for the expression of a heterologous gene.

As used herein, multiplicity of infection (MOI) refers to the ratio of viral particles to cells used for infection. For example, infection at a MOI of 1 mean that virus is added to a sample of cells at a ratio of 1 virus particle to one cell.

As used herein a “gene expression cassette” or “expression cassette” is a nucleic acid construct, containing nucleic acid elements that are capable of effecting expression of a gene in hosts that are compatible with such sequences. Expression cassettes include at least promoters and optionally, transcription termination signals. Typically, the expression cassette includes a nucleic acid to be transcribed operably linked to a promoter. Expression cassettes can contain genes that encode, for example, a therapeutic gene product, or a detectable protein or a selectable marker gene.

As used herein, a heterologous nucleic acid (also referred to as exogenous nucleic acid or foreign nucleic acid) refers to a nucleic acid that is not normally produced in vivo by an organism or virus from which it is expressed or that is produced by an organism or a virus but is at a different locus, or that mediates or encodes mediators that alter expression of endogenous nucleic acid, such as DNA, by affecting transcription, translation, or other regulatable biochemical processes. Hence, heterologous nucleic acid is often not normally endogenous to a virus into which it is introduced. Heterologous nucleic acid can refer to a nucleic acid molecule from another virus in the same organism or another organism, including the same species or another species. Heterologous nucleic acid, however, can be endogenous, but is nucleic acid that is expressed from a different locus or altered in its expression or sequence (e.g., a plasmid). Thus, heterologous nucleic acid includes a nucleic acid molecule not present in the exact orientation or position as the counterpart nucleic acid molecule, such as DNA, is found in a genome. Generally, although not necessarily, such nucleic acid encodes RNA and proteins that are not normally produced by the virus or in the same way in the virus in which it is expressed. Any nucleic acid, such as DNA, that one of skill in the art recognizes or considers as heterologous, exogenous or foreign to the virus in which the nucleic acid is expressed is herein encompassed by heterologous nucleic acid. Examples of heterologous nucleic acid include, but are not limited to, nucleic acid that encodes exogenous peptides/proteins, including diagnostic and/or therapeutic agents. Proteins that are encoded by heterologous nucleic acid can be expressed within the virus, secreted, or expressed on the surface of the virus in which the heterologous nucleic acid has been introduced.

As used herein, a heterologous protein or heterologous polypeptide (also referred to as exogenous protein, exogenous polypeptide, foreign protein or foreign polypeptide) refers to a protein that is not normally produced by a virus.

As used herein, operative linkage of heterologous nucleic acids to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of a nucleic acid, such as DNA, with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or transcription, it can be necessary to remove, add or alter 5′ untranslated portions of the clones to eliminate extra, potentially inappropriate, alternative translation initiation (i.e., start) codons or other sequences that can interfere with or reduce expression, either at the level of transcription or translation. In addition, consensus ribosome binding sites can be inserted immediately 5′ of the start codon and can enhance expression (see, e.g., Kozak J. Biol. Chem. 266: 19867-19870 (1991) and Shine and Delgarno, Nature 254(5495):34-38 (1975)). The desirability of (or need for) such modification can be empirically determined.

As used herein, a heterologous promoter refers to a promoter that is not normally found in the wild-type organism or virus or that is at a different locus as compared to a wild-type organism or virus. A heterologous promoter is often not endogenous to a virus into which it is introduced, but has been obtained from another virus or prepared synthetically. A heterologous promoter can refer to a promoter from another virus in the same organism or another organism, including the same species or another species. A heterologous promoter, however, can be endogenous, but is a promoter that is altered in its sequence or occurs at a different locus (e.g., at a different location in the genome or on a plasmid). Thus, a heterologous promoter includes a promoter not present in the exact orientation or position as the counterpart promoter is found in a genome.

A synthetic promoter is a heterologous promoter that has a nucleotide sequence that is not found in nature. A synthetic promoter can be a nucleic acid molecule that has a synthetic sequence or a sequence derived from a native promoter or portion thereof. A synthetic promoter also can be a hybrid promoter composed of different elements derived from different native promoters.

As used herein, a “reporter gene” is a gene that encodes a reporter molecule that can be detected when expressed by a virus provided herein or encodes a molecule that modulates expression of a detectable molecule, such as nucleic acid molecule or a protein, or modulates an activity or event that is detectable. Hence reporter molecules include, nucleic acid molecules, such as expressed RNA molecules, and proteins.

As used herein, a “heterologous reporter gene” is a reporter gene that is not natively present in a virus or is a gene that is present at a different locus than in its native locus in a virus. Heterologous reporter genes can contain nucleic acid that is not endogenous to the virus into which it is introduced, but has been obtained from another virus or cell or prepared synthetically. Heterologous reporter genes, however, can be endogenous, but contain nucleic acid that is expressed from a different locus or altered in its expression or sequence. Generally, such reporter genes encode RNA and proteins that are not normally produced by the virus or that are not produced under the same regulatory schema, such as the promoter.

As used herein, a “reporter protein” or “reporter gene product” refers to any detectable protein or product expressed by a reporter gene. Reporter proteins can be expressed from endogenous or heterologous genes. Exemplary reporter proteins are provided herein and include, for example, receptors or other proteins that can specifically bind to a detectable compound, proteins that can emit a detectable signal such as a fluorescence signal, and enzymes that can catalyze a detectable reaction or catalyze formation of a detectable product. Reporter gene products also can include detectable nucleic acids.

As used herein, a reporter virus is a virus that expresses or encodes a reporter gene or a reporter protein or a detectable protein or moiety. It is a virus that is detectable in a cell. As used herein, an oncolytic reporter virus is an oncolytic virus that expresses or encodes a reporter gene or a reporter protein or a detectable protein or moiety.

As used herein, detecting an oncolytic reporter virus means detecting tumor cells infected by the virus by one or more methods that detect a reporter gene product encoded by the virus that is expressed during infection of the tumor cell. Such methods include, but are not limited to detection of proteins such fluorescent proteins, luminescent proteins or proteins that bind to detectable ligands or antibodies.

As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak excitation spectrum at 490 nm or about 490 nm and peak emission spectrum at 510 nm or about 510 nm (expressed herein as excitation/emission 490 nm/510 nm). A variety of FPs that emit at various wavelengths are known in the art. Exemplary FPs include, but are not limited to, a violet fluorescent protein (VFP; peak excitation/emission at or about 355 nm/424 nm), a blue fluorescent protein (BFP; peak excitation/emission at or about 380-400 nm/450 nm), cyan fluorescent protein (CFP; peak excitation/emission at or about 430-460 nm/480-490 nm), green fluorescent protein (GFP; peak excitation/emission at or about 490 nm/510 nm), yellow fluorescent protein (YFP; peak excitation/emission at or about 515 nm/530 nm), orange fluorescent protein (OFP; peak excitation/emission at or about 550 nm/560 nm), red fluorescent protein (RFP; peak excitation/emission at or about 560-590 nm/580-610 nm), far-red fluorescent protein (peak excitation/emission at or about 590 nm/630-650 nm), or near-infrared fluorescent protein (peak excitation/emission at or about 690 nm/713 nm). Extending the spectrum of available colors of fluorescent proteins to blue, cyan, orange, yellow and red variants provides a method for multicolor tracking of proteins.

As used herein, Aequorea GFP refers to GFPs from the genus Aequorea and to mutants or variants thereof. Such variants and GFPs from other species, such as Anthozoa reef coral, Anemonia sea anemone, Renilla sea pansy, Galaxea coral, Acropora brown coral, Trachyphyllia and Pectimidae stony coral and other species are well known and are available and known to those of skill in the art.

As used herein, luminescence refers to the detectable electromagnetic (EM) radiation, generally, ultraviolet (UV), infrared (IR) or visible EM radiation that is produced when the excited product of an exergonic chemical process reverts to its ground state with the emission of light. Chemiluminescence is luminescence that results from a chemical reaction. Bioluminescence is chemiluminescence that results from a chemical reaction using biological molecules (or synthetic versions or analogs thereof) as substrates and/or enzymes. Fluorescence is luminescence in which light of a visible color is emitted from a substance under stimulation or excitation by light or other forms radiation such as ultraviolet (UV), infrared (IR) or visible EM radiation.

As used herein, chemiluminescence refers to a chemical reaction in which energy is specifically channeled to a molecule causing it to become electronically excited and subsequently to release a photon, thereby emitting visible light. Temperature does not contribute to this channeled energy. Thus, chemiluminescence involves the direct conversion of chemical energy to light energy.

As used herein, bioluminescence, which is a type of chemiluminescence, refers to the emission of light by biological molecules, particularly proteins. The essential condition for bioluminescence is molecular oxygen, either bound or free in the presence of an oxygenase, a luciferase, which acts on a substrate, a luciferin. Bioluminescence is generated by an enzyme or other protein (luciferase) that is an oxygenase that acts on a substrate luciferin (a bioluminescence substrate) in the presence of molecular oxygen and transforms the substrate to an excited state, which, upon return to a lower energy level releases the energy in the form of light.

As used herein, the substrates and enzymes for producing bioluminescence are generically referred to as luciferin and luciferase, respectively. When reference is made to a particular species thereof, for clarity, each generic term is used with the name of the organism from which it derives such as, for example, click beetle luciferase or firefly luciferase.

As used herein, luciferase refers to oxygenases that catalyze a light emitting reaction. For instance, bacterial luciferases catalyze the oxidation of flavin mononucleotide (FMN) and aliphatic aldehydes, which reaction produces light. Another class of luciferases, found among marine arthropods, catalyzes the oxidation of Cypridina (Vargula) luciferin and another class of luciferases catalyzes the oxidation of Coleoptera luciferin. Thus, luciferase refers to an enzyme or photoprotein that catalyzes a bioluminescent reaction (a reaction that produces bioluminescence). The luciferases, such as firefly and Gaussia and Renilla luciferases, are enzymes which act catalytically and are unchanged during the bioluminescence generating reaction. The luciferase photoproteins, such as the aequorin photoprotein to which luciferin is non-covalently bound, are changed, such as by release of the luciferin, during bioluminescence generating reaction. The luciferase is a protein, or a mixture of proteins (e.g., bacterial luciferase), that occurs naturally in an organism or a variant or mutant thereof, such as a variant produced by mutagenesis that has one or more properties, such as thermal stability, that differ from the naturally-occurring protein. Luciferases and modified mutant or variant forms thereof are well known. For purposes herein, reference to luciferase refers to either the photoproteins or luciferases.

Reference, for example, to Renilla luciferase refers to an enzyme isolated from member of the genus Renilla or an equivalent molecule obtained from any other source, such as from another related copepod, or that has been prepared synthetically. It is intended to encompass Renilla luciferases with conservative amino acid substitutions that do not substantially alter activity. Conservative substitutions of amino acids are known to those of skill in the art and can be made generally without altering the biological activity of the resulting molecule. Those of skill in the art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

As used herein, bioluminescence substrate refers to the compound that is oxidized in the presence of a luciferase and any necessary activators and generates light. These substrates are referred to as luciferins herein, are substrates that undergo oxidation in a bioluminescence reaction. These bioluminescence substrates include any luciferin or analog thereof or any synthetic compound with which a luciferase interacts to generate light. Typical substrates include those that are oxidized in the presence of a luciferase or protein in a light-generating reaction. Bioluminescence substrates, thus, include those compounds that those of skill in the art recognize as luciferins. Luciferins, for example, include firefly luciferin, Cypridina (also known as Vargula) luciferin (coelenterazine), bacterial luciferin, as well as synthetic analogs of these substrates or other compounds that are oxidized in the presence of a luciferase in a reaction the produces bioluminescence.

As used herein, capable of conversion into a bioluminescence substrate refers to being susceptible to chemical reaction, such as oxidation or reduction, which yields a bioluminescence substrate. For example, the luminescence producing reaction of bioluminescent bacteria involves the reduction of a flavin mononucleotide group (FMN) to reduced flavin mononucleotide (FMNH₂) by a flavin reductase enzyme. The reduced flavin mononucleotide (substrate) then reacts with oxygen (an activator) and bacterial luciferase to form an intermediate peroxy flavin that undergoes further reaction, in the presence of a long-chain aldehyde, to generate light. With respect to this reaction, the reduced flavin and the long chain aldehyde are bioluminescence substrates.

As used herein, a bioluminescence generating system refers to the set of reagents required to conduct a bioluminescent reaction. Thus, the specific luciferase, luciferin and other substrates, solvents and other reagents that can be required to complete a bioluminescent reaction form a bioluminescence system. Thus a bioluminescence generating system refers to any set of reagents that, under appropriate reaction conditions, yield bioluminescence. Appropriate reaction conditions refer to the conditions necessary for a bioluminescence reaction to occur, such as pH, salt concentrations and temperature. In general, bioluminescence systems include a bioluminescence substrate, luciferin, a luciferase, which includes enzymes luciferases and photoproteins and one or more activators. A specific bioluminescence system can be identified by reference to the specific organism from which the luciferase derives; for example, the Renilla bioluminescence system includes a Renilla luciferase, such as a luciferase isolated from Renilla or produced using recombinant methods or modifications of these luciferases. This system also includes the particular activators necessary to complete the bioluminescence reaction, such as oxygen and a substrate with which the luciferase reacts in the presence of the oxygen to produce light.

As used herein, the term “modified” with reference to a gene refers to a gene encoding a gene product, having one or more truncations, mutations, insertions or deletions; to a deleted gene; or to a gene encoding a gene product that is inserted (e.g., into the chromosome or on a plasmid, phagemid, cosmid, and phage), typically accompanied by at least a change in function of the modified gene product or virus.

As used herein, a “non-essential gene or region” of a virus genome is a location or region that can be modified by insertion, deletion, or mutation without inhibiting the infection life cycle of the virus. Modification of a “non-essential gene or region” is intended to encompass modifications that have no effect on the virus life cycle and modifications that attenuate or reduce the toxicity of the virus, but do not completely inhibit infection, replication and production of new virus.

As used herein, an “attenuated virus” refers to a virus that has been modified to alter one or more properties of the virus that affect, for example, virulence, toxicity, or pathogenicity of the virus compared to a virus without such modification. Typically, the viruses for use in the methods provided herein are attenuated viruses with respect to the wild-type form of the virus.

As used herein, an “attenuated LIVP virus” with reference to LIVP refers to a virus that exhibits reduced or less virulence, toxicity or pathogenicity compared to LIVP.

As used herein, “toxicity” (also referred to as virulence or pathogenicity herein) with reference to a virus refers to the deleterious or toxic effects to a host upon administration of the virus. For an oncolytic virus, such as LIVP, the toxicity of a virus is associated with its accumulation in non-tumorous organs or tissues, which can impact the survival of the host or result in deleterious or toxic effects. Toxicity can be measured by assessing one or more parameters indicative of toxicity. These include accumulation in non-tumorous tissues and effects on viability or health of the subject to whom it has been administered, such as effects on weight.

As used herein, “reduced toxicity” means that the toxic or deleterious effects upon administration of the virus to a host are attenuated or lessened compared to a host that is administered with another reference or control virus. For purposes herein, exemplary of a reference or control virus with respect to toxicity is the LIVP virus designated GLV-1h68 (described, for example, in U.S. Pat. No. 7,588,767; see, also SEQ ID NO:1) or a virus that is the same as the virus administered except not including a particular modification that reduces toxicity. Whether toxicity is reduced or lessened can be determined by assessing the effect of a virus and, if necessary, a control or reference virus, on a parameter indicative of toxicity. It is understood that when comparing the activity of two or more different viruses, the amount of virus (e.g. pfu) used in an in vitro assay or administered in vivo is the same or similar and the conditions (e.g. in vivo dosage regime) of the in vitro assay or in vivo assessment are the same or similar. For example, when comparing effects upon in vivo administration of a virus and a control or reference virus the subjects are the same species, size, gender and the virus is administered in the same or similar amount under the same or similar dosage regime. In particular, a virus with reduced toxicity can mean that upon administration of the virus to a host, such as for the treatment of a disease, the virus does not accumulate in non-tumorous organs and tissues in the host to an extent that results in damage or harm to the host, or that impacts survival of the host to a greater extent than the disease being treated does or to a greater extent than a control or reference virus does. For example, a virus with reduced toxicity includes a virus that does not result in death of the subject over the course of treatment.

As used herein, accumulation of a virus in a particular tissue refers to the distribution of the virus in particular tissues of a host organism after a time period following administration of the virus to the host, long enough for the virus to infect the host's organs or tissues. As one skilled in the art will recognize, the time period for infection of a virus will vary depending on the virus, the organ(s) or tissue(s), the immunocompetence of the host and dosage of the virus. Generally, accumulation can be determined at time points from about less than 1 day, about 1 day to about 2, 3, 4, 5, 6 or 7 days, about 1 week to about 2, 3 or 4 weeks, about 1 month to about 2, 3, 4, 5, 6 months or longer after infection with the virus. For purposes herein, the viruses preferentially accumulate in immunoprivileged tissue, such as inflamed tissue or tumor tissue, but are cleared from other tissues and organs, such as non-tumor tissues, in the host to the extent that toxicity of the virus is mild or tolerable and at most, not fatal.

As used herein, “preferential accumulation” refers to accumulation of a virus at a first location at a higher level than accumulation at a second location (i.e., the concentration of viral particles, or titer, at the first location is higher than the concentration of viral particles at the second location). Thus, a virus that preferentially accumulates in immunoprivileged tissue (tissue that is sheltered from the immune system), such as inflamed tissue, and tumor tissue, relative to normal tissues or organs, refers to a virus that accumulates in immunoprivileged tissue, such as tumor, at a higher level (i.e., concentration or viral titer) than the virus accumulates in normal tissues or organs.

As used herein, the terms immunoprivileged cells and immunoprivileged tissues refer to cells and tissues, such as solid tumors, which are sequestered from the immune system. Generally, administration of a virus to a subject elicits an immune response that clears the virus from the subject. Immunoprivileged sites, however, are shielded or sequestered from the immune response, permitting the virus to survive and generally to replicate. Immunoprivileged tissues include proliferating tissues, such as tumor tissues.

As used herein, “anti-tumor activity” or “anti-tumorigenic” refers to virus strains that prevent or inhibit the formation or growth of tumors in vitro or in vivo in a subject. Anti-tumor activity can be determined by assessing a parameter or parameters indicative of anti-tumor activity.

As used herein, “greater” or “improved” activity with reference to anti-tumor activity or anti-tumorigenicity means that a virus strain is capable of preventing or inhibiting the formation or growth of tumors in vitro or in vivo in a subject to a greater extent than a reference or control virus or to a greater extent than absence of treatment with the virus. Whether anti-tumor activity is “greater” or “improved” can be determined by assessing the effect of a virus and, if necessary, a control or reference virus, on a parameter indicative of anti-tumor activity. It is understood that when comparing the activity of two or more different viruses, the amount of virus (e.g. pfu) used in an in vitro assay or administered in vivo is the same or similar, and the conditions (e.g. in vivo dosage regime) of the in vitro assay or in vivo assessment are the same or similar.

As used herein, “genetic therapy” or “gene therapy” involves the transfer of heterologous nucleic acid, such as DNA, into certain cells, target cells, of a mammal, particularly a human, with a disorder or conditions for which such therapy is sought. The nucleic acid, such as DNA, is introduced into the selected target cells, such as directly or in a vector or other delivery vehicle, in a manner such that the heterologous nucleic acid, such as DNA, is expressed and a therapeutic product encoded thereby is produced. Alternatively, the heterologous nucleic acid, such as DNA, can in some manner mediate expression of DNA that encodes the therapeutic product, or it can encode a product, such as a peptide or RNA that in some manner mediates, directly or indirectly, expression of a therapeutic product. Genetic therapy also can be used to deliver nucleic acid encoding a gene product that replaces a defective gene or supplements a gene product produced by the mammalian or the cell in which it is introduced. The introduced nucleic acid can encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous nucleic acid, such as DNA, encoding the therapeutic product can be modified prior to introduction into the cells of the afflicted host in order to enhance or otherwise alter the product or expression thereof. Genetic therapy also can involve delivery of an inhibitor or repressor or other modulator of gene expression.

As used herein, the terms overproduce or overexpress when used in reference to a substance, molecule, compound or composition made in a cell refers to production or expression at a level that is greater than a baseline, normal or usual level of production or expression of the substance, molecule, compound or composition by the cell. A baseline, normal or usual level of production or expression includes no production/expression or limited, restricted or regulated production/expression. Such overproduction or overexpression is typically achieved by modification of cell.

As used herein, a tumor, also known as a neoplasm, is an abnormal mass of tissue that results when cells proliferate at an abnormally high rate. Tumors can show partial or total lack of structural organization and functional coordination with normal tissue. Tumors can be benign (not cancerous), or malignant (cancerous). As used herein, a tumor is intended to encompass hematopoietic tumors as well as solid tumors.

Malignant tumors can be broadly classified into three major types. Carcinomas are malignant tumors arising from epithelial structures (e.g. breast, prostate, lung, colon, pancreas). Sarcomas are malignant tumors that originate from connective tissues, or mesenchymal cells, such as muscle, cartilage, fat or bone. Leukemias and lymphomas are malignant tumors affecting hematopoietic structures (structures pertaining to the formation of blood cells) including components of the immune system. Other malignant tumors include, but are not limited to, tumors of the nervous system (e.g. neurofibromatomas), germ cell tumors, and plastic tumors.

As used herein, a disease or disorder refers to a pathological condition in an organism resulting from, for example, infection or genetic defect, and characterized by identifiable symptoms. An exemplary disease as described herein is a neoplastic disease, such as cancer.

As used herein, proliferative disorders include any disorders involving abnormal proliferation of cells (i.e. cells proliferate more rapidly compared to normal tissue growth), such as, but not limited to, neoplastic diseases.

As used herein, neoplastic disease refers to any disorder involving cancer, including tumor development, growth, metastasis and progression.

As used herein, cancer is a term for diseases caused by or characterized by any type of malignant tumor, including metastatic cancers, lymphatic tumors, and blood cancers. Exemplary cancers include, but are not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma, epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia or Wilm's tumor. Exemplary cancers commonly diagnosed in humans include, but are not limited to, cancers of the bladder, brain, breast, bone marrow, cervix, colon/rectum, kidney, liver, lung/bronchus, ovary, pancreas, prostate, skin, stomach, thyroid, or uterus. Exemplary cancers commonly diagnosed in dogs, cats, and other pets include, but are not limited to, lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma (e.g., granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma, follicular lymphoma, intestinal lymphosarcoma, fibrosarcoma and pulmonary squamous cell carcinoma. Exemplary cancers diagnosed in rodents, such as a ferret, include, but are not limited to, insulinoma, lymphoma, sarcoma, neuroma, pancreatic islet cell tumor, gastric MALT lymphoma and gastric adenocarcinoma. Exemplary neoplasias affecting agricultural livestock include, but are not limited to, leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lymphadenitis (CLA): chronic, infectious, contagious disease of sheep and goats caused by the bacterium Corynebacterium pseudotuberculosis, and contagious lung tumor of sheep caused by jaagsiekte.

As used herein, an aggressive cancer refers to a cancer characterized by a rapidly growing tumor or tumors. Typically the tumor(s) is actively metastasizing or is at risk of metastasizing. Aggressive cancer typically refer to metastatic cancers that spread to multiple locations in the body.

As used herein, an in vivo method refers to any method that is performed within the living body of a subject.

As used herein, an in vitro method refers to any method that is performed outside the living body of a subject.

As used herein, an ex vivo method refers to a method performed on a sample obtained from a subject.

As used herein, the term “therapeutic virus” refers to a virus that is administered for the treatment of a disease or disorder, such as a neoplastic disease, such as cancer, a tumor and/or a metastasis or inflammation or wound or diagnosis thereof and or both. Generally, a therapeutic virus herein is one that exhibits anti-tumor activity and minimal toxicity.

As used herein, a disease or disorder refers to a pathological condition in an organism resulting from, for example, infection or genetic defect, and characterized by identifiable symptoms.

As used herein, treatment of a subject that has a neoplastic disease, including a tumor or metastasis, means any manner of treatment in which the symptoms of having the neoplastic disease are ameliorated or otherwise beneficially altered. Typically, treatment of a tumor or metastasis in a subject encompasses any manner of treatment that results in slowing of tumor growth, lysis of tumor cells, reduction in the size of the tumor, prevention of new tumor growth, or prevention of metastasis of a primary tumor, including inhibition vascularization of the tumor, tumor cell division, tumor cell migration or degradation of the basement membrane or extracellular matrix.

As used herein, therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, efficacy means that upon systemic administration of an oncolytic virus, the virus will colonize tumor cells and replicate. In particular, it will replicate sufficiently so that tumor cells released into circulation will contain virus. Colonization and replication in tumor cells is indicative that the treatment is or will be an effective treatment.

As used herein, effective treatment with a virus is one that can increase survival compared to the absence of treatment therewith. For example, a virus is an effective treatment if it stabilizes disease, causes tumor regression, decreases severity of disease or slows down or reduces metastasizing of the tumor.

As used herein, therapeutic agents are agents that ameliorate the symptoms of a disease or disorder or ameliorate the disease or disorder. Therapeutic agents can be any molecule, such as a small molecule, a peptide, a polypeptide, a protein, an antibody, an antibody fragment, a DNA, or a RNA. Therapeutic agent, therapeutic compound, or therapeutic regimens include conventional drugs and drug therapies, including vaccines for treatment or prevention (i.e., reducing the risk of getting a particular disease or disorder), which are known to those skilled in the art and described elsewhere herein. Therapeutic agents for the treatment of neoplastic disease include, but are not limited to, moieties that inhibit cell growth or promote cell death, that can be activated to inhibit cell growth or promote cell death, or that activate another agent to inhibit cell growth or promote cell death. Therapeutic agents for use in the methods provided herein can be, for example, an anticancer agent. Exemplary therapeutic agents include, for example, therapeutic microorganisms, such as therapeutic viruses and bacteria, chemotherapeutic compounds, cytokines, growth factors, hormones, photosensitizing agents, radionuclides, toxins, antimetabolites, signaling modulators, anticancer antibiotics, anticancer antibodies, anti-cancer oligopeptides, anti-cancer oligonucleotide (e.g., antisense RNA and siRNA), angiogenesis inhibitors, radiation therapy, or a combination thereof.

As used herein, an anti-cancer agent or compound (used interchangeably with “anti-tumor or anti-neoplastic agent”) refers to any agents, or compounds, used in anti-cancer treatment. These include any agents, when used alone or in combination with other compounds or treatments, that can alleviate, reduce, ameliorate, prevent, or place or maintain in a state of remission of clinical symptoms or diagnostic markers associated with neoplastic disease, tumors and cancer, and can be used in methods, combinations and compositions provided herein.

As used herein, a “chemotherapeutic agent” is any drug or compound that is used in anti-cancer treatment. Exemplary of such agents are alkylating agents, nitrosoureas, antitumor antibiotics, antimetabolites, antimitotics, topoisomerase inhibitors, monoclonal antibodies, and signaling inhibitors. Exemplary chemotherapeutic agent include, but are not limited to, chemotherapeutic agents, such as Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. The term “chemotherapeutic agent” can be used interchangeably with the term “anti-cancer agent” when referring to drugs or compounds for the treatment of cancer. As used herein, reference to a chemotherapeutic agent includes combinations or a plurality of chemotherapeutic agents unless otherwise indicated.

As used herein, an anti-metastatic agent is an agent that ameliorates the symptoms of metastasis or ameliorates metastasis. Generally, anti-metastatic agents directly or indirectly inhibit one or more steps of metastasis, including but not limited to, degradation of the basement membrane and proximal extracellular matrix, which leads to tumor cell detachment from the primary tumor, tumor cell migration, tumor cell invasion of local tissue, tumor cell division and colonization at the secondary site, organization of endothelial cells into new functioning capillaries in a tumor, and the persistence of such functioning capillaries in a tumor. Anti-metastatic agents include agents that inhibit the metastasis of a cell from a primary tumor, including release of the cell from the primary tumor and establishment of a secondary tumor, or that inhibits further metastasis of a cell from a site of metastasis. Treatment of a tumor bearing subject with anti-metastatic agents can result in, for example, the delayed appearance of secondary (i.e. metastatic) tumors, slowed development of primary or secondary tumors, decreased occurrence of secondary tumors, slowed or decreased severity of secondary effects of neoplastic disease, arrested tumor growth and regression.

As used herein, an effective amount of a virus or compound for treating a particular disease is an amount that is sufficient to ameliorate, or in some manner reduce the symptoms associated with the disease. Such an amount can be administered as a single dosage or can be administered in multiple dosages according to a regimen, whereby it is effective. The amount can cure the disease but, typically, is administered in order to ameliorate the symptoms of the disease. Repeated administration can be required to achieve the desired amelioration of symptoms.

As used herein, a compound produced in a tumor refers to any compound that is produced in the tumor or tumor environment by virtue of the presence of an introduced virus, generally a recombinant virus, expressing one or more gene products. For example, a compound produced in a tumor can be, for example, an encoded polypeptide or RNA, a metabolite, or compound that is generated by a recombinant polypeptide and the cellular machinery of the tumor.

As used herein, the term “ELISA” refers to enzyme-linked immunosorbent assay. Numerous methods and applications for carrying out an ELISA are well known in the art, and provided in many sources (See, e.g., Crowther, “Enzyme-Linked Immunosorbent Assay (ELISA),” in Molecular Biomethods Handbook, Rapley et al. [eds.], pp. 595-617, Hzumana Press, Inc., Totowa, N.J. [1998]; Harlow and Lane (eds.), Antibodies: A Laboratory Manual, Cold Spring Harbor Laboratory Press [1988]; and Ausubel et al. (eds.), Current Protocols in Molecular Biology, Ch. 11, John Wiley & Sons, Inc., New York [1994]; and Newton, et al. (2006) Neoplasia. 8:772-780). A “direct ELISA” protocol involves a target-binding molecule, such as a cell, cell lysate, or isolated protein, first bound and immobilized to a microtiter plate well. A “sandwich ELISA” involves a target-binding molecule attached to the substrate by capturing it with an antibody that has been previously bound to the microtiter plate well. The ELISA method detects an immobilized ligand-receptor complex (binding) by use of fluorescent detection of fluorescently labeled ligands or an antibody-enzyme conjugate, where the antibody is specific for the antigen of interest, such as a phage virion, while the enzyme portion allows visualization and quantitation by the generation of a colored or fluorescent reaction product. The conjugated enzymes commonly used in the ELISA include horseradish peroxidase, urease, alkaline phosphatase, glucoamylase or O-galactosidase. The intensity of color development is proportional to the amount of antigen present in the reaction well.

As used herein, a delivery vehicle for administration refers to a lipid-based or other polymer-based composition, such as liposome, micelle or reverse micelle, that associates with an agent, such as a virus provided herein, for delivery into a host subject.

As used herein, a “diagnostic agent” refer to any agent that can be applied in the diagnosis or monitoring of a disease or health-related condition. The diagnostic agent can be any molecule, such as a small molecule, a peptide, a polypeptide, a protein, an antibody, an antibody fragment, a DNA, or a RNA.

As used herein, a detectable label or detectable moiety or diagnostic moiety (also imaging label, imaging agent, or imaging moiety) refers to an atom, molecule or composition, wherein the presence of the atom, molecule or composition can be directly or indirectly measured. Detectable labels can be used to image one or more of any of the viruses provided herein. Detectable labels can be used in any of the methods provided herein. Detectable labels include, for example, chemiluminescent moieties, bioluminescent moieties, fluorescent moieties, radionuclides, and metals. Methods for detecting labels are well known in the art. Such a label can be detected, for example, by visual inspection, by fluorescence spectroscopy, by reflectance measurement, by flow cytometry, by X-rays, by a variety of magnetic resonance methods such as magnetic resonance imaging (MRI) and magnetic resonance spectroscopy (MRS). Methods of detection also include any of a variety of tomographic methods including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography, and ultrasonic tomography. Direct detection of a detectable label refers to, for example, measurement of a physical phenomenon of the detectable label itself, such as energy or particle emission or absorption of the label itself, such as by X-ray or MRI. Indirect detection refers to measurement of a physical phenomenon of an atom, molecule or composition that binds directly or indirectly to the detectable label, such as energy or particle emission or absorption, of an atom, molecule or composition that binds directly or indirectly to the detectable label. In a non-limiting example of indirect detection, a detectable label can be biotin, which can be detected by binding to avidin. Non-labeled avidin can be administered systemically to block non-specific binding, followed by systemic administration of labeled avidin. Thus, included within the scope of a detectable label or detectable moiety is a bindable label or bindable moiety, which refers to an atom, molecule or composition, wherein the presence of the atom, molecule or composition can be detected as a result of the label or moiety binding to another atom, molecule or composition. Exemplary detectable labels include, for example, metals such as colloidal gold, iron, gadolinium, and gallium-67, fluorescent moieties, and radionuclides. Exemplary fluorescent moieties and radionuclides are provided elsewhere herein.

As used herein, a radionuclide, a radioisotope or radioactive isotope is used interchangeably to refer to an atom with an unstable nucleus. The nucleus is characterized by excess energy which is available to be imparted either to a newly-created radiation particle within the nucleus, or else to an atomic electron. The radionuclide, in this process, undergoes radioactive decay, and emits a gamma ray and/or subatomic particles. Such emissions can be detected in vivo by method such as, but not limited to, positron emission tomography (PET), single-photon emission computed tomography (SPECT) or planar gamma imaging. Radioisotopes can occur naturally, but also can be artificially produced. Exemplary radionuclides for use in in vivo imaging include, but are not limited to, ¹¹C, ¹¹F, ¹³C, ¹³N, ¹⁵N, ¹⁵0, ¹⁸F, ¹⁹F, ³²P, ⁵²Fe, ⁵¹Cr, ⁵⁵Co, ⁵⁵Fe, ⁵⁷Co, ⁵⁸Co, ⁵⁷Ni, ⁵⁹Fe ⁶⁰Co, ⁶⁴Cu, ⁶⁷Ga, ⁶⁸Ga, ⁶⁰Cu(II), ⁶⁷Cu(i), ⁹⁹Tc, ⁹⁰Y, ⁹⁹Tc, ¹⁰³Pd, ¹⁰⁶Ru, ¹¹¹In, ¹¹⁷Lu, ¹²³I, ¹²⁵I, ¹²⁴I, ¹³¹I, ¹³⁷Cs, ¹⁵³Gd, ¹⁵³Sm, ¹⁸⁶Re, ¹⁸⁸Re, ¹⁹²Ir, ¹⁹⁸Au, ²¹¹At, ²¹²Bi, ²¹³Bi and ²⁴¹Am. Radioisotopes can be incorporated into or attached to a compound, such as a metabolic compound. Exemplary radionuclides that can be incorporated or linked to a metabolic compound, such as nucleoside analog, include, but are not limited to, ¹²³I, ¹²⁴I, ¹²⁵I, ¹³¹I, ¹⁸F, ¹⁹F, ¹¹C, ¹³C, ¹⁴C, ⁷⁵Br, ⁷⁶Br, and ³H.

As used herein, magnetic resonance imaging (MRI) refers to the use of a nuclear magnetic resonance spectrometer to produce electronic images of specific atoms and molecular structures in solids, especially human cells, tissues, and organs. MRI is non-invasive diagnostic technique that uses nuclear magnetic resonance to produce cross-sectional images of organs and other internal body structures. The subject lies inside a large, hollow cylinder containing a strong electromagnet, which causes the nuclei of certain atoms in the body (such as, for example, ¹H, ¹³C and ¹⁹F) to align magnetically. The subject is then subjected to radio waves, which cause the aligned nuclei to flip; when the radio waves are withdrawn the nuclei return to their original positions, emitting radio waves that are then detected by a receiver and translated into a two-dimensional picture by computer. For some MRI procedures, contrast agents such as gadolinium are used to increase the accuracy of the images.

As used herein, an X-ray refers to a relatively high-energy photon, or a stream of such photons, having a wavelength in the approximate range from 0.01 to 10 nanometers. X-rays also refer to photographs taken with x-rays.

As used herein, a compound conjugated to a moiety refers to a complex that includes a compound bound to a moiety, where the binding between the compound and the moiety can arise from one or more covalent bonds or non-covalent interactions such as hydrogen bonds, or electrostatic interactions. A conjugate also can include a linker that connects the compound to the moiety. Exemplary compounds include, but are not limited to, nanoparticles and siderophores. Exemplary moieties, include, but are not limited to, detectable moieties and therapeutic agents.

As used herein, “modulate” and “modulation” or “alter” refer to a change of an activity of a molecule, such as a protein. Exemplary activities include, but are not limited to, biological activities, such as signal transduction. Modulation can include an increase in the activity (i.e., up-regulation or agonist activity), a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, an agent or compound that modulates the activity of a protein or expression of a gene or nucleic acid either decreases or increases or otherwise alters the activity of the protein or, in some manner, up- or down-regulates or otherwise alters expression of the nucleic acid in a cell.

As used herein, “nucleic acids” include DNA, RNA and analogs thereof, including peptide nucleic acids (PNA) and mixtures thereof. Nucleic acids can be single or double-stranded. Nucleic acids can encode gene products, such as, for example, polypeptides, regulatory RNAs, microRNAs, siRNAs and functional RNAs.

As used herein, a sequence complementary to at least a portion of an RNA, with reference to antisense oligonucleotides, means a sequence of nucleotides having sufficient complementarity to be able to hybridize with the RNA, generally under moderate or high stringency conditions, forming a stable duplex; in the case of double-stranded antisense nucleic acids, a single strand of the duplex DNA (i.e., dsRNA) can thus be assayed, or triplex formation can be assayed. The ability to hybridize depends on the degree of complementarity and the length of the antisense nucleic acid. Generally, the longer the hybridizing nucleic acid, the more base mismatches with an encoding RNA it can contain and still form a stable duplex (or triplex, as the case can be). One skilled in the art can ascertain a tolerable degree of mismatch by use of standard procedures to determine the melting point of the hybridized complex.

As used herein, a peptide refers to a polypeptide that is greater than or equal to 2 amino acids in length, and less than or equal to 40 amino acids in length.

As used herein, the amino acids which occur in the various sequences of amino acids provided herein are identified according to their known, three-letter or one-letter abbreviations (Table 1). The nucleotides which occur in the various nucleic acid fragments are designated with the standard single-letter designations used routinely in the art.

As used herein, an “amino acid” is an organic compound containing an amino group and a carboxylic acid group. A polypeptide contains two or more amino acids. For purposes herein, amino acids include the twenty naturally-occurring amino acids, non-natural amino acids and amino acid analogs (i.e., amino acids wherein the α-carbon has a side chain).

As used herein, “amino acid residue” refers to an amino acid formed upon chemical digestion (hydrolysis) of a polypeptide at its peptide linkages. The amino acid residues described herein are presumed to be in the “L” isomeric form. Residues in the “D” isomeric form, which are so designated, can be substituted for any L-amino acid residue as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243: 3557-3559 (1968), and adopted 37 C.F.R. §§1.821-1.822, abbreviations for amino acid residues are shown in Table 1:

TABLE 1
Table of Amino Acid Correspondence

SYMBOL

| 1-Letter | 3-Letter | AMINO ACID |
|---|---|---|
| Y | Tyr | Tyrosine |
| G | Gly | Glycine |
| F | Phe | Phenylalanine |
| M | Met | Methionine |
| A | Ala | Alanine |
| S | Ser | Serine |
| I | Ile | Isoleucine |
| L | Leu | Leucine |
| T | Thr | Threonine |
| V | Val | Valine |
| P | Pro | Proline |
| K | Lys | Lysine |
| H | His | Histidine |
| Q | Gln | Glutamine |
| E | Glu | Glutamic acid |
| Z | Glx | Glu and/or Gln |
| W | Trp | Tryptophan |
| R | Arg | Arginine |
| D | Asp | Aspartic acid |
| N | Asn | Asparagine |
| B | Asx | Asn and/or Asp |
| C | Cys | Cysteine |
| X | Xaa | Unknown or other |

All amino acid residue sequences represented herein by formulae have a left to right orientation in the conventional direction of amino-terminus to carboxyl-terminus. In addition, the phrase “amino acid residue” is defined to include the amino acids listed in the Table of Correspondence (Table 1) and modified and unusual amino acids, such as those referred to in 37 C.F.R. §§1.821-1.822, and incorporated herein by reference. Furthermore, a dash at the beginning or end of an amino acid residue sequence indicates a peptide bond to a further sequence of one or more amino acid residues, to an amino-terminal group such as NH₂ or to a carboxyl-terminal group such as COOH.

As used herein, the “naturally occurring α-amino acids” are the residues of those 20 α-amino acids found in nature which are incorporated into protein by the specific recognition of the charged tRNA molecule with its cognate mRNA codon in humans. Non-naturally occurring amino acids thus include, for example, amino acids or analogs of amino acids other than the 20 naturally-occurring amino acids and include, but are not limited to, the D-stereoisomers of amino acids. Exemplary non-natural amino acids are described herein and are known to those of skill in the art.

As used herein, the term polynucleotide means a single- or double-stranded polymer of deoxyribonucleotides or ribonucleotide bases read from the 5′ to the 3′ end. Polynucleotides include RNA and DNA, and can be isolated from natural sources, synthesized in vitro, or prepared from a combination of natural and synthetic molecules. The length of a polynucleotide molecule is given herein in terms of nucleotides (abbreviated “nt”) or base pairs (abbreviated “bp”). The term nucleotides is used for single- and double-stranded molecules where the context permits. When the term is applied to double-stranded molecules it is used to denote overall length and will be understood to be equivalent to the term base pairs. It will be recognized by those skilled in the art that the two strands of a double-stranded polynucleotide can differ slightly in length and that the ends thereof can be staggered; thus all nucleotides within a double-stranded polynucleotide molecule may not be paired. Such unpaired ends will, in general, not exceed 20 nucleotides in length.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g. Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exists a number of methods to measure identity between two polynucleotide or polypeptides, the term “identity” is well known to skilled artisans (Carrillo, H. and Lipton, D., SIAM J Applied Math 48:1073 (1988)).

As used herein, homologous (with respect to nucleic acid and/or amino acid sequences) means about greater than or equal to 25% sequence homology, typically greater than or equal to 25%, 40%, 50%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo and Lipman (1988) SIAM J Applied Math 48:1073). By sequence homology, the number of conserved amino acids is determined by standard alignment algorithms programs, and can be used with default gap penalties established by each supplier. Substantially homologous nucleic acid molecules hybridize typically at moderate stringency or at high stringency all along the length of the nucleic acid of interest. Also contemplated are nucleic acid molecules that contain degenerate codons in place of codons in the hybridizing nucleic acid molecule.

Whether any two molecules have nucleotide sequences or amino acid sequences that are at least 60%, 70%, 80%, 85%, 90%, 95%, 96%, 97%, 98% or 99% “identical” or “homologous” can be determined using known computer algorithms such as the “FASTA” program, using for example, the default parameters as in Pearson et al. (1988) Proc. Natl. Acad. Sci. USA 85:2444 (other programs include the GCG program package (Devereux, J., et al. Nucleic Acids Research 12(I):387 (1984)), BLASTP, BLASTN, FASTA (Altschul, S. F., et al. J Mol Biol 215:403 (1990)); Guide to Huge Computers, Martin J. Bishop, ed., Academic Press, San Diego, 1994, and Carrillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result is independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application. Complementary, when referring to two nucleotide sequences, means that the two sequences of nucleotides are capable of hybridizing, typically with less than 25%, 15% or 5% mismatches between opposed nucleotides. If necessary, the percentage of complementarity will be specified. Typically the two molecules are selected such that they will hybridize under conditions of high stringency.

As used herein, substantially identical to a product means sufficiently similar so that the property of interest is sufficiently unchanged so that the substantially identical product can be used in place of the product.

As used herein, it also is understood that the terms “substantially identical” or “similar” varies with the context as understood by those skilled in the relevant art.

As used herein, an allelic variant or allelic variation references any of two or more alternative forms of a gene occupying the same chromosomal locus. Allelic variation arises naturally through mutation, and can result in phenotypic polymorphism within populations. Gene mutations can be silent (no change in the encoded polypeptide) or can encode polypeptides having altered amino acid sequence. The term “allelic variant” also is used herein to denote a protein encoded by an allelic variant of a gene. Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide. Reference to an allelic variant herein generally refers to variations n proteins among members of the same species.

As used herein, “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for that gene or allele. When a subject has two different alleles of a gene, the subject is said to be heterozygous for the gene. Alleles of a specific gene can differ from each other in a single nucleotide or several nucleotides, and can include modifications such as substitutions, deletions and insertions of nucleotides. An allele of a gene also can be a form of a gene containing a mutation.

As used herein, species variants refer to variants in polypeptides among different species, including different mammalian species, such as mouse and human. Generally, species variants have 70%, 75%, 80%, 85%, 90%, 91%, 92%, 93%, 94%, 95%, 96%, 97%, 98% or sequence identity. Corresponding residues between and among species variants can be determined by comparing and aligning sequences to maximize the number of matching nucleotides or residues, for example, such that identity between the sequences is equal to or greater than 95%, equal to or greater than 96%, equal to or greater than 97%, equal to or greater than 98% or equal to greater than 99%. The position of interest is then given the number assigned in the reference nucleic acid molecule. Alignment can be effected manually or by eye, particularly, where sequence identity is greater than 80%.

As used herein, a human protein is one encoded by a nucleic acid molecule, such as DNA, present in the genome of a human, including all allelic variants and conservative variations thereof. A variant or modification of a protein is a human protein if the modification is based on the wildtype or prominent sequence of a human protein.

As used herein, a splice variant refers to a variant produced by differential processing of a primary transcript of genomic DNA that results in more than one type of mRNA.

As used herein, modification is in reference to modification of a sequence of amino acids of a polypeptide or a sequence of nucleotides in a nucleic acid molecule and includes deletions, insertions, and replacements (e.g. substitutions) of amino acids and nucleotides, respectively. Exemplary of modifications are amino acid substitutions. An amino-acid substituted polypeptide can exhibit 65%, 70%, 80%, 85%, 90%, 91%, 92%, 93%, 94%, 95%, 96%, 97%, 98% or more sequence identity to a polypeptide not containing the amino acid substitutions. Amino acid substitutions can be conservative or non-conservative. Generally, any modification to a polypeptide retains an activity of the polypeptide. Methods of modifying a polypeptide are routine to those of skill in the art, such as by using recombinant DNA methodologies.

As used herein, suitable conservative substitutions of amino acids are known to those of skill in the art and can be made generally without altering the biological activity of the resulting molecule. Those of skill in the art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224). Such substitutions can be made in accordance with those set forth in Table 2 as follows:

TABLE 2
Table of Exemplary Conservative Amino Acid Substitutions

| Original residue | Exemplary Conservative Substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, the term promoter means a portion of a gene containing DNA sequences that provide for the binding of RNA polymerase and initiation of transcription. Promoter sequences are commonly, but not always, found in the 5′ non-coding region of genes.

As used herein, isolated or purified polypeptide or protein or biologically-active portion thereof is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound, however, can be a mixture of stereoisomers. In such instances, further purification might increase the specific activity of the compound.

Hence, reference to a substantially purified polypeptide, refers to preparations of proteins that are substantially free of cellular material includes preparations of proteins in which the protein is separated from cellular components of the cells from which it is isolated or recombinantly-produced. In one example, the term substantially free of cellular material includes preparations of enzyme proteins having less that about 30% (by dry weight) of non-enzyme proteins (also referred to herein as a contaminating protein), generally less than about 20% of non-enzyme proteins or 10% of non-enzyme proteins or less that about 5% of non-enzyme proteins. When the enzyme protein is recombinantly produced, it also is substantially free of culture medium, i.e., culture medium represents less than about or at 20%, 10% or 5% of the volume of the enzyme protein preparation.

As used herein, the term substantially free of chemical precursors or other chemicals includes preparations of enzyme proteins in which the protein is separated from chemical precursors or other chemicals that are involved in the synthesis of the protein. The term includes preparations of enzyme proteins having less than about 30% (by dry weight), 20%, 10%, 5% or less of chemical precursors or non-enzyme chemicals or components.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant means or using recombinant DNA methods means the use of the well known methods of molecular biology for expressing proteins encoded by cloned DNA.

As used herein, a DNA construct is a single- or double-stranded, linear or circular DNA molecule that contains segments of DNA combined and juxtaposed in a manner not found in nature. DNA constructs exist as a result of human manipulation, and include clones and other copies of manipulated molecules.

As used herein, a DNA segment is a portion of a larger DNA molecule having specified attributes. For example, a DNA segment encoding a specified polypeptide is a portion of a longer DNA molecule, such as a plasmid or plasmid fragment, which, when read from the 5′ to 3′ direction, encodes the sequence of amino acids of the specified polypeptide.

As used herein, vector (or plasmid) refers to a nucleic acid construct that contains discrete elements that are used to introduce heterologous nucleic acid into cells for either expression of the nucleic acid or replication thereof. The vectors typically remain episomal, but can be designed to effect stable integration of a gene or portion thereof into a chromosome of the genome. Selection and use of such vectors are well known to those of skill in the art.

As used herein, an expression vector includes vectors capable of expressing DNA that is operatively linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, the term “viral vector” is used according to its art-recognized meaning. It refers to a nucleic acid vector that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, poxvirus vectors (e.g., vaccinia vectors), retroviral vectors, lentivirus vectors, herpes virus vectors (e.g., HSV), baculovirus vectors, cytomegalovirus (CMV) vectors, papillomavirus vectors, simian virus (SV40) vectors, semliki forest virus vectors, phage vectors, adenoviral vectors and adeno-associated viral (AAV) vectors.

As used herein equivalent, when referring to two sequences of nucleic acids, means that the two sequences in question encode the same sequence of amino acids or equivalent proteins. When equivalent is used in referring to two proteins or peptides, it means that the two proteins or peptides have substantially the same amino acid sequence with only amino acid substitutions that do not substantially alter the activity or function of the protein or peptide. When equivalent refers to a property, the property does not need to be present to the same extent (e.g., two peptides can exhibit different rates of the same type of enzymatic activity), but the activities are usually substantially the same.

As used herein, a composition refers to any mixture. It can be a solution, suspension, liquid, powder, paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination, optionally, including instructions for use of the combination and/or other reactions and components for such use.

As used herein, the singular forms “a,” “an” and “the” include plural referents unless the context clearly dictates otherwise.

As used herein, ranges and amounts can be expressed as “about” or “approximately” a particular value or range. “About” or “approximately” also includes the exact amount. Hence, “about 5 milliliters” means “about 5 milliliters” and also “5 milliliters.” Generally “about” includes an amount that would be expected to be within experimental error.

As used herein, “about the same” means within an amount that one of skill in the art would consider to be the same or to be within an acceptable range of error. For example, typically, for pharmaceutical compositions, within at least 1%, 2%, 3%, 4%, 5% or 10% is considered about the same. Such amount can vary depending upon the tolerance for variation in the particular composition by subjects.

As used herein, “optional” or “optionally” means that the subsequently described event or circumstance does or does not occur, and that the description includes instances where said event or circumstance occurs and instances where it does not.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

### B. OVERVIEW

Provided herein are methods of treatment with antibiotics to increase the therapeutic efficacy of viral therapy, such as oncolytic viral therapy and gene therapy.

The methods employ antibiotics to deplete commensal gut bacteria. It is shown herein that doing so improves viral therapy.

1. Gut Bacteria and Immune Response

Commensal intestinal bacteria play a role in modulating'immune responses against bacterial or viral infections (see, e.g., Macpherson and Harris, (2004) Nat Rev Immunol, 4(6):478-785) There are estimated 100 trillion bacteria in the human intestine. Commensal gut bacteria contribute to the development and regulation of the mammalian immune system (Hill et al., (2010) Annu Rev Immunol, 28:623-667). Depletion of gut bacteria by antibiotics has been reported to impair the normal development and function of nature killer cells (NK), dendritic cells (Dc) and macrophages (Mac) when exposed to pathogens (Ganal et al., (2012) Immunity 37:171-186). The gut bacterial population was found to participate in regulating the generation of virus-specific CD4 and CD8 T cells and antibody responses against respiratory influenza virus infection (Ichinohe et al. (2011) Proc. Natl. Acad. Sci. U.S.A. 108(13):5354-5359). In contrast, other studies have shown that viruses are depend upon commensal bacteria for infection and replication (Wilks and Golovkina (2012) PLoS Pathogens 8:e1002681). For example, depletion of gut bacteria prevents mouse mammary tumor virus (MMTV) infection (Kane et al. (2011) Nature 334:245-249) and intestinal bacteria promote replication and systemic pathogenesis of poliovirus and reovirus (Kuss et al. (2011) Nature 334:249-252).

2. Viral Therapy

Viral therapy includes, for example, oncolytic virotherapy for the treatment of cancer and tumors and gene therapy, in which viruses are used to deliver the DNA into cells for treatment of various diseases and conditions. Among the challenges presented by viral therapy is the systemic delivery of virus to target tumors. As shown herein, administration of antimicrobial agents that eliminate commensal gut microbes improve the efficacy of oncolytic viral therapy.

Among the methods provided herein are methods for reducing immune response to viruses. The methods provided herein temporarily weaken the immune response at time of virus infection, thereby improving the efficacy of virotherapy. As shown herein, administration of antimicrobial agents that eliminate commensal gut microbes improve the efficacy of viral therapy, including oncolytic viral therapy for treatment of tumors, cancers and metastases.

3. Methods of Treatment with Antibiotics Increase the Therapeutic Efficacy of Viral Therapy

As described herein and in the examples provided herein, administration of antibiotics that eliminate commensal gut microbes improves the efficacy of viral therapy, such as oncolytic viral therapy. This is exemplified herein with a therapeutic vaccinia virus. In a mouse xenograft model of human lung cancer, treatment with antibiotics and vaccinia viruses resulted in increased viral replication in tumors but not in healthy organs, increased survival rate and reduced weight loss. In human cancer patients, treatment of cancer patients with antibiotics and the LIVP strain vaccinia virus designated GLV-1h68 resulted in prolonged viral efficacy. For example, a prolonged inherent (in situ) intraperitoneal production of progeny viral particles was observed for up to 22 days when the patients were treated with antibiotics as opposed to only 8-12 days when the patient did not receive antibiotic therapy. GLV-1h68 contains a reporter gene encoding β-glucuronidase which can be used to monitor viral activity. β-Glucuronidase activity was observed 59 days after virus administration in the patient receiving antibiotics but was only observed after 9 days in the patient that only was administered virus. Further, the patient receiving both antibiotics and viral therapy had decreased numbers of cells/ascites and increased LDH levels indicating cell lysis. Finally, the a patient receiving both viral therapy and antibiotics had a prolonged inflammatory response, indicated by fever, CRP levels and leukocyte counts but lymphocyte counts were consistently lower in the patient receiving antibiotics.

### C. ANTIBIOTICS

Any antibiotic effective for inhibiting the growth of or killing gut bacteria can be used in the methods provided herein. The antibiotics are not chemotherapeutic antibiotics. Antibiotics for use in the methods provided herein, include, but are not limited to, penicillins such as penicillin, benzylpenicillin (penicillin G), procaine benzylpenicillin (procaine penicillin), benzathine benzylpenicillin (benzathine penicillin), phenoxymethylpenicillin (penicillin V), amoxicillin, ampicillin, azlocillin, carbenicillin, cloxacillin, dicloxacillin, flucloxacillin, mezlocillin, methicillin, nafcillin, oxacillin, temocillin and ticarcillin, penicillin combinations such as amoxicillin/clavulanate, ampicillin/sulbactam, piperacillin/tazobactam and ticarcillin/clavulanate, tetracyclines such as demeclocycline, doxycycline, minocycline, oxytetracycline and tetracycline, β-lactam antibiotics (Cephems) including cephalosporins, cephamycins and carbapenems such as cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, loracarbef, cefbuperazone, cefmetazole, cefminox, cefotetan, cefoxitin, cefotiam, cefcapene, cefdaloxime, cefdinir, cefditoren, cefetamet, cefixime, cefmenoxime, cefodizime, cefotaxime, cefovecin, cefpimizole, cefpodoxime, cefteram, ceftibuten, ceftiofur, ceftiolene, ceftizoxime, ceftriaxone, cefoperazone, ceftazidime, latamoxef, cefclidine, cefepime, cefluprenam, cefoselis, cefozopran, cefpirome, cefquinome, flomoxef, ceftobiprole, ceftaroline, cefaloram, cefaparole, cefcanel, cefedrolor, cefempidone, cefetrizole, cefivitril, cefmepidium, cefoxazole, cefrotil, cefsumide, ceftioxide and cefuracetime, ertapenem, doripenem, imipenem, imipenem/cilastatin, meropenem, panipenem/betamipron, biapenem, razupenem and tebipenem, carbacephems such as loracarbef, glycopeptides such as teicoplanin, vancomycin, bleomycin, ramoplanin, decaplanin and telavancin, aminoglycosides such as streptomycin, gentamicin, kanamycin, neomycin, netilmicin, tobramycin, spectinomycin, paromomycin, framycetin, ribostamycin, amikacin, arbekacin, bekanamycin, dibekacin, rhodostreptomycin, apramycin, hygromycin B, paromomycin sulfate, sisomicin, isepamicin, verdamicin and astromicin, ansamycins, such as geldanamycin, herbimycin and rifaximin, macrolides such as azithromycin, clarithromycin, dirithromycin, erythromycin, roxithromycin, telithromycin, carbomycin A, josamycin, kitasamycin, midecamycin, midecamycin acetate, oleandomycin, solithromycin, spiramycin, troleandomycin, tylosin and tylocine, ketolides such as telithromycin, cethromycin, solithromycin, spiramycin, ansamycin, oleandomycin, carbomycin and tylosin, monobactams such as aztreonam, nitrofurans such as furazolidone and nitrofurantoin, sulfonamides such as mafenide, sulfamethoxazole, sulfisomidine, sulfadiazine, silver sulfadiazine, sulfamethoxine, sulfamethizole, sulfanilamide, sulfasalazine, sulfisoxazole, trimethoprim-sulfamethoxazole, sulfonamidochrysoidine, sulfacetamide, sulfadoxine and dichlorphenamide, lincosamides such as clindamycin and lincomycin, lipopeptides such as daptomycin, polypeptides such as bacitracin, colistin and polymyxin B, quinolones such as moxifloxacin, ciprofloxacin, levofloxacin, cinoxacin, nalidixic acid, oxolinic acid, piromidic acid, pipemidic acid, rosoxacin, enoxacin, fleroxacin, lomefloxacin, nadifloxacin, norfloxacin, ofloxacin, pefloxacin, rufloxacin, balofloxacin, grepafloxacin, pazufloxacin, sparfloxacin, tosufloxacin, clinafloxacin, gatifloxacin, gemifloxacin, moxifloxacin, sitafloxacin, trovafloxacin and prulifloxacin, drugs against mycobacteria such as clofazimine, dapsone, capreomycin, cycloserine, ethambutol, ethionamide, isoniazid, pyrazinamide, rifampicin, rifabutin, rifapentine and streptomycin, oxazolidinones such as linezolid, posizolid, radezolid, cycloserine and torezolid, and other antibiotics such as arsphenamine, chloramphenicol, fosfomycin, fusidic acid, metronidazole, tazobactam, mupirocin, platensimycin, quinupristin/dalfopristin, thiamphenicol, tigecycline, tinidazole and trimethoprim.

Exemplary antibiotics for use in the methods provided herein include, but are not limited to, penicillin, streptomycin, ampicillin, neomycin, metronidazole, vancomycin, tazobactam and meropenem. In some examples, a combination of two or more antibiotics, such as 2, 3, 4, 5, 6 or more antibiotics may be used in the provided methods. In one example, a penicillin-streptomycin solution can be used in the provided methods. In another example, a mixture of ampicillin, neomycin, metronidazole and vancomycin can be used in the methods provided herein. In yet another example, a mixture of tazobactam, meropenem and vancomycin can be used in the provided methods.

In some examples, a combination of an antibiotic and an antimycotic can be used in the methods provided herein. The antimycotic can be administered together with, before, or after administration of the antibiotic or with the virus or before or after the virus in an amount effective for treatment of any fungal infection. Antimycotics include, but are not limited to, polyene antifungals such as amphotericin B, candicidin, filipin, hamycin, natamycin, nystatin, rimocidin, imidazole antifungals, such as bifonazole, butoconazole, clotrimazole, econazole, fenticonazole, isoconazole, ketoconazole, miconazole, omoconazole, oxiconazole, sertaconazole, sulconazole and tioconazole, triazoles such as albaconazole, fluconazole, isavuconazole, itraconazole, posaconazole, ravuconazole, terconazole and voriconazole, thiazoles such as abafungin, allylamines such as amorolfine, butenafine, naftifine and terbinafine, echinocandins such as anidulafungin, caspofungin and micafungin, and other antifungals such as ciclopirox, flucytosine or 5-fluorocytosine, griseofulvin, haloprogin, polygodial, tolnaftate, undecylenic acid and crystal violet. An exemplary antimycotic is amphotericin B. For example, an antibiotic-antimycotic combination for use in the methods provided herein includes penicillin, streptomycin and amphotericin B.

Administration and Dosages

Any mode of administration of an antibiotic to a subject can be used, provided the mode of administration permits the antibiotic to effect, e.g., kill, commensal or gut bacteria. Modes of administration can include, but are not limited to, systemic, parenteral, intravenous, intraperitoneal, subcutaneous, intramuscular, transdermal, intradermal, intra-arterial (e.g., hepatic artery infusion), intravesicular perfusion, intrapleural, intraarticular, topical, intratumoral, intralesional, endoscopic, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, or ocular administration. In some examples, an antibiotic is administered by injection, such as intraperitoneally or intravenously. In other examples, the antibiotic is administered orally, by oral gavage, via drinking water. One skilled in the art can select any mode of administration compatible with the subject and antibiotic, and that also is likely to result in the antibiotic effecting commensal or gut bacteria.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the potency and nature of the antibiotic as can be determined by one skilled in the art.

In the present methods, the antibiotic can be administered in any amount that permits the antibiotic to effect, e.g., kill, commensal or gut bacteria. Dosages for antibiotics and their effects on gut bacteria are well known. Generally, the amount of antibiotic administered is an amount between at or about 1 mg and at or about 10 g, such as between at or about 1 mg and at or about 1000 mg, or at or about 1 mg and at or about 500 mg, or at or about 1 mg and at or about 250 mg, or at or about 1 mg and at or about 100 mg, or at or about 1 mg and at or about 50 mg, or at or about 1 mg and at or about 10 mg, or at or about 50 mg and at or about 5 g, or at or about 50 mg and at or about 1 g, or at or about 50 mg and at or about 500 mg, or at or about 50 mg and at or about 250 mg, or at or about 50 mg and at or about 100 mg, or at or about 100 mg and at or about 10 g, or at or about 100 mg and at or about 5 g, or at or about 100 mg and at or about 2.5, or at or about 100 mg and at or about 1, or at or about 100 mg and at or about 500 mg, or at or about 100 mg and at or about 250 mg, or at or about 500 mg and at or about 10 g, or at or about 500 mg and at or about 5 g, or at or about 500 mg and at or about 2.5, or at or about 500 mg and at or about 1 g, or at or about 1 g and at or about 10 g, or at or about 1 g and at or about 5 g, or at or about 1 g and at or about 2.5 g, or at or about 2.5 g and at or about 10 g, or at or about 2.5 g and at or about 5 g, or at or about 5 g and at or about 10 g, or is, or is about or at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975 or 1000 mg, 1.1, 1.2, 1.3, 1.4, 1.5, 1.6, 1.7, 1.8, 1.9, 2, 2.5, 3, 3.5, 4, 4.5, 5, 5.5, 6, 6.5, 7, 7.5, 8, 8.5, 9, 9.5, 10 g or more. In the dosage regime, the antibiotic can be administered as a single administration or multiple times over the cycle of administration. Hence, the methods provided herein can include a single administration of an antibiotic to a subject or multiple administrations of an antibiotic to a subject. In other examples, an antibiotic can be administered on different occasions, separated in time typically by hours or days. For example, an antibiotic can be administered two times, three time, four times, five times, or six times or more with one or more hours between administration, such as 1, 2, 3, 4, 5, 6, 7, 8, 9, 10, 11, 12, 18 or 24 or more hours between administration. Separate administrations can extend the effect of killing of the bacteria.

When separate administrations are performed, each administration can be a dosage amount that is the same or different relative to other administration dosage amounts. In one example, all administration dosage amounts are the same. In other examples, a first dosage amount can be a larger dosage amount than one or more subsequent dosage amounts, for example, at least 10× larger, at least 100× larger, or at least 1000× larger than subsequent dosage amounts. In one example of a method of separate administrations in which the first dosage amount is greater than one or more subsequent dosage amounts, all subsequent dosage amounts can be the same, smaller amount relative to the first administration. Separate administrations can include any number of two or more administrations, including two, three, four, five or six administrations. One skilled in the art can readily determine the number of administrations to perform or the desirability of performing one or more additional administrations according to methods known in the art for monitoring antibiotic efficacy.

Exemplary therapeutically effective amounts of the composition depend upon the virus and antibiotic in the composition and the subject to whom the composition is administered. Typically, single dosage amounts are between or about between 1 mg and 10 g, inclusive; or between or about between 1 mg and 1 gm, inclusive, or at or about at least 500 mg and at or about or at least 5 g; or is or is at least about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975 or 1000 mg, 1.5, 2, 2.5, 3, 3.5, 4, 4.5, or 5 g.

### D. VIRUSES

Viruses for use in the methods provided herein include viruses for gene therapy, including but not limited to, retroviruses, adenoviruses, lentiviruses, herpes simplex viruses, vaccinia viruses, poxviruses and adeno-associated viruses (AAV). Among these viruses are oncolytic viruses, including but not limited to, Newcastle Disease viruses, parvoviruses, vaccinia viruses, reoviruses, measles viruses, vesicular stomatitis viruses (VSV), oncolytic adenoviruses and herpes viruses (HSV). Exemplary of viruses for use in the methods provided herein are oncolytic viruses, described in further detail below. The methods herein are applicable to any viral therapy, since the effect described is not dependent on the particular virus, but requires administration of an antibiotic resulting in its consequent effects on the gut bacteria and on the immune system.

1. Exemplary Oncolytic Viruses

Oncolytic viruses are characterized by their largely tumor cell specific replication, resulting in tumor cell lysis and efficient tumor regression. Oncolytic viruses effect treatment by colonizing or accumulating in tumor cells, including metastatic tumor cells such as circulating tumor cells, and replicating. They provide an effective weapon in the tumor treatment arsenal. Oncolytic viruses include Newcastle Disease virus, parvovirus, vaccinia virus, reovirus, measles virus, vesicular stomatitis virus (VSV), oncolytic adenoviruses and herpes viruses (HSV). In many cases, tumor selectivity is an inherent property of the virus, such as vaccinia viruses and other oncolytic viruses. Generally oncolytic viruses effect treatment by replicating in tumors or tumor cells resulting in lysis.

Oncolytic viruses also include viruses that have been genetically altered to attenuate their virulence, to improve their safety profile, enhance their tumor specificity, and they have also been equipped with additional genes, for example cytotoxins, cytokines, prodrug converting enzymes to improve the overall efficacy of the viruses (see, e.g., Kirn et al., (2009) Nat Rev Cancer 9:64-71; Garcia-Aragoncillo et al., (2010) Curr Opin Mol Ther 12:403-411; see U.S. Pat. Nos. 7,588,767, 7,588,771, 7,662,398, 7,754,221, 8,021,662, 8,052,962, 8,052,962, 8,066,984, 8,221,769 and U.S. Pat. Publ. Nos. 2011/0300176, 2007/0202572, 2007/0212727, 2010/0062016, 2009/0098529, 2009/0053244, 2009/0155287, 2009/0117034, 2010/0233078, 2009/0162288, 2010/0196325, 2009/0136917, 2011/0064650, 2003/0059400, 2004/0234455, 2005/0069491, 2009/0117049, 2009/0117048, 2009/0117047, 2009/0123382, 2003/0228261, 2004/0213741, 2005/0249670, 2012/0308484 and 2012/0244068.

For example, other activities can be introduced and/or anti-tumor activity can be enhanced by including nucleic acid encoding a heterologous gene product that is a therapeutic and/or diagnostic agent or agents. In some examples, the oncolytic viruses provide oncolytic therapy of a tumor cell without the expression of a therapeutic gene. In other examples, the oncolytic viruses can express one or more genes whose products are useful for tumor therapy. For example, a virus can express proteins that cause cell death or whose products cause an anti-tumor immune response. Such genes can be considered therapeutic genes. A variety of therapeutic gene products, such as toxic or apoptotic proteins, or siRNA, are known in the art, and can be used with the oncolytic viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound. Exemplary thereof are gene products selected from among an anticancer agent, an anti-metastatic agent, an antiangiogenic agent, an immunomodulatory molecule, an antigen, a cell matrix degradative gene, genes for tissue regeneration and reprogramming human somatic cells to pluripotency, and other genes described herein or known to one of skill in the art. In these examples, the tumor-specific replication process is capable of directly killing the infected tumor cells (oncolytic viruses) and/or strongly amplifying the copy number of the therapeutic gene carried by the viral vector.

Exemplary therapeutic genes that can be inserted into any oncolytic virus are described herein in Section D.3. and exemplified with respect to vaccinia virus (e.g. LIVP and Western Reserve). It is understood that an oncolytic virus can be modified to include nucleic acid sequences encoding any of the therapeutic genes described in Section D.3. or any known to one of skill in the art. The sequence of nucleotides encoding a gene is typically inserted into or in place of a non-essential gene or region in the genome of the virus.

Thus, oncolytic viruses for use herein include viruses that contain nucleic acid encoding another heterologous gene product that is a therapeutic and/or diagnostic agent or agents. Exemplary of such oncolytic viruses are viruses derived from the Lister strain, such as LIVP, including any containing nucleic acid encoding a heterologous gene product (e.g. GLV-1h68 and derivatives thereof). Such viruses are further described in detail in Section D.1.a.i. Among other therapeutic vaccinia viruses are the virus designated JX-594, which is a vaccinia virus that expresses GM-CSF described, for example, in U.S. Pat. No. 6,093,700, and the Wyeth strain vaccinia virus designated JX-594, which is a TK-deleted vaccinia virus that expresses GM-CSF (see, International PCT Publication No WO 2004/014314, U.S. Pat. No. 5,364,773; Mastrangelo et al. (1998) Cancer Gene Therapy 6:409-422; Kim et al. (2006) Molecular Therapeutics 14:361-370). Other oncolytic viruses include, but are not limited to, JX-954 (Parato et al. (2012) Mol. Ther., 20:749-58); ColoAd1 (Kuhn et al. (2008) PLoS One, 3:e2409; MV-CEA and MV-NIS (Msaouel et al. (2009) Curr. Opin. Mol. Ther., 11:43-53); Synco-B18R (Fu et al. (2012) Mol. Ther., 20:1871-81); OncoVEX GM-CSF (Kaufman et al. (2010) Future Oncol. 6:941-9), Reo-001 (Reolysin®, Galanis et al. (2012) Mol. Ther., 20:1998-2003); NTX-010 (Morton et al. (2010) Pediatr Blood Cancer, 55:295-303); and Coxsackieviruses A13, A15, A18, A20 and A21 (e.g. CAVATAK™, which is coxsackievirus A21.)

In addition, adenoviruses, such as the ONYX viruses and others, have been modified, such as be deletion of EA1 genes, so that they selectively replicate in cancerous cells, and, thus, are oncolytic. Adenoviruses also have been engineered to have modified tropism for tumor therapy and also as gene therapy vectors. Exemplary of such is ONYX-015, H101 and Ad5ΔCR (Hallden and Portella (2012) Expert Opin Ther Targets, 16:945-58) and TNFerade (McLoughlin et al. (2005) Ann. Surg. Oncol., 12:825-30). A conditionally replicative adenovirus Oncorine®, which is approved in China.

Any virus can be modified to remove or disrupt native genes that cause disease and insert heterologous nucleic acid molecules using standard cloning methods known in the art and described elsewhere herein. For example, the sequence of nucleotides encoding a heterologous protein is inserted into or in place of a non-essential gene or region in the genome of an unmodified oncolytic virus or is inserted into in or in place of nucleic acid encoding a heterologous gene product in the genome of an unmodified oncolytic virus. Any of the oncolytic viruses described above or in Section D.1.a further below, or otherwise known in the art, can be used as an unmodified virus herein for insertion of nucleic acid encoding a heterologous gene product.

a. Poxviruses—Vaccinia Viruses

Vaccinia viruses are oncolytic viruses that possess a variety of features that make them particularly suitable for use in wound and cancer gene therapy. For example, vaccinia is a cytoplasmic virus, thus, it does not insert its genome into the host genome during its life cycle. Unlike many other viruses that require the host's transcription machinery, vaccinia virus can support its own gene expression in the host cell cytoplasm using enzymes encoded in the viral genome. Vaccinia viruses also have a broad host and cell type range. In particular vaccinia viruses can accumulate in immunoprivileged cells or immunoprivileged tissues, including tumors and/or metastases, and also including wounded tissues and cells. Yet, unlike other oncolytic viruses, vaccinia virus can typically be cleared from the subject to whom the viruses are administered by activity of the subject's immune system, and hence are less toxic than other viruses such as adenoviruses. Thus, while the viruses can typically be cleared from the subject to whom the viruses are administered by activity of the subject's immune system, viruses can nevertheless accumulate, survive and proliferate in immunoprivileged cells and tissues such as tumors because such immunoprivileged areas are isolated from the host's immune system.

Vaccinia viruses also can be easily modified by insertion of heterologous genes. This can result in the attenuation of the virus and/or permit delivery of therapeutic proteins. For example, vaccinia virus genome has a large carrying capacity for foreign genes, where up to 25 kb of exogenous DNA fragments (approximately 12% of the vaccinia genome size) can be inserted. The genomes of several of the vaccinia strains have been completely sequenced, and many essential and nonessential genes identified. Due to high sequence homology among different strains, genomic information from one vaccinia strain can be used for designing and generating modified viruses in other strains. Finally, the techniques for production of modified vaccinia strains by genetic engineering are well established (Moss, (1993) Curr. Opin. Genet. Dev. 3:86-90; Broder and Earl, (1999) Mol. Biotechnol. 13:223-245; Timiryasova et al., (2001) Biotechniques 31:534-540).

Various vaccinia viruses have been demonstrated to exhibit antitumor activities. In one study, for example, nude mice bearing nonmetastatic colon adenocarcinoma cells were systemically injected with a WR strain of vaccinia virus modified by having a vaccinia growth factor deletion and an enhanced green fluorescence protein inserted into the thymidine kinase locus. The virus was observed to have antitumor effect, including one complete response, despite a lack of exogenous therapeutic genes in the modified virus (McCart et al. (2001) Cancer Res 1:8751-8757). In another study, vaccinia melanoma oncolysate (VMO) was injected into sites near melanoma positive lymph nodes in a Phase III clinical trial of melanoma patients. As a control, New York City Board of Health strain vaccinia virus (VV) was administered to melanoma patients. The melanoma patients treated with VMO had a survival rate better than that for untreated patients, but similar to patients treated with the VV control (Kim et al. (2001) Surgical Oncol 10:53-59).

LIVP strains of vaccinia virus also have been used for the diagnosis and therapy of tumors, and for the treatment of wounded and inflamed tissues and cells (see e.g. Zhang et al. (2007) Surgery, 142:976-983; Lin et al. (2008) J. Clin. Endocrinol., Metab., 93:4403-7; Kelly et al. (2008) Hum gene Ther., 19:774-782; Yu et al. (2009) Mol Cancer Ther., 8:141-151; Yu et al. (2009) Mol Cancer, 8:45; U.S. Pat. Nos. 7,588,767 and 8,052,968; and U.S. Patent Publication No. US20040234455). For example, when intravenously administered, LIVP strains have been demonstrated to accumulate in internal tumors at various loci in vivo, and have been demonstrated to effectively treat human tumors of various tissue origin, including, but not limited to, breast tumors, thyroid tumors, pancreatic tumors, metastatic tumors of pleural mesothelioma, squamous cell carcinoma, lung carcinoma and ovarian tumors. LIVP strains of vaccinia, including attenuated forms thereof, exhibit less toxicity than WR strains of vaccinia virus, and results in increased and longer survival of treated tumor-bearing animal models (see e.g. U.S. Patent Publication No. US20110293527).

Vaccinia is a cytoplasmic virus, thus, it does not insert its genome into the host genome during its life cycle. Vaccinia virus has a linear, double-stranded DNA genome of approximately 180,000 base pairs in length that is made up of a single continuous polynucleotide chain (Baroudy et al. (1982) Cell, 28:315-324). The structure is due to the presence of 10,000 base pair inverted terminal repeats (ITRs). The ITRs are involved in genome replication. Genome replication is believed to involve self-priming, leading to the formation of high molecular weight concatemers (isolated from infected cells) which are subsequently cleaved and repaired to make virus genomes. See, e.g., Traktman, P., Chapter 27, Poxvirus DNA Replication, pp. 775-798, in DNA Replication in Eukaryotic Cells, Cold Spring Harbor Laboratory Press (1996). The genome encodes for approximately 250 genes. In general, the nonsegmented, noninfectious genome is arranged such that centrally located genes are essential for virus replication (and are thus conserved), while genes near the two termini effect more peripheral functions such as host range and virulence. Vaccinia viruses practice differential gene expression by utilizing open reading frames (ORFs) arranged in sets that, as a general principle, do not overlap.

Vaccinia virus possesses a variety of features for use in cancer gene therapy and vaccination including broad host and cell type range, and low toxicity. For example, while most oncolytic viruses are natural pathogens, vaccinia virus has a unique history in its widespread application as a smallpox vaccine that has resulted in an established track record of safety in humans. Toxicities related to vaccinia administration occur in less than 0.1% of cases, and can be effectively addressed with immunoglobulin administration. In addition, vaccinia virus possesses a large carrying capacity for foreign genes (up to 25 kb of exogenous DNA fragments (approximately 12% of the vaccinia genome size) can be inserted into the vaccinia genome), high sequence homology among different strains for designing and generating modified viruses in other strains, and techniques for production of modified vaccinia strains by genetic engineering are well established (Moss (1993) Curr. Opin. Genet. Dev. 3: 86-90; Broder and Earl (1999) Mol. Biotechnol. 13: 223-245; Timiryasova et al. (2001) Biotechniques 31: 534-540). Vaccinia virus strains have been shown to specifically colonize solid tumors, while not infecting other organs (see, e.g., Zhang et al. (2007) Cancer Res 67:10038-10046; Yu et al., (2004) Nat Biotech 22:313-320; Heo et al., (2011) Mol Ther 19:1170-1179; Liu et al. (2008) Mol Ther 16:1637-1642; Park et al., (2008) Lancet Oncol, 9:533-542).

A variety of vaccinia virus strains are available for modification by insertion of nucleic acid encoding melanin producing enzymes, including, but not limited to, Western Reserve (WR) (SEQ ID NO:21), Copenhagen (SEQ ID NO:21), Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Exemplary of known viruses are set forth in Table 3. Exemplary of vaccinia viruses for use in the methods provided herein include, but are not limited to, Lister strain or LIVP strain of vaccinia viruses, WR strains, or modified forms thereof. LIVP generally exhibits less virulence than the WR strain. Also, for example, a recombinant derivative of LIVP, designated GLV-1h68 (set forth in SEQ ID NO:1; GenBank Acc. No. EU410304) and GLV-1h64 (set forth in SEQ ID NO:10) exhibit tumor targeting properties and an improved safety profile compared to its parental LIVP strain (set forth in SEQ ID NO:20) and the WR strain (Zhang et al. (2009) Mol. Genet. Genomics, 282:417-435).

TABLE 3
Vaccinia Virus Strains

| Name | Abbreviation | GenBank Accession No. |
|---|---|---|
| Vaccinia virus strain Western | WR | AY243312 |
| Reserve | | |
| Vaccinia virus strain | COP | M35027 |
| Copenhagen | | |
| Vaccinia Lister major strain | LIST | AY678276 |
| Vaccinia Lister isolate LC16MO | LC | AY678277 |
| Vaccinia Lister clone VACV107 | VACV107 | DQ121394 |
| Vaccinia virus strain | ACAM | AY313847 |
| ACAM2000 | | |
| Vaccinia virus strain DUKE | DUKE | DQ439815; Li et al. |
| | | (2006) Virology J, 3: 88 |
| Vaccinia virus strain Ankara | MVA | U94848 |
| Vaccinia virus Clone3 | CLONE3 | AY138848 |

Lister and LIVP Strains

Exemplary vaccinia viruses are Lister or LIVP vaccinia viruses. Lister (also referred to as Elstree) vaccinia virus is available from any of a variety of sources. For example, the Elstree vaccinia virus is available at the ATCC under Accession Number VR-1549. The Lister vaccinia strain has high transduction efficiency in tumor cells with high levels of gene expression.

The vaccinia virus provided in the compositions and methods herein can be based on modifications to the Lister strain of vaccinia virus. LIVP is a vaccinia strain derived from Lister (ATCC Catalog No. VR-1549). As described elsewhere herein, the LIVP strain can be obtained from the Lister Institute of Viral Preparations, Moscow, Russia; the Microorganism Collection of FSR1 SRC VB Vector; or can be obtained from the Moscow Ivanovsky Institute of Virology (C0355 K0602). The LIVP strain was used for vaccination throughout the world, particularly in India and Russia, and is widely available. LIVP, including derivatives thereof, such as GLV-1h68 and derivatives of GLV-1h68, and production thereof are described, for example, in U.S. Pat. Nos. 7,588,767, 7,588,771, 7,662,398 and 7,754,221 and U.S. Patent Publication Nos. 2007/0202572, 2007/0212727, 2010/0062016, 2009/0098529, 2009/0053244, 2009/0155287, 2009/0117034, 2010/0233078, 2009/0162288, 2010/0196325, 2009/0136917, 2011/0064650; Zhang et al. (2009) Mol. Genet. Genomics, 282:417-435). A sequence of a parental genome of LIVP is set forth in SEQ ID NO:20.

LIVP strains for use in the methods provided herein also include clonal strains that are derived from LIVP and that can be present in a virus preparation propagated from LIVP. The LIVP clonal strains have a genome that differs from the parental sequence set forth in SEQ ID NO:20. The clonal strains provided herein exhibit greater anti-tumorigenicity and/or reduced toxicity compared to the recombinant or modified virus strain designated GLV-1h68 (having a genome set forth in SEQ ID NO:1).

The LIVP and clonal strains have a sequence of nucleotides that have at least 70%, such as at least 75%, 80%, 85% or 90% sequence identity to SEQ ID NO:2 or 20. For example, the clonal strains have a sequence of nucleotides that has at least 91%, 92%, 93%, 94%, 95%, 95%, 97%, 98%, 99%, 99.1%, 99.2%, 99.3%, 99.4%, 99.5%, 99.6%, 99.7%, 99.8%, 99.9% or 100% sequence identity to SEQ ID NO:2 or 20. Such LIVP clonal viruses include viruses that differ in one or more open reading frames (ORF) compared to the parental LIVP strain that has a sequence of amino acids set forth in SEQ ID NO:2 or 20. The LIVP clonal virus strains provided herein can contain a nucleotide deletion or mutation in any one or more nucleotides in any ORF compared to SEQ ID NO:2 or 20, or can contain an addition or insertion of viral DNA compared to SEQ ID NO:2 or 20.

LIVP strains in the compositions provided herein include those that have a nucleotide sequence corresponding to nucleotides 2,256-181,114 of SEQ ID NO:3, nucleotides 11,243-182,721 of SEQ ID NO:4, nucleotides 6,264-181,390 of SEQ ID NO:5, nucleotides 7,044-181,820 of SEQ ID NO:6, nucleotides 6,674-181,409 of SEQ ID NO:7, nucleotides 6,716-181,367 of SEQ ID NO:8 or nucleotides 6,899-181,870 of SEQ ID NO:9, or to a complement thereof. In some examples, the LIVP strain for use in the compositions and methods is a clonal strain of LIVP or a modified form thereof containing a sequence of nucleotides that has at least 97%, 98%, 99% or more sequence identity to a sequence of nucleotides 2,256-181,114 of SEQ ID NO:3, nucleotides 11,243-182,721 of SEQ ID NO:4, nucleotides 6,264-181,390 of SEQ ID NO:5, nucleotides 7,044-181,820 of SEQ ID NO:6, nucleotides 6,674-181,409 of SEQ ID NO:7, nucleotides 6,716-181,367 of SEQ ID NO:8 or nucleotides 6,899-181,870 of SEQ ID NO:9. LIVP clonal strains provided herein generally also include terminal nucleotides corresponding to a left and/or right inverted terminal repeat (ITR). Exemplary LIVP strains include but are not limited to virus strains designated LIVP 1.1.1 having a genome containing a sequence of nucleotides set forth in SEQ ID NO:3 or a sequence of nucleotides that exhibits at least 97% sequence identity to SEQ ID NO:3; a virus strain designated LIVP 2.1.1 having a genome containing a sequence of nucleotides set forth in SEQ ID NO:4 or a sequence of nucleotides that exhibits at least 97%, 98%, 99% or more sequence identity to SEQ ID NO:4; a virus strain designated LIVP 4.1.1 having a genome containing a sequence of nucleotides set forth in SEQ ID NO:5 or a sequence of nucleotides that exhibits at least 97%, 98%, 99% or more sequence identity to SEQ ID NO:5; a virus strain designated LIVP 5.1.1 having a genome containing a sequence of nucleotides set forth in SEQ ID NO:6 or a sequence of nucleotides that exhibits at least 97%, 98%, 99% or more sequence identity to SEQ ID NO:6; a virus strain designated LIVP 6.1.1 having a sequence of nucleotides set forth in SEQ ID NO:7 or a sequence of nucleotide that exhibits at least 97%, 98%, 99% or more sequence identity to SEQ ID NO:7; a virus strain designated LIVP 7.1.1 having a genome containing a sequence of nucleotides set forth in SEQ ID NO:8 or a sequence of nucleotides that exhibits at least 97%, 98%, 99% or more sequence identity to SEQ ID NO:8; or a virus strain designated LIVP 8.1.1 having a genome containing a sequence of nucleotides set forth in SEQ ID NO:9 or a sequence of nucleotides that exhibits at least 97%, 98%, 99% or more sequence identity to SEQ ID NO:9.

i. Modified Vaccinia Viruses

Modified or recombinant vaccinia strains containing heterologous nucleic acid encoding a gene product or products have been or can be generated from any of a variety of vaccinia virus strains, including, but not limited to, Western Reserve (WR) (SEQ ID NO:21), Copenhagen (SEQ ID NO:22), Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. Such strains, or modified strains thereof, can be used in the methods provided herein.

For example, recombinant vaccinia viruses, such as LIVP viruses, have been generated and are known in the art. Exemplary modified or recombinant vaccinia viruses for use in the methods provided herein are those derived from the Lister strain, and in particular the attenuated Lister strain LIVP. The modified LIVP viruses can be modified by insertion, deletion or amino acid replacement of heterologous nucleic acid compared to an LIVP strain having a genome set forth in any one of SEQ ID NOS:2-9 or 20, or having a genome that exhibits at least 97%, 98%, 99% or more sequence identity to any of SEQ ID NOS:2-9 or 20.

For example, known modified or recombinant LIVP viruses include GLV-1h68 or derivatives thereof. GLV-1h68 (also named RVGL21, SEQ ID NO:1; described in U.S. Pat. Pub. No. 2005-0031643, now U.S. Pat. Nos. 7,588,767, 7,588,771 and 7,662,398) is an attenuated virus of the LIVP strain containing a genome set forth in SEQ ID NO:20 that contains DNA insertions in gene loci F14.5L (also designated in LIVP as F3) gene locus, thymidine kinase (TK) gene locus, and hemagglutinin (HA) gene locus with expression cassettes encoding detectable marker proteins. Specifically, GLV-1h68 contains an expression cassette containing a Ruc-GFP cDNA molecule (a fusion of DNA encoding Renilla luciferase and DNA encoding GFP) under the control of a vaccinia synthetic early/late promoter P_{SEL} ((P_{SEL})Ruc-GFP) inserted into the F14.5L gene locus; an expression cassette containing a DNA molecule encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) inserted into the TK gene locus (the resulting virus does not express transferrin receptor protein since the DNA molecule encoding the protein is positioned in the reverse orientation for transcription relative to the promoter in the cassette); and an expression cassette containing a DNA molecule encoding β-glucuronidase under the control of the vaccinia late promoter P₁₁ₖ ((P₁₁ₖ)gusA) inserted into the HA gene locus.

Other recombinant LIVP viruses are derived from GLV-1h68 and contain heterologous DNA that encodes a gene product or products (see e.g. see e.g. U.S. Pub. Nos. US2003-0059400, US2003-0228261, US2007-0202572, US2007-0212727, US2009-0117034, US2009-0098529, US2009-0053244, US2009-0155287, US2009-0081639, US2009-0136917, US2009-0162288, US2010-0062016, US2010-0233078 and US2010-0196325; U.S. Pat. Nos. 7,588,767, 7,588,771, 7,662,398 and 7,754,221 and 7,763,420; and International Pub. No. WO 2009/139921). Exemplary of such recombinant viruses include those set forth in Table 4, including but not limited to, GLV-1h64 (set forth in SEQ ID NO:10); GLV-1h188 (SEQ ID NO:11), GLV-1h189 (SEQ ID NO:12), GLV-1h190 (SEQ ID NO:13), GLV-1h253 (SEQ ID NO:14), and GLV-1h254 (SEQ ID NO:15); GLV-1h311 (SEQ ID NO:16); GLV-1h312 (SEQ ID NO:17); GLV-1h330 (SEQ ID NO:18); or GLV-1h354 (SEQ ID NO:19).

Modified vaccinia viruses also include viruses that are modified by introduction of heterologous nucleic acid into an LIVP strain containing a genome set forth in any of SEQ ID NOS:3-9, or a genome that exhibits at least 97%, 98%, 99% or more sequence identity to any of SEQ ID NOS:3-9.

Table 4 sets forth exemplary viruses, the reference or parental vaccinia virus (e.g. LIVP set forth in SEQ ID NO:2 or 20 or GLV-1h68 set forth in SEQ ID NO:1) and the resulting genotype. The exemplary modifications of the Lister strain can be adapted to other vaccinia viruses (e.g., Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health). Any of these viruses, and other oncolytic viruses known in the art, can be used in the methods provided herein.

TABLE 4
Recombinant Viruses

| | | Genotype | | | | |
|---|---|---|---|---|---|---|
| Virus | Parent | | J2R | A56R | | |
| Name | Virus | F14.5L | (TK locus) | (HA locus) | A34R | A35R |

LIVP- & GLV-1h68-derived Virus Strains

| | | | | | | |
|---|---|---|---|---|---|---|
| GLV-1h68 | LIVP | (PSEL)Ruc- | (PSEL)rTrfR- | (P11)gusA | wt | wt |
| | | GFP | (P7.5)lacZ | | | |
| GLV-1i69 | GLV-1h68 | (PSEL)Ruc- | (PSEL)rTrfR- | (P11)gusA | A34R | wt |
| | | GFP | (P7.5)lacZ | | from | |
| | | | | | IHD-J | |
| GLV-1h70 | GLV-1h68 | (PSEL)Ruc- | (PSEL)rTrfR- | ko | wt | wt |
| | | GFP | (P7.5)lacZ | | | |
| GLV-1h71 | GLV-1h68 | ko | (PSEL)rTrfR- | (P11)gusA | wt | wt |
| | | | (P7.5)lacZ | | | |
| GLV-1h72 | GLV-1h68 | (PSEL)Ruc- | ko | (P11)gusA | wt | wt |
| | | GFP | | | | |
| GLV-1h73 | GLV-1h70 | ko | (PSEL)rTrfR- | ko | wt | wt |
| | | | (P7.5)lacZ | | | |
| GLV-1h74 | GLV-1h73 | ko | ko | ko | wt | wt |
| GLV-1h76 | GLV-1h68 | (PSEL)Ruc- | (PSE)GM-CSF | (P11)gusA | wt | wt |
| | | GFP | | | | |
| GLV-1h77 | GLV-1h68 | (PSEL)Ruc- | (PSEL)GM-CSF | (P11)gusA | wt | wt |
| | | GFP | | | | |
| GLV-1h78 | GLV-1h68 | (PSEL)Ruc- | (PSL)GM-CSF | (P11)gusA | wt | wt |
| | | GFP | | | | |
| GLV-1h79 | GLV-1h68 | (PSEL)Ruc- | (PSEL)mMCP-1 | (P11)gusA | wt | wt |
| | | GFP | | | | |
| GLV-1h80 | GLV-1h68 | (PSEL)Ruc- | (PSL)mMCP-1 | (P11)gusA | wt | wt |
| | | GFP | | | | |
| GLV-1h81 | GLV-1h68 | (PSEL)Ruc- | (PSEL)rTrfR- | (PSEL)hk5 | wt | wt |
| | | GFP | (P7.5)lacZ | | | |
| GLV-1h82 | GLV-1h22 | (PSEL)Ruc- | (PSEL)TrfR- | (PSEL)ftn | wt | wt |
| | | GFP | (P7.5)lacZ | | | |
| GLV-1h83 | GLV-1h68 | (PSEL)Ruc- | (PSEL)rTrfR- | (PSEL)ftn | wt | wt |
| | | GFP | (P7.5)lacZ | | | |
| GLV-1h84 | GLV-1h68 | ko | (PSEL)CBG99- | ko | wt | wt |
| | | | mRFP1 | | | |
| GLV-1h85 | GLV-1h72 | ko | ko | (P11)gusA | wt | wt |
| GLV-1h86 | GLV-1h72 | (PSEL)Ruc- | ko | ko | wt | wt |
| | | GFP | | | | |
| GLV-1j87 | GLV-1h68 | (PSEL)Ruc- | (PSEL)rTrfR- | (P11)gusA | wt | ko |
| | | GFP | (P7.5)lacZ | | | |
| GLV-1j88 | GLV-1h73 | ko | (PSEL)rTrfR- | ko | wt | ko |
| | | | (P7.5)lacZ | | | |
| GLV-1j89 | GLV-1h74 | ko | ko | ko | wt | ko |
| GLV-1h90 | GLV-1h68 | (PSEL)Ruc- | (PSEL)rTrfR- | (PSE)sIL- | wt | wt |
| | | GFP | (P7.5)lacZ | 6R/IL-6 | | |
| GLV-1h91 | GLV-1h68 | (PSEL)Ruc- | (PSEL)rTrfR- | (PSEL)sIL- | wt | wt |
| | | GFP | (P7.5)lacZ | 6R/IL-6 | | |
| GLV-1h92 | GLV-1h68 | (PSEL)Ruc- | (PSEL)rTrfR- | (PSL)sIL- | wt | wt |
| | | GFP | (P7.5)lacZ | 6R/IL-6 | | |
| GLV-1h93 | GLV-1h68 | (PSEL)Ruc- | (PSEL)rTrfR- | (PSE)FCU1 | wt | wt |
| | | GFP | (P7.5)lacZ | | | |
| GLV-1h94 | GLV-1h68 | (PSEL)Ruc- | (PSEL)rTrfR- | (PSEL)FCU1 | wt | wt |
| | | GFP | (P7.5)lacZ | | | |
| GLV-1h95 | GLV-1h68 | (PSEL)Ruc- | (PSEL)rTrfR- | (PSL)FCU1 | wt | wt |
| | | GFP | (P7.5)lacZ | | | |
| GLV-1h96 | GLV-1h68 | (PSE)IL-24 | (PSEL)rTrfR- | (P11)gusA | wt | wt |
| | | | (P7.5)lacZ | | | |
| GLV-1h97 | GLV-1h68 | (PSEL)IL-24 | (PSEL)rTrfR- | (P11)gusA | wt | wt |
| | | | (P7.5)lacZ | | | |
| GLV-1h98 | GLV-1h68 | (PSL)IL-24 | (PSEL)rTrfR- | (P11)gusA | wt | wt |
| | | | (P7.5)lacZ | | | |
| GLV-1h99 | GLV-1h68 | (PSE)hNET | (PSEL)rTrfR- | (P11)gusA | wt | wt |
| | | | (P7.5)lacZ | | | |
| GLV-1h100 | GLV-1h68 | (PSEL)Ruc- | (PSE)hNET | (P11)gusA | wt | wt |
| | | GFP | | | | |
| GLV-1h101 | GLV-1h68 | (PSEL)Ruc- | (PSL)hNET | (P11)gusA | wt | wt |
| | | GFP | | | | |
| GLV-1h102 | GLV-1h68 | (PSEL)Ruc- | (PSEL)rTrfR- | (PSE)hDMT | wt | wt |
| | | GFP | (P7.5)lacZ | | | |
| GLV-1h103 | GLV-1h68 | (PSEL)Ruc- | (PSL)hMCP1 | (P11)gusA | wt | wt |
| | | GFP | | | | |
| GLV-1h104 | GLV-1h68 | (PSEL)Ruc- | (PSE)tTF-RGD | (P11)gusA | wt | wt |
| | | GFP | | | | |
| GLV-1h105 | GLV-1h68 | (PSEL)Ruc- | (PSE/L)tTF- | (P11)gusA | wt | wt |
| | | GFP | RGD | | | |
| GLV-1h106 | GLV-1h68 | (PSEL)Ruc- | (PSL)tTF-RGD | (P11)gusA | wt | wt |
| | | GFP | | | | |
| GLV-1h107 | GLV-1h68 | (PSEL)Ruc- | (PSE)G6-FLAG | (P11)gusA | wt | wt |
| | | GFP | | | | |
| GLV-1h108 | GLV-1h68 | (PSEL)Ruc- | (PSEL)G6- | (P11)gusA | wt | wt |
| | | GFP | FLAG | | | |
| GLV-1h109 | GLV-1h68 | (PSEL)Ruc- | (PSL)G6-FLAG | (P11)gusA | wt | wt |
| | | GFP | | | | |
| GLV-1h110 | GLV-1h68 | (PSEL)Ruc- | (PSEL)rTrfR- | (PSE)bfr | wt | wt |
| | | GFP | (P7.5)lacZ | | | |
| GLV-1h111 | GLV-1h68 | (PSEL)Ruc- | (PSEL)rTrfR- | (PSEL)bfr | wt | wt |
| | | GFP | (P7.5)lacZ | | | |
| GLV-1h112 | GLV-1h68 | (PSEL)Ruc- | (PSEL)rTrfR- | (PSL)bfr | wt | wt |
| | | GFP | (P7.5)lacZ | | | |
| GLV-1h113 | GLV-1h68 | (PSEL)Ruc- | (PSEL)rTrfR- | (PSEL)bfrₒₚₜ | wt | wt |
| | | GFP | (P7.5)lacZ | | | |
| GLV-1h114 | GLV-1h68 | (PSEL)Ruc- | (PSEL)rTrfR- | (PSE)mtr | wt | wt |
| | | GFP | (P7.5)lacZ | | | |
| GLV-1h115 | GLV-1h68 | (PSEL)Ruc- | (PSEL)rTrfR- | (PSEL)mtr | wt | wt |
| | | GFP | (P7.5)lacZ | | | |
| GLV-1h116 | GLV-1h68 | (PSEL)Ruc- | (PSE)mMnSOD | (P11)gusA | wt | wt |
| | | GFP | | | | |
| GLV-1h117 | GLV-1h68 | (PSEL)Ruc- | (PSEL)mMnSOD | (P11)gusA | wt | wt |
| | | GFP | | | | |
| GLV-1h118 | GLV-1h68 | (PSEL)Ruc- | (PSL)mMnSOD | (P11)gusA | wt | wt |
| | | GFP | | | | |
| GLV-1h119 | GLV-1h68 | (PSEL)Ruc- | (PSE)mIP-10 | (P11)gusA | wt | wt |
| | | GFP | | | | |
| GLV-1h120 | GLV-1h68 | (PSEL)Ruc- | (PSEL)mIP-10 | (P11)gusA | wt | wt |
| | | GFP | | | | |
| GLV-1h121 | GLV-1h68 | (PSEL)Ruc- | (PSL)mIP-10 | (P11)gusA | wt | wt |
| | | GFP | | | | |
| GLV-1h122 | GLV-1h68 | (PSEL)Ruc- | (PSE)mLIGHT | (P11)gusA | wt | wt |
| | | GFP | | | | |
| GLV-1h123 | GLV-1h68 | (PSEL)Ruc- | (PSE/L)mLIGHT | (P11)gusA | wt | wt |
| | | GFP | | | | |
| GLV-1h124 | GLV-1h68 | (PSEL)Ruc- | (PSL)mLIGHT | (P11)gusA | wt | wt |
| | | GFP | | | | |
| GLV-1h125 | GLV-1h68 | (PSEL)Ruc- | (PSE)CBP | (P11)gusA | wt | wt |
| | | GFP | | | | |
| GLV-1h126 | GLV-1h68 | (PSEL)Ruc- | (PSEL)CBP | (P11)gusA | wt | wt |
| | | GFP | | | | |
| GLV-1h127 | GLV-1h68 | (PSEL)Ruc- | (PSL)CBP | (P11)gusA | wt | wt |
| | | GFP | | | | |
| GLV-1h128 | GLV-1h68 | (PSEL)Ruc- | (PSE)P60 | (P11)gusA | wt | wt |
| | | GFP | | | | |
| GLV-h129 | GLV-1h68 | (PSEL)Ruc- | (PSEL)P60 | (P11)gusA | wt | wt |
| | | GFP | | | | |
| GLV-1h130 | GLV-1h68 | (PSEL)Ruc- | (PSL)P60 | (P11)gusA | wt | wt |
| | | GFP | | | | |
| GLV-1h131 | GLV-1h68 | (PSEL)Ruc- | (PSE)hFLH | (P11)gusA | wt | wt |
| | | GFP | | | | |
| GLV-1h132 | GLV-1h68 | (PSEL)Ruc- | (PSEL)hFLH | (P11)gusA | wt | wt |
| | | GFP | | | | |
| GLV-1h133 | GLV-1h68 | (PSEL)Ruc- | (PSL)hFLH | (P11)gusA | wt | wt |
| | | GFP | | | | |
| GLV-1h134 | GLV-1h68 | (PSEL)CBG99- | (PSEL)rTrfR- | (P11)gusA | wt | wt |
| | | mRFP1 | (P7.5)lacZ | | | |
| GLV-1h135 | GLV-1h68 | wt | (PSEL)rTrfR- | (P11)gusA | wt | wt |
| | | | (P7.5)lacZ | | | |
| GLV-1h136 | GLV-1h68 | (PSEL)Ruc- | (PSE)PEDF | (P11)gusA | wt | wt |
| | | GFP | | | | |
| GLV-1h137 | GLV-1h68 | (PSEL)Ruc- | (PSEL)PEDF | (P11)gusA | wt | wt |
| | | GFP | | | | |
| GLV-1h138 | GLV-1h68 | (PSEL)Ruc- | (PSL)PEDF | (P11)gusA | wt | wt |
| | | GFP | | | | |
| GLV-1h139 | GLV-1h68 | (PSEL)Ruc- | (PSEL)rTrfR- | (PSE)hNET | wt | wt |
| | | GFP | (P7.5)lacZ | | | |
| GLV-1h140 | GLV-1h68 | (PSEL)Ruc- | (PSE)CYP11B1 | (P11)gusA | wt | wt |
| | | GFP | | | | |
| GLV-1h141 | GLV-1h68 | (PSEL)Ruc- | (PSEL)CYP11B1 | (P11)gusA | wt | wt |
| | | GFP | | | | |
| GLV-1h142 | GLV-1h68 | (PSEL)Ruc- | (PSL)CYP11B1 | (P11)gusA | wt | wt |
| | | GFP | | | | |
| GLV-1h143 | GLV-1h68 | (PSEL)Ruc- | (PSE)CYP11B2 | (P11)gusA | wt | wt |
| | | GFP | | | | |
| GLV-1h144 | GLV-1h68 | (PSEL)Ruc- | (PSEL)CYP11B2 | (P11)gusA | wt | wt |
| | | GFP | | | | |
| GLV-1h145 | GLV-1h68 | (PSEL)Ruc- | (PSL)CYP11B2 | (P11)gusA | wt | wt |
| | | GFP | | | | |
| GLV-1h146 | GLV-1h100 | (PSEL)Ruc- | (PSE)hNET | (PSE)IL-24 | wt | wt |
| | | GFP | | | | |
| GLV-1h147 | GLV-1h68 | (PSEL)Ruc- | (PSE)HACE1 | (P11)gusA | wt | wt |
| | | GFP | | | | |
| GLV-1h148 | GLV-1h68 | (PSEL)Ruc- | (PSEL)HACE1 | (P11)gusA | wt | wt |
| | | GFP | | | | |
| GLV-1h149 | GLV-1h68 | (PSEL)Ruc- | (PSL)HACE1 | (P11)gusA | wt | wt |
| | | GFP | | | | |
| GLV-1h150 | GLV-1h101 | (PSEL)Ruc- | (PSL)hNET | (PSE)IL-24 | wt | wt |
| | | GFP | | | | |
| GLV-1h151 | GLV-1h68 | (PSEL) Ruc- | (PSE/L)TfR- | (PSE)hNIS | wt | wt |
| | | GFP | (P7.5)lacZ | | | |
| GLV-1h153 | GLV-1h68 | (PSEL) Ruc- | (PSEL)TfR- | (PSE)hNISa | wt | wt |
| | | GFP | (P7.5)lacZ | | | |
| GLV-1h154 | GLV-1h22 | (PSEL) Ruc- | (PSEL)TfR- | (PSEL)bfrₒₚₜ | wt | wt |
| | | GFP | (P7.5)lacZ | | | |
| GLV-1h155 | GLV-1h22 | (PSEL) Ruc- | (PSEL)TfR- | (PSEL)hFH | wt | wt |
| | | GFP | (P7.5)lacZ | | | |
| GLV-1h156 | GLV-1h113 | (PSEL) Ruc- | (PSEL)mtr | (PSEL)bfrₒₚₜ | wt | wt |
| | | GFP | | | | |
| GLV-1h157 | GLV-1h68 | (PSEL) Ruc- | (PSEL)mtr | (PSEL)hFH | wt | wt |
| | | GFP | | | | |
| GLV-1h158 | GLV-1h68 | (PSEL) Ruc- | (PSEL)TfR- | (PSEL)G6-scAb | wt | wt |
| | | GFP | (P7.5)lacZ | | | |
| GLV-1h159 | GLV-1h68 | (PSEL) Ruc- | (PSEL)TfR- | (PSL)G6-scAb | wt | wt |
| | | GFP | (P7.5)lacZ | | | |
| GLV-1h160 | GLV-1h68 | (PSEL)Ruc- | (PSEL)luxAB | (P11)gusA | wt | wt |
| | | GFP | | | | |
| GLV-1h161 | GLV-1h68 | (PSEL)Ruc- | (PSEL)TfR- | (PSEL)luxCD | wt | wt |
| | | GFP | (P7.5)lacZ | | | |
| GLV-1h162 | GLV-1h68 | (PSEL)luxE | (PSEL)rTrfR- | (P11)gusA | wt | wt |
| | | | (P7.5)lacZ | | | |
| GLV-1h163 | GLV-1h100 | (PSEL)Ruc- | (PSE)hNET | (PSEL)G6-scAb | wt | wt |
| | | GFP | | | | |
| GLV-1h164 | GLV-1h100 | (PSEL)Ruc- | (PSE)hNET | (PSL)G6-scAb | wt | wt |
| | | GFP | | | | |
| GLV-1h165 | GLV-1h68 | (PSEL)Ruc- | (PSE)nAG | (P11)gusA | wt | wt |
| | | GFP | | | | |
| GLV-1h166 | GLV-1h68 | (PSEL)Ruc- | (PSEL)NAG | (P11)gusA | wt | wt |
| | | GFP | | | | |
| GLV-1h167 | GLV-1h68 | (PSEL)Ruc- | (PSL)nAG | (P11)gusA | wt | wt |
| | | GFP | | | | |
| GLV-1h168 | GLV-1h68 | (PSEL)Ruc- | (PSE)RLN | (P11)gusA | wt | wt |
| | | GFP | | | | |
| GLV-1h169 | GLV-1h68 | (PSEL)Ruc- | (PSEL)RLN | (P11)gusA | wt | wt |
| | | GFP | | | | |
| GLV-1h170 | GLV-1h68 | (PSEL)Ruc- | (PSL)RLN | (P11)gusA | wt | wt |
| | | GFP | | | | |
| GLV-1h171 | GLV-1h68 | (PSEL)Ruc- | (PSE)NM23A | (P11)gusA | wt | wt |
| | | GFP | | | | |
| GLV-1h172 | GLV-1h68 | (PSEL)Ruc- | (PSEL)NM23A | (P11)gusA | wt | wt |
| | | GFP | | | | |
| GLV-1h173 | GLV-1h68 | (PSEL)Ruc- | (PSL)NM23 | (P11)gusA | wt | wt |
| | | GFP | | | | |
| GLV-1h174 | GLV-1h68 | (PSEL)Ruc- | (PSE)NPPA1 | (P11)gusA | wt | wt |
| | | GFP | | | | |
| GLV-1h175 | GLV-1h68 | (PSEL)Ruc- | (PSEL)NPPA1 | (P11)gusA | wt | wt |
| | | GFP | | | | |
| GLV-1h176 | GLV-1h68 | (PSEL)Ruc- | (PSL)NPPA1 | (P11)gusA | wt | wt |
| | | GFP | | | | |
| GLV-1h177 | GLV-1h68 | (PSEL)Ruc- | (PSE)STAT1α | (P11)gusA | wt | wt |
| | | GFP | | | | |
| GLV-1h178 | GLV-1h68 | (PSEL)Ruc- | (PSEL)STAT1α | (P11)gusA | wt | wt |
| | | GFP | | | | |
| GLV-1h179 | GLV-1h68 | (PSEL)Ruc- | (PSL)STAT1α | (P11)gusA | wt | wt |
| | | GFP | | | | |
| GLV-1h180 | GLV-1h68 | (PSEL)Ruc- | (PSE)CPG2 | (P11)gusA | wt | wt |
| | | GFP | | | | |
| GLV-1h181 | GLV-1h68 | (PSEL)Ruc- | (PSEL)CPG2 | (P11)gusA | wt | wt |
| | | GFP | | | | |
| GLV-1h182 | GLV-1h68 | (PSEL)Ruc- | (PSL)CPG2 | (P11)gusA | wt | wt |
| | | GFP | | | | |
| GLV-1h183 | GLV-1h68 | (PSEL)Ruc- | (PSE)Ecad | (P11)gusA | wt | wt |
| | | GFP | | | | |
| GLV-1h184 | GLV-1h68 | (PSEL) Ruc- | (PSE/L)TfR- | (PSE)magA | wt | wt |
| | | GFP | (P7.5)lacZ | | | |
| GLV-1h185 | GLV-1h68 | (PSEL)Ruc- | (PSL)Ecad | (P11)gusA | wt | wt |
| | | GFP | | | | |
| GLV-1h186 | GLV-1h68 | (PSEL) Ruc- | (PSEL)TfR- | (PSEL)FTL | wt | wt |
| | | GFP | (P7.5)lacZ | 498-499InsTC | | |
| GLV-1h187 | GLV-1h68 | (PSEL) Ruc- | (PSEL)TfR- | (PSEL)FTL | wt | wt |
| | | GFP | (P7.5)lacZ | | | |
| GLV-1h188 | GLV-1h68 | (PSEL) Ruc- | (PSEL)TfR- | (PSE)FUKW | wt | wt |
| | | GFP | (P7.5)lacZ | | | |
| GLV-1h189 | GLV-1h68 | (PSEL) Ruc- | (PSEL)TfR- | (PSEL)FUKW | wt | wt |
| | | GFP | (P7.5)lacZ | | | |
| GLV-1h190 | GLV-1h68 | (PSEL) Ruc- | (PSEL)TfR- | (PSL)FUKW | wt | wt |
| | | GFP | (P7.5)lacZ | | | |
| GLV-1h191 | GLV-1h68 | (PSEL)Ruc- | (PSE)STAT1β | (P11)gusA | wt | wt |
| | | GFP | | | | |
| GLV-1h192 | GLV-1h68 | (PSEL)Ruc- | (PSEL)STAT1β | (P11)gusA | wt | wt |
| | | GFP | | | | |
| GLV-1h193 | GLV-1h68 | (PSEL)Ruc- | (PSL)STAT1β | (P11)gusA | wt | wt |
| | | GFP | | | | |
| GLV-1h194 | GLV-1h161 | (PSE)luxE | (PSEL)TfR- | (PSEL)luxCD | wt | wt |
| | | | (P7.5)lacZ | | | |
| GLV-1h195 | GLV-1h161 | (PSEL)Ruc- | (PSE)luxAB | (PSEL)luxCD | wt | wt |
| | | GFP | | | | |
| GLV-1h196 | GLV-1h68 | (PSEL)Ruc- | (PSE)181a | (P11)gusA | wt | wt |
| | | GFP | | | | |
| GLV-1h197 | GLV-1h68 | | | | | |
| GLV-1h198 | GLV-1h68 | (PSEL)Ruc- | (PSL)181a | (P11)gusA | wt | wt |
| | | GFP | | | | |
| GLV-1h199 | GLV-1h68 | (PSEL)Ruc- | (PSE)335 | (P11)gusA | wt | wt |
| | | GFP | | | | |
| GLV-1h201 | GLV-1h68 | (PSEL)Ruc- | (PSL)335 | (P11)gusA | wt | wt |
| | | GFP | | | | |
| GLV-1h202 | GLV-1h68 | | | | | |
| GLV-1h203 | GLV-1h68 | (PSEL)Ruc- | (PSEL)126 | (P11)gusA | wt | wt |
| | | GFP | | | | |
| GLV-1h204 | GLV-1h68 | | | | | |
| GLV-1h205 | GLV-1h68 | (PSEL) Ruc- | (PSEL)TfR- | (PSE)NANOG | wt | wt |
| | | GFP | (P7.5)lacZ | | | |
| GLV-1h208 | GLV-1h68 | (PSEL) Ruc- | (PSEL)TfR- | (PSE)Oct4 | wt | wt |
| | | GFP | (P7.5)lacZ | | | |
| GLV-1h210 | GLV-1h68 | (PSEL)Ruc- | (P7.5E)hEPO | (P11)gusA | wt | wt |
| | | GFP | | | | |
| GLV-1h211 | GLV-1h68 | (PSEL)Ruc- | (PSE)hEPO | (P11)gusA | wt | wt |
| | | GFP | | | | |
| GLV-1h212 | GLV-1h68 | (PSEL)Ruc- | (PSEL)hEPO | (P11)gusA | wt | wt |
| | | GFP | | | | |
| GLV-1h213 | GLV-1h68 | (PSEL)Ruc- | (PSL)hEPO | (P11)gusA | wt | wt |
| | | GFP | | | | |
| GLV-1h214 | GLV-1h68 | (PSEL)Ruc- | (PSE)OspF | (P11)gusA | wt | wt |
| | | GFP | | | | |
| GLV-1h215 | GLV-1h68 | (PSEL)Ruc- | (PSE)OspG | (P11)gusA | wt | wt |
| | | GFP | | | | |
| GLV-1h216 | GLV-1h68 | (PSEL)Ruc- | (PSEL)OspG | (P11)gusA | wt | wt |
| | | GFP | | | | |
| GLV-1h217 | GLV-1h68 | (PSEL)Ruc- | (PSL)OspG | (P11)gusA | wt | wt |
| | | GFP | | | | |
| GLV-1h218 | GLV-1h84 | ko | (PSEL)CBG99- | (PSE)RLN | wt | wt |
| | | | mRFP1 | | | |
| GLV-1h219 | GLV-1h84 | ko | (PSEL)CBG99- | (PSEL)RLN | wt | wt |
| | | | mRFP1 | | | |
| GLV-1h220 | GLV-1h84 | ko | (PSEL)CBG99- | (PSL)RLN | wt | wt |
| | | | mRFP1 | | | |
| GLV-1h221 | GLV-1h160 | (PSE)luxE | (PSEL)luxAB | (P11)gusA | wt | wt |
| GLV-1h222 | GLV-1h68 | (PSEL)Ruc- | (PSE)Ngn3 | (P11)gusA | wt | wt |
| | | GFP | | | | |
| GLV-1h223 | GLV-1h68 | (PSEL)Ruc- | (PSEL)Ngn3 | (P11)gusA | wt | wt |
| | | GFP | | | | |
| GLV-1h224 | GLV-1h68 | (PSEL)Ruc- | (PSL)Ngn3 | (P11)gusA | wt | wt |
| | | GFP | | | | |
| GLV-1h225 | GLV-1h68 | (PSEL)Ruc- | (PSE)hADH | (P11)gusA | wt | wt |
| | | GFP | | | | |
| GLV-1h226 | GLV-1h68 | (PSEL)Ruc- | (PSEL)hADH | (P11)gusA | wt | wt |
| | | GFP | | | | |
| GLV-1h227 | GLV-1h68 | (PSEL)Ruc- | (PSL)hADH | (P11)gusA | wt | wt |
| | | GFP | | | | |
| GLV-1h228 | GLV-1h194 | (PSE)luxE | (PSE)luxAB | (PSEL)luxCD | wt | wt |
| GLV-1h229 | GLV-1h195 | (PSEL)luxE | (PSE)luxAB | (PSEL)luxCD | wt | wt |
| GLV-1h230 | GLV-1h68 | (PSEL)Ruc- | (PSE)Myc-CTR1 | (P11)gusA | wt | wt |
| | | GFP | | | | |
| GLV-1h231 | GLV-1h68 | (PSEL)Ruc- | (PSL)Myc-CTR1 | (P11)gusA | wt | wt |
| | | GFP | | | | |
| GLV-1h232 | GLV-1h68 | (PSEL)Ruc- | (PSE)CTR1 | (P11)gusA | wt | wt |
| | | GFP | | | | |
| GLV-1h233 | GLV-1h68 | (PSEL)Ruc- | (PSE)mPEDF | (P11)gusA | wt | wt |
| | | GFP | | | | |
| GLV-1h234 | GLV-1h68 | (PSEL)Ruc- | (PSEL)mPEDF | (P11)gusA | wt | wt |
| | | GFP | | | | |
| GLV-1h235 | GLV-1h68 | (PSEL)Ruc- | (PSL)mPEDF | (P11)gusA | wt | wt |
| | | GFP | | | | |
| GLV-1h236 | GLV-1h73 | (PSEL)Ruc- | rtfr(PEL) | (PSE)WTCDC6 | wt | wt |
| | | GFP | (P7.5)lacZ | | | |
| GLV-1h237 | GLV-1h73 | (PSEL)Ruc- | rtfr(PEL) | (PSE)MutCDC6 | wt | wt |
| | | GFP | (P7.5)lacZ | | | |
| GLV-1h238 | GLV-1h68 | (PSEL) Ruc- | (PSEL)TfR- | (PSL)CBG99- | wt | wt |
| | | GFP | (P7.5)lacZ | mRFP1 | | |
| GLV-1h239 | GLV-1h68 | (PSEL)Ruc- | (PSE)GLAF-3 | (P11)gusA | wt | wt |
| | | GFP | | | | |
| GLV-1h240 | GLV-1h68 | (PSEL)Ruc- | (PSEL)GLAF-3 | (P11)gusA | wt | wt |
| | | GFP | | | | |
| GLV-1h241 | GLV-1h68 | (PSEL)Ruc- | (PSL)GLAF-3 | (P11)gusA | wt | wt |
| | | GFP | | | | |
| GLV-1h242 | GLV-1h68 | (PSEL)Ruc- | (PE)luxABCDE | (P11)gusA | wt | wt |
| | | GFP | | | | |
| GLV-1h243 | GLV-1h242 | (PSEL)Ruc- | (PE)luxABCDE | (PSE)frp | wt | wt |
| | | GFP | | | | |
| GLV-1h244 | GLV-1h189 | (PSEL) Ruc- | (PSE)hNISa | (PSEL)FUKW | wt | wt |
| | | GFP | | | | |
| GLV-1h245 | GLV-1h189 | (PSEL) Ruc- | (PSEL)hNISa | (PSEL)FUKW | wt | wt |
| | | GFP | | | | |
| GLV-1h246 | GLV-1h189 | (PSEL) Ruc- | (PSL)hNISa | (PSEL)FUKW | wt | wt |
| | | GFP | | | | |
| GLV-1h247 | GLV-1h68 | (PSEL) Ruc- | (PSEL)TfR- | (PSE)IFP | wt | wt |
| | | GFP | (P7.5)lacZ | | | |
| GLV-1h248 | GLV-1h68 | (PSEL) Ruc- | (PSEL)TfR- | (PSEL)IFP | wt | wt |
| | | GFP | (P7.5)lacZ | | | |
| GLV-1h249 | GLV-1h68 | (PSEL) Ruc- | (PSEL)TfR- | (PSL)IFP | wt | wt |
| | | GFP | (P7.5)lacZ | | | |
| GLV-1h250 | GLV-1h190 | (PSEL) Ruc- | (PSEL)TfR- | (PSL)FUKW | wt | wt |
| | | GFP | (P7.5)lacZ | | | |
| GLV-1h251 | GLV-1h68 | (PSE)hNISa | (PSEL)rTrfR- | (P11)gusA | wt | wt |
| | | | (P7.5)lacZ | | | |
| GLV-1h252 | GLV-1h68 | (PSEL)Ruc- | (PSE)hNISa | (P11)gusA | wt | wt |
| | | GFP | | | | |
| GLV-1h253 | GLV-1h71 | ko | (PSEL)TfR- | (PSE)FUKW | wt | wt |
| | | | (P7.5)lacZ | | | |
| GLV-1h254 | GLV-1h71 | ko | (PSEL)TfR- | (PSL)FUKW | wt | wt |
| | | | (P7.5)lacZ | | | |
| GLV-1h255 | GLV-1h68 | (PSEL)Ruc- | (PSE)hMMP9 | (P11)gusA | wt | wt |
| | | GFP | | | | |
| GLV-1h256 | GLV-1h68 | (PSEL)Ruc- | (PSL)hMMP9 | (P11)gusA | wt | wt |
| | | GFP | | | | |
| GLV-1h257 | GLV-1h68 | (PSEL) Ruc- | (PSEL)TfR- | (PSE)mNep- | wt | wt |
| | | GFP | (P7.5)lacZ | tune | | |
| GLV-1h258 | GLV-1h68 | (PSEL) Ruc- | (PSEL)TfR- | (PSEL)mNep- | wt | wt |
| | | GFP | (P7.5)lacZ | tune | | |
| GLV-1h259 | GLV-1h68 | (PSEL) Ruc- | (PSEL)TfR- | (PSL)mNep- | wt | wt |
| | | GFP | (P7.5)lacZ | tune | | |
| GLV-1h260 | GLV-1h68 | (PSE)mNep- | (PSEL)rTrfR- | (P11)gusA | wt | wt |
| | | tune | (P7.5)lacZ | | | |
| GLV-1h261 | GLV-1h68 | (PSEL)mNep- | (PSEL)rTrfR- | (P11)gusA | wt | wt |
| | | tune | (P7.5)lacZ | | | |
| GLV-1h262 | GLV-1h68 | (PSL)mNep- | (PSEL)rTrfR- | (P11)gusA | wt | wt |
| | | tune | (P7.5)lacZ | | | |
| GLV-1h263 | GLV-1h164 | (PSE)mNep- | (PSE)hNET | (PSL)G6-scAb | wt | wt |
| | | tune | | | | |
| GLV-1h264 | GLV-1h164 | (PSEL)mNep- | (PSE)hNET | (PSL)G6-scAb | wt | wt |
| | | tune | | | | |
| GLV-1h265 | GLV-1h164 | (PSL)mNep- | (PSE)hNET | (PSL)G6-scAb | wt | wt |
| | | tune | | | | |
| GLV-1h266 | GLV-1h189 | (PSEL) Ruc- | (PSE)AlstR | (PSEL)FUKW | wt | wt |
| | | GFP | | | | |
| GLV-1h267 | GLV-1h189 | (PSEL) Ruc- | (PSEL)AlstR | (PSEL)FUKW | wt | wt |
| | | GFP | | | | |
| GLV-1h268 | GLV-1h189 | (PSEL) Ruc- | (PSL)AlstR | (PSEL)FUKW | wt | wt |
| | | GFP | | | | |
| GLV-1h269 | GLV-1h189 | (PSEL) Ruc- | (PSE)PEPR1 | (PSEL)FUKW | wt | wt |
| | | GFP | | | | |
| GLV-1h270 | GLV-1h189 | (PSEL) Ruc- | (PSEL)PEPR1 | (PSEL)FUKW | wt | wt |
| | | GFP | | | | |
| GLV-1h271 | GLV-1h189 | (PSEL) Ruc- | (PSL)PEPR1 | (PSEL)FUKW | wt | wt |
| | | GFP | | | | |
| GLV-1h272 | GLV-1h189 | (PSEL) Ruc- | (PSE)LAT4 | (PSEL)FUKW | wt | wt |
| | | GFP | | | | |
| GLV-1h273 | GLV-1h189 | (PSEL) Ruc- | (PSEL)LAT4 | (PSEL)FUKW | wt | wt |
| | | GFP | | | | |
| GLV-1h274 | GLV-1h189 | (PSEL) Ruc- | (PSL)LAT4 | (PSEL)FUKW | wt | wt |
| | | GFP | | | | |
| GLV-1h275 | GLV-1h189 | (PSEL) Ruc- | (PSE)Cyp51 | (PSEL)FUKW | wt | wt |
| | | GFP | | | | |
| GLV-1h276 | GLV-1h189 | (PSEL) Ruc- | (PSEL)Cyp51 | (PSEL)FUKW | wt | wt |
| | | GFP | | | | |
| GLV-1h277 | GLV-1h189 | (PSEL) Ruc- | (PSL)Cyp51 | (PSEL)FUKW | wt | wt |
| | | GFP | | | | |
| GLV-1h284 | GLV-1h189 | (PSEL)Ruc- | (PSE)BMP4 | (PSEL)FUKW | wt | wt |
| | | GFP | | | | |
| GLV-1h285 | GLV-1h189 | (PSEL)Ruc- | (PSEL)BMP4 | (PSEL)FUKW | wt | wt |
| | | GFP | | | | |
| GLV-1h286 | GLV-1h189 | (PSEL)Ruc- | (PSL)BMP4 | (PSEL)FUKW | wt | wt |
| | | GFP | | | | |
| GLV-1h311 | GLV-1h68 | (P_{SEL})Ruc-GFP | (P_{SL})tetO-CBG99- | (P₁₁ₖ)gusA | wt | wt |
| | | | mRFP | | | |
| GLV-1h312 | GLV-1h311 | (P_{7.5k}) -TetR | (P_{SL})tetO-CBG99- | (P₁₁ₖ)gusA | wt | wt |
| | | | mRFP | | | |
| GLV-1h330 | GLV-1h68 | (P_{7.5})tetR | (P_{SEL})rTrfR- | (P₁₁ₖ)gusA | wt | wt |
| | | | (P_{7.5k})LacZ | | | |
| GLV-1h354 | GLV-1h311 | (P_{SEL})tetR | (P_{SL})tetO-CBG99 | (P₁₁ₖ)gusA | wt | wt |
| | | | mRFP1 | | | |

b. Other Oncolytic Viruses

Oncolytic viruses for use in the methods provided here are well known to one of skill in the art and include, for example, vesicular stomatitis virus, see, e.g., U.S. Pat. Nos. 7,731,974, 7,153,510, 6,653,103 and U.S. Pat. Pub. Nos. 2010/0178684, 2010/0172877, 2010/0113567, 2007/0098743, 20050260601, 20050220818 and EP Pat. Nos. 1385466, 1606411 and 1520175; herpes simplex virus, see, e.g., U.S. Pat. Nos. 7,897,146, 7731,952, 7,550,296, 7,537,924, 6,723,316, 6,428,968 and U.S. Pat. Pub. Nos. 2011/0177032, 2011/0158948, 2010/0092515, 2009/0274728, 2009/0285860, 2009/0215147, 2009/0010889, 2007/0110720, 2006/0039894 and 20040009604; retroviruses, see, e.g., U.S. Pat. Nos. 6,689,871, 6,635,472, 5,851,529, 5,716,826, 5,716,613 and U.S. Pat. Pub. No. 20110212530; and adeno-associated viruses, see, e.g., U.S. Pat. Nos. 8,007,780, 7,968,340, 7,943,374, 7,906,111, 7,927,585, 7,811,814, 7,662,627, 7,241,447, 7,238,526, 7,172,893, 7,033,826, 7,001,765, 6,897,045, and 6,632,670.

3. Modification of Viruses

The large genome size of poxviruses, such as the vaccinia viruses in the compositions provided herein, allows large inserts of heterologous DNA and/or multiple inserts of heterologous DNA to be incorporated into the genome (Smith and Moss (1983) Gene 25(1):21-28). Unmodified vaccinia viruses for use in the methods provided herein also can contain genes encoding other heterologous gene products. Thus, the vaccinia viruses in the compositions and methods provided herein can be modified by insertion of 1, 2, 3, 4, 5, 6, 7, 8, 9, 10 or more heterologous DNA molecules. Generally, the one or more heterologous DNA molecules are inserted into a non-essential region of the virus genome. For example, the one or more heterologous DNA molecules are inserted into a locus of the virus genome that is non-essential for replication in proliferating cells, such as tumor cells. Exemplary insertion sites are provided herein below and are known in the art. In some examples, the virus can be modified to express an exogenous or heterologous gene. Exemplary exogenous gene products include proteins and RNA molecules. The modified viruses can express a therapeutic gene product, a detectable gene product, a gene product for manufacturing or harvesting, an antigenic gene product for antibody harvesting, or a viral gene product. The characteristics of such gene products are described herein and elsewhere.

In some examples, the viruses can be modified to express two or more gene products, such as 2, 3, 4, 5, 6, 7, 8, 9, 10 or more gene products, where any combination of the two or more gene products can be one or more detectable gene products, therapeutic gene products, gene products for manufacturing or harvesting or antigenic gene products for antibody harvesting or a viral gene product. In one example, a virus can be modified to express an anticancer gene product. In another example, a virus can be modified to express two or more gene products for detection or two or more therapeutic gene products. In some examples, one or more proteins involved in biosynthesis of a luciferase substrate can be expressed along with luciferase. When two or more exogenous genes are introduced, the genes can be regulated under the same or different regulatory sequences, and the genes can be inserted in the same or different regions of the viral genome, in a single or a plurality of genetic manipulation steps. In some examples, one gene, such as a gene encoding a detectable gene product, can be under the control of a constitutive promoter, while a second gene, such as a gene encoding a therapeutic gene product, can be under the control of an inducible promoter. Methods for inserting two or more genes in to a virus are known in the art and can be readily performed for a wide variety of viruses using a wide variety of exogenous genes, regulatory sequences, and/or other nucleic acid sequences.

The heterologous DNA can be an exemplary gene, including any from the list of human genes and genetic disorders authored and edited by Dr. Victor A. McKusick and his colleagues at Johns Hopkins University and elsewhere, and developed for the World Wide Web by NCBI, the National Center for Biotechnology Information; online, Mendelian Inheritance in Man, OMIM™ Center for Medical Genetics, Johns Hopkins University (Baltimore, Md.), and National Center for Biotechnology Information, National Library of Medicine (Bethesda, Md.), 1999; and those available in public databases, such as PubMed and GenBank (see, e.g., (ncbi.nlm.nih.gov/entrez/query.fcgi?db=OMIM).

In particular, viruses provided herein can be modified to express an anti-tumor antibody, an anti-metastatic gene or metastasis suppressor genes; cell matrix degradative genes; hormones; growth factors; immune modulatory molecules, including a cytokine, such as interleukins or interferons, a chemokine, including CXC chemokines, costimulatory molecules; ribozymes; transporter protein; antibody or fragment thereof; antisense RNA; siRNA; microRNAs; protein ligands; a mitosis inhibitor protein; an antimitotic oligopeptide; an anti-cancer polypeptide; anti-cancer antibiotics; angiogenesis inhibitors; anti-angiogenic factors; tissue factors; a prodrug converting enzyme; genes for tissue regeneration and reprogramming human somatic cells to pluripotency; enzymes that modify a substrate to produce a detectable product or signal or are detectable by antibodies; a viral attenuation factors; a superantigen; proteins that can bind a contrasting agent, chromophore, or a compound of ligand that can be detected; tumor suppressors; cytotoxic protein; cytostatic protein; genes for optical imaging or detection including luciferase, a fluorescent protein such as a green fluorescent protein (GFP) or GFP-like protein, a red fluorescent protein (RFP), a far-red fluorescent protein, a near-infrared fluorescent protein, a yellow fluorescent protein (YFP), an orange fluorescent protein (OFP), a cerulean fluorescent protein (CFP), or a blue fluorescent protein (BFP), and phycobiliproteins from certain cyanobacteria and eukaryotic algae, including phycoerythrins (red) and the phycocyanins (blue); genes for PET imaging; genes for MRI imaging; or genes to alter attenuation of the viruses.

a. Heterologous Nucleic Acid and Exemplary Modifications

Exemplary heterologous genes for modification of viruses herein are known in the art (see e.g. U.S. Pub. Nos. US2003-0059400, US2003-0228261, US2009-0117034, US2009-0098529, US2009-0053244, US2009-0081639 and US2009-0136917; U.S. Pat. Nos. 7,588,767 and 7,763,420; and International Pub. No. WO 2009/139921). A non-limiting description of exemplary genes encoding heterologous proteins for modification of virus strains is set forth in the following table. The sequence of the gene and encoded proteins are known to one of skill in the art from the literature. Hence, provided herein are virus strains, including any of the clonal viruses provided herein, that contain nucleotides encoding any of the heterologous proteins listed in Table 5.

TABLE 5
Exemplary Genes and Gene Products

```
Detectable gene products
  Optical Imaging
  Luciferase
```

bacterial luciferase
luciferase (from Vibrio harveyi or Vibrio fischeri)

luxA
luxB
luxC
luxD
luxE
luxAB
luxCD
luxABCDE

firefly luciferase
Renilla luciferase from Renilla reniformis
Gaussia luciferase
luciferases found among marine arthropods
luciferases that catalyze the oxidation of Cypridina (Vargula) luciferin
luciferases that catalyze the oxidation of Coleoptera luciferin

luciferase photoproteins

aequorin photoprotein to which luciferin is non-covalently bound

click beetle luciferase

CBG99
(CBG99)mRFP1

Fusion Proteins

Ruc-GFP

Fluorescent Proteins
GFP

aequorin from Aequorea victoria
GFP from Aequorea victoria
GFP from Aequorea coerulescens
GFP from the anthozoan coelenterates Renilla reniformis and Renilla koellikeri (sea
pansies)
Emerald (Invitrogen, Carlsbad, CA)
EGFP (Clontech, Palo Alto, CA)
Azami-Green (MBL International, Woburn, MA)
Kaede (MBL International, Woburn, MA)
ZsGreen1 (Clontech, Palo Alto, CA)
CopGFP (Evrogen/Axxora, LLC, San Diego, CA)
Anthozoa reef coral
Anemonia sea anemone
Renilla sea pansy
Galaxea coral
Acropora brown coral
Trachyphyllia stony coral
Pectiniidae stony coral
GFP-like proteins

RFP

RFP from the corallimorph Discosoma (DsRed) (Matz et al. (1999) Nature Biotechnology
17: 969-973)
Heteractis reef coral, Actinia or Entacmaea sea anemone
RFPs from Discosoma variants

mRFP1 (Wang et al. (2004) Proc. Natl. Acad. Sci. U.S.A.101: 16745-9)
mCherry (Wang et al. (2004) PNAS USA.101(48): 16745-9)
tdTomato (Wang et al. (2004) PNAS USA.101(48): 16745-9)
mStrawberry (Wang et al. (2004) PNAS USA.101(48): 16745-9)
mTangerine (Wang et al. (2004) PNAS USA.101(48): 16745-9)
DsRed2 (Clontech, Palo Alto, CA)
DsRed-T1 (Bevis and Glick (2002) Nat. Biotechnol. 20: 83-87)
Anthomedusae J-Red (Evrogen)
Anemonia AsRed2 (Clontech, Palo Alto, CA)

far-red fluorescent protein

TurboFP635
mNeptune monomeric far-red fluorescent protein
Actinia AQ143 (Shkrob et al. (2005) Biochem J. 392(Pt 3): 649-54)
Entacmaea eqFP611 (Wiedenmann et al. (2002) PNAS USA. 99(18): 11646-51)
Discosoma variants

mPlum (Wang et al.. (2004) PNAS USA.101(48): 16745-9)
mRasberry (Wang et al. (2004) PNAS USA.101(48): 16745-9)
Heteractis HcRed1 and t-HcRed (Clontech, Palo Alto, CA)

IFP (infrared fluorescent protein)
near-infrared fluorescent protein
YFP

EYFP (Clontech, Palo Alto, CA)
YPet (Nguyen and Daugherty (2005) Nat Biotechnol. 23(3): 355-60)
Venus (Nagai et al. (2002) Nat. Biotechnol. 20(1): 87-90)
ZsYellow (Clontech, Palo Alto, CA)
mCitrine (Wang et al. (2004) PNAS USA.101(48): 16745-9)

OFP

cOFP (Stratagene, La Jolla, CA)
mKO (MBL International, Woburn, MA)
mOrange (Wang et al.. (2004) PNAS USA.101(48): 16745-9)

CFP

Cerulean (Rizzo (2004) Nat Biotechnol. 22(4): 445-9)
mCFP (Wang et al. (2004) PNAS USA.101(48): 16745-9)
AmCyan1 (Clontech, Palo Alto, CA)
MiCy (MBL International, Woburn, MA)
CyPet (Nguyen and Daugherty (2005) Nat Biotechnol. 23(3): 355-60)

BFP

EBFP (Clontech, Palo Alto, CA);
phycobiliproteins from certain cyanobacteria and eukaryotic algae, phycoerythrins (red)
and the phycocyanins (blue)

R-Phycoerythrin (R-PE)
B-Phycoerythrin (B-PE)
Y-Phycoerythrin (Y-PE)
C-Phycocyanin (P-PC)
R-Phycocyanin (R-PC)
Phycoerythrin 566 (PE 566)
Phycoerythrocyanin (PEC)
Allophycocyanin (APC)
frp Flavin Reductase
CBP Coelenterazine-binding protein 1
PET imaging

Cyp11B1 transcript variant 1
Cyp11B1 transcript variant 2
Cyp11B2
AlstR
PEPR-1
LAT-4 (SLC43A2)
Cyp51 transcript variant 1
Cyp51 transcript variant 2

Transporter proteins
Solute carrier transporter protein families (SLC)

SLC1 solute carrier 1 transporter protein family
SLC1A1, SLC1A2, SLC1A3, SLC1A4, SLC1A5, SLC1A6, SLC1A7
SLC2 solute carrier 2 transporter protein family
SLC2A1, SLC2A2, SLC2A3, SLC2A4, SLC2A5, SLC2A6, SLC2A7, SLC2A8, SLC2A9,
SLC2A10, SLC2A11, SLC2A12, SLC2A13, SLC2A14)
SLC3 solute carrier 3 transporter protein family
SLC3A1, SLC3A2
SLC 4 solute carrier 4 transporter protein family
SLC4A1, SLC4A2, SLC4A3, SLC4A4, SLC4A5, SLC4A6, SLC4A7, SLC4A8, SLC4A9,
SLC4A10, SLC4A11
SLC5 solute carrier 5 transporter protein family
SLC5A1 sodium/glucose cotransporter 1
SLC5A2 sodium/glucose cotransporter 2
SLC5A3 sodium/myo-inositol cotransporter
SLC5A4 low affinity sodium-glucose cotransporter
SLC5A5 sodium/iodide cotransporter
SLC5A6 sodium-dependent multivitamin transporter
SLC5A7 high affinity choline transporter 1
SLC5A8 sodium-coupled monocarboxylate transporter 1
SLC5A9 sodium/glucose cotransporter 4
SLC5A10 sodium/glucose cotransporter 5, isoform 1
sodium/glucose cotransporter 5, isoform 2
sodium/glucose cotransporter 5, isoform 3
sodium/glucose cotransporter 5, isoform 4
SLC5A11 sodium/myo-inositol cotransporter 2, isoform 1
sodium/myo-inositol cotransporter 2, isoform 2
sodium/myo-inositol cotransporter 2, isoform 3
sodium/myo-inositol cotransporter 2, isoform 4
SLC5A12 sodium-coupled monocarboxylate transporter 2, isoform 1
sodium-coupled monocarboxylate transporter 2, isoform 2

Sodium Iodide Symporter (NIS)
hNIS (NM_000453)
hNIS (BC105049)
hNIS (BC105047)
hNIS (non-functional hNIS variant containing an additional 11 aa)

SLC6 solute carrier 6 transporter protein family
SLC6A1 sodium- and chloride-dependent GABA transporter 1
SLC6A2 norepinephrine transporter (sodium-dependent noradrenaline transporter)
SLC6A3 sodium-dependent dopamine transporter
SLC6A4 sodium-dependent serotonin transporter
SLC6A5 sodium- and chloride-dependent glycine transporter 1
SLC6A6 sodium-and chloride-dependent taurine transporter
SLC6A7 sodium-dependent proline transporter
SLC6A8 sodium- and chloride-dependent creatine transporter
SLC6A9 sodium- and chloride-dependent glycine transporter 1, isoform 1
sodium- and chloride-dependent glycine transporter 1, isoform 2
sodium- and chloride-dependent glycine transporter 1, isoform 3
SLC6A10 sodium- and chloride-dependent creatine transporter 2
SLC6A11 sodium- and chloride-dependent GABA transporter 3
SLC6A12 sodium- and chloride-dependent betaine transporter
SLC6A13 sodium- and chloride-dependent GABA transporter 2
SLC6A14 Sodium- and chloride-dependent neutral and basic amino acid transporter B(0+)
SLC6A15 Orphan sodium- and chloride-dependent neurotransmitter transporter NTT73
SLC6A16 Orphan sodium- and chloride-dependent neurotransmitter transporter NTT5
SLC6A17 Orphan sodium- and chloride-dependent neurotransmitter transporter NTT4
Sodium SLC6A18 Sodium- and chloride-dependent transporter XTRP2
SLC6A19 Sodium-dependent neutral amino acid transporter B(0)
SLC6A20 Sodium- and chloride-dependent transporter XTRP3
Norepinephrine Transporter (NET)

Human Net (hNET) transcript variant 1 (NM_001172504)
Human Net (hNET) transcript variant 2 (NM_001172501)
Human Net (hNET) transcript variant 3 (NM_001043)
Human Net (hNET) transcript variant 4 (NM_001172502)
Non-Human Net

SLC7 solute carrier 7 transporter protein family
SLC7A1, SLC7A2, SLC7A3, SLC7A4, SLC7A5, SLC7A6, SLC7A7, SLC7A8, SLC7A9,
SLC7A10, SLC7A11, SLC7A13, SLC7A14
SLC8 solute carrier 8 transporter protein family
SLC8A1, SLC8A2, SLC8A3
SLC9 solute carrier 9 transporter protein family
SLC9A1, SLC9A2, SLC9A3, SLC9A4, SLC9A5, SLC9A6, SLC9A7, SLC9A8, SLC9A9,
SLC9A10, SLC9A11
SLC10 solute carrier 10 transporter protein family
SLC10A1, SLC10A2, SLC10A3, SLC10A4, SLC10A5, SLC10A6, SLC10A7
SLC11 solute carrier 11 transporter protein family
SLC11A1
SCL11A2 or hDMT

SLC11A2 transcript variant 4
SLC11A2 transcript variant 1
SLC11A2 transcript variant 2
SLC11A2 transcript variant 3
SLC11A2 transcript variant 5
SLC11A2 transcript variant 6
SLC11A2 transcript variant 7

SLC12 solute carrier 12 transporter protein family
SLC12A1, SLC12A1, SLC12A2, SLC12A3, SLC12A4, SLC12A5, SLC12A6, SLC12A7,
SLC12A8, SLC12A9
SLC13 solute carrier 13 transporter protein family
SLC13A1, SLC13A2, SLC13A3, SLC13A4, SLC13A5
SLC14 solute carrier 14 transporter protein family
SLC14A1, SLC14A2
SLC15 solute carrier 15 transporter protein family
SLC15A1, SLC15A2, SLC15A3, SLC15A4
SLC16 solute carrier 16 transporter protein family
SLC16A1, SLC16A2, SLC16A3, SLC16A4, SLC16A5, SLC16A6, SLC16A7, SLC16A8,
SLC16A9, SLC16A10, SLC16A11, SLC16A12, SLC16A13, SLC16A14
SLC17 solute carrier 17 transporter protein family
SLC17A1, SLC17A2, SLC17A3, SLC17A4, SLC17A5, SLC17A6, SLC17A7, SLC17A8
SLC18 solute carrier 18 transporter protein family
SLC18A1, SLC18A2, SLC18A3
SLC19 solute carrier 19 transporter protein family
SLC19A1, SLC19A2, SLC19A3
SLC20 solute carrier 20 transporter protein family
SLC20A1, SLC20A2
SLC21 solute carrier 21 transporter protein family

subfamily 1; SLCO1A2, SLCO1B1, SLCO1B3, SLCO1B4, SLCO1C1
subfamily 2; SLCO2A1, SLCO2B1
subfamily 3; SLCO3A1
subfamily 4; SLCO4A1, SLCO4C1
subfamily 5; SLCO5A1

SLC22 solute carrier 22 transporter protein family
SLC22A1, SLC22A2, SLC22A3, SLC22A4, SLC22A5, SLC22A6, SLC22A7, SLC22A8,
SLC22A9, SLC22A10, SLC22A11, SLC22A12, SLC22A13, SLC22A14, SLC22A15,
SLC22A16, SLC22A17, SLC22A18, SLC22A19, SLC22A20
SLC23 solute carrier 23 transporter protein family
SLC23A1, SLC23A2, SLC23A3, SLC23A4
SLC24 solute carrier 24 transporter protein family
SLC24A1, SLC24A2, SLC24A3, SLC24A4, SLC24A5, SLC24A6
SLC25 solute carrier 25 transporter protein family
SLC25A1, SLC25A2, SLC25A3, SLC25A4, SLC25A5, SLC25A6, SLC25A7, SLC25A8,
SLC25A9, SLC25A10, SLC25A11, SLC25A12, SLC25A13, SLC25A14, SLC25A15,
SLC25A16, SLC25A17, SLC25A18, SLC25A19, SLC25A20, SLC25A21, SLC25A22,
SLC25A23, SLC25A24, SLC25A25, SLC25A26, SLC25A27, SLC25A28, SLC25A29,
SLC25A30, SLC25A31, SLC25A32, SLC25A33, SLC25A34, SLC25A35, SLC25A36,
SLC25A37, SLC25A38, SLC25A39, SLC25A40, SLC25A41, SLC25A42, SLC25A43,
SLC25A44, SLC25A45, SLC25A46
SLC26 solute carrier 26 transporter protein family
SLC26A1, SLC26A2, SLC26A3, SLC26A4, SLC26A5, SLC26A6, SLC26A7, SLC26A8,
SLC26A9, SLC26A10, SLC26A11
SLC27 solute carrier 27 transporter protein family
SLC27A1, SLC27A2, SLC27A3, SLC27A4, SLC27A5, SLC27A6
SLC28 solute carrier 28 transporter protein family
SLC28A1, SLC28A2, SLC28A3
SLC29 solute carrier 29 transporter protein family
SLC29A1, SLC29A2, SLC29A3, SLC29A4
SLC30 solute carrier 30 transporter protein family
SLC30A1, SLC30A2, SLC30A3, SLC30A4, SLC30A5, SLC30A6, SLC30A7, SLC30A8,
SLC30A9, SLC30A10
SLC31 solute carrier 31 transporter protein family
SLC31A1
SLC32 solute carrier 32 transporter protein family
SLC32A1
SLC33 solute carrier 33 transporter protein family
SLC33A1
SLC34 solute carrier 34 transporter protein family
SLC34A1, SLC34A2, SLC34A3
SLC35 solute carrier 35 transporter protein family

subfamily A; SLC35A1, SLC35A2, SLC35A3, SLC35A4, SLC35A5
subfamily B; SLC35B1, SLC35B2, SLC35B3, SLC35B4
subfamily C; SLC35C1, SLC35C2
subfamily D; SLC35D1, SLC35D2, SLC35D3
subfamily E; SLC35E1, SLC35E2, SLC35E3, SLC35E4

SLC36 solute carrier 36 transporter protein family
SLC36A1, SLC36A2, SLC36A3, SLC36A4
SLC37 solute carrier 37 transporter protein family
SLC37A1, SLC37A2, SLC37A3, SLC37A4
SLC38 solute carrier 38 transporter protein family
SLC38A1, SLC38A2, SLC38A3, SLC38A4, SLC38A5, SLC38A6
SLC39 solute carrier 39 transporter protein family
SLC39A1, SLC39A2, SLC39A3, SLC39A4, SLC39A5, SLC39A6, SLC39A7, SLC39A8,
SLC39A9, SLC39A10, SLC39A11, SLC39A12, SLC39A13, SLC39A14
SLC40 solute carrier 40 transporter protein family
SLC40A1
SLC41 solute carrier 41 transporter protein family
SLC41A1, SLC41A2, SLC41A3
SLC42 solute carrier 42 transporter protein family
RHAG, RhBG, RhCG
SLC43 solute carrier 43 transporter protein family
SLC43A1
SLC43A2
SLC43A3
SLC44 solute carrier 44 transporter protein family
SLC44A1, SLC44A2, SLC44A3, SLC44A4, SLC44A5
SLC45 solute carrier 45 transporter protein family
SLC45A1, SLC45A2, SLC54A3, SLC45A4
SLC46 solute carrier 46 transporter protein family
SLC46A1, SLC46A2
SLC47 solute carrier 47 transporter protein family
SLC47A1, SLC47A2

MRI Imaging

Human transferrin receptor
Human transferrin receptor
Mouse transferrin receptor
Human ferritin light chain (FTL)
Human ferritin heavy chain
FTL 498-199InsTC, a mutated form of the ferritin light chain
Bacterial ferritin

E. coli
E. coli strain K12
S. aureus strain MRSA252
S. aureus strain NCTC 8325
H. pylori B8

bacterioferritin
codon optimized bacterioferritin
MagA

Enzymes that modify a substrate to produce a detectable product or signal, or are detectable
by antibodies

alpha-amylase
alkaline phosphatase
secreted alkaline phosphatase
peroxidase
T4 lysozyme
oxidoreductase
pyrophosphatase

Therapeutic genes
therapeutic gene product
antigens

tumor specific antigens
tumor-associated antigens
tissue-specific antigens
bacterial antigens
viral antigens
yeast antigens
fungal antigens
protozoan antigens
parasite antigens
mitogens

an antibody or fragment thereof
virus-specific antibodies
antisense RNA
siRNA

siRNA directed against expression of a tumor-promoting gene

an oncogene
growth factor
angiogenesis promoting gene
a receptor

siRNA molecule directed against expression of any gene essential for cell growth, cell
replication or cell survival.
siRNA molecule directed against expression of any gene that stabilizes the cell membrane
or otherwise limits the number of tumor cell antigens released from the tumor cell.

protein ligands
an antitumor oligopeptide
an antimitotic peptide

tubulysin,
phomopsin
hemiasterlin
taltobulin (HTI-286, 3)
cryptophycin

a mitosis inhibitor protein
an antimitotic oligopeptide
an anti-cancer polypeptide antibiotic
anti-cancer antibiotics

tissue factors

Tissue Factor (TF)
αvβ3-integrin RGD fusion protein

Immune modulatory molecules

GM-CSF
MCP-1 or CCL2 (Monocyte Chemoattractant Protein-1) Human
MCP-1 murine
IP-10 or Chemokine ligand 10 (CXCL10)
LIGHT
P60 or SEQSTM1 (Sequestosome 1 transcript variant 1)
P60 or SEQSTM1 (Sequestosome 1 transcript variant 3)
P60 or SEQSTM1 (Sequestosome 1 transcript variant 2)
OspF
OspG
STAT1alpha
STAT1beta

Interleukins

IL-18 (Interleukin-18)
IL-11 (Interleukin-11)
IL-6 (Interleukin-6)
sIL-6R-IL-6
interleukin-12
interleukin-1
interleukin-2
IL-24 (Interleukin-24)
IL-24 transcript variant 1
IL-24 transcript variant 4
IL-24 transcript variant 5
IL-4
IL-8
IL-10

chemokines

IP-10 (CXCL)
Thrombopoietin
members of the C—X—C and C-C chemokine families
RANTES
MIP1-alpha
MIP1-beta
MIP-2

CXC chemokines

GROα
GROβ (MIP-2)
GROγ
ENA-78
LDGF-PPBP
GCP-2
PF4
Mig
IP-10
SDF-1α/β
BUNZO/STRC33
I-TAC
BLC/BCA-1
MDC
TECK
TARC
HCC-1
HCC-4
DC-CK1
MIP-3α
MIP-3β
MCP-2
MCP-3 (Monocyte Chemoattractant Protein-3, CCL7)
MCP-4
MCP-5 (Monocyte Chemoattractant Protein-5; CCL12)
Eotaxin (CCL11)
Eotaxin-2/MPIF-2
I-309
MIP-5/HCC-2
MPIF-1
6Ckine
CTACK
MEC
lymphotactin
fractalkine

Immunoglobulin superfamily of cytokines

B7.1
B7.2

Anti-angiogenic genes/angiogenesis inhibitors

Human plasminogen k5 domain (hK5)
PEDF (SERPINF1) (Human)
PEDF (mouse)
anti-VEGF single chain antibody (G6)
anti-DLL4 s.c. antibody GLAF-3
tTF-RGD (truncated human tissue factor protein fused to an RGD peptide)

viral attenuation factors

Interferons

IFN-γ
IFN-α
IFN-β

Antibody or scFv

Therapeutic antibodies (i.e. anticancer antibodies)

Rituximab (RITUXAN)
ADEPT
Trastuzumab (Herceptin)
Tositumomab (Bexxar)
Cetuximab (Erbitux)
Ibritumomab (90Y-Ibritumomab tiuxetan; Zevalin)
Alemtuzumab (Campath-1H)
Epratuzumab (Lymphocide)
Gemtuzumab ozogamicin (Mylotarg)
Bevacizumab (Avastin) and Edrecolomab (Panorex)
Infliximab

Metastasis suppressor genes

NM23 or NME1 Isoform a
NM23 or NME1 Isoform b

Anti-metastatic genes

E-Cad
Gelsolin
LKB1 (STK11)
RASSF1
RASSF2
RASSF3
RASSF4
RASSF5
RASSF6
RASSF7
RASSF8
Syk
TIMP-1 (Tissue Inhibitor of Metalloproteinase Type-1)
TIMP-2 (Tissue Inhibitor of Metalloproteinase Type-2)
TIMP-3 (Tissue Inhibitor of Metalloproteinase Type-3)
TIMP-4 (Tissue Inhibitor of Metalloproteinase Type-4)
BRMS-1
CRMP-1
CRSP3
CTGF
DRG1
KAI1
KiSS1 (kisspeptin)
kisspeptin fragments

kisspeptin-10
kisspeptin-13
kisspeptin-14
kisspeptin-54

Mkk4
Mkk6
Mkk7
RKIP
RHOGDI2
SSECKS
TXNIP/VDUP1

Cell matrix-degradative genes

Relaxin 1
hMMP9

Hormones

Human Erythropoietin (EPO)

MicroRNAs

pre-miRNA 181a (sequence inserted into viral genome)
miRNA 181a
mmu-miR-181a MIMAT0000210 mature miRNA 181a
pre-miRNA 126 (sequence inserted into the vial genome)
miRNA 126
hsa-miR-126 MI000471
hsa-miR-126 MIMAT0000445
pre-miRNA 335 (sequence inserted into the viral genome)
miRNA 335
hsa-miR-335 MI0000816
hsa-miR-335 MIMAT0000765

Genes for tissue regeneration and reprogramming Human somatic cells to pluripotency

nAG
Oct4
NANOG
Ngn (Neogenin 1) transcript variant 1
Ngn (Neogenin 1) transcript variant 2
Ngn (Neogenin 1) transcript variant 3
Ngn3
Pdx1
Mafa

Additional Genes

Myc-CTR1
FCU1
mMnSOD
HACE1
nppa1
GCP-2 (Granulocyte Chemotactic Protein-2, CXCL6)
hADH
Wildtype CDC6
Mut CDC6
GLAF-3 anti-DLL4 scFv
GLAF-4 anti-FAP (Fibroblast Activation Protein) scFv (Brocks et al., (2001) Mol.
Medicine 7(7): 461-469)
GLAF-5 anti-FAP scFv
BMP4
wildtype F14.5L

Other Proteins

WT1
p53
Pseudomonas exotoxin
diphtheria toxin
Arf or p16
Bax
Herpes simplex virus thymidine kinase
E. coli purine nucleoside phosphorylase
angiostatin
endostatin
Rb
BRCA1
cystic fibrosis transmembrane regulator (CFTR)
Factor VIII
low density lipoprotein receptor
alpha-galactosidase
beta-glucocerebrosidase
insulin
parathyroid hormone
alpha-1-antitrypsin
rsCD40L
Fas-ligand
TRAIL
TNF
microcin E492
xanthine guanine phosphoribosyltransferase (XGPRT)
E. coli guanine phosphoribosyltransferase (gpt)
hyperforin
endothelin-1 (ET-1)
connective tissue growth factor (CTGF)
vascular endothelial growth factor (VEGF)
cyclooxygenase
COX-2
cyclooxygenase-2 inhibitor
MPO (Myeloperoxidase)
Apo A1 (Apolipoprotein A1)
CRP (C Reactive Protein)
Fibrinogen
SAP (Serum Amyloid P)
FGF-basic (Fibroblast Growth Factor-basic)
PPAR-agonist
PE37/TGF-alpha fusion protein

Replacement of the A34R gene with another A34R gene from a different strain in order to
increase the EEV form of the virus

A34R from VACV IHD-J
A34R with a mutation at codon 151 (Lys 151 to Asp)
A34R with a mutation at codon 151 (Lys 151 to Glu)

Non-coding Sequence

Non-proteins
Non-coding nucleic acid

Ribozymes

Group I introns
Group II introns
RNaseP
hairpin ribozymes
hammerhead ribozymes

Prodrug converting enzymes

varicella zoster thymidine kinase
cytosine deaminase
purine nucleoside phosphorylase (e.g., from E. coli)
beta lactamase
carboxypeptidase G2
carboxypeptidase A
cytochrome P450

cytochrome P450-2B1
cytochrome P450-4B1

horseradish peroxidase
nitroreductase
rabbit carboxylesterase
mushroom tyrosinase
beta galactosidase (lacZ) (i.e., from E. coli)
beta glucuronidase (gusA)
thymidine phosphorylase
deoxycytidine kinase
linamarase

Proteins detectable by antibodies

chloramphenicol acetyl transferase
hGH

Viral attenuation factors

virus-specific antibodies

mucins
thrombospondin

tumor necrosis factors (TNFs)

TNFα

Superantigens
Toxins

diphtheria toxin
Pseudomonas exotoxin
Escherichia coli Shiga toxin
Shigella toxin
Escherichia coli Verotoxin 1
Toxic Shock Syndrome Toxin 1
Exfoliating Toxins (EXft)
Streptococcal Pyrogenic Exotoxin (SPE) A, B and C
Clostridial Perfringens Enterotoxin (CPET)
staphylococcal enterotoxins
SEA, SEB, SEC1, SEC2, SED, SEE and SEH
Mouse Mammary Tumor Virus proteins (MMTV)
Streptococcal M proteins
Listeria monocytogenes antigen p60
mycoplasma arthritis superantigens

Proteins that can bind a contrasting agent, chromophore, or a compound or ligand that can be
detected

siderophores

enterobactin
salmochelin
yersiniabactin
aerobactin

Growth Factors

platelet-derived growth factor (PDG-F)
keratinocyte growth factor (KGF)
insulin-like growth factor-1 (IGF-1)
insulin-like growth factor-binding proteins (IGFBPs)
transforming growth factor (TGF-alpha)
Growth factors for blood cells

Granulocyte Colony Stimulating Factor (G-CSF)

growth factors that can boost platelets

Other Groups

BAC (Bacterial Artificial Chromosome) encoding several or all proteins of a specific

pathway, e.g. wound healing-pathway

MAC (Mammalian Artificial Chromosome) encoding several or all proteins of a specific

pathway, e.g. wound healing-pathway

tumor antigen
RNAi
ligand binding proteins
proteins that can induce a signal detectable by MRI
angiogenins
photosensitizing agents
anti-metabolites
signaling modulators
chemotherapeutic compounds
lipases
proteases
pro-apoptotic factors
anti-cancer vaccine

antigen vaccines
whole cell vaccines (i.e., dendritic cell vaccines)
DNA vaccines
anti-idiotype vaccines

tumor suppressors
cytotoxic protein
cytostatic proteins
costimulatory molecules

cytokines and chemokines

cancer growth inhibitors
gene therapy
BCG vaccine for bladder cancer

Proteins that interact with host cell proteins

i. Diagnostic or Reporter Gene Products

In some examples, the viruses provided herein contain nucleic acids that encode a detectable protein or a protein capable of inducing a detectable signal. Expression of such proteins allows detection of the virus in vitro and in vivo. A variety of detectable gene products, such as detectable proteins are known in the art, and can be used with the viruses provided herein.

Exemplary of such proteins are enzymes that can catalyze a detectable reaction or catalyze formation of a detectable product, such as, for example, luciferases, such as a click beetle luciferase, a Renilla luciferase, a firefly luciferase or beta-glucuronidase (GusA). Also exemplary of such proteins are proteins that emit a detectable signal, including fluorescent proteins, such as a green fluorescent protein (GFP) or a red fluorescent protein (RFP). A variety of DNA sequences encoding proteins that can emit a detectable signal or that can catalyze a detectable reaction, such as luminescent or fluorescent proteins, are known and can be used in the viruses and methods provided herein. Transformation and expression of these genes in viruses can permit detection of viral infection, for example, using a low light and/or fluorescence imaging camera.

Exemplary genes encoding light-emitting proteins include, for example, genes from bacterial luciferase from Vibrio harveyi (Belas et al., Science 218 (1982), 791-793), bacterial luciferase from Vibrio fischeri (Foran and Brown, Nucleic acids Res. 16 (1988), 177), firefly luciferase (de Wet et al., (1987) Mol. Cell. Biol. 7:725-737), aequorin from Aequorea victoria (Prasher et al., (19897) Biochem. 26:1326-1332), Renilla luciferase from Renilla reniformis (Lorenz et al, (1991) Proc Natl Acad Sci USA 88:4438-4442). The luxA and luxB genes of bacterial luciferase can be fused to produce the fusion gene (Fab₂), which can be expressed to produce a fully functional luciferase protein (Escher et al., (1989) Proc Natl Acad Sci USA 86:6528-6532). In some examples, luciferases expressed by viruses can require exogenously added substrates such as decanal or coelenterazine for light emission. In other examples, viruses can express a complete lux operon, which can include proteins that can provide luciferase substrates such as decanal. For example, viruses containing the complete lux operon sequence, when injected intraperitoneally, intramuscularly, or intravenously, allowed the visualization and localization of microorganisms in live mice indicating that the luciferase light emission can penetrate the tissues and can be detected externally (Contag et al. (1995) Mol. Microbiol. 18: 593-603).

Exemplary fluorescent proteins include green fluorescent protein from Aequorea victoria (Prasher et al., Gene 111: 229-233 (1987), and GFP variants and variants of GFP-like proteins. Such fluorescent proteins include monomeric, dimeric and tetrameric fluorescent proteins. Exemplary monomeric fluorescent proteins include, but are not limited to: violet fluorescent proteins, such as for example, Sirius; blue fluorescent proteins, such as for example, Azurite, EBFP, SBFP2, EBFP2, TagBFP; cyan fluorescent proteins, such as for example, mTurquoise, eCFP, Cerulean (Rizzo, (2004) Nat. Biotechnol. 22(4):445-449), SCFP, TagCFP, mTFP1, mCFP, AmCyan1, MiCy, CyPet (Nguyen and Daugherty, (2005) Nat Biotechnol. 23(3):355-360); green fluorescent proteins, such as for example, GFP, mUkG1, aAG1, AcGFP1, TagGFP2, EGFP, mWasabi, EmGFP (Emerald), Azami-Green, Kaede, ZsGreen1 and CopGFP; yellow fluorescent proteins, such as for example; TagYFP, EYFP, Topaz, SYFP2, YPet (Nguyen and Daugherty, (2005) Nat. Biotechnol. 23(3):355-360), Venus (Nagai et al. (2002) Nat. Biotechnol. 20(1):87-90), mCitrine; orange fluorescent proteins, such as for example, cOFP, mKO, mKO2, mOrange, mOrange2, red fluorescent proteins, such as for example, Discosoma RFP (DsRed) isolated from the corallimorph Discosoma (Matz et al. (1999) Nature Biotechnology 17: 969-973), mRFP1, TagRFP, TagRFPt, Discosoma variants mStrawberry, mRuby, mCherry, tdTomato, mTangerine, DsRed2, DsRed-T1 (Bevis and Glick, (2002) Nat. Biotechnol., 20: 83-87), Anthomedusae J-Red, Anemonia AsRed2; far red fluorescent proteins, such as for example, Actinia AQ143 (Shkrob et al. (2005) Biochem J. 392(Pt 3):649-54), Entacmaea eqFP611 (Wiedenmann et al. (2002) Proc. Natl. Acad. Sci. USA. 99(18):11646-11651), Discosoma variants mRasberry, mKate2, mPlum, and mNeptune, Heteractis HcRed1 and t-HcRed; and fluorescent proteins having an increased stokes shift (i.e. >100 nm distance between excitation and emission spectra), such as for example, Sapphire, T-Sapphire, mAmetrine, and mKeima; Near-infrared FPs, such as and IFP1.4 (Scherbo et al. (2007) Nat Methods 4:741-746), eqFP650 and eqFP670. Exemplary dimeric and tetrameric fluorescent proteins include, but are not limited to: AmCyan1, Midori-Ishi Cyan, copGFP (ppluGFP2), TurboGFP, ZsGreen, TurboYFP, ZsYellow1, TurboRFP, tdTomato, DsRed2, DsRed-Express, DsRed-Express2, DsRed-Max, AsRed2, TurboFP602, RFP611, Katushka (TurboFP635), Katushka2, and AQ143. Excitation and emission spectra for exemplary fluorescent proteins are well-known in the art (see also e.g. Chudakov et al. (2010) Physiol Rev 90, 1102-1163).

Exemplary detectable proteins also include proteins that can bind a contrasting agent, chromophore, or a compound or ligand that can be detected, such as a transferrin receptor or a ferritin; and reporter proteins, such as E. coli β-galactosidase, β-glucuronidase, xanthine-guanine phosphoribosyltransferase (gpt).

Also exemplary of detectable proteins are gene products that can specifically bind a detectable compound, including, but not limited to receptors, metal binding proteins (e.g., siderophores, ferritins, transferrin receptors), ligand binding proteins, and antibodies. Also exemplary of detectable proteins are transporter proteins that can bind to and transport detectable molecules. Such molecules can be used for detection of the virus, such as for applications involving imaging. Any of a variety of detectable compounds can be used, and can be imaged by any of a variety of known imaging methods. Exemplary compounds include receptor ligands and antigens for antibodies. The ligand can be labeled according to the imaging method to be used. Exemplary imaging methods include, but are not limited to, X-rays, magnetic resonance methods, such as magnetic resonance imaging (MRI) and magnetic resonance spectroscopy (MRS), and tomographic methods, including computed tomography (CT), computed axial tomography (CAT), electron beam computed tomography (EBCT), high resolution computed tomography (HRCT), hypocycloidal tomography, positron emission tomography (PET), single-photon emission computed tomography (SPECT), spiral computed tomography and ultrasonic tomography.

Labels appropriate for X-ray imaging are known in the art, and include, for example, Bismuth (III), Gold (III), Lanthanum (III) or Lead (II); a radioactive ion, such as ⁶⁷Copper, ⁶⁷Gallium, ⁶⁸Gallium, ¹¹¹Indium, ¹¹³Indium, ¹²³Iodine, ¹²⁵Iodine, ¹³¹Iodine, ¹⁹⁷Mercury, ²⁰³Mercury, ¹⁸⁶Rhenium, ¹⁸⁸Rhenium, ⁹⁷Rubidium, ¹⁰³Rubidium, ⁹⁹Technetium or ⁹⁰Yttrium; a nuclear magnetic spin-resonance isotope, such as Cobalt (II), Copper (II), Chromium (III), Dysprosium (III), Erbium (III), Gadolinium (III), Holmium (III), Iron (II), lion (III), Manganese (II), Neodymium (III), Nickel (II), Samarium (III), Terbium (III), Vanadium (II) or Ytterbium (III); or rhodamine or fluorescein.

Labels appropriate for magnetic resonance imaging are known in the art, and include, for example, gadolinium chelates and iron oxides. Use of chelates in contrast agents is known in the art. Labels appropriate for tomographic imaging methods are known in the art, and include, for example, β-emitters such as ¹¹C, ¹³N, ¹⁵O or ⁶⁴Cu or γ-emitters such as ¹²³I. Other exemplary radionuclides that can, be used, for example, as tracers for PET include ⁵⁵Co, ⁶⁷Ga, ⁶⁸Ga, ⁶⁰Cu(II), ⁶⁷Cu(II), ⁵⁷Ni, ⁵²Fe and ¹⁸F (e.g., ¹⁸F-fluorodeoxyglucose (FDG)). Examples of useful radionuclide-labeled agents are a ⁶⁴Cu-labeled engineered antibody fragment (Wu et al. (2002) PNAS USA 97: 8495-8500), ⁶⁴Cu-labeled somatostatin (Lewis et al. (1999) J. Med. Chem. 42: 1341-1347), ⁶⁴Cu-pyruvaldehyde-bis(N4-methylthiosemicarbazone) (⁶⁴Cu-PTSM) (Adonai et al. (2002) PNAS USA 99: 3030-3035), ⁵²Fe-citrate (Leenders et al. (1994) J. Neural. Transm. Suppl. 43: 123-132), ⁵²Fe/^{52m}Mn-citrate (Calonder et al. (1999) J. Neurochem. 73: 2047-2055) and ⁵²Fe-labeled iron (III) hydroxide-sucrose complex (Beshara et al. (1999) Br. J. Haematol. 104: 288-295, 296-302).

Exemplary of detectable proteins are transporter proteins that can bind to and transport detectable molecules, such as human epinephrine transporter (hNET) or sodium iodide symporter (NIS) that can bind to and transport detectable molecules, such as MIBG and other labeled molecules (e.g., Na¹²⁵I), into the cell.

The viruses can be modified for purposes of using the viruses for imaging, including for the purpose of dual imaging in vitro and/or in vivo to detect two or more detectable gene products, gene products that produce a detectable signal, gene products that can bind a detectable compound, or gene products that can bind other molecules to form a detectable product. In some examples, the two or more gene products are expressed by different viruses, whereas in other examples the two or more gene products are produced by the same virus. For example, a virus can express a gene product that emits a detectable signal and also express a gene product that catalyzes a detectable reaction. In other examples, a virus can express one or more gene products that emit a detectable signal, one or more gene products that catalyze a detectable reaction, one or more gene products that can bind a detectable compound or that can form a detectable product, or any combination thereof. Any combination of such gene products can be expressed by the viruses provided herein and can be used in combination with any of the methods provided herein. Imaging of such gene products can be performed, for example, by various imaging methods as described herein and known in the art (e.g., fluorescence imaging, MRI, PET, among many other methods of detection). Imaging of gene products can also be performed using the same method, whereby gene products are distinguished by their properties, such as by differences in wavelengths of light emitted. For example, a virus can express more than one fluorescent protein that differs in the wavelength of light emitted (e.g., a GFP and an RFP). In another non-limiting example, an RFP can be expressed with a luciferase. In yet other non-limiting examples, a fluorescent gene product can be expressed with a gene product, such as a ferritin or a transferrin receptor, used for magnetic resonance imaging. A virus expressing two or more detectable gene products or two or more viruses expressing two or more detectable gene products can be imaged in vitro or in vivo using such methods. In some examples the two or more gene products are expressed as a single polypeptide, such as a fusion protein. For example a fluorescent protein can be expressed as a fusion protein with a luciferase protein.

ii. Therapeutic Gene Products

The viruses for use in the methods provided herein can contain a heterologous nucleic acid molecule that encodes one or more therapeutic gene products. Therapeutic gene products include products that cause cell death or cause an anti-tumor immune response. A variety of therapeutic gene products, such as toxic or apoptotic proteins, or siRNA, are known in the art, and can be used with the viruses provided herein. The therapeutic genes can act by directly killing the host cell, for example, as a channel-forming or other lytic protein, or by triggering apoptosis, or by inhibiting essential cellular processes, or by triggering an immune response against the cell, or by interacting with a compound that has a similar effect, for example, by converting a less active compound to a cytotoxic compound.

Exemplary therapeutic gene products that can be expressed by the viruses provided herein include, but are not limited to, gene products (i.e., proteins and RNAs), including those useful for tumor therapy, such as, but not limited to, an anticancer agent, an antimetastatic agent, or an antiangiogenic agent. For example, exemplary proteins useful for tumor therapy include, but are not limited to, tumor suppressors, cytostatic proteins and costimulatory molecules, such as a cytokine, a chemokine, or other immunomodulatory molecules, an anticancer antibody, such as a single-chain antibody, antisense RNA, siRNA, prodrug converting enzyme, a toxin, a mitosis inhibitor protein, an antitumor oligopeptide, an anticancer polypeptide antibiotic, an angiogenesis inhibitor, or tissue factor. For example, a large number of therapeutic proteins that can be expressed for tumor treatment in the viruses and methods provided herein are known in the art, including, but not limited to, a transporter, a cell-surface receptor, a cytokine, a chemokine, an apoptotic protein, a mitosis inhibitor protein, an antimitotic oligopeptide, an antiangiogenic factor (e.g., hk5), angiogenesis inhibitors (e.g., plasminogen kringle 5 domain, anti-vascular endothelial growth factor (VEGF) scAb, tTF-RGD, truncated human tissue factor-αᵥβ₃-integrin RGD peptide fusion protein), anticancer antibodies, such as a single-chain antibody (e.g., an antitumor antibody or an antiangiogenic antibody, such as an anti-VEGF antibody or an anti-epidermal growth factor receptor (EGFR) antibody), a toxin, a tumor antigen, a prodrug converting enzyme, a ribozyme, RNAi, and siRNA.

Additional therapeutic gene products that can be expressed by the oncolytic reporter viruses include, but are not limited to, cell matrix degradative genes, such as but not limited to, relaxin-1 and MMP9, and genes for tissue regeneration and reprogramming human somatic cells to pluripotency, such as but not limited to, nAG, Oct4, NANOS, Neogenin-1, Ngn3, Pdx1 and Mafa.

Costimulatory molecules for the methods provided herein include any molecules which are capable of enhancing immune responses to an antigen/pathogen in vivo and/or in vitro. Costimulatory molecules also encompass any molecules which promote the activation, proliferation, differentiation, maturation or maintenance of lymphocytes and/or other cells whose function is important or essential for immune responses.

An exemplary, non-limiting list of therapeutic proteins includes tumor growth suppressors such as IL-24, WT1, p53, Pseudomonas exotoxin, diphtheria toxin, Arf, Bax, HSV TK, E. coli purine nucleoside phosphorylase, angiostatin and endostatin, p16, Rb, BRCA1, cystic fibrosis transmembrane regulator (CFTR), Factor VIII, low density lipoprotein receptor, beta-galactosidase, alpha-galactosidase, beta-glucocerebrosidase, insulin, parathyroid hormone, alpha-1-antitrypsin, rsCD40L, Fas-ligand, TRAIL, TNF, antibodies, microcin E492, diphtheria toxin, Pseudomonas exotoxin, Escherichia coli Shiga toxin, Escherichia coli Verotoxin 1, and hyperforin. Exemplary cytokines include, but are not limited to, chemokines and classical cytokines, such as the interleukins, including for example, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factor (GM-CSF), erythropoietin and exemplary chemokines including, but not limited to CXC chemokines such as IL-8, GROα, GROβ, GROγ, ENA-78, LDGF-PBP, GCP-2, PF4, Mig, IP-10, SDF-1α/β, BUNZO/STRC33, I-TAC, BLC/BCA-1; CC chemokines such as MIP-1α, MIP-1β, MDC, TECK, TARC, RANTES, HCC-1, HCC-4, DC-CK1, MIP-3α, MIP-3β, MCP-1, MCP-2, MCP-3, MCP-4, Eotaxin, Eotaxin-2/MPIF-2, 1-309, MIP-5/HCC-2, MPIF-1, 6Ckine, CTACK, MEC; lymphotactin; and fractalkine. Exemplary other costimulatory molecules include immunoglobulin superfamily of cytokines, such as B7.1, B7.2.

Exemplary therapeutic proteins that can be expressed by the viruses provided herein and used in the methods provided herein include, but are not limited to, erythropoietin (e.g., SEQ ID NO:23), an anti-VEGF single chain antibody (e.g., SEQ ID NO:24), a plasminogen K5 domain (e.g., SEQ ID NO:25), a human tissue factor-αvβ3-integrin RGD fusion protein (e.g., SEQ ID NO:26), interleukin-24 (e.g., SEQ ID NO:27), or immune stimulators, such as SIL-6-SIL-6 receptor fusion protein (e.g., SEQ ID NO:28).

In some examples, the viruses provided herein can express one or more therapeutic gene products that are proteins that convert a less active compound into a compound that causes tumor cell death. Exemplary methods of conversion of such a prodrug compound include enzymatic conversion and photolytic conversion. A large variety of protein/compound pairs are known in the art, and include, but are not limited to, Herpes simplex virus thymidine kinase/ganciclovir, Herpes simplex virus thymidine kinase/(E)-5-(2-bromovinyl)-2′-deoxyuridine (BVDU), varicella zoster thymidine kinase/ganciclovir, varicella zoster thymidine kinase/BVDU, varicella zoster thymidine kinase/(E)-5-(2-bromovinyl)-1-beta-D-arabinofuranosyluracil (BVaraU), cytosine deaminase/5-fluorouracil, cytosine deaminase/5-fluorocytosine, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid (CMDA), carboxypeptidase A/methotrexate-phenylamine, cytochrome P450/acetaminophen, cytochrome P450-2B1/cyclophosphamide, cytochrome P450-4B1/2-aminoanthracene, 4-ipomeanol, horseradish peroxidase/indole-3-acetic acid, nitroreductase/CB 1954, rabbit carboxylesterase/7-ethyl-10-[4-(1-piperidino)-1-piperidino]carbonyloxy-camptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin glucuronide, thymidine phosphorylase/5′-deoxy-5-fluorouridine, deoxycytidine kinase/cytosine arabinoside, and linamarase/linamarin.

Other therapeutic gene products that can be expressed by the viruses provided herein include siRNA and microRNA molecules. The siRNA and/or microRNA molecule can be directed against expression of a tumor-promoting gene, such as, but not limited to, an oncogene, growth factor, angiogenesis promoting gene, or a receptor. The siRNA and/or microRNA molecule also can be directed against expression of any gene essential for cell growth, cell replication or cell survival. The siRNA and/or microRNA molecule also can be directed against expression of any gene that stabilizes the cell membrane or otherwise limits the number of tumor cell antigens released from the tumor cell. Design of an siRNA or microRNA can be readily determined according to the selected target of the siRNA; methods of siRNA and microRNA design and down-regulation of genes are known in the art, as exemplified in U.S. Pat. Pub. Nos. 2003-0198627 and 2007-0044164, and Zeng et al., (2002) Molecular Cell 9:1327-1333.

Therapeutic gene products include viral attenuation factors, such as antiviral proteins. Antiviral proteins or peptides can be expressed by the viruses provided herein. Expression of antiviral proteins or peptides can control viral pathogenicity.

Exemplary viral attenuation factors include, but are not limited to, virus-specific antibodies, mucins, thrombospondin, and soluble proteins such as cytokines, including, but not limited to TNFα, interferons (for example IFNα, IFNβ, or IFNγ) and interleukins (for example IL-1, IL-12 or IL-18).

Another exemplary therapeutic gene product that can be expressed by the viruses provided herein is a protein ligand, such as antitumor oligopeptide. Antitumor oligopeptides are short protein peptides with high affinity and specificity to tumors. Such oligopeptides could be enriched and identified using tumor-associated phage libraries (Akita et al. (2006) Cancer Sci. 97(10):1075-1081). These oligopeptides have been shown to enhance chemotherapy (U.S. Pat. No. 4,912,199). The oligopeptides can be expressed by the viruses provided herein. Expression of the oligopeptides can elicit anticancer activities on their own or in combination with other chemotherapeutic agents. An exemplary group of antitumor oligopeptides is antimitotic peptides, including, but not limited to, tubulysin (Khalil et al. (2006) Chembiochem. 7(4):678-683), phomopsin, hemiasterlin, taltobulin (HTI-286, 3), and cryptophycin. Tubulysin is from myxobacteria and can induce depletion of cell microtubules and trigger the apoptotic process. The antimitotic peptides can be expressed by the viruses provide herein and elicit anticancer activities on their own or in combination with other therapeutic modalities.

Another exemplary therapeutic gene product that can be expressed by the viruses provided herein is an anti-metastatic agent that inhibits one or more steps of the metastatic cascade. The encoded anti-metastatic agents include agents that inhibit invasion of local tissue, inhibit intravasation into the bloodstream or lymphatics, inhibit cell survival and transport through the bloodstream or lymphatics as emboli or potentially single cells, inhibit cell lodging in microvasculature at the secondary site, inhibit growth into microscopic lesions and subsequently into overt metastatic lesions, and/or inhibit metastasis formation and growth within the primary tumor, where the inhibition of metastasis formation is not a consequence of inhibition of primary tumor growth.

Exemplary anti-metastatic agents expressed by the viruses provided herein can directly or indirectly inhibit one or more steps of the metastatic cascade. Exemplary anti-metastatic agents include, but are not limited to, the following: BRMS-1 (Breast Cancer Metastasis Suppressor 1), CRMP-1 (Collapsin Response Mediator Protein-1), CRSP-3 (Cofactor Required for Spl transcriptional activation subunit 3), CTGF (Connective Tissue Growth Factor), DRG-1 (Developmentally-regulated GTP-binding protein 1), E-Cad (E-cadherin), gelsolin, KAI1, KiSS1 (Kisspeptin 1/Metastin), kispeptin-10, kispeptin-13, kispeptin-14, kispeptin-54, LKB1 (STK11 (serine/threonine kinase 11)), JNKK1/MKK4 (c-Jun-NH2-Kinase Kinase/Mitogen activated Kinase Kinase 4), MKK6 (mitogen activated kinase kinase 6), MKK7 (mitogen activated kinase kinase 7), Nm23 (NDP Kinase A), RASSF1-8 (Ras association (RalGDS/AF-6) domain family members), RKIP (Raf kinase inhibitor protein), RhoGDI2 (Rho GDP dissociation inhibitor 2), SSECKS (src-suppressed C-kinase substrate), Syk, TIMP-1 (Tissue inhibitor of metalloproteinase-1), TIMP-2 (Tissue inhibitor of metalloproteinase-2), TIMP-3 (Tissue inhibitor of metalloproteinase-3), TIMP-4 (Tissue inhibitor of metalloproteinase-4), TXNIP/VDUP1 (Thioredoxin-interacting protein). Such list of anti-metastatic agents is not meant to be limiting. Any gene product that can suppress metastasis formation via a mechanism that is independent of inhibition of growth within the primary tumor is encompassed by the designation of an anti-metastatic agent or metastasis suppressor and can be expressed by a virus as provided herein. One of skill in the art can identify anti-metastatic genes and can construct a virus expressing one or more anti-metastatic genes for therapy.

Another exemplary therapeutic gene product that can be expressed by the viruses provided herein is a protein that sequesters molecules or nutrients needed for tumor growth. For example, the virus can express one or more proteins that bind iron, transport iron, or store iron, or a combination thereof. Increased iron uptake and/or storage by expression of such proteins not only, increases contrast for visualization and detection of a tumor or tissue in which the virus accumulates, but also depletes iron from the tumor environment. Iron depletion from the tumor environment removes a vital nutrient from the tumors, thereby deregulating iron hemostasis in tumor cells and delaying tumor progression and/or killing the tumor.

Additionally, iron, or other labeled metals, can be administered to a tumor-bearing subject, either alone, or in a conjugated form. An iron conjugate can include, for example, iron conjugated to an imaging moiety or a therapeutic agent. In some cases, the imaging moiety and therapeutic agent are the same, e.g., a radionuclide. Internalization of iron in the tumor, wound, area of inflammation or infection allows the internalization of iron alone, a supplemental imaging moiety, or a therapeutic agent (which can deliver cytotoxicity specifically to tumor cells or deliver the therapeutic agent for treatment of the wound, area of inflammation or infection). These methods can be combined with any of the other methods provided herein.

The administered virus also can be modified to stimulate humoral and/or cellular immune response in the subject, such as the induction of cytotoxic T lymphocytes responses. For example, the virus can provide prophylactic and therapeutic effects against a tumor infected by the virus or other infectious diseases, by rejection of cells from tumors or lesions using viruses that express immunoreactive antigens (Earl et al., Science 234: 728-831 (1986); Lathe et al., Nature (London) 32: 878-880 (1987)), cellular tumor-associated antigens (Bernards et al., Proc. Natl. Acad. Sci. USA 84: 6854-6858 (1987); Estin et al., Proc. Natl. Acad. Sci. USA 85: 1052-1056 (1988); Kantor et al., J. Natl. Cancer Inst. 84: 1084-1091 (1992); Roth et al., Proc. Natl. Acad. Sci. USA 93: 4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol. 3: 495-500 (1996)), or other therapeutic proteins.

iii. Antigens

For example, the viruses provided herein can be modified to express one or more antigens. Sustained release of the antigen can result in an immune response by the viral-infected host, in which the host can develop antibodies against the antigen and/or the host can develop an immune response against cells expressing the antigen. Exemplary antigens include, but are not limited to, tumor specific antigens, tumor-associated antigens, tissue-specific antigens, bacterial antigens, viral antigens, yeast antigens, fungal antigens, protozoan antigens, parasite antigens and mitogens. Superantigens are antigens that can activate a large immune response, often brought about by a large response of T cells. A variety of superantigens are known in the art including, but not limited to, diphtheria toxin, staphylococcal enterotoxins (SEA, SEB, SEC1, SEC2, SED, SEE and SEH), Toxic Shock Syndrome Toxin 1, Exfoliating Toxins (EXft), Streptococcal Pyrogenic Exotoxin A, B and C(SPE A, B and C), Mouse Mammary Tumor Virus proteins (MMTV), Streptococcal M proteins, Clostridial Perfringens Enterotoxin (CPET), Listeria monocytogenes antigen p60, and mycoplasma arthritis superantigens.

Since many superantigens also are toxins, if expression of a virus of reduced toxicity is desired, the superantigen can be modified to retain at least some of its superantigenicity while reducing its toxicity, resulting in a compound such as a toxoid. A variety of recombinant superantigens and toxoids of superantigens are known in the art, and can readily be expressed in the viruses provided herein. Exemplary toxoids include toxoids of diphtheria toxin, as exemplified in U.S. Pat. No. 6,455,673 and toxoids of Staphylococcal enterotoxins, as exemplified in U.S. Pat. Pub. No. 2003-0009015.

iv. Modifications to Alter Attenuation of the Viruses

The toxicity of the viruses can be modulated. For example, viruses provided herein can be attenuated by addition, deletion and/or modification of nucleic acid in the viral genome. In particular, viruses can be attenuated by increasing transcriptional or translational load. In one example, the virus is attenuated by addition of heterologous nucleic acid that contains an open reading frame that encodes one or more gene products (e.g. a diagnostic gene product or a therapeutic gene product as described above). In another example, the virus is attenuated by modification of heterologous nucleic acid that contains an open reading frame that encodes one or more gene products. In a further example, the heterologous nucleic acid is modified by increasing the length of the open reading frame, removal of all or part of the open reading frame or replacement of all or part of the open reading frame. Such modifications can affect viral toxicity by disruption of one or more viral genes or by increasing or decreasing the transcriptional and/or translational load on the virus (see, e.g., International Patent Publication No. WO 2008/100292).

In another example, the virus can be attenuated by modification or replacement of one or more promoters contained in the virus. Such promoters can be replaced by stronger or weaker promoters, where replacement results in a change in the attenuation of the virus. In one example, a promoter of a virus provided herein is replaced with a natural promoter. In one example, a promoter of a virus provided herein is replaced with a synthetic promoter. Exemplary promoters that can replace a promoter contained in a virus can be a viral promoter, such as a vaccinia viral promoter, and can include a vaccinia early, intermediate, early/late or late promoter. Additional exemplary viral promoters are provided herein and known in the art and can be used to replace a promoter contained in a virus.

In another example, the virus is attenuated by modification of a heterologous nucleic acid contained in the virus by removal or all or a portion of a first heterologous nucleic acid molecule and replacement by a second heterologous nucleic acid molecule, where replacement changes the level of attenuation of the virus. The second heterologous nucleic acid molecule can contain a sequence of nucleotides that encodes a protein or can be a non-coding nucleic acid molecule. In some examples, the second heterologous nucleic acid molecule contains an open reading frame operably linked to a promoter. The second heterologous nucleic acid molecule can contain one or more open reading frames or one or more promoters. Further, the one or more promoters of the second heterologous nucleic acid molecule can be one or more stronger promoters or one or more weaker promoters, or can be a combination or both.

Attenuated vaccinia viruses are known in the art and are described, for example, in U.S. Patent Pub. Nos. US 2005-0031643 now U.S. Pat. Nos. 7,588,767, 7,588,771 and 7,662,398, US 2008-0193373, US 2009-0098529, US 2009-0053244, US 2009-0155287, US 2009-0081639, US 2009-0117034 and US 2009-0136917, and International Patent Pub. Nos. WO 2005/047458, WO 2008/100292 and WO 2008/150496.

Viruses provided herein also can contain a modification that alters its infectivity or resistance to neutralizing antibodies. In one non-limiting example deletion of the A35R gene in a vaccinia LIVP strain can decrease the infectivity of the virus. In some examples, the viruses provided herein can be modified to contain a deletion of the A35R gene. Exemplary methods for generating such viruses are described in PCT Publication No. WO2008/100292, which describes vaccinia LIVP viruses GLV-1j87, GLV-1j88 and GLV-1j89, which contain deletion of the A35R gene.

In another non-limiting example, replacement of viral coat proteins (e.g., A34R, which encodes a viral coat glycoprotein) with coat proteins from either more virulent or less virulent virus strains can increase or decrease the clearance of the virus from the subject. In one example, the A34R gene in a vaccinia LIVP strain can be replaced with the A34R gene from vaccinia IHD-J strain. Such replacement can increase the extracellular enveloped virus (EEV) form of vaccinia virus and can increase the resistance of the virus to neutralizing antibodies.

b. Control of Heterologous Gene Expression

In some examples, the heterologous nucleic acid also can contain one or more regulatory sequences to regulate expression of an open reading frame encoding the heterologous RNA and/or protein. Suitable regulatory sequences which, for example, are functional in a mammalian host cell are well known in the art. Expression can also be influenced by one or more proteins or RNA molecules expressed by the virus. Gene regulatory elements, such as promoters and enhancers, possess cell type specific activities and can be activated by certain induction factors (e.g., hormones, growth factors, cytokines, cytostatic agents, irradiation, heat shock) via responsive elements. A controlled and restricted expression of these genes can be achieved using such regulatory elements as internal promoters to drive the expression of therapeutic genes in viral vector constructs.

For example, the one or more heterologous nucleic acid molecules can be operably linked to a promoter for expression of the heterologous RNA and/or protein. For example, a heterologous nucleic acid that is operably linked to a promoter is also called an expression cassette. Hence, viruses provided herein can have the ability to express one or more heterologous genes. Gene expression can include expression of a protein encoded by a gene and/or expression of an RNA molecule encoded by a gene. In some embodiments, the viruses provided herein can express exogenous genes at levels high enough that permit harvesting products of the exogenous genes from the tumor. Exemplary promoters for the expression of heterologous genes are known in the art. The heterologous nucleic acid can be operatively linked to a native promoter or a heterologous promoter that is not native to the virus. Any suitable promoters, including synthetic and naturally-occurring and modified promoters, can be used. Exemplary promoters include synthetic promoters, including synthetic viral and animal promoters. Native promoter or heterologous promoters include, but are not limited to, viral promoters, such as vaccinia virus and adenovirus promoters.

In one example, the promoter is a poxvirus promoter, such as, for example, a vaccinia virus promoter. Vaccinia viral promoters for the expression of one or more heterologous genes can be synthetic or natural promoters, and include vaccinia early, intermediate, early/late and late promoters. Exemplary vaccinia viral promoters for controlling heterologous gene expression include, but are not limited to, P_{7.5k}, P₁₁ₖ, P_{SE}, P_{SEL}, P_{SL}, H5R, TK, P28, C11R, G8R, F17R, 13L, 18R, A1L, A2L, A3L, H1L, H3L, H5L, H6R, H8R, D1R, D4R, D5R, D9R, D11L, D12L, D13L, M1L, N2L, P4b or K1 promoters. Other viral promoters include, but are not limited to, adenovirus late promoter, Cowpox ATI promoter, or T7 promoter. Strong late promoters can be used to achieve high levels of expression of the heterologous genes. Early and intermediate-stage promoters also can be used. In one example, the promoters contain early and late promoter elements, for example, the vaccinia virus early/late promoter P_{7.5k}, vaccinia late promoter P₁₁ₖ, a synthetic early/late vaccinia P_{SEL} promoter (Patel et al., (1988) Proc. Natl. Acad. Sci. USA 85:9431-9435; Davison and Moss, (1989) J Mol Biol 210:749-769; Davison et al. (1990) Nucleic Acids Res. 18:4285-4286; Chakrabarti et al. (1997), BioTechniques 23:1094-1097). The viruses provided herein can exhibit differences in characteristics, such as attenuation, as a result of using a stronger promoter versus a weaker promoter. For example, in vaccinia, synthetic early/late and late promoters are relatively strong promoters, whereas vaccinia synthetic early, P_{7.5k} early/late, P_{7.5k} early, and P₂₈ late promoters are relatively weaker promoters (see e.g., Chakrabarti et al. (1997) BioTechniques 23(6):1094-1097). Combinations of different promoters can be used to express different gene products in the same virus or two different viruses.

Expression of heterologous genes can be controlled by a constitutive promoter, or by an inducible promoter. For example, gene expression can be made inducible using a tetracycline-regulated promoter, whereby transcription is reversibly turned on or off in the presence of tetracycline or one of its derivative (e.g. doxycycline). In such a system, in the absence of an inducer, a tetracycline repressor (TetR) binds to the tet operator (tetO) to repress the activity of the promoter placed near the operator. In the presence of an inducer that binds to TetR, a conformational change occurs that prevents TetR from remaining bound to the operator, thereby permitting gene transcription.

In other examples, organ or tissue-specific expression can be controlled by regulatory sequences. In order to achieve expression only in the target organ, for example, a tumor to be treated, the foreign nucleotide sequence can be linked to a tissue specific promoter and used for gene therapy. Such promoters are well known to those skilled in the art (see, e.g., Zimmermann et al., Neuron 12: 11-24 (1994); Vidal et al., EMBO J. 9: 833-840 (1990); Mayford et al., Cell 81: 891-904 (1995); and Pinkert et al., Genes & Dev. 1: 268-76 (1987)).

As is known in the art, regulatory sequences can permit constitutive expression of the exogenous gene or can permit inducible expression of the exogenous gene. Further, the regulatory sequence can permit control of the level of expression of the exogenous gene. In some examples, such as gene product manufacture and harvesting, the regulatory sequence can result in constitutive, high levels of gene expression. In some examples, such as anti-(gene product) antibody harvesting, the regulatory sequence can result in constitutive, lower levels of gene expression. In tumor therapy examples, a therapeutic protein can be under the control of an internally inducible promoter or an externally inducible promoter.

Hence, expression of heterologous genes can be controlled by a constitutive promoter or by an inducible promoter. Inducible promoters can be used to provide tissue specific expression of the heterologous gene or can be inducible by the addition of a regulatory molecule to provide temporal specific induction of the promoter. In some examples, inducible expression can be under the control of cellular or other factors present in a tumor cell or present in a virus-infected tumor cell. In further examples, inducible expression can be under the control of an administrable substance, including IPTG, RU486 or other known induction compounds. Additional regulatory sequences can be used to control the expression of the one or more heterologous genes inserted the virus. Any of a variety of regulatory sequences are available to one skilled in the art according to known factors and design preferences.

c. Methods for Generating Modified Viruses

The viruses for use in the methods provided herein can be modified by insertion, deletion, replacement or mutation as described herein, for example insertion or replacement of heterologous nucleic acid, using standard methodologies well known in the art for modifying viruses. Methods for modification include, for example, in vitro recombination techniques, synthetic methods, direct cloning, and in vivo recombination methods as described, for example, in Sambrook et al., Molecular Cloning: A Laboratory Manual, 2nd edition, Cold Spring Harbor Laboratory Press, cold Spring Harbor N.Y. (1989), and in the Examples disclosed herein.

For example, generation of recombinant viruses, including recombinant vaccinia virus, is well known in the art, and typically involves the generation of gene cassettes or transfer vectors using standard techniques in molecular biology (see, e.g., U.S. Pat. No. 7,588,767 and US2009-0053244-A1, which describe exemplary methods of generating recombinant LIVP vaccinia viruses). Such techniques include various nucleic acid manipulation techniques, nucleic acid transfer protocols, nucleic acid amplification protocols, and other molecular biology techniques known in the art.

For example, point mutations or small insertions or deletions can be introduced into a gene of interest through the use of oligonucleotide mediated site-directed mutagenesis. In another example, homologous recombination can be used to introduce a mutation in the nucleic acid sequence or insertion or deletion of a nucleic acid molecule into a target sequence of interest. In some examples, mutations, insertions or deletions of nucleic acid in a particular gene can be selected for using a positive or negative selection pressure. See, e.g., Current Techniques in Molecular Biology, (Ed. Ausubel, et al.).

Nucleic acid amplification protocols include, but are not limited to, the polymerase chain reaction (PCR), or amplification via viruses or organisms, such as, but not limited to, bacteria, yeast, insect or mammalian cells. Use of nucleic acid tools such as plasmids, vectors, promoters and other regulating sequences, are well known in the art for a large variety of viruses and cellular organisms.

Nucleic acid transfer protocols include calcium chloride transformation/transfection, electroporation, liposome mediated nucleic acid transfer, N-[1-(2,3-dioloyloxy)propyl]-N,N,N-trimethylammonium methylsulfate meditated transformation, and others. Further a large variety of nucleic acid tools are available from many different sources, including various commercial sources. One skilled in the art will be readily able to select the appropriate tools and methods for genetic modifications of any particular virus according to the knowledge in the art and design choice.

Hence, any of a variety of modifications can be readily accomplished using standard molecular biological methods known in the art. The modifications will typically be one or more truncations, deletions, mutations or insertions of the viral genome. In one example, the modification can be specifically directed to a particular sequence in the viral genome. The modifications can be directed to any of a variety of regions of the viral genome, including, but not limited to, a regulatory sequence, a gene-encoding sequence, an intergenic sequence, a sequence without a known role, or a non-essential region of the viral genome. Any of a variety of regions of viral genomes that are available for modification are readily known in the art for many viruses, including LIVP.

Heterologous nucleic acid molecules are typically inserted into the viral genome in an intergenic region or in a locus that encodes a nonessential viral gene product. Insertion of heterologous nucleic acid at such sites generally does not significantly affect viral infection or replication in the target tissue. Exemplary insertion sites are known in the art and include, but are not limited to, J2R (thymidine kinase (TK)), A56R (hemagglutinin (HA)), F14.5L, vaccinia growth factor (VGF), A35R, N1L, E2L/E3L, K1L/K2L, superoxide dismutase locus, 7.5K, C7-K1L (host range gene region), B13R+B14R (hemorrhagic region), A26L (A type inclusion body region (ATI)) or 14L (large subunit, ribonucleotide reductase) gene loci. Insertion sites for the viruses provided herein also include sites that correspond to intragenic regions described in other poxviruses such as Modified Vaccinia Ankara (MVA) virus (exemplary sites set forth in U.S. Pat. No. 7,550,147), NYVAC (exemplary sites set forth in U.S. Pat. No. 5,762,938).

Methods for the generation of recombinant viruses using recombinant DNA techniques are well known in the art (e.g., see U.S. Pat. Nos. 4,769,330; 4,603,112; 4,722,848; 4,215,051; 5,110,587; 5,174,993; 5,922,576; 6,319,703; 5,719,054; 6,429,001; 6,589,531; 6,573,090; 6,800,288; 7,045,313; He et al. (1998) PNAS 95(5): 2509-2514; Racaniello et al., (1981) Science 214: 916-919; and Hruby et al., (1990) Clin Micro Rev. 3:153-170). Methods for the generation of recombinant vaccinia viruses are well known in the art (e.g., see Hruby et al., (1990) Clin Micro Rev. 3:153-170, U.S. Pat. Pub. No. 2005-0031643, now U.S. Pat. Nos. 7,588,767, 7,588,771, 7,662,398 and U.S. Pat. No. 7,045,313).

For example, generating a recombinant vaccinia virus that expresses a heterologous gene product typically includes the use of a recombination plasmid which contains the heterologous nucleic acid, optionally operably linked to a promoter, with vaccinia virus DNA sequences flanking the heterologous nucleic acid to facilitate homologous recombination and insertion of the gene into the viral genome. Generally, the viral DNA flanking the heterologous gene is complementary to a non-essential segment of vaccinia virus DNA, such that the gene is inserted into a nonessential location. The recombination plasmid can be grown in and purified from Escherichia coli and introduced into suitable host cells, such as, for example, but not limited to, CV-1, BSC-40, BSC-1 and TK-143 cells. The transfected cells are then superinfected with vaccinia virus which initiates a replication cycle. The heterologous DNA can be incorporated into the vaccinia viral genome through homologous recombination, and packaged into infection progeny. The recombinant viruses can be identified by methods known in the art, such as by detection of the expression of the heterologous gene product, or by using positive or negative selection methods (U.S. Pat. No. 7,045,313).

In another example, the recombinant vaccinia virus that expresses a heterologous gene product can be generated by direct cloning (see, e.g. U.S. Pat. No. 6,265,183 and Scheiflinger et al. (1992) Proc. Natl. Acad. Sci. USA 89: 9977-9981). In such methods, the heterologous nucleic acid, optionally operably linked to a promoter, is flanked by restriction endonuclease cleavage sites for insertion into a unique restriction endonuclease site in the target virus. The virus DNA is purified using standard techniques and is cleaved with the sequence-specific restriction endonuclease, where the sequence is a unique site in the virus genome. Any unique site in the virus genome can be employed provided that modification at the site does not interfere with viral replication. For example, in vaccinia virus strain LIVP, the NotI restriction site is located in the ORF encoding the F14.5L gene with unknown function (Mikryukov et al., Biotekhnologiya 4: 442-449 (1988)). Table 7 provides a summary of unique restriction sites contained in exemplary LIVP strains and designates the nucleotide position of each. Such LIVP strains can be modified herein by direct cloning and insertion of heterologous DNA into the site or sites. Generally, insertion is in a site that is located in a non-essential region of the virus genome. For example, exemplary modifications herein include insertion of a foreign DNA sequence into the NotI digested virus DNA.

TABLE 7
Unique restriction endonuclease cleavage sites in LIVP clonal
isolates

| Restriction | | | | | | | | | |
|---|---|---|---|---|---|---|---|---|---|
| Enzyme/Site | | | | | | | | | LIVP |
| | SEQ | 1.1.1 | 2.1.1 | 4.1.1 | 5.1.1 | 6.1.1 | 7.1.1 | 8.1.1 | Parental |
| Name/ | ID | (SEQ ID | (SEQ ID | (SEQ ID | (SEQ ID | (SEQ ID | (SEQ ID | (SEQ ID | (SEQ ID |
| sequence | NO | NO: 3) | NO: 4) | NO: 5) | NO: 6) | NO: 7) | NO: 8) | NO: 9) | NO: 20) |
| SbfI | 64 | 40033/ | 40756/ | 39977/ | 40576/ | 40177/ | 40213/ | 40493/ | 38630/ |
| CCTGCAGG | | 40029 | 40752 | 39973 | 40572 | 40173 | 40209 | 40489 | 38626 |
| NotI | 65 | 42989/ | 43712/ | 42933/ | 43532/ | 43133/ | 43169/ | 43449/ | 41586/ |
| GCGGCCGC | | 42998 | 43716 | 42937 | 43536 | 43137 | 43173 | 43453 | 41590 |
| SgrAI | 66 | 114365/ | 115107/ | 114308/ | 114924/ | 114489/ | 114548/ | 114845/ | 112975/ |
| CRCCGGYG | | 114369 | 115111 | 114312 | 114928 | 114493 | 114552 | 114849 | 112979 |
| SmaI | 67 | 159260 | NA | NA | NA | NA | NA | NA | NA |
| CCCGGG | | | | | | | | | |
| TspMI | 68 | 159258/ | NA | NA | NA | NA | NA | NA | NA |
| CCCGGG | | 159262 | | | | | | | |
| XmaI | 69 | 159258/ | NA | NA | NA | NA | NA | NA | NA |
| CCCGGG | | 159262 | | | | | | | |
| ApaI | 70 | 180516/ | NA | 180377/ | 181027/ | 180638/ | 180596/ | 180972/ | NA |
| CCCGGG | | 180512 | | 180373 | 181023 | 180634 | 180592 | 180968 | |
| PspOMI | 71 | 180512/ | NA | 180373/ | 181023/ | 180634/ | 180592/ | 180968/ | NA |
| CCCGGG | | 180516 | | 180377 | 181027 | 180638 | 180596 | 180972 | |

In some examples, the virus genomic DNA is first modified by homologous recombination to introduce one or more unique restriction sites in the virus (see, e.g. Mackett et al. (1984) J. Virol. 857-864). Following cleavage with the restriction endonuclease, the cleaved DNA is optionally treated with a phosphatase to remove a phosphate moiety from an end of the DNA segment that is produced by cleavage with the endonuclease. Typically, a plasmid vector is generated that contains the heterologous DNA for insertion flanked by the restriction sites. Prior to insertion into the virus, the heterologous DNA is excised from the plasmid by cleavage with the sequence specific restriction endonuclease. The heterologous DNA is then ligated to the cleaved viral DNA and is packaged in a permissive cell line by infection of the cells with a helper virus, such as, but not limited to a fowlpox virus or a PUV-inactivated helper vaccinia virus, and transfection of the ligated DNA into the infected cells.

In some examples, the methods involve homologous recombination and/or use of unique restriction sites in the virus. For example, a recombinant LIVP vaccinia virus with an insertion, for example, in the F14.5L gene (e.g., in the Not I restriction site of an LIVP isolate) can be prepared by the following steps: (a) generating (i) a vaccinia shuttle/transfer plasmid containing the modification (e.g. a gene expression cassette or a modified F14.5L gene) inserted at a restriction site, X (e.g. Not 1), where the restriction site in the vector is flanked by parental virus sequences of the target insertion site and (ii) an LIVP virus DNA digested at restriction site X (e.g. Not I) and optionally dephosphorylated; (b) infecting cells with PUV-inactivated helper vaccinia virus and transfecting the infected host cells with a mixture of the constructs of (i) and (ii) of step a; and (c) isolating the recombinant vaccinia viruses from the transfectants. One skilled in the art knows how to perform such methods (see, e.g., Timiryasova et al. (Biotechniques 31: 534-540 (2001)). Typically, the restriction site X is a unique restriction site in the virus as described above.

In one example, the methods include introducing into the viruses one or more genetic modifications, followed by screening the viruses for properties reflective of the modification or for other desired properties. In some examples, the modification can be fully or partially random, whereupon selection of any particular modified virus can be determined according to the desired properties of the modified the virus.

4. Methods of Producing Viruses

Viruses for use in the methods provided herein can be produced by methods known to one of skill in the art. Typically, the virus is propagated in host cells, quantified and prepared for storage before finally being prepared in the compositions described herein. The virus can be propagated in suitable host cells to enlarge the stock, the concentration of which is then determined. In some examples, the infectious titer is determined, such as by plaque assay. The total number of viral particles also can be determined. The viruses are stored in conditions that promote stability and integrity of the virus, such that loss of infectivity over time is minimized. In some examples, a large amount of virus is produced and stored in small aliquots of known concentration that can be used for multiple procedures over an extended period of time. Conditions that are most suitable for various viruses will differ, and are known in the art, but typically include freezing or drying, such as by lyophilization. The viruses can be stored at a concentration of 10⁵-10¹⁰ pfu/mL, for example, 10⁷-10⁹ pfu/mL, such as at least or about or is 10⁶ pfu/mL, 10⁷ pfu/mL, 10⁸ pfu/mL or 10⁹ pfu/mL. Immediately prior to preparing compositions provided herein, the stored viruses can be reconstituted (if dried for storage) and diluted in an appropriate medium or solution. The following sections provide exemplary methods that can be used for the production and preparation of viruses for use in preparing viruses in the compositions provided herein.

a. Host Cells for Propagation

Virus strains can be propagated in an appropriate host cell. Such cells can be a group of a single type of cells or a mixture of different types of cells. Host cells can include cultured cell lines, primary cells, and proliferative cells. These host cells can include any of a variety of animal cells, such as mammalian, avian and insect cells and tissues that are susceptible to the virus, such as vaccinia virus, infection, including chicken embryo, rabbit, hamster, and monkey kidney cells. Suitable host cells include, but are not limited to, hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts, and cell lines including, for example, CV-1, BSC40, Vero, and BSC-1, and human HeLa cells. Typically, viruses are propagated in cell lines that that can be grown at monolayers or in suspension. For example, exemplary cell lines for the propagation of vaccinia viruses include, but are not limited to, CV-1, BSC40, Vero, BGM, BSC-1 and RK-13 cells. Purification of the cultured strain from the system can be effected using standard methods.

b. Concentration Determination

The concentration of virus in a solution, or virus titer, can be determined by a variety of methods known in the art. In some methods, a determination of the number of infectious virus particles is made (typically termed plaque forming units (PFU)), while in other methods, a determination of the total number of viral particles, either infectious or not, is made. Methods that calculate the number of infectious virions include, but are not limited to, the plaque assay, in which titrations of the virus are grown on cell monolayers and the number of plaques is counted after several days to several weeks, and the endpoint dilution method, which determines the titer within a certain range, such as one log. Methods that determine the total number of viral particles, including infectious and non-infectious, include, but are not limited to, immunohistochemical staining methods that utilize antibodies that recognize a viral antigen and which can be visualized by microscopy or FACS analysis; optical absorbance, such as at 260 nm; and measurement of viral nucleic acid, such as by PCR, RT-PCR, or quantitation by labeling with a fluorescent dye.

c. Storage Methods

Once the virus has been purified (or to a desired purity) and the titer has been determined, the virus can be stored in conditions which optimally maintain its infectious integrity. Typically, viruses are stored in the dark, because light serves to inactivate the viruses over time. Viral stability in storage is usually dependent upon temperatures. Although some viruses are thermostable, most viruses are not stable for more than a day at room temperature, exhibiting reduced viability (Newman et al., (2003) J. Inf. Dis. 187:1319-1322). Vaccinia virus is generally stable at refrigerated temperatures, and can be stored in solution at 4° C., frozen at, for example −20° C., −70° C. or −80° C., or lyophilized with little loss of viability (Newman et al., (2003) J. Inf. Dis. 187:1319-1322, Hruby et al., (1990) Clin. Microb. Rev. 3:153-170). Methods and conditions suitable for the storage of particular viruses are known in the art, and can be used to store the viruses used in the methods presented herein.

For short-term storage of viruses, for example, 1 day, 2 days, 4 days or 7 days, temperatures of approximately 4° C. are generally recommended. For long-term storage, most viruses can be kept at −20° C., −70° C. or −80° C. When frozen in a simple solution such as PBS or Tris solution (20 mM Tris pH 8.0, 200 NaCl, 2-3% glycerol or sucrose) at these temperatures, the virus can be stable for 6 months to a year, or even longer. Repeated freeze-thaw cycles are generally avoided, however, since it can cause a decrease in viral titer. The virus also can be frozen in media containing other supplements in the storage solution which can further preserve the integrity of the virus. For example, the addition of serum or bovine serum albumin (BSA) to a viral solution stored at −80° C. can help retain virus viability for longer periods of time and through several freeze-thaw cycles.

In other examples, the virus sample is dried for long-term storage at ambient temperatures. Viruses can be dried using various techniques including, but not limited to, freeze-drying, foam-drying, spray-drying and desiccation. Water is a reactant in nearly all of the destructive pathways that degrade viruses in storage. Further, water acts as a plasticizer, which allows unfolding and aggregation of proteins. Since water is a participant in almost all degradation pathways, reduction of the aqueous solution of viruses to a dry powder provides an alternative composition methodology to enhance the stability of such samples. Lyophilization, or freeze-drying, is a drying technique used for storing viruses (see, e.g., Croyle et al., (1998) Pharm. Dev. Technol., 3(3), 973-383). There are three stages to freeze-drying; freezing, primary drying and secondary drying. During these stages, the material is rapidly frozen and dehydrated under high vacuum. Once lyophilized, the dried virus can be stored for long periods of time at ambient temperatures, and reconstituted with an aqueous solution when needed. Various stabilizers can be included in the solution prior to freeze-drying to enhance the preservation of the virus. For example, it is known that high molecular weight structural additives, such as serum, serum albumin or gelatin, aid in preventing viral aggregation during freezing, and provide structural and nutritional support in the lyophilized or dried state. Amino acids such as arginine and glutamate, sugars, such as trehalose, and alcohols such as mannitol, sorbitol and inositol, can enhance the preservation of viral infectivity during lyophilization and in the lyophilized state. When added to the viral solution prior to lyophilization, urea and ascorbic acid can stabilize the hydration state and maintain osmotic balance during the dehydration period. Typically, a relatively constant pH of about 7.0 is maintained throughout lyophilization.

Other methods for the storage of viruses at ambient, refrigerated or freezing temperatures are known in the art, and include, but are not limited to, those described in U.S. Pat. Nos. 5,149,653; 6,165,779; 6,255,289; 6,664,099; 6,872,357; and 7,091,030; and in U.S. Pat. Pub. Nos. 2003-0153065, 2004-0038410 and 2005-0032044.

d. Preparation of Virus

Immediately prior to use, the virus can be prepared at an appropriate concentration in suitable media, and can be maintained at a cool temperature, such as on ice, until use. If the virus was lyophilized or otherwise dried for storage, then it can be reconstituted in an appropriate aqueous solution. The aqueous solution in which the virus is prepared is typically the medium used in the assay (e.g., DMEM or RPMI) or one that is compatible, such as a buffered saline solution (e.g., PBS, TBS, Hepes solution). For pharmaceutical applications, the virus can be immediately prepared or reconstituted in a pharmaceutical solution. Numerous pharmaceutically acceptable solutions for use are well known in the art (see e.g. Remington's Pharmaceutical Sciences (18^{th} edition) ed. A. Gennaro, 1990, Mack Publishing Co., Easton, Pa.). In one example, the viruses can be diluted in a physiologically acceptable solution, such as sterile saline or sterile buffered saline, with or without an adjuvant or carrier. In other examples, the pharmaceutical solution can contain a component that provides viscosity (e.g. glycerol) and/or component that has bactericidal properties (e.g. phenol). The virus can be reconstituted or diluted to provide the desired concentration or amount. The particular concentration can be empirically determined by one of skill in the art depending on the particular application.

### E. METHODS OF TREATMENT WITH ANTIBIOTICS FOR INCREASING THE THERAPEUTIC EFFICACY OF VIRAL THERAPY

Provided herein are methods for increasing the therapeutic efficacy of viral therapy by administering antibiotics. The methods involve administering a viral therapy and an antibiotic that is effective against commensal gut bacteria. The viral therapy can be oncolytic viral therapy, e.g., the administration of an oncolytic virus, or can be gene therapy whereby a virus is administered to provide heterologous nucleic acid to a subject. Administration of the antibiotic with the viral therapy increases the therapeutic efficacy of the viral therapy. For example, treatment with an antibiotic and an oncolytic virus results in prolonged viral efficacy as compared to administration of an oncolytic virus alone. Exemplary antibiotics and viruses for use in the methods provided are described in sections C and D, respectively, above. Exemplary therapeutic uses of viruses, including oncolytic viruses, are described in section E.1. below.

In some examples, the methods provided herein for increasing the therapeutic efficacy of viral therapy can be used to treat cancer or tumors. Such methods involve administering an oncolytic virus effective against cancer or tumors and an antibiotic that is effective against commensal gut bacterial. Administration of the antibiotic with the oncolytic virus weakens the immune response at the time of viral infection thereby improving the efficacy of the oncolytic virus therapy for treating the cancer or tumor. The methods provided herein can be used for the treatment of cancers and tumors, such as, but not limited to, acute lymphoblastic leukemia, acute lymphoblastic leukemia, acute myeloid leukemia, acute promyelocytic leukemia, adenocarcinoma, adenoma, adrenal cancer, adrenocortical carcinoma, AIDS-related cancer, AIDS-related lymphoma, anal cancer, appendix cancer, astrocytoma, basal cell carcinoma, bile duct cancer, bladder cancer, bone cancer, osteosarcoma/malignant fibrous histiocytoma, brainstem glioma, brain cancer, carcinoma, cerebellar astrocytoma, cerebral astrocytoma/malignant glioma, ependymoma, medulloblastoma, supratentorial primitive neuroectodermal tumor, visual pathway or hypothalamic glioma, breast cancer, bronchial adenoma/carcinoid, Burkitt lymphoma, carcinoid tumor, carcinoma, central nervous system lymphoma, cervical cancer, chronic lymphocytic leukemia, chronic myelogenous leukemia, chronic myeloproliferative disorder, colon cancer, cutaneous T-cell lymphoma, desmoplastic small round cell tumor, endometrial cancer, ependymoma, epidermoid carcinoma, esophageal cancer, Ewing's sarcoma, extracranial germ cell tumor, extragonadal germ cell tumor, extrahepatic bile duct cancer, eye cancer/intraocular melanoma, eye cancer/retinoblastoma, gallbladder cancer, gallstone tumor, gastric/stomach cancer, gastrointestinal carcinoid tumor, gastrointestinal stromal tumor, giant cell tumor, glioblastoma multiforme, glioma, hairy-cell tumor, head and neck cancer, heart cancer, hepatocellular/liver cancer, Hodgkin lymphoma, hyperplasia, hyperplastic corneal nerve tumor, in situ carcinoma, hypopharyngeal cancer, intestinal ganglioneuroma, islet cell tumor, Kaposi's sarcoma, kidney/renal cell cancer, laryngeal cancer, leiomyoma tumor, lip and oral cavity cancer, liposarcoma, liver cancer, non-small cell lung cancer, small cell lung cancer, lymphomas, macroglobulinemia, malignant carcinoid, malignant fibrous histiocytoma of bone, malignant hypercalcemia, malignant melanomas, marfanoid habitus tumor, medullary carcinoma, melanoma, merkel cell carcinoma, mesothelioma, metastatic skin carcinoma, metastatic squamous neck cancer, mouth cancer, mucosal neuromas, multiple myeloma, mycosis fungoides, myelodysplastic syndrome, myeloma, myeloproliferative disorder, nasal cavity and paranasal sinus cancer, nasopharyngeal carcinoma, neck cancer, neural tissue cancer, neuroblastoma, oral cancer, oropharyngeal cancer, osteosarcoma, ovarian cancer, ovarian epithelial tumor, ovarian germ cell tumor, pancreatic cancer, parathyroid cancer, penile cancer, pharyngeal cancer, pheochromocytoma, pineal astrocytoma, pineal germinoma, pineoblastoma, pituitary adenoma, pleuropulmonary blastoma, polycythemia vera, primary brain tumor, prostate cancer, rectal cancer, renal cell tumor, reticulum cell sarcoma, retinoblastoma, rhabdomyosarcoma, salivary gland cancer, seminoma, Sezary syndrome, skin cancer, small intestine cancer, soft tissue sarcoma, squamous cell carcinoma, squamous neck carcinoma, stomach cancer, supratentorial primitive neuroectodermal tumor, testicular cancer, throat cancer, thymoma, thyroid cancer, topical skin lesion, trophoblastic tumor, urethral cancer, uterine/endometrial cancer, uterine sarcoma, vaginal cancer, vulvar cancer, Waldenstrom's macroglobulinemia and Wilm's tumor.

In some examples of the methods provided herein, the methods further include the step of administering one or more additional anti-cancer therapies. Exemplary anti-cancer therapies that can be administered for cancer therapy in the methods provided include, but are not limited to, chemotherapeutic compounds (e.g., toxins, alkylating agents, nitrosoureas, anticancer antibiotics, antimetabolites, antimitotics, topoisomerase inhibitors), cytokines, growth factors, hormones, photosensitizing agents, radionuclides, signaling modulators, anticancer antibodies, anticancer oligopeptides, anticancer oligonucleotides (e.g., antisense RNA and siRNA), angiogenesis inhibitors, radiation therapy, or a combination thereof. Exemplary chemotherapeutic compounds include, but are not limited to, Ara-C, cisplatin, carboplatin, paclitaxel, doxorubicin, gemcitabine, camptothecin, irinotecan, cyclophosphamide, 6-mercaptopurine, vincristine, 5-fluorouracil, and methotrexate. Anticancer agents include anti-metastatic agents. In some examples, the anti-cancer agent is an oncolytic virus, such as an LIVP vaccinia virus.

In some examples, the virus is administered at a therapeutic dosage, for example, at a dosage of between at or about 1×10⁶ pfu to at or about 1×10¹⁴ pfu, such as at least, or about or at 1×10⁶ pfu, 1×10⁷ pfu or 1×10⁸ pfu, 1×10⁹ pfu, 1×10¹⁰ pfu, 1×10¹¹ pfu, 1×10¹² pfu, 1×10¹³ pfu, or 1×10¹⁴ pfu.

In the provided methods, the antibiotic can be administered prior to, at the same time as, after, during, or intermittently with administration of the virus to the subject. In some examples of the methods, the antibiotic is administered prior to administration of the virus to the subject. For example, the antibiotic is administered at least, at about or at 1, 2, 3, 4, 5, 6, 7, 8, 9, 10, 11, 12, 13, 14, 15, 16, 17, 18, 19, 20, 21, 22, 23, 24, 36 or 48 or more hours prior to administration of the virus to the subject. In other examples of the methods, the antibiotic is administered concurrent with, or at the same time as, administration of the virus to the subject. In yet other examples of the methods, the antibiotic is administered after administration of the virus to the subject. For example, the antibiotic is administered at least, at about or at ¼, ½, 1, 2, 3, 4, 5, 6, 7, 8, 9, 10, 11, 12, 13, 14, 15, 16, 17, 18, 19, 20, 21, 22, 23, 24 or more hours, 2, 3, 4, 5, 6, 7, 8, 9, 10, 11, 12, 13, 14 or more days after administration of the virus to the subject. The antibiotic can be administered once, or can be administered several times over the cycle of administration of the virus. For example, the antibiotic can be administered 1, 2, 3, 4, 5, 6, 7, 8, 9, 10 or more times over the cycle of administration of the virus.

The methods provided herein for use in treating cancers or tumors can be used in combination with one or more additional methods for detecting or monitoring a cancer or tumor or monitoring an anti-cancer therapy. For example, a tumor or metastasis can be detected by physical examination of subject, laboratory tests, such as blood or urine tests, imaging and genetic testing, such as testing for gene mutations that are known to cause cancer. A tumor or metastasis can be detected using in vivo imaging techniques, such as digital X-ray radiography, mammography, CT (computerized tomography) scanning, MRI (magnetic resonance imaging), ultrasonography and PET (positron emission tomography) scanning. Alternatively, a tumor can be detected using tumor markers in blood, serum or urine, that is, by monitoring substances produced by tumor cells or by other cells in the body in response to cancer. For example, prostate specific antigen (PSA) levels are used to detect prostate cancer in men. Additionally, tumors can be detected and monitored by biopsy.

Any of a variety of monitoring steps can be used to monitor an anti-cancer therapy, including, but not limited to, monitoring tumor size, monitoring anti-(tumor antigen) antibody titer, monitoring anti-virus antibody titer, monitoring the presence and/or size of metastases, monitoring the subject's lymph nodes, monitoring the subject's weight or other health indicators including blood or urine markers, monitoring expression of a detectable gene product, and monitoring titer of the oncolytic reporter virus, in a tumor, tissue or organ of a subject.

1. Therapeutic Methods

The viruses provided herein, including the clonal virus strains, for example, can be used for the treatment of proliferative disorders or conditions, including the treatment (such as inhibition) of cancerous cells, neoplasms, tumors, metastases, cancer stem cells, and other immunoprivileged cells or tissues, such as wounded or inflamed tissues. The viruses provided herein preferentially accumulate in tumors or metastases. In some examples, the administration of a virus provided herein results in a slowing of tumor growth. In other examples, the administration of a virus provided herein results in a decrease in tumor volume, including elimination or eradication of the tumor. The therapeutic methods and uses provided herein, however, do not require the administered virus to kill tumor cells or decrease the tumor size. Instead, the methods provided herein include administering to a subject a virus provided herein that can cause or enhance an anti-tumor immune response in the subject. In some examples, the viruses provided herein can be administered to a subject without causing viral-induced disease in the subject. In some examples, the viruses can elicit an anti-tumor immune response in the subject, where typically the viral-mediated anti-tumor immune response can develop, for example, over several days, a week or more, 10 days or more, two weeks or more, or a month or more. In some exemplary methods, the virus can be present in the tumor, and can cause an anti-tumor immune response without the virus itself causing enough tumor cell death to prevent tumor growth. In some examples, the tumor is a monotherapeutic tumor or monotherapeutic cancer, where the tumor or cancer does not decrease in volume when treated with the virus or a therapeutic agent alone.

In some examples, the therapeutic methods provided herein inhibit tumor growth in a subject, where the methods include administering to a subject a virus that can accumulate in a tumor and/or metastasis, and can cause or enhance an anti-tumor immune response. The anti-tumor immune response induced as a result of tumor or metastases-accumulated viruses can result in inhibition of tumor growth.

In some examples, the therapeutic methods provided herein inhibit growth or formation of a metastasis in a subject, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in inhibition of metastasis growth or formation.

In other examples, the therapeutic methods provided herein decrease the size of a tumor and/or metastasis in a subject, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in a decrease in the size of the tumor and/or metastasis.

In some examples, the therapeutic methods provided herein eliminate a tumor and/or metastasis from a subject, where the methods include administering to a subject a virus provided herein that can accumulate in a tumor and/or metastasis, and can cause or enhance an anti-tumor immune response. The anti-tumor immune response induced as a result of tumor or metastasis-accumulated viruses can result in elimination of the tumor and/or metastasis from the subject.

Methods of reducing or inhibiting tumor growth, inhibiting metastasis growth and/or formation, decreasing the size of a tumor or metastasis, eliminating a tumor or metastasis and/or cancer stem cell or other tumor therapeutic methods provided herein include causing or enhancing an anti-tumor immune response in the host. The immune response of the host, being anti-tumor in nature, can be mounted against tumors and/or metastases in which viruses have accumulated, and can also be mounted against tumors and/or metastases in which viruses have not accumulated, including tumors and/or metastases that form after administration of the virus to the subject. Accordingly, a tumor and/or metastasis whose growth or formation is inhibited, or whose size is decreased, or that is eliminated, can be a tumor and/or metastasis in which the viruses have accumulated, or also can be a tumor and/or metastasis in which the viruses have not accumulated. Accordingly, provided herein are methods of reducing or inhibiting tumor growth, inhibiting metastasis growth and/or formation, decreasing the size of a tumor or metastasis, eliminating a tumor or metastasis, or other tumor therapeutic methods, where the method includes administering to a subject a virus provided herein, where the virus accumulates in at least one tumor or metastasis and causes or enhances an anti-tumor immune response in the subject, and the immune response also is mounted against a tumor and/or metastasis in which the virus cell did not accumulate. In another example, methods are provided for inhibiting or preventing recurrence of a neoplastic disease or inhibiting or preventing new tumor growth, where the methods include administering to a subject a virus provided herein that can accumulate in a tumor and/or metastasis, and can cause or enhance an anti-tumor immune response, and the anti-tumor immune response can inhibit or prevent recurrence of a neoplastic disease or inhibit or prevent new tumor growth.

The tumor or neoplastic disease therapeutic methods provided herein, such as methods of reducing or inhibiting tumor growth, inhibiting metastasis growth and/or formation, decreasing the size of a tumor or metastasis, eliminating a tumor or metastasis, or other tumor therapeutic methods, also can include administering to a subject a virus provided herein that can cause tumor cell lysis or tumor cell death. Such a virus can be the same virus as the virus that can cause or enhance an anti-tumor immune response in the subject. Viruses, such as the viruses provided herein, can cause cell lysis or tumor cell death as a result of expression of an endogenous gene or as a result of an exogenous gene. Endogenous or exogenous genes can cause tumor cell lysis or inhibit cell growth as a result of direct or indirect actions, as is known in the art, including lytic channel formation or activation of an apoptotic pathway. Gene products, such as exogenous gene products can function to activate a prodrug to an active, cytotoxic form, resulting in cell death where such genes are expressed.

Such methods of tumor and/or metastasis treatment can include administration of a virus provided herein for therapy, such as for gene therapy, for cancer gene therapy, or for vaccine therapy. Such a virus can be used to stimulate humoral and/or cellular immune response, induce strong cytotoxic T lymphocytes responses in subjects who can benefit from such responses. For example, the virus can provide prophylactic and therapeutic effects against a tumor infected by the virus or other infectious diseases, by rejection of cells from tumors or lesions using viruses that express immunoreactive antigens (Earl et al., Science 234:728-831 (1986); Lathe et al., Nature (London) 32:878-880 (1987)), cellular tumor-associated antigens (Bernards et al., Proc. Natl. Acad. Sci. USA 84:6854-6858 (1987); Estin et al., Proc. Natl. Acad. Sci. USA 85:1052-1056 (1988); Kantor et al., J. Natl. Cancer Inst. 84: 1084-1091 (1992); Roth et al., Proc. Natl. Acad. Sci. USA 93:4781-4786 (1996)) and/or cytokines (e.g., IL-2, IL-12), costimulatory molecules (B7-1, B7-2) (Rao et al., J. Immunol. 156: 3357-3365 (1996); Chamberlain et al., Cancer Res. 56: 2832-2836 (1996); Oertli et al., J. Gen. Virol. 77: 3121-3125 (1996); Qin and Chatterjee, Human Gene Ther. 7: 1853-1860 (1996); McAneny et al., Ann. Surg. Oncol. 3: 495-500 (1996)), or other therapeutic proteins.

As shown previously, solid tumors can be treated with viruses, such as vaccinia viruses, resulting in an enormous tumor-specific virus replication, which can lead to tumor protein antigen and viral protein production in the tumors (U.S. Patent Publication No. 2005-0031643, now U.S. Pat. Nos. 7,588,767, 7,588,771, 7,662,398), which provide and exemplify the GLV-1h68 virus and derivatives thereof. Vaccinia virus administration to mice resulted in lysis of the infected tumor cells and a resultant release of tumor-cell-specific antigens. Continuous leakage of these antigens into the body led to a very high level of antibody titer (in approximately 7-14 days) against tumor proteins, viral proteins, and the virus encoded engineered proteins in the mice. The newly synthesized anti-tumor antibodies and the enhanced macrophage, neutrophils count were continuously delivered via the vasculature to the tumor and thereby provided for the recruitment of an activated immune system against the tumor. The activated immune system then eliminated the foreign compounds of the tumor including the viral particles. This interconnected release of foreign antigens boosted antibody production and continuous response of the antibodies against the tumor proteins to function like an autoimmunizing vaccination system initiated by vaccinia viral infection and replication, followed by cell lysis, protein leakage and enhanced antibody production. Thus, the viruses provided herein and the viruses generated using the methods provided herein can be administered in a complete process that can be applied to all tumor systems with immunoprivileged tumor sites as site of privileged viral growth, which can lead to tumor elimination by the host's own immune system.

In one example, the tumor treated is a cancer such as pancreatic cancer, non-small cell lung cancer, multiple myeloma or leukemia, although the cancer is not limited in this respect, and other metastatic diseases can be treated by the combinations provided herein. For example, the tumor treated can be a solid tumor, such as of the lung and bronchus, breast, colon and rectum, kidney, stomach, esophagus, liver and intrahepatic bile duct, urinary bladder, brain and other nervous system, head and neck, oral cavity and pharynx, cervix, uterine corpus, thyroid, ovary, testes, prostate, malignant melanoma, cholangiocarcinoma, thymoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS. Exemplary tumors include, for example, pancreatic tumors, ovarian tumors, lung tumors, colon tumors, prostate tumors, cervical tumors and breast tumors. In one example, the tumor is a carcinoma such as, for example, an ovarian tumor or a pancreatic tumor.

In other examples, methods are provided for immunizing a subject, where the methods include administering to the subject a virus that expresses one or more antigens against which antigens the subject will develop an immune response. The immunizing antigens can be endogenous to the virus, such as vaccinia antigens on a vaccinia virus used to immunize against smallpox, measles, mumps, or the immunizing antigens can be exogenous antigens expressed by the virus, such as influenza or HIV antigens expressed on a viral capsid surface. In the case of smallpox, for example, a tumor specific protein antigen can be carried by an attenuated vaccinia virus (encoded by the viral genome) for a smallpox vaccine. Thus, the viruses provided herein, including the modified vaccinia viruses can be used as vaccines.

In some examples, provided herein are methods for eliciting or enhancing antibody production against a selected antigen or a selected antigen type in a subject, where the methods include administering to a subject a virus that can accumulate in a tumor and/or metastasis, and can cause release of a selected antigen or selected antigen type from the tumor, resulting in antibody production against the selected antigen or selected antigen type. Any of a variety of antigens can be targeted in the methods provided herein, including a selected antigen such as an exogenous gene product expressed by the virus, or a selected antigen type such as one or more tumor antigens release from the tumor as a result of viral infection of the tumor (e.g., by lysis, apoptosis, secretion or other mechanism of causing antigen release from the tumor).

In some examples, it can be desirable to maintain release of the selected antigen or selected antigen type over a series of days, for example, at least a week, at least ten days, at least two weeks or at least a month. Provided herein are methods for providing a sustained antigen release within a subject, where the methods include administering to a subject a virus that can accumulate in a tumor and/or metastasis, and can cause sustained release of an antigen, resulting in antibody production against the antigen. The sustained release of antigen can result in an immune response by the viral-infected host, in which the host can develop antibodies against the antigen, and/or the host can mount an immune response against cells expressing the antigen, including an immune response against tumor cells. Thus, the sustained release of antigen can result in immunization against tumor cells. In some examples, the viral-mediated sustained antigen release-induced immune response against tumor cells can result in complete removal or killing of all tumor cells.

2. Pharmaceutical Compositions, Combinations and Kits

Provided herein are pharmaceutical compositions, combinations and kits for practicing the methods provided herein. For example, provide herein are pharmaceutical compositions containing an antibiotic, a virus and a pharmaceutical carrier. Combinations can include, for example, an antibiotic and two or more viruses; an antibiotic, a virus and a detectable compound; an antibiotic, a virus and a therapeutic compound; an antibiotic, a virus and a viral expression modulating compound; or any combination thereof. Kits can include one or more pharmaceutical compositions or combinations provided herein, and one or more components, such as instructions for use, a device for administering the pharmaceutical composition or combination to a subject, a device for administering a therapeutic or diagnostic compound to a subject or a device for detecting a virus in a subject.

The pharmaceutical compositions, combinations, and kits provided herein can be used to increase the effectiveness of therapeutic viral therapy for the treatment of tumors, for example, by containing an antibiotic, such as an antibiotic that is not an anti-cancer antibiotic, that inhibits the growth of or kills commensal gut bacteria to thereby reduce the number of gut bacteria. Thus, the pharmaceutical compositions, combinations and kits typically contain therapeutically effective amounts of the virus and antibiotic. Therapeutically effective amounts for virus and antibiotic, provided in compositions, combinations, and kits, depend upon the virus and antibiotic in the composition and the subject to whom the composition is administered. Exemplary therapeutic effective amounts of virus and antibiotic are described above in the current section and in Section C, respectively.

An antibiotic and virus contained in a pharmaceutical composition, combination or kit can include any antibiotic or virus provided herein. The pharmaceutical compositions, combinations or kits can include one or more additional viruses that can be selected from a viruses provided herein, or other therapeutic or diagnostic virus, such as any oncolytic virus provided herein.

a. Pharmaceutical Compositions

Provided herein are pharmaceutical compositions containing an antibiotic, a virus and a suitable pharmaceutical carrier. The pharmaceutical compositions provided herein can be formulated for single dose or multiple dose administration. For example, pharmaceutical composition formulated for multiple dosage administration can be diluted to a desired dose for single dosage administration.

Exemplary therapeutically effective amounts of the composition depend upon the virus and antibiotic in the composition and the subject to whom the composition is administered. Exemplary therapeutic effective amounts of virus and antibiotic are described above in the current section and in Section C, respectively. Typically, single dosage amounts of the pharmaceutical compositions provided are between or about between at least 1 mg and at least 10 g, inclusive; or between or about between at least 1 mg and at least 1 gm, inclusive; or between or about at least 500 mg and at or about at least 5 g; or is or is at least about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975 or 1000 mg, 1.5, 2, 2.5, 3, 3.5, 4, 4.5, or 5 g.

A pharmaceutically acceptable carrier, for the provided compositions, includes a solid, semi-solid or liquid material that acts as a vehicle carrier or medium for the virus. Pharmaceutical compositions provided herein can be formulated in various forms, for example in solid, semi-solid, aqueous, liquid, powder or lyophilized form. Exemplary pharmaceutical compositions containing a virus provided herein include, but are not limited to, sterile injectable solutions, sterile packaged powders, eye drops, tablets, pills, powders, lozenges, sachets, cachets, elixirs, suspensions, emulsions, solutions, syrups, aerosols (as a solid or in a liquid medium), ointments, soft and hard gelatin capsules, and suppositories.

Examples of suitable pharmaceutical carriers are known in the art and include, but are not limited to, water, buffers, saline solutions, phosphate buffered saline solutions, various types of wetting agents, sterile solutions, alcohols, gum arabic, vegetable oils, benzyl alcohols, gelatin, glycerin, carbohydrates, such as lactose, sucrose, dextrose, amylose or starch, sorbitol, mannitol, magnesium stearate, talc, silicic acid, viscous paraffin, perfume oil, fatty acid monoglycerides and diglycerides, pentaerythritol fatty acid esters, hydroxy methylcellulose, powders, among others. Pharmaceutical compositions provided herein can contain other additives including, for example, antioxidants, preserving agents, analgesic agents, binders, disintegrants, coloring, diluents, excipients, extenders, glidants, solubilizers, stabilizers, tonicity agents, vehicles, viscosity agents, flavoring agents, sweetening agents, emulsions, such as oil/water emulsions, emulsifying and suspending agents, such as acacia, agar, alginic acid, sodium alginate, bentonite, carbomer, carrageenan, carboxymethylcellulose, cellulose, cholesterol, gelatin, hydroxyethyl cellulose, hydroxypropyl cellulose, hydroxypropyl methylcellulose, methylcellulose, octoxynol 9, oleyl alcohol, povidone, propylene glycol monostearate, sodium lauryl sulfate, sorbitan esters, stearyl alcohol, tragacanth, xanthan gum, and derivatives thereof, solvents, and miscellaneous ingredients, such as, but not limited to, crystalline cellulose, microcrystalline cellulose, citric acid, dextrin, liquid glucose, lactic acid, lactose, magnesium chloride, potassium metaphosphate, starch, among others. Such carriers and/or additives can be formulated by conventional methods and can be administered to the subject at a suitable dose. Stabilizing agents such as lipids, nuclease inhibitors, polymers, and chelating agents can preserve the compositions from degradation within the body. Other suitable formulations for use in a pharmaceutical composition can be found, for example, in Remington: The Science and Practice of Pharmacy (2005, Twenty-first edition, Gennaro & Gennaro, eds., Lippincott Williams and Wilkins).

Pharmaceutical formulations that include a virus provided herein for injection or mucosal delivery typically include aqueous solutions of the virus provided in a suitable buffer for injection or mucosal administration or lyophilized forms of the virus for reconstitution in a suitable buffer for injection or mucosal administration. Such formulations optionally can contain one or more pharmaceutically acceptable carriers and/or additives as described herein or known in the art. Liquid compositions for oral administration generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Pharmaceutical compositions provided herein can be formulated to provide quick, sustained or delayed released of a virus as described herein by employing procedures known in the art. For preparing solid compositions such as tablets, a virus provided herein is mixed with a pharmaceutical carrier to form a solid composition. Optionally, tablets or pills are coated or otherwise compounded to provide a dosage form affording the advantage of prolonged action in the subject. For example, a tablet or pill contains an inner dosage and an outer dosage component, the latter being in the form of an envelope over the former. The two components can be separated by an enteric layer, for example, which serves to resist disintegration in the stomach and permit the inner component to pass intact into the duodenum or to be delayed in release. A variety of materials are used for such enteric layers or coatings, including, for example, a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. These liquid or solid compositions optionally can contain suitable pharmaceutically acceptable excipients and/or additives as described herein or known in the art. Such compositions are administered, for example, by the oral or nasal respiratory route for local or systemic effect. Compositions in pharmaceutically acceptable solvents are nebulized by use of inert gases. Nebulized solutions are inhaled, for example, directly from the nebulizing device, from an attached face mask tent, or from an intermittent positive pressure breathing machine. Solution, suspension, or powder compositions are administered, orally or nasally, for example, from devices which deliver the formulation in an appropriate manner such as, for example, use of an inhaler.

Pharmaceutical compositions provided herein can be formulated for transdermal delivery via a transdermal delivery devices (“patches”). Such transdermal patches are used to provide continuous or discontinuous infusion of a virus provided herein. The construction and use of transdermal patches for the delivery of pharmaceutical agents are performed according to methods known in the art. See, for example, U.S. Pat. No. 5,023,252. Such patches are constructed for continuous, pulsatile, or on-demand delivery of a virus provided herein.

Colloidal dispersion systems that can be used for delivery of viruses include macromolecule complexes, nanocapsules, microspheres, beads and lipid-based systems including oil-in-water emulsions (mixed), micelles, liposomes and lipoplexes. An exemplary colloidal system is a liposome. Organ-specific or cell-specific liposomes can be used in order to achieve delivery only to the desired tissue. The targeting of liposomes can be carried out by the person skilled in the art by applying commonly known methods. This targeting includes passive targeting (utilizing the natural tendency of the liposomes to distribute to cells of the RES in organs which contain sinusoidal capillaries) or active targeting (for example, by coupling the liposome to a specific ligand, for example, an antibody, a receptor, sugar, glycolipid and protein by methods know to those of skill in the art). Monoclonal antibodies can be used to target liposomes to specific tissues, for example, tumor tissue, via specific cell-surface ligands.

b. Combinations

Provided are combinations of an antibody, a virus and an additional agent, such as a second virus or other therapeutic or diagnostic agent. A combination can include an antibody and a virus with one or more additional viruses, including, for example, one or more additional diagnostic or therapeutic viruses. A combination can contain pharmaceutical compositions containing a virus provided herein or host cells containing a virus as described herein. A combination also can include any antibody, virus or reagent for effecting treatment or diagnosis in accord with the methods provided herein such as, for example, an antiviral or chemotherapeutic agent. Combinations also can contain a compound used for the modulation of gene expression from endogenous or heterologous genes encoded by the virus.

Combinations provided herein can contain an antibody, a virus and a therapeutic compound. Therapeutic compounds for the compositions provided herein can be, for example, an anti-cancer or chemotherapeutic compound. Exemplary therapeutic compounds include, for example, cytokines, growth factors, photosensitizing agents, radionuclides, toxins, siRNA molecules, enzyme/pro E drug pairs, anti-metabolites, signaling modulators, anti-cancer antibiotics, anti-cancer antibodies, angiogenesis inhibitors, chemotherapeutic compounds, antimetastatic compounds or a combination of any thereof. Viruses provided herein can be combined with an anti-cancer compound, such as a platinum coordination complex. Exemplary platinum coordination complexes include, for example, cisplatin, carboplatin, oxaliplatin, DWA2114R, NK121, IS 3 295, and 254-S. Exemplary chemotherapeutic agents also include, but are not limited to, methotrexate, vincristine, adriamycin, non-sugar containing chloroethylnitrosoureas, 5-fluorouracil, mitomycin C, bleomycin, doxorubicin, dacarbazine, taxol, fragyline, Meglamine GLA, valrubicin, carmustine, polifeprosan, MM1270, BAY 12-9566, RAS farnesyl transferase inhibitor, farnesyl transferase inhibitor, MMP, MTA/LY231514, lometrexol/LY264618, Glamolec, CI-994, TNP-470, Hycamtin/topotecan, PKC412, Valspodar/PSC833, Novantrone/mitoxantrone, Metaret/suramin, BB-94/batimastat, E7070, BCH-4556, CS-682, 9-AC, AG3340, AG3433, Incel/VX-710, VX-853, ZD0101, IS1641, ODN 698, TA 2516/marimastat, BB2516/marimastat, CDP 845, D2163, PD183805, DX8951f, Lemonal (DP-2202), FK 317, picibanil/OK-432, valrubicin/AD 32, strontium-89/Metastron, Temodal/temozolomide, Yewtaxan/paclitaxel, Taxol/paclitaxel, Paxex/paclitaxel, Cyclopax/oral paclitaxel, Xeloda/capecitabine, Furtulon/doxifluridine, oral taxoids, SPU-077/cisplatin, HMR 1275/flavopiridol, CP-358 (774)/EGFR, CP-609 (754)/RAS oncogene inhibitor, BMS-182751/oral platinum, UFT(Tegafur/Uracil), Ergamisol/levamisole, Campto/levamisole, Eniluracil/776C85/5FU enhancer, Camptosar/irinotecan, Tomudex/raltitrexed, Leustatin/cladribine, Caelyx/liposomal doxorubicin, Myocet/liposomal doxorubicin, Doxil/liposomal doxorubicin, Evacet/liposomal doxorubicin, Fludara/fludarabine, Pharmorubicin/epirubicin, DepoCyt, ZD 1839, LU 79553/Bis-Naphthalimide, LU 103793/Dolastatin, Gemzar/gemcitabine, ZD 0473/AnorMED, YM 116, Iodine seeds, CDK4 and CDK2 inhibitors, PARP inhibitors, D4809/dexifosfamide, Ifex/Mesnex/ifosfamide, Vumon/teniposide, Paraplatin/carboplatin, Platinol/cisplatin, VePesid/Eposin/Etopophos/etoposide, ZD 9331, Taxotere/docetaxel, prodrugs of guanine arabinoside, taxane analogs, nitrosoureas, alkylating agents such as melphalan and cyclophosphamide, aminoglutethimide, asparaginase, busulfan, carboplatin, chlorambucil, cytarabine HCl, dactinomycin, daunorubicin HCl, estramustine phosphate sodium, etoposide (VP16-213), floxuridine, fluorouracil (5-FU), flutamide, hydroxyurea (hydroxycarbamide), ifosfamide, interferon alfa-2a, interferon alfa-2b, leuprolide acetate (LHRH-releasing factor analogue), lomustine (CCNU), mechlorethamine HCl (nitrogen mustard), mercaptopurine, mesna, mitotane (o,p′-DDD), mitoxantrone HCl, octreotide, plicamycin, procarbazine HCl, streptozocin, tamoxifen citrate, thioguanine, thiotepa, vinblastine sulfate, amsacrine (m-AMSA), azacitidine, erythropoietin, hexamethylmelamine (HMM), interleukin 2, mitoguazone (methyl-GAG; methyl glyoxal bis-guanylhydrazone; MGBG), pentostatin (2′ deoxycoformycin), semustine (methyl-CCNU), teniposide (VM-26) and vindesine sulfate. Additional exemplary therapeutic compounds for the use in pharmaceutical compositions and combinations provided herein can be found elsewhere herein (see e.g., Section I for exemplary cytokines, growth factors, photosensitizing agents, radionuclides, toxins, siRNA molecules, enzyme/pro-drug pairs, anti-metabolites, signaling modulators, anti-cancer antibiotics, anti-cancer antibodies, angiogenesis inhibitors, and chemotherapeutic compounds).

In some examples, the combination can include additional therapeutic compounds such as, for example, compounds that are substrates for enzymes encoded and expressed by the virus, or other therapeutic compounds provided herein or known in the art to act in concert with a virus. For example, the virus can express an enzyme that converts a prodrug into an active chemotherapy drug for killing the cancer cell. Hence, combinations provided herein can contain a therapeutic compound, such as a prodrug. An exemplary virus/therapeutic compound combination can include a virus encoding Herpes simplex virus thymidine kinase with the prodrug ganciclovir. Additional exemplary enzyme/pro-drug pairs, for the use in combinations provided include, but are not limited to, varicella zoster thymidine kinase/ganciclovir, cytosine deaminase/5-fluorouracil, purine nucleoside phosphorylase/6-methylpurine deoxyriboside, beta lactamase/cephalosporin-doxorubicin, carboxypeptidase G2/4-[(2-chloroethyl)(2-mesyloxyethyl)amino]benzoyl-L-glutamic acid, cytochrome P450/acetaminophen, horseradish peroxidase/indole-3-acetic acid, nitroreductase/CB 1954, rabbit carboxylesterase/7-ethyl-10-[4-(1-piperidino)-1-piperidino]carbonyloxycamptothecin (CPT-11), mushroom tyrosinase/bis-(2-chloroethyl)amino-4-hydroxyphenylaminomethanone 28, beta galactosidase/1-chloromethyl-5-hydroxy-1,2-dihydro-3H-benz[e]indole, beta glucuronidase/epirubicin-glucuronide, thymidine phosphorylase/5′-deoxy-5-fluorouridine, deoxycytidine kinase/cytosine arabinoside, beta-lactamase and linamarase/linamarin. Additional exemplary prodrugs, for the use in combinations can also be found elsewhere herein (see e.g., Section I). Any of a variety of known combinations provided herein or otherwise known in the art can be included in the combinations provided herein.

In some examples, the combination can include compounds that can kill or inhibit viral growth or toxicity. Such compounds can be used to alleviate one or more adverse side effects that can result from viral infection (see, e.g. U.S. Patent Pub. No. US 2009-0162288-A1). Combinations provided herein can contain antifungal, anti-parasitic or antiviral compounds for treatment of infections. In some examples, the antiviral compound is a chemotherapeutic agent that inhibits viral growth or toxicity. Exemplary antifungal agents which can be included in a combination with a virus provided herein include, but are not limited to, amphotericin B, dapsone, fluconazole, flucytosine, griseofulvin, itraconazole, ketoconazole, miconazole, clotrimazole, nystatin, and combinations thereof. Exemplary antiviral agents can be included in a combination with a virus provided herein include, but are not limited to, cidofovir, alkoxyalkyl esters of cidofovir (CDV), cyclic CDV, and (S)-9-(3-hydroxy-2 phosphonylmethoxypropyl)adenine, 5-(dimethoxymethyl)-2′-deoxyuridine, isatin-beta-thiosemicarbazone, N-methanocarbathymidine, brivudine, 7-deazaneplanocin A, ST-246, Gleevec, 2′-beta-fluoro-2′,3′-dideoxyadenosine, indinavir, nelfinavir, ritonavir, nevirapine, AZT, ddI, ddC, and combinations thereof. Typically, combinations with an antiviral agent contain an antiviral agent known to be effective against the virus of the combination. For example, combinations can contain a vaccinia virus with an antiviral compound, such as cidofovir, alkoxyalkyl esters of cidofovir, ganciclovir, acyclovir, ST-246, Gleevec, and derivatives thereof.

In some examples, the combination can include a detectable compound. A detectable compound can include, for example, a ligand, substrate or other compound that can interact with and/or bind specifically to a protein or RNA encoded and expressed by the virus, and can provide a detectable signal, such as a signal detectable by tomographic, spectroscopic, magnetic resonance, or other known techniques. In some examples, the protein or RNA is an exogenous protein or RNA. In some examples, the protein or RNA expressed by the virus modifies the detectable compound where the modified compound emits a detectable signal. Exemplary detectable compounds can be, or can contain, an imaging agent such as a magnetic resonance, ultrasound or tomographic imaging agent, including a radionuclide. The detectable compound can include any of a variety of compounds as provided elsewhere herein or are otherwise known in the art. Exemplary proteins that can be expressed by the virus and a detectable compound combinations employed for detection include, but are not limited to luciferase and luciferin, β-galactosidase and (4,7,10-tri(acetic acid)-1-(2-β-galactopyranosylethoxy)-1,4,7,10-tetraazacyclododecane) gadolinium (Egad), and other combinations known in the art.

In some examples, the combination can include a gene expression modulating compound that regulates expression of one or more genes encoded by the virus. Compounds that modulate gene expression are known in the art, and include, but are not limited to, transcriptional activators, inducers, transcriptional suppressors, RNA polymerase inhibitors and RNA binding compounds such as siRNA or ribozymes. Any of a variety of gene expression modulating compounds known in the art can be included in the combinations provided herein. Typically, the gene expression modulating compound included with a virus in the combinations provided herein will be a compound that can bind, inhibit or react with one or more compounds, active in gene expression such as a transcription factor or RNA of the virus of the combination. An exemplary virus/expression modulator combinations can be a virus encoding a chimeric transcription factor complex having a mutant human progesterone receptor fused to a yeast GAL4 DNA-binding domain an activation domain of the herpes simplex virus protein VP16 and also containing a synthetic promoter containing a series of GAL4 recognition sequences upstream of the adenovirus major late E1B TATA box, where the compound can be RU486 (see, e.g., Yu et al., (2002) Mol Genet Genomics 268:169-178). A variety of other virus/expression modulator combinations known in the art also can be included in the combinations provided herein.

In some examples, the combination can contain nanoparticles. Nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen.

In some examples, the combination can contain one or more additional therapeutic and/or diagnostic viruses or other therapeutic and/or diagnostic microorganism (e.g. therapeutic and/or diagnostic bacteria) for diagnosis or treatment. Exemplary therapeutic and/or diagnostic viruses are known in the art and include, but are not limited to, therapeutic and/or diagnostic poxviruses, herpesviruses, adenoviruses, adeno-associated viruses, and reoviruses. Exemplary of such oncolytic viruses are described herein above.

c. Kits

The viruses, cells, pharmaceutical compositions or combinations provided herein can be packaged as kits. Kits can optionally include one or more components such as instructions for use, devices and additional reagents, and components, such as tubes, containers and syringes for practice of the methods. Exemplary kits can include an antibody, a virus, and can optionally include instructions for use, a device for detecting a virus in a subject, a device for administering the antibody to a subject, a device for administering the virus to a subject, or a device for administering an additional agent or compound to a subject.

In one example, a kit can contain instructions. Instructions typically include a tangible expression describing the virus and, optionally, other components included in the kit, and methods for administration, including methods for determining the proper state of the subject, the proper dosage amount, and the proper administration method, for administering the virus and antibiotic. Instructions can also include guidance for monitoring the subject over the duration of the treatment time.

In another example, a kit can contain a device for detecting a virus in a subject. Devices for detecting a virus in a subject can include a low light imaging device for detecting light, for example, emitted from luciferase, or fluoresced from fluorescent protein, such as a green or red fluorescent protein, a magnetic resonance measuring device such as an MRI or NMR device, a tomographic scanner, such as a PET, CT, CAT, SPECT or other related scanner, an ultrasound device, or other device that can be used to detect a protein expressed by the virus within the subject. Typically, the device of the kit will be able to detect one or more proteins expressed by the virus of the kit. Any of a variety of kits containing viruses and detection devices can be included in the kits provided herein, for example, a virus expressing luciferase and a low light imager or a virus expressing fluorescent protein, such as a green or red fluorescent protein, and a low light imager.

Kits provided herein also can include a device for administering a virus and antibiotic to a subject. Any of a variety of devices known in the art for administering medications, pharmaceutical compositions and vaccines can be included in the kits provided herein. Exemplary devices include, but are not limited to, a hypodermic needle, an intravenous needle, a catheter, a needle-less injection device, an inhaler and a liquid dispenser, such as an eyedropper. For example, a virus or antibiotic to be delivered systemically, for example, by intravenous injection, can be included in a kit with a hypodermic needle and syringe. Typically, the device for administering a virus or antibiotic of the kit will be compatible with the virus of the kit; for example, a needle-less injection device such as a high pressure injection device can be included in kits with viruses not damaged by high pressure injection, but is typically not included in kits with viruses damaged by high pressure injection.

Kits provided herein also can include a device for administering an additional agent or compound to a subject. Any of a variety of devices known in the art for administering medications to a subject can be included in the kits provided herein. Exemplary devices include, but are not limited to, a hypodermic needle, an intravenous needle, a catheter, a needle-less injection device, an inhaler and a liquid dispenser, such as an eyedropper. Typically the device for administering the compound of the kit will be compatible with the desired method of administration of the compound. For example, a compound to be delivered systemically or subcutaneously can be included in a kit with a hypodermic needle and syringe.

The kits provided herein also can include any device for applying energy to a subject, such as electromagnetic energy. Such devices include, but are not limited to, a laser, light-emitting diodes, fluorescent lamps, dichroic lamps, and a light box. Kits also can include devices to effect internal exposure of energy to a subject, such as an endoscope or fiber optic catheter.

3. Dosages and Administration

A virus provided herein can be administered to a subject, including a subject having a tumor or having neoplastic cells, or a subject to be immunized, or a subject for gene therapy. An administered virus can be a virus provided herein or any other virus generated using the methods provided herein. In some examples, the virus administered is a virus containing a characteristic such as attenuated pathogenicity, low toxicity, preferential accumulation in tumor, ability to activate an immune response against tumor cells, high immunogenicity, replication competence and ability to express exogenous proteins, and combinations thereof.

a. Steps prior to administering the virus

In some examples, one or more steps can be performed prior to administration of the virus to the subject. Any of a variety of preceding steps can be performed, including, but not limited to diagnosing the subject with a condition appropriate for virus administration, determining the immunocompetence of the subject, immunizing the subject, treating the subject with a chemotherapeutic agent, treating the subject with radiation, or surgically treating the subject.

For examples that include administering a virus to a tumor-bearing subject for therapeutic purposes, the subject has typically been previously diagnosed with a neoplastic condition. Diagnostic methods also can include determining the type of neoplastic condition, determining the stage of the neoplastic conditions, determining the size of one or more tumors in the subject, determining the presence or absence of metastatic or neoplastic cells in the lymph nodes of the subject, or determining the presence of metastases of the subject. Some examples of therapeutic methods for administering a virus to a subject can include a step of determination of the size of the primary tumor or the stage of the neoplastic disease, and if the size of the primary tumor is equal to or above a threshold volume, or if the stage of the neoplastic disease is at or above a threshold stage, a virus is administered to the subject. In a similar example, if the size of the primary tumor is below a threshold volume, or if the stage of the neoplastic disease is at or below a threshold stage, the virus is not yet administered to the subject; such methods can include monitoring the subject until the tumor size or neoplastic disease stage reaches a threshold amount, and then administering the virus to the subject. Threshold sizes can vary according to several factors, including rate of growth of the tumor, ability of the virus to infect a tumor, and immunocompetence of the subject. Generally the threshold size will be a size sufficient for a virus to accumulate and replicate in or near the tumor without being completely removed by the host's immune system, and will typically also be a size sufficient to sustain a virus infection for a time long enough for the host to mount an immune response against the tumor cells, typically about one week or more, about ten days or more, or about two weeks or more. Exemplary threshold tumor sizes for viruses, such as vaccinia viruses, are at least about 100 mm³, at least about 200 mm³, at least about 300 mm³, at least about 400 mm³, at least about 500 mm³, at least about 750 mm³, at least about 1000 mm³, or at least about 1500 mm³. Threshold neoplastic disease stages also can vary according to several factors, including specific requirement for staging a particular neoplastic disease, aggressiveness of growth of the neoplastic disease, ability of the virus to infect a tumor or metastasis, and immunocompetence of the subject. Generally the threshold stage will be a stage sufficient for a virus to accumulate and replicate in a tumor or metastasis without being completely removed by the host's immune system, and will typically also be a size sufficient to sustain a virus infection for a time long enough for the host to mount an immune response against the neoplastic cells, typically about one week or more, about ten days or more, or about two weeks or more. Exemplary threshold stages are any stage beyond the lowest stage (e.g., Stage I or equivalent), or any stage where the primary tumor is larger than a threshold size, or any stage where metastatic cells are detected.

In other examples, prior to administering to the subject a virus, the immunocompetence of the subject can be determined. The methods of administering a virus to a subject provided herein can include causing or enhancing an immune response in a subject. Accordingly, prior to administering a virus to a subject, the ability of a subject to mount an immune response can be determined. Any of a variety of tests of immunocompetence known in the art can be performed in the methods provided herein. Exemplary immunocompetence tests can examine ABO hemagglutination titers (IgM), leukocyte adhesion deficiency (LAD), granulocyte function (NBT), T and B cell quantitation, tetanus antibody titers, salivary IgA, skin test, tonsil test, complement C3 levels, and factor B levels, and lymphocyte count. One skilled in the art can determine the desirability to administer a virus to a subject according to the level of immunocompetence of the subject, according to the immunogenicity of the virus, and, optionally, according to the immunogenicity of the neoplastic disease to be treated. Typically, a subject can be considered immunocompetent if the skilled artisan can determine that the subject is sufficiently competent to mount an immune response against the virus.

In some examples, the subject can be immunized prior to administering to the subject a virus according to the methods provided herein. Immunization can serve to increase the ability of a subject to mount an immune response against the virus, or increase the speed at which the subject can mount an immune response against a virus. Immunization also can serve to decrease the risk to the subject of pathogenicity of the virus. In some examples, the immunization can be performed with an immunization virus that is similar to the therapeutic virus to be administered. For example, the immunization virus can be a replication-incompetent variant of the therapeutic virus. In other examples, the immunization material can be digests of the therapeutic virus to be administered. Any of a variety of methods for immunizing a subject against a known virus are known in the art and can be used herein. In one example, vaccinia viruses treated with, for example, 1 microgram of psoralen and ultraviolet light at 365 nm for 4 minutes, can be rendered replication incompetent. In another example, the virus can be selected as the same or similar to a virus against which the subject has been previously immunized, e.g., in a childhood vaccination.

In another example, the subject can have administered thereto a virus without any previous steps of cancer treatment such as chemotherapy, radiation therapy or surgical removal of a tumor and/or metastases. The methods provided herein take advantage of the ability of the viruses to enter or localize near a tumor, where the tumor cells can be protected from the subject's immune system; the viruses can then proliferate in such an immunoprotected region and can also cause the release, typically a sustained release, of tumor antigens from the tumor to a location in which the subject's immune system can recognize the tumor antigens and mount an immune response. In such methods, existence of a tumor of sufficient size or sufficiently developed immunoprotected state can be advantageous for successful administration of the virus to the tumor, and for sufficient tumor antigen production. If a tumor is surgically removed, the viruses may not be able to localize to other neoplastic cells (e.g., small metastases) because such cells have not yet have matured sufficiently to create an immunoprotective environment in which the viruses can survive and proliferate, or even if the viruses can localize to neoplastic cells, the number of cells or size of the mass can be too small for the viruses to cause a sustained release of tumor antigens in order for the host to mount an anti-tumor immune response. Thus, for example, provided herein are methods of treating a tumor or neoplastic disease in which viruses are administered to a subject with a tumor or neoplastic disease without removing the primary tumor, or to a subject with a tumor or neoplastic disease in which at least some tumors or neoplastic cells are intentionally permitted to remain in the subject. In other typical cancer treatment methods such as chemotherapy or radiation therapy, such methods typically have a side effect of weakening the subject's immune system. This treatment of a subject by chemotherapy or radiation therapy can reduce the subject's ability to mount an anti-tumor immune response. Thus, for example, provided herein are methods of treating a tumor or neoplastic disease in which viruses are administered to a subject with a tumor or neoplastic disease without treating the subject with an immune system-weakening therapy, such as chemotherapy or radiation therapy.

In an alternative example, prior to administration of a virus to the subject, the subject can be treated in one or more cancer treatment steps that do not remove the primary tumor or that do not weaken the immune system of the subject. A variety of more sophisticated cancer treatment methods are being developed in which the tumor can be treated without surgical removal or immune-system weakening therapy. Exemplary methods include administering a compound that decreases the rate of proliferation of the tumor or neoplastic cells without weakening the immune system (e.g., by administering tumor suppressor compounds or by administering tumor cell-specific compounds) or administering an angiogenesis-inhibiting compound. Thus, combined methods that include administering a virus to a subject can further improve cancer therapy. Thus, provided herein are methods of administering a virus to a subject, along with prior to or subsequent to, for example, administering a compound that slows tumor growth without weakening the subject's immune system or a compound that inhibits vascularization of the tumor.

b. Mode of Administration

Any mode of administration of a virus to a subject can be used, provided the mode of administration permits the virus to enter a tumor or metastasis or reach a desired target. Modes of administration can include, but are not limited to, systemic, parenteral, intravenous, intraperitoneal, subcutaneous, intramuscular, transdermal, intradermal, intra-arterial (e.g., hepatic artery infusion), intravesicular perfusion, intrapleural, intraarticular, topical, intratumoral, intralesional, endoscopic, multipuncture (e.g., as used with smallpox vaccines), inhalation, percutaneous, subcutaneous, intranasal, intratracheal, oral, intracavity (e.g., administering to the bladder via a catheter, administering to the gut by suppository or enema), vaginal, rectal, intracranial, intraprostatic, intravitreal, aural, or ocular administration. In some examples, a diagnostic or therapeutic agent as described elsewhere herein also can be similarly administered.

One skilled in the art can select any mode of administration compatible with the subject, virus and antibiotic, and that also is likely to result in the virus reaching tumors and/or metastases and the antibiotic effecting commensal or gut bacteria. The route of administration can be selected by one skilled in the art according to any of a variety of factors, including the nature of the disease, the kind of tumor, and the particular virus contained in the pharmaceutical composition. Administration to the target site can be performed, for example, by ballistic delivery, as a colloidal dispersion system, or systemic administration can be performed by injection into an artery.

c. Dosages and Dosage Regime

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular virus to be administered, duration and route of administration, the kind and stage of the disease, for example, tumor size, and other treatments or compounds, such as chemotherapeutic drugs, being administered concurrently. In addition to the above factors, such levels can be affected by the infectivity of the virus, and the nature of the virus, as can be determined by one skilled in the art.

In the present methods, appropriate minimum dosage levels and dosage regimes of viruses can be levels sufficient for the virus to survive, grow and replicate in a tumor or metastasis. Generally, the virus is administered in an amount that is at least or about or is 1×10⁵ pfu at least one time over a cycle of administration. Exemplary minimum levels for administering a virus to a 65 kg human can include at least about 1×10⁵ plaque forming units (pfu), at least about 5×10⁵ pfu, at least about 1×10⁶ pfu, at least about 5×10⁶ pfu, at least about 1×10⁷ pfu, at least about 1×10⁸ pfu, at least about 1×10⁹ pfu, or at least about 1×10¹⁰ pfu. For example, the virus is administered in an amount that is at least or about or is 1×10⁵ pfu, 1×10⁶ pfu, 1×10⁷ pfu, 1×10⁸ pfu, 1×10⁹ pfu, 1×10¹° pfu, 1×10¹¹ pfu, 1×10¹² pfu, 1×10¹³ pfu, or 1×10¹⁴ pfu at least one time over a cycle of administration.

In the dosage regime, the amount of virus can be administered as a single administration or multiple times over the cycle of administration. Hence, the methods provided herein can include a single administration of a virus to a subject or multiple administrations of a virus to a subject. In some examples, a single administration is sufficient to establish a virus in a tumor, where the virus can proliferate and can cause or enhance an anti-tumor response in the subject; such methods do not require additional administrations of a virus in order to cause or enhance an anti-tumor response in a subject, which can result, for example in inhibition of tumor growth, inhibition of metastasis growth or formation, reduction in tumor or size, elimination of a tumor or metastasis, inhibition or prevention of recurrence of a neoplastic disease or new tumor formation, or other cancer therapeutic effects.

In other examples, a virus can be administered on different occasions, separated in time typically by at least one day. For example, a virus can be administered two times, three time, four times, five times, or six times or more, with one day or more, two days or more, one week or more, or one month or more time between administrations. Separate administrations can increase the likelihood of delivering a virus to a tumor or metastasis, where a previous administration has been ineffective in delivering a virus to a tumor or metastasis. Separate administrations can increase the locations on a tumor or metastasis where virus proliferation can occur or can otherwise increase the titer of virus accumulated in the tumor, which can increase the scale of release of antigens or other compounds from the tumor in eliciting or enhancing a host's anti-tumor immune response, and also can, optionally, increase the level of virus-based tumor lysis or tumor cell death. Separate administrations of a virus can further extend a subject's immune response against viral antigens, which can extend the host's immune response to tumors or metastases in which viruses have accumulated, and can increase the likelihood of a host mounting an anti-tumor immune response.

When separate administrations are performed, each administration can be a dosage amount that is the same or different relative to other administration dosage amounts. In one example, all administration dosage amounts are the same. In other examples, a first dosage amount can be a larger dosage amount than one or more subsequent dosage amounts, for example, at least 10× larger, at least 100× larger, or at least 1000× larger than subsequent dosage amounts. In one example of a method of separate administrations in which the first dosage amount is greater than one or more subsequent dosage amounts, all subsequent dosage amounts can be the same, smaller amount relative to the first administration.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a virus, where the number of administrations can be determined by monitoring the subject, and, based on the results of the monitoring, determining whether or not to provide one or more additional administrations. Deciding on whether or not to provide one or more additional administrations can be based on a variety of monitoring results, including, but not limited to, indication of tumor growth or inhibition of tumor growth, appearance of new metastases or inhibition of metastasis, the subject's anti-virus antibody titer, the subject's anti-tumor antibody titer, the overall health of the subject, the weight of the subject, the presence of virus solely in tumor and/or metastases, the presence of virus in normal tissues or organs.

The time period between administrations can be any of a variety of time periods. The time period between administrations can be a function of any of a variety of factors, including monitoring steps, as described in relation to the number of administrations, the time period for a subject to mount an immune response, the time period for a subject to clear the virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

For example, an amount of virus is administered two times, three times, four times, five times, six times or seven times over a cycle of administration. The amount of virus can be administered on the first day of the cycle, the first and second day of the cycle, each of the first three consecutive days of the cycle, each of the first four consecutive days of the cycle, each of the first five consecutive days of the cycle, each of the first six consecutive days of the cycle, or each of the first seven consecutive days of the cycle. Generally, the cycle of administration is 7 days, 14 days, 21 days or 28 days. Depending on the responsiveness or prognosis of the patient the cycle of administration is repeated over the course of several months or years.

Generally, appropriate maximum dosage levels or dosage regimes of viruses are levels that are not toxic to the host, levels that do not cause splenomegaly of 3 times or more, levels that do not result in colonies or plaques in normal tissues or organs after about 1 day or after about 3 days or after about 7 days.

d. Combination Therapy

Also provided are methods in which an additional therapeutic substance, such as a different therapeutic virus or a therapeutic compound is administered. These can be administered simultaneously, sequentially or intermittently with the antibiotic and the virus. The additional therapeutic substance can interact with the virus or a gene product thereof, or the additional therapeutic substance can act independently of the virus.

Combination therapy treatment has advantages in that: 1) it avoids single agent resistance; 2) in a heterogeneous tumor population, it can kill cells by different mechanisms; and 3) by selecting drugs with non-overlapping toxicities, each agent can be used at full dose to elicit maximal efficacy and synergistic effect. Combination therapy can be done by combining a diagnostic/therapeutic virus with one or more of the following anti-cancer agents: chemotherapeutic agents, therapeutic antibodies, siRNAs, toxins, enzyme-prodrug pairs or radiation.

i. Administering a Plurality of Viruses

Methods are provided for administering to a subject an antibiotic and two or more viruses. Administration can be effected simultaneously, sequentially or intermittently. The plurality of viruses can be administered as a single composition or as two or more compositions. The two or more viruses can include at least two viruses. In a particular example, where there are two viruses, both viruses are vaccinia viruses. In another example, one virus is a vaccinia virus and the second virus is any one of an adenovirus, an adeno-associated virus, a retrovirus, a herpes simplex virus, a reovirus, a mumps virus, a foamy virus, an influenza virus, a myxoma virus, a vesicular stomatitis virus, or any other virus described herein or known in the art. Viruses can be chosen based on the pathway on which they act. For example, a virus that targets an activated Ras pathway can be combined with a virus that targets tumor cells defective in p53 expression.

The plurality of viruses can be provided as combinations of compositions containing and/or as kits that include the viruses packaged for administration and optionally including instructions therefore. The compositions can contain the viruses formulated for single dosage administration (i.e., for direct administration) and can require dilution or other additions.

In one example, at least one of the viruses is a modified virus such as those provided herein, having a characteristic such as low pathogenicity, low toxicity, preferential accumulation in tumor, ability to activate an immune response against tumor cells, immunogenic, replication competent, ability to express exogenous proteins, and combinations thereof. The viruses can be administered at approximately the same time, or can be administered at different times. The viruses can be administered in the same composition or in the same administration method, or can be administered in separate composition or by different administration methods.

The time period between administrations can be any time period that achieves the desired effects, as can be determined by one skilled in the art. Selection of a time period between administrations of different viruses can be determined according to parameters similar to those for selecting the time period between administrations of the same virus, including results from monitoring steps, the time period for a subject to mount an immune response, the time period for a subject to clear virus from normal tissue, or the time period for virus proliferation in the tumor or metastasis. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the virus from normal tissue; for example, the time period can be more than the time period for a subject to clear the virus from normal tissue, such as more than about a day, more than about two days, more than about three days, more than about five days, or more than about a week. In another example, the time period can be a function of the time period for virus proliferation in the tumor or metastasis; for example, the time period can be more than the amount of time for a detectable signal to arise in a tumor or metastasis after administration of a virus expressing a detectable marker, such as about 3 days, about 5 days, about a week, about ten days, about two weeks, or about a month.

ii. Therapeutic Compounds

Any therapeutic or anti-cancer agent can be used as the second, therapeutic or anti-cancer agent in the combined cancer treatment methods provided herein. The methods can include administering one or more therapeutic compounds to the subject in addition to administering a virus or plurality thereof to a subject. Therapeutic compounds can act independently, or in conjunction with the virus, for tumor therapeutic effects.

Therapeutic compounds that can act independently include any of a variety of known chemotherapeutic compounds that can inhibit tumor growth, inhibit metastasis growth and/or formation, decrease the size of a tumor or metastasis, eliminate a tumor or metastasis, without reducing the ability of a virus to accumulate in a tumor, replicate in the tumor, and cause or enhance an anti-tumor immune response in the subject.

Therapeutic compounds that act in conjunction with the viruses include, for example, compounds that alter the expression of the viruses or compounds that can interact with a virally-expressed gene, or compounds that can inhibit virus proliferation, including compounds toxic to the virus. Therapeutic compounds that can act in conjunction with the virus include, for example, therapeutic compounds that increase the proliferation, toxicity, tumor cell killing or immune response eliciting properties of a virus, and also can include, for example, therapeutic compounds that decrease the proliferation, toxicity or cell killing properties of a virus. Optionally, the therapeutic agent can exhibit or manifest additional properties, such as, properties that permit its use as an imaging agent, as described elsewhere herein.

Therapeutic compounds also include, but are not limited to, chemotherapeutic agents, nanoparticles, radiation therapy, siRNA molecules, enzyme/pro-drug pairs, photosensitizing agents, toxins, microwaves, a radionuclide, an angiogenesis inhibitor, a mitosis inhibitor protein (e.g., cdc6), an antitumor oligopeptide (e.g., antimitotic oligopeptides, high affinity tumor-selective binding peptides), a signaling modulator, anti-cancer antibiotics, or a combination thereof.

Exemplary photosensitizing agents include, but are not limited to, for example, indocyanine green, toluidine blue, aminolevulinic acid, texaphyrins, benzoporphyrins, phenothiazines, phthalocyanines, porphyrins such as sodium porfimer, chlorins such as tetra(m-hydroxyphenyl)chlorin or tin(IV) chlorin e6, purpurins such as tin ethyl etiopurpurin, purpurinimides, bacteriochlorins, pheophorbides, pyropheophorbides or cationic dyes. In one example, a vaccinia virus, such as a vaccinia virus provided herein, is administered to a subject having a tumor, cancer or metastasis in combination with a photosensitizing agent.

Radionuclides, which depending up the radionuclide, amount and application can be used for diagnosis and/or for treatment. They include, but are not limited to, for example, a compound or molecule containing ³²Phosphorus, ⁶⁰Cobalt, ⁹⁰Yttrium, ⁹⁹Technitium, ¹⁰³Palladium, ¹⁰⁶Ruthenium, ¹¹¹Indium, ¹¹⁷Lutetium, ¹²⁵Iodine, ¹³¹Iodine, ¹³⁷Cesium, ¹⁵³Samarium, ¹⁸⁶Rhenium, ¹⁸⁸Rhenium, ¹⁹²Iridium, ¹⁹⁸Gold, ²¹¹Astatine, ²¹²Bismuth or ²¹³Bismuth. In one example, a vaccinia virus, such as a vaccinia virus provided herein, is administered to a subject having a tumor, cancer or metastasis in combination with a radionuclide.

Toxins include, but are not limited to, chemotherapeutic compounds such as, but not limited to, 5-fluorouridine, calicheamicin and maytansine. Signaling modulators include, but are not limited to, for example, inhibitors of macrophage inhibitory factor, toll-like receptor agonists and stat3 inhibitors. In one example, a vaccinia virus, such as a vaccinia virus provided herein, is administered to a subject having a tumor, cancer or metastasis in combination with a toxin or a signaling modulator.

Combination therapy between chemotherapeutic agents and therapeutic viruses can be effective/curative in situations when single agent treatment is not effective. Chemotherapeutic compounds include, but are not limited to, alkylating agents such as thiotepa and cyclophosphamide; alkyl sulfonates such as busulfan, improsulfan and piposulfan; aziridines such as benzodepa, carboquone, meturedepa and uredepa; ethylenimine and methylmelamines, including altretamine, triethylenemelamine, triethylenephosphoramide, triethylenethiophosphoramide and trimethylmelamine nitrogen mustards such as chlorambucil, chlornaphazine, chlorophosphamide, estramustine, ifosfamide, mechlorethamine, mechlorethamine oxide hydrochloride, melphalan, novobiocin, phenesterine, prednimustine, trofosfamide, uracil mustard; nitrosoureas such as carmustine, chlorozotocin, fotemustine, lomustine, nimustine, ranimustine; antibiotics such as aclacinomycins, actinomycin, anthramycin, azaserine, bleomycins, cactinomycin, calicheamicin, carubicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatrexate; defosfamide; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, e.g., paclitaxel and docetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; Navelbine; Novantrone; teniposide; daunomycin; aminopterin; Xeloda; ibandronate; CPT11; topoisomerase inhibitor RFS 2000; difluoromethylornithine (DMFO); retinoic acid; esperamycins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone and toremifene (Fareston); and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods. Chemotherapeutic agents also include new classes of targeted chemotherapeutic agents such as, for example, imatinib (sold by Novartis under the trade name Gleevec in the United States), gefitinib (developed by AstraZeneca under the trade name Iressa) and erlotinib. Particular chemotherapeutic agents include, but are not limited to, cisplatin, carboplatin, oxaliplatin, DWA2114R, NK121, IS 3 295, 254-S, vincristine, prednisone, doxorubicin and L-asparaginase; mechlorethamine, vincristine, procarbazine and prednisone (MOPP), cyclophosphamide, vincristine, procarbazine and prednisone (C-MOPP), bleomycin, vinblastine, gemcitabine and 5-fluorouracil. Exemplary chemotherapeutic agents are, for example, cisplatin, carboplatin, oxaliplatin, DWA2114R, NK121, IS 3 295, and 254-S. In a non-limiting example, a vaccinia virus, such as a vaccinia virus provided herein, is administered to a subject having a tumor, cancer or metastasis in combination with a platinum coordination complex, such as cisplatin, carboplatin, oxaliplatin, DWA2114R, NK121, IS 3 295, and 254-S. Tumors, cancers and metastasis can be any of those provided herein, and in particular, can be a pancreatic tumor, an ovarian tumor, a lung tumor, a colon tumor, a prostate tumor, a cervical tumor or a breast tumor; exemplary tumors are pancreatic and ovarian tumors. Tumors, cancers and metastasis can be a monotherapy-resistant tumor such as, for example, one that does not respond to therapy with virus alone or anti-cancer agent alone, but that does respond to therapy with a combination of virus and anti-cancer agent. Typically, a therapeutically effective amount of virus is systemically administered to the subject and the virus localizes and accumulates in the tumor. Subsequent to administering the virus, the subject is administered a therapeutically effective amount of an anti-cancer agent, such as cisplatin. In one example, cisplatin is administered once-daily for five consecutive days. One of skill in the art could determine when to administer the anti-cancer agent subsequent to the virus using, for example, in vivo animal models. Using the methods provided herein, administration of a virus and anti-cancer agent, such as cisplatin can cause a reduction in tumor volume, can cause tumor growth to stop or be delayed or can cause the tumor to be eliminated from the subject. The status of tumors, cancers and metastasis following treatment can be monitored using any of the methods provided herein and known in the art.

Exemplary anti-cancer antibiotics include, but are not limited to, anthracyclines such as doxorubicin hydrochloride (adriamycin), idarubicin hydrochloride, daunorubicin hydrochloride, aclarubicin hydrochloride, epirubicin hydrochloride and pirarubicin hydrochloride, phleomycins such as phleomycin and peplomycin sulfate, mitomycins such as mitomycin C, actinomycins such as actinomycin D, zinostatin stimalamer and polypeptides such as neocarzinostatin. In one example, a vaccinia virus, such as a vaccinia virus provided herein, is administered to a subject having a tumor, cancer or metastasis in combination with an anti-cancer antibiotic.

In one example, nanoparticles can be designed such that they carry one or more therapeutic agents provided herein. Additionally, nanoparticles can be designed to carry a molecule that targets the nanoparticle to the tumor cells. In one non-limiting example, nanoparticles can be coated with a radionuclide and, optionally, an antibody immunoreactive with a tumor-associated antigen. In one example, a vaccinia virus, such as a vaccinia virus provided herein, is administered to a subject having a tumor, cancer or metastasis in combination with a nanoparticle carrying any of the therapeutic agents provided herein.

Radiation therapy has become a foremost choice of treatment for a majority of cancer patients. The wide use of radiation treatment stems from the ability of gamma-irradiation to induce irreversible damage in targeted cells with the preservation of normal tissue function. Ionizing radiation triggers apoptosis, the intrinsic cellular death machinery in cancer cells, and the activation of apoptosis seems to be the principal mode by which cancer cells die following exposure to ionizing radiation. In one example, a vaccinia virus, such as a vaccinia virus provided herein, is administered to a subject having a tumor, cancer or metastasis in combination with radiation therapy.

Thus, provided herein are methods of administering to a subject one or more therapeutic compounds that can act in conjunction with the virus to increase the proliferation, toxicity, tumor cell killing, or immune response eliciting properties of a virus. Also provided herein are methods of administering to a subject one or more therapeutic compounds that can act in conjunction with the virus to decrease the proliferation, toxicity, or cell killing properties of a virus. Therapeutic compounds to be administered can be any of those provided herein or in the art.

Therapeutic compounds that can act in conjunction with the virus to increase the proliferation, toxicity, tumor cell killing or immune response eliciting properties of a virus are compounds that can alter gene expression, where the altered gene expression can result in an increased killing of tumor cells or an increased anti-tumor immune response in the subject. A gene expression-altering compound can, for example, cause an increase or decrease in expression of one or more viral genes, including endogenous viral genes and/or exogenous viral genes. For example, a gene expression-altering compound can induce or increase transcription of a gene in a virus such as an exogenous gene that can cause cell lysis or cell death, that can provoke an immune response, that can catalyze conversion of a prodrug-like compound, or that can inhibit expression of a tumor cell gene. Any of a wide variety of compounds that can alter gene expression are known in the art, including IPTG and RU486. Exemplary genes whose expression can be up-regulated include proteins and RNA molecules, including toxins, enzymes that can convert a prodrug to an anti-tumor drug, cytokines, transcription regulating proteins, siRNA and ribozymes. In another example, a gene expression-altering compound can inhibit or decrease transcription of a gene in a virus such as a heterologous gene that can reduce viral toxicity or reduces viral proliferation. Any of a variety of compounds that can reduce or inhibit gene expression can be used in the methods provided herein, including siRNA compounds, transcriptional inhibitors or inhibitors of transcriptional activators. Exemplary genes whose expression can be down-regulated include proteins and RNA molecules, including viral proteins or RNA that suppress lysis, nucleotide synthesis or proliferation, and cellular proteins or RNA molecules that suppress cell death, immunoreactivity, lysis, or viral replication.

In another example, therapeutic compounds that can act in conjunction with the virus to increase the proliferation, toxicity, tumor cell killing, or immune response eliciting properties of a virus are compounds that can interact with a virally expressed gene product, and such interaction can result in an increased killing of tumor cells or an increased anti-tumor immune response in the subject. A therapeutic compound that can interact with a virally-expressed gene product can include, for example a prodrug or other compound that has little or no toxicity or other biological activity in its subject-administered form, but after interaction with a virally expressed gene product, the compound can develop a property that results in tumor cell death, including but not limited to, cytotoxicity, ability to induce apoptosis, or ability to trigger an immune response. In one non-limiting example, the virus carries an enzyme into the cancer cells. Once the enzyme is introduced into the cancer cells, an inactive form of a chemotherapy drug (i.e., a prodrug) is administered. When the inactive prodrug reaches the cancer cells, the enzyme converts the prodrug into the active chemotherapy drug, so that it can kill the cancer cell. Thus, the treatment is targeted only to cancer cells and does not affect normal cells. The prodrug can be administered concurrently with, or sequentially to, the virus. A variety of prodrug-like substances are known in the art and an exemplary set of such compounds are disclosed elsewhere herein, where such compounds can include ganciclovir, 5-fluorouracil, 6-methylpurine deoxyriboside, cephalosporin-doxorubicin, 4-[(2-chloroethyl)(2-mesyloxyethyl)amino]benzoyl-L-glutamic acid, acetaminophen, indole-3-acetic acid, CB1954, 7-ethyl-10-[4-(1-piperidino)-1-piperidino]carbonyloxycamptothecin, bis-(2-chloroethyl)amino-4-hydroxyphenyl-aminomethanone 28, 1-chloromethyl-5-hydroxy-1,2-dihydro-3H-benz[e]indole, epirubicin-glucuronide, 5′-deoxy-5-fluorouridine, cytosine arabinoside, linamarin, and a nucleoside analogue (e.g., fluorouridine, fluorodeoxyuridine, fluorouridine arabinoside, cytosine arabinoside, adenine arabinoside, guanine arabinoside, hypoxanthine arabinoside, 6-mercaptopurineriboside, thioguanine riboside, nebularine, 5-iodouridine, 5-iododeoxyuridine, 5-bromodeoxyuridine, 5-vinyldeoxyuridine, 9-[(2-hydroxy)ethoxy]methylguanine (acyclovir), 9-[(2-hydroxy-1-hydroxymethyl)-ethoxy]methylguanine (DHPG), azauridine, azacytidine, azidothymidine, dideoxyadenosine, dideoxycytidine, dideoxyinosine, dideoxyguanosine, dideoxythymidine, 3′-deoxyadenosine, 3′-deoxycytidine, 3′-deoxyinosine, 3′-deoxyguanosine, 3′-deoxythymidine).

In another example, therapeutic compounds that can act in conjunction with the virus to decrease the proliferation, toxicity or cell killing properties of a virus are compounds that can inhibit viral replication, inhibit viral toxins or cause viral death. A therapeutic compound that can inhibit viral replication, inhibit viral toxins, or cause viral death can generally include a compound that can block one or more steps in the viral life cycle, including, but not limited to, compounds that can inhibit viral DNA replication, viral RNA transcription, viral coat protein assembly, outer membrane or polysaccharide assembly. Any of a variety of compounds that can block one or more steps in a viral life cycle are known in the art, including any known antiviral compound (e.g., cidofovir), viral DNA polymerase inhibitors, viral RNA polymerase inhibitors, inhibitors of proteins that regulate viral DNA replication or RNA transcription. In another example, a virus can contain a gene encoding a viral life cycle protein, such as DNA polymerase or RNA polymerase that can be inhibited by a compound that is, optionally, non-toxic to the host organism.

In addition to combination therapy between chemotherapeutic agents and a virus provided herein, other more complex combination therapy strategies could be applied as well. For example, a combination therapy can include chemotherapeutic agents, therapeutic antibodies, and a virus provided herein. Alternatively, another combination therapy can be the combination of radiation, therapeutic antibodies, and a virus provided herein. Therefore, the concept of combination therapy also can be based on the application of a virus provided herein virus along with one or more of the following therapeutic modalities, namely, chemotherapeutic agents, radiation therapy, therapeutic antibodies, hyper- or hypothermia therapy, siRNA, diagnostic/therapeutic bacteria, diagnostic/therapeutic mammalian cells, immunotherapy, and/or targeted toxins (delivered by antibodies, liposomes and nanoparticles).

Effective delivery of each components of the combination therapy is an important aspect of the methods provided herein. In accordance with one aspect, the modes of administration discussed below exploit one of more of the key features: (i) delivery of a virus provided herein to the tumors by a mode of administration effect to achieve highest titer of virus and highest therapeutic effect; (ii) delivery of any other mentioned therapeutic modalities to the tumor by a mode of administration to achieve the optimal therapeutic effect. The dose scheme of the combination therapy administered is such that the combination of the two or more therapeutic modalities is therapeutically effective. Dosages will vary in accordance with such factors as the age, health, sex, size and weight of the patient, the route of administration, the toxicity of the drugs, frequency of treatment and the relative susceptibilities of the cancer to each of the therapeutic modalities.

For combination therapies with chemotherapeutic compounds, dosages for the administration of such compounds are known in the art or can be determined by one skilled in the art according to known clinical factors (e.g., subject's species, size, body surface area, age, sex, immunocompetence, and general health, duration and route of administration, the kind and stage of the disease, for example, tumor size, and other viruses, treatments, or compounds, such as other chemotherapeutic drugs, being administered concurrently). In addition to the above factors, such levels can be affected by the infectivity of the virus, and the nature of the virus, as can be determined by one skilled in the art. For example, cisplatin (also called cis-platinum, platinol; cis-diamminedichloroplatinum; and cDDP) is representative of a broad class of water-soluble, platinum coordination compounds frequently employed in the therapy of testicular cancer, ovarian tumors and a variety of other cancers. (See, e.g., Blumenreich et al. (1985) Cancer 55(5): 1118-1122; Forastiere et al. (2001) J. Clin. Oncol. 19(4): 1088-1095). Methods of employing cisplatin clinically are well known in the art. For example, cisplatin has been administered in a single day over a six hour period, once per month, by slow intravenous infusion. For localized lesions, cisplatin can be administered by local injection. Intraperitoneal infusion can also be employed. Cisplatin can be administered in doses as low as 10 mg/m² per treatment if part of a multi-drug regimen, or if the patient has an adverse reaction to higher dosing. In general, a clinical dose is from about 30 to about 120 or 150 mg/m² per treatment.

Typically, platinum-containing chemotherapeutic agents are administered parenterally, for example by slow intravenous infusion, or by local injection, as discussed above. The effects of intralesional (intra-tumoral) and IP administration of cisplatin is described in (Nagase et al. (1987) Cancer Treat. Rep. 71(9): 825-829; and Theon et al. (1993) J. Am. Vet. Med. Assoc. 202(2): 261-267).

In one exemplary example, the virus is administered once, 2-6 times or more with 0-60 days apart each administration, followed by 1-30 days where no anti-cancer treatment, then cisplatin is administered daily for 1-5 days, followed by 1-30 days where no anti-cancer treatment is administered. Each component of the therapy, virus or cisplatin treatment, or the virus and cisplatin combination therapy can be repeated. In another exemplary example, cisplatin is administered daily for 1 to 5 days, followed by 1-10 days where no anti-cancer treatment is administered, then the virus is administered once or 2-6 times with 0-60 days apart. Such treatment scheme can be repeated. In another exemplary example, cisplatin is administered daily for 1 to 5 days, followed by 1-10 days where no anti-cancer treatment is administered, then the virus is administered once or 2-6 times with 0-60 days apart. This is followed by 5-60 days where no anti-cancer treatment is administered, then cisplatin is administered again for 1-5 days. Such treatment scheme can be repeated.

Gemcitabine (GEMZAR®) is another compound employed in the therapy of breast cancer, non-small cell lung cancer, and pancreatic cancer. Gemcitabine is a nucleoside analogue that exhibits antitumor activity. Methods of employing gemcitabine clinically are well known in the art. For example, gemcitabine has been administered by intravenous infusion at a dose of 1000 mg/m² over 30 minutes once weekly for up to 7 weeks (or until toxicity necessitates reducing or holding a dose), followed by a week of rest from treatment of pancreatic cancer. Subsequent cycles can include infusions once weekly for 3 consecutive weeks out of every 4 weeks. Gemcitabine has also been employed in combination with cisplatin in cancer therapy.

In one exemplary example, the virus is administered once or 2-6 times with 0-60 days apart, followed by 1-30 days where no anti-cancer treatment is administered, then gemcitabine is administered 1-7 times with 0-30 days apart, followed by 1-30 days where no anti-cancer treatment is administered. Such treatment scheme can be repeated. In another exemplary example, gemcitabine is administered 1-7 times with 0-30 days apart, followed by 1-10 days where no anti-cancer treatment is administered, then the virus is administered once or 2-6 times with 0-60 days apart. This is followed by 5-60 days where no anti-cancer treatment is administered. Such treatment scheme can be repeated. In another exemplary example, gemcitabine is administered 1-7 times with 0-30 days apart, followed by 1-10 days where no anti-cancer treatment is administered, then the virus is administered once or 2-6 times with 0-60 days apart. This is followed by 5-60 days where no anti-cancer treatment is administered, then gemcitabine is administered again for 1-7 times with 0-30 days apart. Such treatment scheme can be repeated.

As will be understood by one of skill in the art, the optimal treatment regimen will vary and it is within the scope of the treatment methods to evaluate the status of the disease under treatment and the general health of the patient prior to, and following one or more cycles of combination therapy in order to determine the optimal therapeutic combination.

iii. Immunotherapies and Biological Therapies

Therapeutic compounds also include, but are not limited to, compounds that exert an immunotherapeutic effect, stimulate or suppress the immune system, carry a therapeutic compound, or a combination thereof. Optionally, the therapeutic agent can exhibit or manifest additional properties, such as, properties that permit its use as an imaging agent, as described elsewhere herein. Such therapeutic compounds include, but are not limited to, anti-cancer antibodies, radiation therapy, siRNA molecules and compounds that suppress the immune system (i.e. immunosuppressors, immunosuppressive agents). In some cases, it is desirable to administer an immunosuppressive agent to a subject to suppress the immune system prior to the administration of the virus in order to minimize any adverse reactions to the virus. Exemplary immunosuppressive agents include, but are not limited to, glucocorticoids, alkylating agents, antimetabolites, interferons and immunosuppressive antibodies (e.g., anti-CD3 and anti-IL2 receptor antibodies).

Immunotherapy also includes for example, immune-stimulating molecules (protein-based or non-protein-based), cells and antibodies. Immunotherapy treatments can include stimulating immune cells to act more effectively or to make the tumor cells or tumor associated antigens recognizable to the immune system (i.e., break tolerance).

Cytokines and growth factors include, but are not limited to, interleukins, such as, for example, interleukin-1, interleukin-2, interleukin-6 and interleukin-12, tumor necrosis factors, such as tumor necrosis factor alpha (TNF-α), interferons such as interferon gamma (IFN-γ), granulocyte macrophage colony stimulating factors (GM-CSF), angiogenins, and tissue factors.

Anti-cancer antibodies include, but are not limited to, ADEPT, Trastuzumab (Herceptin®), Tositumomab (Bexxar®), Cetuximab (Erbitux®), Ibritumomab (Zevalin®), Alemtuzumab (Campath-1H, Campath®), Epratuzumab (LymphoCide), Gemtuzumab ozogamicin (Mylotarg®), Bevacimab (Avastin®), Tarceva® (Erlotinib), SUTENT® (sunitinib malate), Panorex™ (Edrecolomab), Rituxan® (Rituximab), Zevalin® (90Y-ibritumomab tiuxetan) and Mylotarg® (Gemtuzumab Ozogamicin).

Thus, provided herein are methods of administering to a subject one or more therapeutic compounds that can act in conjunction with the virus to stimulate or enhance the immune system, thereby enhancing the effect of the virus. Such immunotherapy can be either delivered as a separate therapeutic modality or could be encoded (if the immunotherapy is protein-based) by the administered virus.

Biological therapies are treatments that use natural body substances or drugs made from natural body substances. They can help to treat a cancer and control side effects caused by other cancer treatments such as chemotherapy. Biological therapies are also sometimes called Biological Response Modifiers (BRMs), biologic agents or simply “biologics” because they stimulate the body to respond biologically (or naturally) to cancer. Immunotherapy is treatment using natural substances that the body uses to fight infection and disease. Because it uses natural substances, immunotherapy is also a biological therapy. There are several types of drugs that come under the term biological therapy: these include, for example, monoclonal antibodies (mAbs), cancer vaccines, growth factors for blood cells, cancer growth inhibitors, anti-angiogenic factors, interferon alpha, interleukin-2 (IL-2), gene therapy and BCG vaccine for bladder cancer

Monoclonal antibodies (mAbs) are of particular interest for treating cancer because of the specificity of binding to a unique antigen and the ability to produce large quantities in the laboratory for mass distribution. Monoclonal antibodies can be engineered to act in the same way as immune system proteins: that is, to seek out and kill foreign matter in your body, such as viruses. Monoclonal antibodies can be designed to recognize epitopes on the surface of cancer cells. The antibodies target specifically bind to the epitopes and either kill the cancer cells or deliver a therapeutic agent to the cancer cell. Methods of conjugating therapeutic agents to antibodies is well-known in the art. Different antibodies have to be made for different types of cancer; for example, rituximab recognizes CD20 protein on the outside of non Hodgkin's lymphoma cells; ADEPT is a treatment using antibodies that recognize bowel (colon) cancer; and Trastuzumab (Herceptin®) recognizes breast cancer cells that produce too much of the protein HER 2 (“HER 2 positive”). Other antibodies include, for example, Tositumomab (Bexxar®), Cetuximab (Erbitux®), Ibritumomab (Zevalin®), Alemtuzumab (Campath-1H), Epratuzumab (LymphoCide), Gemtuzumab ozogamicin (Mylotarg®) and Bevacimab (Avastin®). Thus, the viruses provided herein can be administered concurrently with, or sequentially to, one or more monoclonal antibodies in the treatment of cancer. In one example, additional therapy is administered in the form of one or more of any of the other treatment modalities provided herein.

Rather than attempting to prevent infection, such as is the case with the influenza virus, cancer vaccines help treat the cancer once it has developed. The aim of cancer vaccines is to stimulate the immune response. Cancer vaccines include, for example, antigen vaccines, whole cell vaccines, dendritic cell vaccines, DNA vaccines and anti-idiotype vaccines. Antigen vaccines are vaccines made from tumor-associated antigens in, or produced by, cancer cells. Antigen vaccines stimulate a subject's immune system to attack the cancer. Whole cell vaccines are vaccines that use the whole cancer cell, not just a specific antigen from it, to make the vaccine. The vaccine is made from a subject's own cancer cells, another subject's cancer cells or cancer cells grown in a laboratory. The cells are treated in the laboratory, usually with radiation, so that they can't grow, and are administered to the subject via injection or through an intravenous drip into the bloodstream so they can stimulate the immune system to attack the cancer. One type of whole cell vaccine is a dendritic cell vaccine, which help the immune system to recognize and attack abnormal cells, such as cancer cells. Dendritic cell vaccines are made by growing dendritic cells alongside the cancer cells in the lab. The vaccine is administered to stimulate the immune system to attack the cancer. Anti-idiotype vaccines are vaccines that stimulate the body to make antibodies against cancer cells. Cancer cells make some tumor-associated antigens that the immune system recognizes as foreign. But because cancer cells are similar to non-cancer cells, the immune system can respond weakly. DNA vaccines boost the immune response. DNA vaccines are made from DNA from cancer cells that carry the genes for the tumor-associated antigens. When a DNA vaccine is injected, it enables the cells of the immune system to recognize the tumor-associated antigens, and activates the cells in the immune system (i.e., breaking tolerance). The most promising results from using DNA vaccines are in treating melanoma. Thus, the viruses provided herein can be administered concurrently with, or sequentially to, a whole cell vaccine in the treatment of cancer. In one example, additional therapy is administered in the form of one or more of any of the other treatment modalities provided herein.

Growth factors are natural substances that stimulate the bone marrow to make blood cells. Recombinant technology can be used to generate growth factors which can be administered to a subject to increase the number of white blood cells, red blood cells and stem cells in the blood. Growth factors used in cancer treatment to boost white blood cells include Granulocyte Colony Stimulating Factor (G-CSF) also called filgrastim (Neupogen) or lenograstim (Granocyte) and Granulocyte and Macrophage Colony Stimulating Factor (GM-CSF), also called molgramostim. A growth factor to help treat anemia is erythropoietin (EPO). EPO encourages the body to make more red blood cells, which in turn, increases hemoglobin levels and the levels of oxygen in body tissues. Other growth factors are being developed which can boost platelets. Thus, the viruses provided herein can be administered concurrently with, or sequentially to, a growth factor such as GM-CSF, in the treatment of cancer. In one example, additional therapy is administered in the form of one or more of any of the other treatment modalities provided herein.

Cancer growth inhibitors use cell-signaling molecules which control the growth and multiplication of cells, such as cancer cells. Drugs that block these signaling molecules can stop cancers from growing and dividing. Cancer growth factors include, but are not limited to, tyrosine kinases. Thus, drugs that block tyrosine kinases are tyrosine kinase inhibitors (TKIs). Examples of TKIs include, but are not limited to, Erlotinib (Tarceva®, OSI-774), Iressa® (Gefitinib, ZD 1839) and Imatinib (Gleevec®, STI 571). Another type of growth inhibitor is Bortezomib (Velcade®) for multiple myeloma and for some other cancers. Velcade is a proteasome inhibitor. Proteasomes are found in all cells and help break down proteins in cells. Interfering with the action of proteasomes causes a buildup of proteins in the cell to toxic levels; thereby killing the cancer cells. Cancer cells are more sensitive to Velcade than normal cells. Thus, the viruses provided herein can be administered concurrently with, or sequentially to, a cancer growth inhibitor, such as Velcade, in the treatment of cancer. In one example, additional therapy is administered in the form of one or more of any of the other treatment modalities provided herein.

Cancers need a blood supply to expand and grow their own blood vessels as they get bigger. Without its own blood supply, a cancer cannot grow due to lack of nutrients and oxygen. Anti-angiogenic drugs stop tumors from developing their own blood vessels. Examples of these types of drugs include, but are not limited to, Thalidomide, mainly for treating myeloma but also in trials for other types of cancer, and Bevacizumab (Avastin), a type of monoclonal antibody that has been investigated for bowel cancer. Thus, the viruses provided herein can be administered concurrently with, or sequentially to, an anti-angiogenic drug in the treatment of cancer. In one example, additional therapy is administered in the form of one or more of any of the other treatment modalities provided herein.

Interferon-alpha (IFN-α) is a natural substance produced in the body, in very small amounts, as part of the immune response. IFN-α is administered as a treatment to boost the immune system and help fight cancers such as renal cell (kidney) cancer, malignant melanoma, multiple myeloma and some types of leukemias. IFN-α works in several ways: it can help to stop cancer cells growing, it can also boost the immune system to help it attack the cancer, and it can affect the blood supply to the cancer cells. Thus, the viruses provided herein can be administered concurrently with, or sequentially to, IFN-α in the treatment of cancer. In one example, additional therapy is administered in the form of one or more of any of the other treatment modalities provided herein.

Administration of IL-2 is a biological therapy drug because it is naturally produced by the immune system. Thus, it is also an immunotherapy. Interleukin 2 is used in treating renal cell (kidney) cancer, and is being tested in clinical trials for several other types of cancers. IL-2 works directly on cancer cells by interfering with cell grow and proliferation; it stimulates the immune system by promoting the growth of killer T cells and other cells that attack cancer cells; and it also stimulates cancer cells to secrete chemoattractants that attract immune system cells. IL-2 is generally administered as a subcutaneous injection just under the skin once daily for 5 days, followed by 2 days rest. The cycle of injections is repeated for 4 weeks followed by a week without treatment. The treatment regimen and the number of cycles administered depends on the type of cancer and how it responds to the treatment. IL-2 can be self-administered or administered by a health professional. Alternatively, IL-2 can be administered intravenously via injection or drip. Thus, the viruses provided herein can be administered concurrently with, or sequentially to, IL-2 in the treatment of cancer. In one example, additional therapy is administered in the form of one or more of any of the other treatment modalities provided herein.

Gene therapy involves treating cancer by blocking abnormal genes in cancer cells, repairing or replacing abnormal genes in cancer cells, encouraging even more genes to become abnormal in cancer cells so that they die or become sensitive to treatment, using viruses to carry treatment-activating enzymes into the cancer cells, or a combination thereof. As a result, cancer cells die due to damage in the cell. Cancer cells develop as a result of several types of mutations in several of their genes. Targeted genes include, but are not limited to, those that encourage the cell to multiply (i.e., oncogenes), genes that stop the cell from multiplying (i.e., tumor suppressor genes) and genes that repair other damaged genes. Gene therapy can involve repair of damaged oncogenes or blocking the proteins that the oncogenes produce. The tumor suppressor gene, p53, is damaged in many human cancers. Viruses have been used to deliver an undamaged p53 gene into cancer cells, and early clinical trials are now in progress looking at treating cancers with modified p53-producing viruses. Gene therapy could be used to replace the damaged DNA repairing genes. In an alternative example, methods of increasing DNA damage within a tumor cell can promote death of the tumor cell or cause increased susceptibility of the tumor cell to other cancer treatments, such as radiotherapy or chemotherapy. Thus, the viruses provided herein can be administered concurrently with, or sequentially to, any of the gene therapy methods provided herein or known in the art in the treatment of cancer. In one example, additional therapy is administered in the form of one or more of any of the other treatment modalities provided herein.

Treatment of early stage bladder cancer is called intravesical treatment, which is mainly used to treat stage T1 bladder cancers that are high grade (grade 3 or G3) or carcinoma in situ of the bladder (also known as T is or CIS). BCG is a vaccine for tuberculosis (TB), which also has been found to be effective in treating CIS and preventing bladder cancers from recurring. In some cases, BCG vaccines have been used for treating grade 2 early bladder cancer. Because bladder cancer can occur anywhere in the bladder lining, it cannot be removed in the same way as the papillary early bladder cancers. Rather a BCG vaccine is administered using intravesical therapy; that is, first, a catheter (tube) put is inserted into the bladder, followed by intra-catheter administration of a BCG vaccine and/or a chemotherapy. BCG treatment occurs weekly for 6 weeks or more depending on the effect on the bladder cancer. BCG treatment of bladder cancer can be combined with other types of treatments, such as administration of chemotherapy (intravesical), IL-2, treatment with drugs that make cells sensitive to light, vitamins, and photodynamic therapy. Thus, the viruses provided herein can be administered concurrently with, or sequentially to, BCG vaccines in the treatment of cancer. In one example, additional therapy is administered in the form of one or more of any of the other treatment modalities provided herein.

e. State of Subject

In another example, the methods provided herein for administering an antibiotic and virus to a subject can be performed on a subject in any of a variety of states, including an anesthetized subject, an alert subject, a subject with elevated body temperature, a subject with reduced body temperature, or other state of the subject that is known to affect the accumulation of a virus in the tumor. As provided herein, it has been determined that a subject that is anesthetized can have a decreased rate of accumulation of a virus in a tumor relative to a subject that is not anesthetized. Further provided herein, it has been determined that a subject with decreased body temperature can have a decreased rate of accumulation of a virus in a tumor relative to a subject with a normal body temperature. Accordingly, provided herein are methods of administering an antibiotic and a virus to a subject, where the methods can include administering an antibiotic and a virus to a subject where the subject is not under anesthesia, such as general anesthesia; for example, the subject can be under local anesthesia, or can be anaesthetized. Also provided herein are methods of administering an antibiotic and a virus to a subject, where the methods can include administering a virus to a subject with altered body temperature, where the alteration of the body temperature can influence the ability of the virus to accumulate in a tumor; typically, a decrease in body temperature can decrease the ability of a virus to accumulate in a tumor. Thus, in one exemplary example, a method is provided for administering a virus to a subject, where the method includes elevating the body temperature of the subject to a temperature above normal, and administering a virus to the subject, where the virus can accumulate in the tumor more readily in the subject with higher body temperature relative to the ability of the virus to accumulate in a tumor of a subject with a normal body temperature. In another example, localized elevations in temperature in the area surrounding the tumor can be used to increase the accumulation of the virus in the tumor.

4. Monitoring Oncolytic Viral Therapy

The methods provided herein can further include one or more steps of monitoring the subject, monitoring the tumor, and/or monitoring the virus administered to the subject. Any of a variety of monitoring steps can be included in the methods provided herein, including, but not limited to, monitoring tumor size, monitoring anti-(tumor antigen) antibody titer, monitoring the presence and/or size of metastases, monitoring the subject's lymph nodes, monitoring the subject's weight or other health indicators including blood or urine markers, monitoring anti-(viral antigen) antibody titer, monitoring viral expression of a detectable gene product, and directly monitoring viral titer in a tumor, tissue or organ of a subject.

The purpose of the monitoring can be simply for assessing the health state of the subject or the progress of therapeutic treatment of the subject, or can be for determining whether or not further administration of the same or a different virus is warranted, or for determining when or whether or not to administer a compound to the subject where the compound can act to increase the efficacy of the therapeutic method, or the compound can act to decrease the pathogenicity of the virus administered to the subject.

a. Monitoring Viral Gene Expression

In some examples, the methods provided herein can include monitoring one or more virally expressed genes. Viruses can express one or more detectable gene products, including but not limited to, detectable proteins (e.g. luminescent or fluorescent proteins) or proteins that induce a detectable signal (e.g. proteins that bind or transport detectable compounds or modify substrates to produce a signal). The infected cells/tissue can thus be imaged by one more optical or non-optical imaging methods.

As provided herein, measurement of a detectable gene product expressed by a virus can provide an accurate determination of the level of virus present in the subject. As further provided herein, measurement of the location of the detectable gene product, for example, by imaging methods including, but not limited to, magnetic resonance, fluorescence, and tomographic methods, can determine the localization of the virus in the subject. Accordingly, the methods provided herein that include monitoring a detectable viral gene product can be used to determine the presence or absence of the virus in one or more organs or tissues of a subject, and/or the presence or absence of the virus in a tumor or metastases of a subject. Further, the methods provided herein that include monitoring a detectable viral gene product can be used to determine the titer of virus present in one or more organs, tissues, tumors or metastases. Methods that include monitoring the localization and/or titer of viruses in a subject can be used for determining the pathogenicity of a virus; since viral infection, and particularly the level of infection, of normal tissues and organs can indicate the pathogenicity of the probe, methods of monitoring the localization and/or amount of viruses in a subject can be used to determine the pathogenicity of a virus. Since methods provided herein can be used to monitor the amount of viruses at any particular location in a subject, the methods that include monitoring the localization and/or titer of viruses in a subject can be performed at multiple time points, and, accordingly can determine the rate of viral replication in a subject, including the rate of viral replication in one or more organs or tissues of a subject; accordingly, the methods of monitoring a viral gene product can be used for determining the replication competence of a virus. The methods provided herein also can be used to quantitate the amount of virus present in a variety of organs or tissues, and tumors or metastases, and can thereby indicate the degree of preferential accumulation of the virus in a subject; accordingly, the viral gene product monitoring methods provided herein can be used in methods of determining the ability of a virus to accumulate in tumor or metastases in preference to normal tissues or organs. Since the viruses used in the methods provided herein can accumulate in an entire tumor or can accumulate at multiple sites in a tumor, and can also accumulate in metastases, the methods provided herein for monitoring a viral gene product can be used to determine the size of a tumor or the number of metastases that are present in a subject. Monitoring such presence of viral gene product in tumor or metastasis over a range of time can be used to assess changes in the tumor or metastasis, including growth or shrinking of a tumor, or development of new metastases or disappearance of metastases, and also can be used to determine the rate of growth or shrinking of a tumor, or development of new metastases or disappearance of metastases, or the change in the rate of growth or shrinking of a tumor, or development of new metastases or disappearance of metastases. Accordingly, the methods of monitoring a viral gene product can be used for monitoring a neoplastic disease in a subject, or for determining the efficacy of treatment of a neoplastic disease, by determining rate of growth or shrinking of a tumor, or development of new metastases or disappearance of metastases, or the change in the rate of growth or shrinking of a tumor, or development of new metastases or disappearance of metastases.

Any of a variety of detectable proteins can be detected in the monitoring methods provided herein; an exemplary, non-limiting list of such detectable proteins includes any of a variety of fluorescent proteins (e.g., green or red fluorescent proteins), any of a variety of luciferases, transferrin or other iron binding proteins; or receptors, binding proteins, and antibodies, where a compound that specifically binds the receptor, binding protein or antibody can be a detectable agent or can be labeled with a detectable substance (e.g., a radionuclide or imaging agent); or transporter proteins (e.g. hNET or hNIS) that can bind to and transport detectable molecules into the cell. Viruses expressing a detectable protein can be detected by a combination of the method provided herein and know in the art. Viruses expressing more than one detectable protein or two or more viruses expressing various detectable protein can be detected and distinguished by dual imaging methods. For example, a virus expressing a fluorescent protein and an iron binding protein can be detected in vitro or in vivo by low light fluorescence imaging and magnetic resonance, respectively. In another example, a virus expressing two or more fluorescent proteins can be detected by fluorescence imaging at different wavelength. In vivo dual imaging can be performed on a subject that has been administered a virus expressing two or more detectable gene products or two or more viruses each expressing one or more detectable gene products.

b. Monitoring Tumor Size

Also provided herein are methods of monitoring tumor and/or metastasis size and location. Tumor and or metastasis size can be monitored by any of a variety of methods known in the art, including external assessment methods or tomographic or magnetic imaging methods. In addition to the methods known in the art, methods provided herein, for example, monitoring viral gene expression, can be used for monitoring tumor and/or metastasis size.

Monitoring size over several time points can provide information regarding the increase or decrease in size of a tumor or metastasis, and can also provide information regarding the presence of additional tumors and/or metastases in the subject. Monitoring tumor size over several time points can provide information regarding the development of a neoplastic disease in a subject, including the efficacy of treatment of a neoplastic disease in a subject.

c. Monitoring Antibody Titer

The methods provided herein also can include monitoring the antibody titer in a subject, including antibodies produced in response to administration of a virus to a subject. The viruses administered in the methods provided herein can elicit an immune response to endogenous viral antigens. The viruses administered in the methods provided herein also can elicit an immune response to exogenous genes expressed by a virus. The viruses administered in the methods provided herein also can elicit an immune response to tumor antigens. Monitoring antibody titer against viral antigens, viral expressed exogenous gene products, or tumor antigens can be used in methods of monitoring the toxicity of a virus, monitoring the efficacy of treatment methods, or monitoring the level of gene product or antibodies for production and/or harvesting.

In one example, monitoring antibody titer can be used to monitor the toxicity of a virus. Antibody titer against a virus can vary over the time period after administration of the virus to the subject, where at some particular time points, a low anti-(viral antigen) antibody titer can indicate a higher toxicity, while at other time points a high anti-(viral antigen) antibody titer can indicate a higher toxicity. The viruses used in the methods provided herein can be immunogenic, and can, therefore, elicit an immune response soon after administering the virus to the subject. Generally, a virus against which a subject's immune system can quickly mount a strong immune response can be a virus that has low toxicity when the subject's immune system can remove the virus from all normal organs or tissues. Thus, in some examples, a high antibody titer against viral antigens soon after administering the virus to a subject can indicate low toxicity of a virus. In contrast, a virus that is not highly immunogenic can infect a host organism without eliciting a strong immune response, which can result in a higher toxicity of the virus to the host. Accordingly, in some examples, a high antibody titer against viral antigens soon after administering the virus to a subject can indicate low toxicity of a virus.

In other examples, monitoring antibody titer can be used to monitor the efficacy of treatment methods. In the methods provided herein, antibody titer, such as anti-(tumor antigen) antibody titer, can indicate the efficacy of a therapeutic method such as a therapeutic method to treat neoplastic disease. Therapeutic methods provided herein can include causing or enhancing an immune response against a tumor and/or metastasis. Thus, by monitoring the anti-(tumor antigen) antibody titer, it is possible to monitor the efficacy of a therapeutic method in causing or enhancing an immune response against a tumor and/or metastasis. The therapeutic methods provided herein also can include administering to a subject a virus that can accumulate in a tumor and can cause or enhance an anti-tumor immune response. Accordingly, it is possible to monitor the ability of a host to mount an immune response against viruses accumulated in a tumor or metastasis, which can indicate that a subject has also mounted an anti-tumor immune response, or can indicate that a subject is likely to mount an anti-tumor immune response, or can indicate that a subject is capable of mounting an anti-tumor immune response.

In other examples, monitoring antibody titer can be used for monitoring the level of gene product or antibodies for production and/or harvesting. As provided herein, methods can be used for producing proteins, RNA molecules or other compounds by expressing an exogenous gene in a virus that has accumulated in a tumor. Further provided herein are methods for producing antibodies against a protein, RNA molecule or other compound produced by exogenous gene expression of a virus that has accumulated in a tumor. Monitoring antibody titer against the protein, RNA molecule or other compound can indicate the level of production of the protein, RNA molecule or other compound by the tumor-accumulated virus, and also can directly indicate the level of antibodies specific for such a protein, RNA molecule or other compound.

d. Monitoring General Health Diagnostics

The methods provided herein also can include methods of monitoring the health of a subject. Some of the methods provided herein are therapeutic methods, including neoplastic disease therapeutic methods. Monitoring the health of a subject can be used to determine the efficacy of the therapeutic method, as is known in the art. The methods provided herein also can include a step of administering to a subject a virus. Monitoring the health of a subject can be used to determine the pathogenicity of a virus administered to a subject. Any of a variety of health diagnostic methods for monitoring disease such as neoplastic disease, infectious disease, or immune-related disease can be monitored, as is known in the art. For example, the weight, blood pressure, pulse, breathing, color, temperature or other observable state of a subject can indicate the health of a subject. In addition, the presence or absence or level of one or more components in a sample from a subject can indicate the health of a subject. Typical samples can include blood and urine samples, where the presence or absence or level of one or more components can be determined by performing, for example, a blood panel or a urine panel diagnostic test. Exemplary components indicative of a subject's health include, but are not limited to, white blood cell count, hematocrit, or reactive protein concentration.

e. Monitoring Coordinated with Treatment

Also provided herein are methods of monitoring a therapy, where therapeutic decisions can be based on the results of the monitoring. Therapeutic methods provided herein can include administering to a subject a virus, where the virus can preferentially accumulate in a tumor and/or metastasis, and where the virus can cause or enhance an anti-tumor immune response. Such therapeutic methods can include a variety of steps including multiple administrations of a particular virus, administration of a second virus, or administration of a therapeutic compound. Determination of the amount, timing or type of virus or compound to administer to the subject can be based on one or more results from monitoring the subject. For example, the antibody titer in a subject can be used to determine whether or not it is desirable to administer a virus or compound, the quantity of virus or compound to administer, and the type of virus or compound to administer, where, for example, a low antibody titer can indicate the desirability of administering additional virus, a different virus, or a therapeutic compound such as a compound that induces viral gene expression. In another example, the overall health state of a subject can be used to determine whether or not it is desirable to administer a virus or compound, the quantity of virus or compound to administer, and the type of virus or compound to administer, where, for example, determining that the subject is healthy can indicate the desirability of administering additional virus, a different virus, or a therapeutic compound such as a compound that induces viral gene expression. In another example, monitoring a detectable virally expressed gene product can be used to determine whether or not it is desirable to administer a virus or compound, the quantity of virus or compound to administer, and the type of virus or compound to administer. Such monitoring methods can be used to determine whether or not the therapeutic method is effective, whether or not the therapeutic method is pathogenic to the subject, whether or not the virus has accumulated in a tumor or metastasis, and whether or not the virus has accumulated in normal tissues or organs. Based on such determinations, the desirability and form of further therapeutic methods can be derived.

In one example, determination of whether or not a therapeutic method is effective can be used to derive further therapeutic methods. Any of a variety of methods of monitoring can be used to determine whether or not a therapeutic method is effective, as provided herein or otherwise known in the art. If monitoring methods indicate that the therapeutic method is effective, a decision can be made to maintain the current course of therapy, which can include further administrations of a virus or compound, or a decision can be made that no further administrations are required. If monitoring methods indicate that the therapeutic method is ineffective, the monitoring results can indicate whether or not a course of treatment should be discontinued (e.g., when a virus is pathogenic to the subject), or changed (e.g., when a virus accumulates in a tumor without harming the host organism, but without eliciting an anti-tumor immune response), or increased in frequency or amount (e.g., when little or no virus accumulates in tumor).

In one example, monitoring can indicate that a virus is pathogenic to a subject. In such instances, a decision can be made to terminate administration of the virus to the subject, to administer lower levels of the virus to the subject, to administer a different virus to a subject, or to administer to a subject a compound that reduces the pathogenicity of the virus. In one example, administration of a virus that is determined to be pathogenic can be terminated. In another example, the dosage amount of a virus that is determined to be pathogenic can be decreased for subsequent administration; in one version of such an example, the subject can be pre-treated with another virus that can increase the ability of the pathogenic virus to accumulate in tumor, prior to re-administering the pathogenic virus to the subject. In another example, a subject can have administered thereto a virus that is pathogenic to the subject; administration of such a pathogenic virus can be accompanied by administration of, for example, an antiviral compound (e.g., cidofovir), pathogenicity attenuating compound (e.g., a compound that down-regulates the expression of a lytic or apoptotic gene product), or other compound that can decrease the proliferation, toxicity, or cell killing properties of a virus, as described herein elsewhere. In one variation of such an example, the localization of the virus can be monitored, and, upon determination that the virus is accumulated in tumor and/or metastases but not in normal tissues or organs, administration of the antiviral compound or pathogenicity attenuating compound can be terminated, and the pathogenic activity of the virus can be activated or increased, but limited to the tumor and/or metastasis. In another variation of such an example, after terminating administration of the antiviral compound or pathogenicity attenuating compound, the presence of the virus and/or pathogenicity of the virus can be further monitored, and administration of such a compound can be reinitiated if the virus is determined to pose a threat to the host by, for example, spreading to normal organs or tissues, releasing a toxin into the vasculature, or otherwise having pathogenic effects reaching beyond the tumor or metastasis.

In another example, monitoring can determine whether or not a virus has accumulated in a tumor or metastasis of a subject. Upon such a determination, a decision can be made to further administer additional virus, a different virus or a compound to the subject. In another example, monitoring the presence of a virus in a tumor can be used in deciding to administer to the subject a compound, where the compound can increase the pathogenicity, proliferation, or immunogenicity of a virus or the compound can otherwise act in conjunction with the virus to increase the proliferation, toxicity, tumor cell killing, or immune response eliciting properties of a virus; in one variation of such an example, the virus can, for example, have little or no lytic or cell killing capability in the absence of such a compound; in a further variation of such an example, monitoring of the presence of the virus in a tumor or metastasis can be coupled with monitoring the absence of the virus in normal tissues or organs, where the compound is administered if the virus is present in tumor or metastasis and not at all present or substantially not present in normal organs or tissues; in a further variation of such an example, the amount of virus in a tumor or metastasis can be monitored, where the compound is administered if the virus is present in tumor or metastasis at sufficient levels.

### F. EXAMPLES

The following examples are included for illustrative purposes only and are not intended to limit the scope of the invention.

### Example 1

### Exemplary Vaccinia Viruses

### A. GLV-1h68

The attenuated vaccinia virus strain GLV-1h68 (SEQ ID NO:1) was purified as previously described (Zhang et al., (2007) Cancer Res 67:10038-10046). This genetically engineered strain, which has been described in U.S. Pat. No. 7,588,767, contains DNA insertions in the F14.5L, thymidine kinase (TK) and hemagglutinin (HA) genes. GLV-1h68 was prepared from the vaccinia virus strain designated LIVP (a vaccinia virus strain, originally derived by adapting the vaccinia Lister strain (ATCC Catalog No. VR-1549) to calf skin (Research Institute of Viral Preparations, Moscow, Russia, Al'tshtein et al. (1983) Dokl. Akad. Nauk USSR 285:696-699). The LIVP strain, whose genome sequence is set forth in SEQ ID NO:2 and from which GLV-1h68 was generated, contains a mutation in the coding sequence of the TK gene, in which a substitution of a guanine nucleotide with a thymidine nucleotide (nucleotide position 80207 of SEQ ID NO:2) introduces a premature STOP codon within the coding sequence.

GLV-1h68 is a recombinant, replication-competent vaccinia virus derived from the vaccinia virus LIVP strain (Lister strain from the Institute of Viral Preparations, Moscow, Russia). As described in U.S. Pat. No. 7,588,767 (see Example 1), GLV-1h68 was generated by inserting expression cassettes encoding detectable marker proteins into the F14.5L (also designated in LIVP as F3) gene, thymidine kinase (TK) gene, and hemagglutinin (HA) gene loci of the vaccinia virus LIVP strain. Specifically, an expression cassette containing a Ruc-GFP cDNA molecule (a fusion of DNA encoding Renilla luciferase and DNA encoding GFP; SEQ ID NO:38 (DNA); SEQ ID NO:39 (protein)) under the control of a vaccinia synthetic early/late promoter P_{SEL} ((P_{SEL})Ruc-GFP) was inserted into the F14.5L gene; an expression cassette containing DNA encoding beta-galactosidase under the control of the vaccinia early/late promoter P_{7.5k} ((P_{7.5k})LacZ) and DNA encoding a rat transferrin receptor positioned in the reverse orientation for transcription relative to the vaccinia synthetic early/late promoter P_{SEL} ((P_{SEL})rTrfR) was inserted into the TK gene (the resulting virus does not express transferrin receptor protein since the DNA encoding the protein is positioned in the reverse orientation for transcription relative to the promoter in the cassette); and an expression cassette containing DNA encoding β-glucuronidase under the control of the vaccinia late promoter P₁₁ₖ ((P₁₁ₖ)gusA) was inserted into the HA gene. The genome of GLV-1h68 has the sequence of nucleotides set forth in SEQ ID NO:1. Insertion of the expression cassettes into the LIVP genome to generate the GLV-1h68 strain resulted in disruption of the coding sequences for each of the F14.5L, TK and HA genes; accordingly, all three genes in the resulting strains are nonfunctional in that they do not encode the corresponding full-length proteins.

Virus was propagated in CV-1 cells, and up to 7×10⁹ plaque-forming unit (pfu)/mL of GLV-1h68 can be purified from 2×10⁸ infected CV-1 cells through sucrose gradients (Joklik WK (1962) Virology 18:9-18).

### B. Modified Vaccinia Viruses

Modified recombinant vaccinia viruses containing heterologous DNA inserted into one or more loci of the vaccinia virus genome were generated via homologous recombination between DNA sequences in the GLV-1h68 genome and a transfer vector using methods described herein and known to those of skill in the art (see, e.g., Falkner and Moss (1990) J. Virol. 64:3108-3111; Chakrabarti et al. (1985) Mol. Cell. Biol. 5:3403-3409; and U.S. Pat. No. 4,722,848). In these methods, the existing target gene in the starting vaccinia virus genome is replaced by an interrupted copy of the gene contained in the transfer vector through two crossover events: a first crossover event of homologous recombination between the vaccinia virus genome and the transfer vector and a second crossover event of homologous recombination between direct repeats within the target locus. The interrupted version of the target gene that is in the transfer vector contains the insertion DNA flanked on each side by DNA corresponding to the left portion of the target gene and right portion of the target gene, respectively. The transfer vector also contains a dominant selection marker, e.g., the E. coli guanine phosphoribosyltransferase (gpt) gene, under the control of a vaccinia virus early promoter (e.g.,P_{7.5kE}). Including such a marker in the vector enables a transient dominant selection process to identify recombinant virus grown under selective pressure that has incorporated the transfer vector within its genome. Because the marker gene is not stably integrated into the genome, it is deleted from the genome in a second crossover event that occurs when selection is removed. Thus, the final recombinant virus contains the interrupted version of the target gene as a disruption of the target loci, but does not retain the selectable marker from the transfer vector.

Homologous recombination between a transfer vector and a starting vaccinia virus genome occurred upon introduction of the transfer vector into cells that have been infected with the starting vaccinia virus. Viruses included GLV-1h74, GLV-1h96, GLV-1h99, GLV-1h108 and GLV-1h163. The construction of these strains is summarized in Table 6, which lists the modified vaccinia virus strains, including the previously described GLV-1h68, their respective genotypes, and the transfer vectors used to engineer the viruses. Construction of the modified vaccinia viruses and the transfer vectors are described in U.S. Patent Pub. Nos. 2009-0117034 and 2009-0098529.

TABLE 6
Modified Vaccinia Viruses

| Name of | Parental | | |
|---|---|---|---|
| Virus | Virus | Transfer Vector | Genotype |
| GLV-1h70 | GLV-1h68 | pNCVVhaT | F14.5L: (P_{SEL})Ruc-GFP |
| | | | TK: (P_{SEL})rTrfR-(P_{7.5k})LacZ |
| | | | HA: HindIII-BamHI |
| GLV-1h73 | GLV-1h70 | pNCVVf14.5lT | F14.5L: BamHI-HindIII |
| | | | TK: (P_{SEL})rTrfR-(P_{7.5k})LacZ |
| | | | HA: HindIII-BamHI |
| GLV-1h74 | GLV-1h73 | pCR-TKLR- | F14.5L: BamHI-Hind III |
| | | gpt2 | TK: SacI-BamHI |
| | | | HA: HindIII-BamHI |
| GLV-1h96 | GLV-1h68 | FSE-IL-24 | F14.5L: (P_{SEL})IL-24 |
| | | | TK: (P_{SEL})rTrfR-(P_{7.5k})LacZ |
| | | | HA: (P₁₁ₖ)gusA |
| GLV-1h99 | GLV-1h68 | FSE-hNET | F14.5L: (P_{SEL})hNET |
| | | | TK: (P_{SEL})rTrfR-(P_{7.5k})LacZ |
| | | | HA: (P₁₁ₖ)gusA |
| GLV-1h100 | GLV-1h68 | TK-SE-hNET3 | F14.5L: (P_{SEL})Ruc-GFP |
| | | | TK: (P_{SE})hNET |
| | | | HA: (P₁₁ₖ)gusA |
| GLV-1h108 | GLV-1h68 | pCR-TK-SEL- | F14.5L: (P_{SEL})Ruc-GFP |
| | | G6-FLAG | TK: (P_{SEL})G6-FLAG |
| | | | HA: (P₁₁ₖ)gusA |
| GLV-1h163 | GLV- | pHA-PSEL- | F14.5L: (P_{SEL})Ruc-GFP |
| | 1h100 | G6-scAb | TK: (P_{SE})hNET |
| | | | HA: (P_{SEL})G6-scAB |

Briefly, the strains listed in Table 6 were generated as follows:

GLV-1h70 was generated by insertion of a short non-coding DNA fragment containing HindIII and BamHI sites into the HA locus of starting strain GLV-1h68 thereby deleting the gusA expression cassette at the HA locus of GLV-1h68. Thus, in strain GLV-1h70, the vaccinia HA gene is interrupted within the coding sequence by a short non-coding DNA fragment.

GLV-1h73 was generated by insertion of a short non-coding DNA fragment containing BamHI and HindIII sites (SEQ ID NO:29) into the F14.5L locus of GLV-1h70 thereby deleting the Ruc-GFP fusion gene expression cassette at the F14.5L locus of GLV-1h70. Thus, in strain GLV-1h73, the vaccinia HA and F14.5L genes are interrupted within the coding sequence by a short non-coding DNA fragment.

GLV-1h74 was generated by insertion of a short non-coding DNA fragment containing Sad and BamHI sites (SEQ ID NO:29) into the TK locus of strain GLV-1h73 thereby deleting the LacZ/rTFr expression cassette at the TK locus of GLV-1h73. Thus, in strain GLV-1h74, the vaccinia HA, F14.5L and TK genes are interrupted within the coding sequence by a short non-coding DNA fragment.

GLV-1h96 was generated by insertion of an expression cassette encoding the IL-24 (SEQ ID NO:27) gene under the control of the vaccinia P_{SE} promoter into the F14.5L locus of starting strain GLV-1h68, thereby deleting the Ruc-GFP fusion gene expression cassette at the F14.5L locus of GLV-1h68. The FSE-IL-24 transfer vector is set forth in SEQ ID NO:30. Thus, in strain GLV-1h96, the vaccinia F14.5L gene is interrupted within the coding sequence by a DNA fragment containing DNA encoding IL-24 operably linked to the vaccinia synthetic early promoter. GLV-1h99 was generated by insertion of an expression cassette encoding the human norepinephrine transporter (hNET; SEQ ID NO:36) gene under the control of the vaccinia P_{SE} promoter into the F14.5L locus of starting strain GLV-1h68, thereby deleting the Ruc-GFP fusion gene expression cassette at the F14.5L locus of starting GLV-1h68. The FSE-hNET transfer vector is set forth in SEQ ID NO:31. Thus, in strain GLV-1h99, the vaccinia F14.5L gene is interrupted within the coding sequence by a DNA fragment containing DNA encoding hNET operably linked to the vaccinia synthetic early promoter.

GLV-1h100 was generated by insertion of an expression cassette encoding hNET under the control of the vaccinia P_{SE} promoter into the TK locus of starting strain GLV-1h68 thereby deleting the LacZ/rTFr expression cassette at the TK locus of starting GLV-1h68. The TK-SE-hNET3 transfer vector is set forth in SEQ ID NO:32. Thus, in strain GLV-1h100, the vaccinia TK gene is interrupted within the coding sequence by a DNA fragment containing DNA encoding hNET operably linked to the vaccinia synthetic early promoter.

GLV-1h108 was generated by insertion of an expression cassette containing DNA encoding G6-FLAG fusion protein under the control of the vaccinia synthetic early/late promoter (P_{SEL}) into the TK locus of strain GLV-1h68 thereby deleting the LacZ/rTFr expression cassette at the TK locus of GLV-1h68. The pCT-TK-SEL-G6-FLAG transfer vector is set forth in SEQ ID NO:33. Strain GLV-1h108 retains the Ruc-GFP expression cassette at the F14.5L locus and the gusA expression cassette at the HA locus.

GLV-1h163 was derived from the GLV-1h100 strain (described in above) by replacement of gusA gene (beta-glucuronidase) by the gene encoding G6-scAB protein (GLAF-2; SEQ ID NO:34) into A56R locus. The GLAF-2 gene is under the control of the VACV synthetic early/late (SEL) promoter. In addition, GLV-1h163 carries the human norepinephrine transporter (NET) under the control of the VACV synthetic early (SE) promoter into J2R locus. Thus, in strain GLV-1h163, the vaccinia TK gene is interrupted within the coding sequence by a DNA fragment encoding hNET operably linked to the vaccinia synthetic early promoter and the vaccinia HA gene is interrupted within the coding sequence by a DNA fragment containing DNA encoding the single chain anti-VEGF antibody (G6-scAB; (SEQ ID NO:35)) operably linked to the vaccinia synthetic early/late promoter.

### Example 2

### Xenograft Tumor Models

Xenograft tumor models, derived from injection of C6 rat glioma cells or A549 human lung cancer cells, were used for the in vivo studies that follow.

### A. C6 Glioma Xenografts

C6 rat glioma cells (ATCC No. CCL-107, Rockville, Md.) were cultured in RPMI-1640 medium (Cellgro, Mediatech, Inc., Herndon, Va.) supplemented with 10% (v/v) fetal bovine serum (FBS) and 1× penicillin/streptomycin under standard cell culture conditions (37° C., 5% CO₂). Subcutaneous glioma tumors were generated by subcutaneous injection of 5×10⁵ C6 glioma cells, in 100 μl phosphate buffered saline (PBS), into the later aspect of the right rear thigh of 5-6 week-old male BALB/c athymic nu⁻/nu⁻ mice (25-30 g body weight).

### B. A549 Lung Cancer Xenografts

Human A549 lung cancer cells (ATCC No. CCL-185) were cultured in RPMI-1640 medium containing 10% FBS and 1% antibiotic-antimycotic solution (PAA Laboratories, Cölbe, Germany) under standard cell culture conditions (37° C., 5% CO₂). A549 xenograft tumors were developed in 5-6 week-old female athymic nude mice (NCI:Hsd:Athymic Nude Foxn1^{nu}, Harlan Borchem, Germany) by implanting 5×10⁶ cells subcutaneously in the hind right flank. Tumor growth was monitored by recording tumor size with a digital caliper. Tumor volume (mm³) was estimated by the formula ½(L×H×W), where L is the length, W is the width, and H is the height of the tumor in millimeters (mm). Treatment as described in Examples 4 and 5 below began after the tumors grew to about 200 mm³ in size (3 weeks).

### Example 3

### Co-Administration of GLV-1h68 and Bacteria

Light emitting E. coli (E. coli/pLITE) strains were created by transforming DH5a cells (ATCC, Rockville, Md.) with the luxCDABE-containing plasmid pLITE-201 (Voisey and Marincs (1998) BioTechniques 24:56-58). The transformed cells were selected by culturing the transformed bacteria in LB medium, supplemented with 100 μg/mL ampicillin, at 37° C. Mice bearing C6 glioma tumors as described in Example 2A were split into 3 groups, with 3 mice in each group. On days 11 and/or day 16, the groups of mice were administered 1×10⁷ plaque forming units (pfu) GLV-1h68 and/or 1×10⁸ colony forming units (cfu) E. coli/pLITE (in stationary phase) in 100 μL phosphate buffered saline (PBS) by intravenous injection using a 1-cc insulin syringe equipped with a 29½-gauge needle through the surgically exposed superficial femoral circumflex vein, according to Table 7 below. After each injection, the incision exposing the vein was re-approximated with 5-0 nylon sutures (Harvard Apparatus, Holliston, Mass.).

TABLE 7

| Group | Tumor day 11 | Tumor day 16 |
|---|---|---|
| 1 | 1 × 10⁷ pfu GLV-1h68 | 1 × 10⁸ cfu E. coli/pLITE |
| 2 | | 1 × 10⁸ cfu E. coli/pLITE + 1 × 10⁷ |
| | | pfu GLV-1h68 |
| 3 | | 1 × 10⁸ cfu E. coli/pLITE |

On tumor day 21, the animals were euthanized, under anesthesia, with Ketamine/Xylazine. The tumor tissues were excised and homogenized using a MagNA Lyser (Roche Applied Science, Indianapolis, Ind.) at 6500 rpm for 30s. Each sample was serially diluted with PBS and plated on selective agar plates with 100 mg/mL ampicillin. Bacterial colonies were counted after overnight incubation at 37° C. and used to calculate bacterial titers per tumor. Differences in the levels of bacterial colonization between groups were analyzed by t-test using SPSS 10.0 software. A P value of less than 0.05 was considered statistically significant.

The mice in Group 1 had on average approximately 5×10⁹ E. coli/pLITE/tumor, mice in group 2 had on average approximately 4×10⁹ E. coli/pLITE/tumor and mice in Group 3 had on average approximately 1×10⁹ E. coli/pLITE/tumor. t-test analysis indicated that Groups 1 and 2 had a significantly greater bacterial titer compared to Group 3. These data suggest that oncolytic VACV precolonization or coinjection may be used in conjunction with bacterial infection to increase the efficacy of bacterial colonization of tumors.

### Example 4

### Effect of Co-Administration of Antibiotics on Vaccinia Virus Treatment of Xenograft Tumors

The effect of co-administration of antibiotics and vaccinia virus was determined in A549 tumor bearing mice.

A. Treatment with Penicillin-Streptomycin

The A549 tumor bearing mice were split into 3 groups, with 10 mice in each group. 5×10⁶ plaque forming units (pfu) GLV-1h68 in 100 μL phosphate buffered saline (PBS) was administered via the retro-orbital (r.o.) sinus vein to mice in all 3 groups on day 0. A penicillin-streptomycin solution (PS) containing 10,000 I.U./mL penicillin and 10,000 μg/mL streptomycin (Cellgro, Cat. No. 30-002-C1) was administered via drinking water or via intraperitoneal injection. Mice in Group 2 were administered antibiotics in drinking water (6 mL PS+600 mL water) 2 times a week for 8 weeks starting on day 4 post viral infection. Mice in Group 3 were administered 200 μL PS/mouse via intraperitoneal injection 3 times a week for 8 weeks starting on day 4 post viral infection. Mice in Group 1 were not administered antibiotics. Mice were observed weekly to assess tumor volume, weight and any signs of toxicity.

The results show treatment with intraperitoneally administered penicillin-streptomycin and GLV-1h68 increased the survival rate and reduced the weight loss of A549 tumor bearing mice as compared to treatment with virus alone. 100% of mice receiving intraperitoneal penicillin-streptomycin were alive 56 days after virus injection as compared to only 20% of mice treated with GLV-1h68 virus only. Mice administered antibiotics via drinking water also had an increased survival rate compared to mice that were not administered antibiotics. Tumor volume decreased in all mice at approximately the same rate. Mice administered antibiotics intraperitoneally gained approximately 10% in net body weight whereas mice administered only virus lost approximately 20% of net body weight by day 49 after virus injection. Mice administered antibiotics via drinking water had exhibited no net change in body weight 49 days after virus injection, but a sharp decrease of 20% was observed by day 56. The results show that GLV-1h68 is effective at shrinking tumor volume in the presence of antibiotics administered intraperitoneally or via drinking water.

B. Treatment with Antibiotics or an Antibiotics-Antimycotic Solution

The A549 tumor bearing mice were split into 19 groups, with 8 mice in each group. 1×10⁷ plaque forming units (pfu) vaccinia virus in 100 μL phosphate buffered saline (PBS) was administered via the tail vain (t.v.) (groups 1-3) or the retro-orbital (r.o.) sinus vein (groups 4-19). Vaccinia viruses tested included GLV-1h68, GLV-1h74, GLV-1h96, GLV-1h99, GLV-1h108 and GLV-1h163. Mice were administered via intraperitoneal injection 200 μL of either a penicillin-streptomycin solution (PS) containing 10,000 I.U./mL penicillin and 10,000 μg/mL streptomycin (Cellgro, Cat. No. 30-002-C1) or an antibiotic-antimycotic solution (PSA) containing 10,000 I.U./mL penicillin, 10,000 vg/mL streptomycin and 25 vg/mL amphotericin B (Cellgro, Cat. No. 30-004-C1) according to Table 8 below for 9 weeks, starting 3 days after virus injection.

TABLE 8
Study Design

| | | Method of virus | | Antibiotic |
|---|---|---|---|---|
| Group | Virus | administration | Antibiotic | administration |
| 1 | GLV-1h68 | t.v. | PSA | 3x/week |
| 2 | GLV-1h68 | t.v. | PSA | 1x/week |
| 3 | GLV-1h68 | t.v. | None | |
| 4 | GLV-1h68 | r.o. | PS | 3x/week |
| 5 | GLV-1h68 | r.o. | PSA | 3x/week |
| 6 | GLV-1h68 | r.o. | PSA | 2x/week |
| 7 | GLV-1h68 | r.o. | PSA | 1x/week |
| 8 | GLV-1h68 | r.o. | None | |
| 9 | GLV-1h74 | r.o. | PSA | 3x/week |
| 10 | GLV-1h74 | r.o. | None | |
| 11 | GLV-1h96 | r.o. | PSA | 3x/week |
| 12 | GLV-1h96 | r.o. | None | |
| 13 | GLV-1h99 | r.o. | PSA | 3x/week |
| 14 | GLV-1h99 | r.o. | None | |
| 15 | GLV-1h108 | r.o. | PSA | 3x/week |
| 16 | GLV-1h108 | r.o. | None | |
| 17 | GLV-1h163 | r.o. | PSA | 3x/week |
| 18 | GLV-1h163 | r.o. | None | |
| 19 | None | | None | |

The mice were monitored weekly for 9 weeks to assess tumor volume, body weight and any signs of toxicity. Untreated, control (group 19) animals were sacrificed after 5 weeks. Tumor volume was estimated as described in Example 2B. The percent relative change in median tumor volume was calculated by the following formula:

Relative change median tumor volume (mtv)=(mtv_{day n}−mtv_{day 0})/mtv_{day 0}×100.

where mtv_{day n} is the median tumor volume on the day measured and mtv_{day 0} is the median tumor volume the day of virus infection (day 0).

Net body weight was using the following formula:

Net body weight=Total body weight−(tumor volume/1000).

The percent relative change in net body weight was calculated using the same formula used to calculate by the relative change in median tumor volume, substituting the values for the median net body weight on the day measured (day n) and the median net body weight the day of virus infection (day 0). The relative change in median tumor volume and the relative change in median net body weight, calculated only for groups which contained at least 3 surviving animals, are set forth in Tables 9 and 10, respectively. Tallies of surviving mice were taken daily and used to calculate the rate of survival for each of the treatment groups. The percent (%) survival over the course of the study is set forth in Table 12 below.

TABLE 9
Relative Change in Median Tumor Volume (%)

| | Days After Virus Injection | | | | | | | | |
|---|---|---|---|---|---|---|---|---|---|
| Group | 7 | 14 | 21 | 28 | 35 | 42 | 49 | 56 | 63 |
| 1 | 168.5 | 302.9 | 232.3 | 118.5 | 13.8 | −5.8 | nd | nd | nd |
| 2 | 173.8 | 313.7 | 273.1 | 137.6 | 24.0 | −4.9 | −13.0 | −50.2 | −22.3 |
| 3 | 126.2 | 206.3 | 161.2 | 61.5 | nd | nd | nd | nd | nd |
| 4 | 111.5 | 283.7 | 486.9 | 529.7 | 488.7 | 448.6 | 402.2 | 279.3 | 184.1 |
| 5 | 149.7 | 327.2 | 637.2 | 617.0 | 648.1 | 581.7 | 463.5 | 316.4 | 195.8 |
| 6 | 131.2 | 370.8 | 571.8 | 636.4 | 667.8 | 497.3 | 343.9 | 282.5 | 264.4 |
| 7 | 87.2 | 264.4 | 474.7 | 506.1 | 423.0 | 329.3 | 207.4 | 108.4 | 96.7 |
| 8 | 113.6 | 317.0 | 683.4 | 793.4 | 696.6 | 577.4 | 407.7 | 304.2 | 263.1 |
| 9 | 155.3 | 407.3 | 532.5 | 416.1 | 178.4 | 9.6 | nd | nd | nd |
| 10 | 116.2 | 261.3 | 385.5 | 285.2 | 177.4 | 75.2 | −15.3 | nd | nd |
| 11 | 124.9 | 296.7 | 530.3 | 522.5 | 434.4 | 294.9 | 281.7 | 165.7 | 134.0 |
| 12 | 126.7 | 436.4 | 602.3 | 624.9 | 517.6 | 395.4 | 281.4 | 118.3 | 104.9 |
| 13 | 127.8 | 345.9 | 368.1 | 306.3 | 186.0 | 110.8 | 16.7 | −14.4 | −23.4 |
| 14 | 144.3 | 254.5 | 347.9 | 287.5 | 205.2 | 166.7 | 85.5 | 78.1 | 11.9 |
| 15 | 59.6 | 111.8 | 167.8 | 189.5 | 141.3 | 105.7 | 87.1 | 65.2 | 57.5 |
| 16 | 55.2 | 92.6 | 206.4 | 165.5 | 136.5 | 51.4 | 29.1 | 35.0 | −17.3 |
| 17 | 79.7 | 109.1 | 263.2 | 292.5 | 230.7 | 211.1 | 160.2 | 115.4 | 77.1 |
| 18 | 83.1 | 115.2 | 167.4 | 193.0 | 108.9 | 51.6 | 38.9 | 19.8 | 21.0 |
| 19 | 95.0 | 220.8 | 644.0 | 945.4 | 1229.6 | nd | nd | nd | nd |

nd = not determined because the number of surviving mice (n) < 3

TABLE 11
Relative Change in Median Net Body Weight (%)

| | Days After Virus Injection | | | | | | | | |
|---|---|---|---|---|---|---|---|---|---|
| Group | 7 | 14 | 21 | 28 | 35 | 42 | 49 | 56 | 63 |
| 1 | −0.6 | −0.8 | −5.1 | −11.1 | −20.6 | −13.0 | nd | nd | nd |
| 2 | −1.2 | 1.3 | 0.6 | −0.8 | −1.5 | −2.5 | 2.6 | −1.3 | 0.1 |
| 3 | 0.7 | −5.9 | −13.3 | −27.0 | nd | nd | nd | nd | nd |
| 4 | 1.8 | 8.2 | 6.5 | 5.6 | 4.4 | 6.3 | 4.4 | 2.4 | −2.1 |
| 5 | 3.8 | 4.9 | 4.3 | 3.0 | 8.7 | 5.9 | 9.0 | 8.3 | 6.2 |
| 6 | 0.3 | 4.3 | 1.8 | 0.4 | 3.6 | 7.8 | 9.1 | 8.2 | 9.8 |
| 7 | 3.0 | 6.4 | −1.0 | −4.4 | −0.2 | 3.0 | 9.6 | 8.6 | 10.5 |
| 8 | 2.0 | 7.4 | 9.1 | 5.5 | 5.7 | 8.3 | 10.1 | 10.9 | 13.1 |
| 9 | 0.6 | 3.5 | −4.9 | −10.8 | −21.8 | −31.1 | nd | nd | nd |
| 10 | −1.8 | −4.5 | −5.5 | −7.4 | −9.6 | −19.3 | −28.6 | nd | nd |
| 11 | −3.3 | 3.8 | 2.7 | 0.6 | 3.0 | 2.2 | 10.8 | 7.6 | 11.4 |
| 12 | −2.9 | 3.8 | −0.3 | −2.1 | 5.1 | −0.8 | 1.7 | −6.3 | −9.0 |
| 13 | 2.5 | 3.6 | 1.4 | −2.8 | −1.3 | 2.0 | −6.4 | −18.1 | −24.7 |
| 14 | −0.8 | 0.7 | −0.7 | −6.2 | −3.8 | −1.4 | 1.5 | 5.0 | 3.7 |
| 15 | 1.4 | 3.4 | 2.7 | 2.7 | 5.3 | 7.8 | 9.9 | 10.8 | 11.8 |
| 16 | 0.1 | 2.6 | −1.1 | −0.7 | −1.1 | 1.7 | 7.6 | 6.6 | −0.7 |
| 17 | 0.2 | 4.2 | −2.0 | 2.9 | 6.6 | 7.8 | 10.4 | 10.3 | 15.4 |
| 18 | 0.8 | 3.5 | −0.5 | 0.9 | 6.3 | 6.2 | 10.2 | 7.8 | 13.4 |
| 19 | 16.1 | 19.6 | 18.2 | 17.8 | 22.3 | nd | nd | nd | nd |

nd = not determined because the number of surviving mice (n) < 3

TABLE 12
Percent (%) Animal Survival

| Days after | | | | | | | | | | | | | | | | | | | |
|---|---|---|---|---|---|---|---|---|---|---|---|---|---|---|---|---|---|---|---|
| virus | Group | | | | | | | | | | | | | | | | | | |
| injection | 1 | 2 | 3 | 4 | 5 | 6 | 7 | 8 | 9 | 10 | 11 | 12 | 13 | 14 | 15 | 16 | 17 | 18 | 19 |
| 0 | 100 | 100 | 100 | 100 | 100 | 100 | 100 | 100 | 100 | 100 | 100 | 100 | 100 | 100 | 100 | 100 | 100 | 100 | 100 |
| 20 | 100 | 100 | 87.5 | 100 | 100 | 100 | 100 | 100 | 100 | 100 | 100 | 100 | 100 | 100 | 100 | 100 | 100 | 100 | 100 |
| 23 | 100 | 100 | 75 | 100 | 87.5 | 100 | 100 | 100 | 100 | 100 | 100 | 100 | 100 | 100 | 100 | 100 | 100 | 100 | 100 |
| 24 | 100 | 100 | 62.5 | 100 | 87.5 | 87.5 | 100 | 100 | 100 | 100 | 100 | 100 | 100 | 100 | 100 | 100 | 100 | 100 | 100 |
| 28 | 100 | 100 | 37.5 | 100 | 87.5 | 87.5 | 100 | 100 | 100 | 100 | 100 | 87.5 | 100 | 100 | 100 | 100 | 100 | 100 | 100 |
| 29 | 100 | 100 | 25 | 100 | 87.5 | 87.5 | 100 | 100 | 100 | 100 | 100 | 87.5 | 100 | 100 | 100 | 100 | 100 | 100 | 100 |
| 30 | 100 | 100 | 25 | 100 | 75 | 87.5 | 100 | 100 | 100 | 100 | 100 | 87.5 | 100 | 87.5 | 100 | 100 | 100 | 100 | 100 |
| 33 | 87.5 | 100 | 25 | 100 | 75 | 87.5 | 100 | 100 | 100 | 100 | 100 | 87.5 | 100 | 87.5 | 100 | 100 | 100 | 100 | 100 |
| 34 | 75 | 100 | 25 | 100 | 75 | 87.5 | 100 | 100 | 100 | 100 | 100 | 87.5 | 100 | 87.5 | 100 | 100 | 100 | 100 | 100 |
| 35 | 75 | 100 | 25 | 100 | 75 | 87.5 | 100 | 100 | 100 | 100 | 100 | 87.5 | 100 | 87.5 | 100 | 100 | 100 | 100 | 100 |
| 36 | 62.5 | 75 | 12.5 | 100 | 75 | 87.5 | 100 | 100 | 62.5 | 87.5 | 87.5 | 87.5 | 87.5 | 62.5 | 100 | 100 | 100 | 100 | |
| 38 | 62.5 | 75 | 12.5 | 100 | 75 | 87.5 | 100 | 100 | 62.5 | 75 | 87.5 | 87.5 | 87.5 | 62.5 | 100 | 100 | 100 | 100 | |
| 39 | 50 | 75 | 12.5 | 100 | 75 | 87.5 | 100 | 100 | 62.5 | 75 | 87.5 | 87.5 | 87.5 | 62.5 | 100 | 100 | 100 | 100 | |
| 42 | 50 | 62.5 | 12.5 | 100 | 75 | 87.5 | 100 | 100 | 62.5 | 62.5 | 87.5 | 87.5 | 75 | 62.5 | 100 | 100 | 100 | 100 | |
| 44 | 50 | 62.5 | 12.5 | 100 | 75 | 87.5 | 87.5 | 87.5 | 25 | 62.5 | 75 | 87.5 | 75 | 62.5 | 100 | 87.5 | 100 | 100 | |
| 47 | 37.5 | 62.5 | 12.5 | 100 | 75 | 87.5 | 87.5 | 87.5 | 25 | 62.5 | 75 | 87.5 | 75 | 62.5 | 100 | 87.5 | 100 | 100 | |
| 48 | 25 | 62.5 | 12.5 | 100 | 75 | 87.5 | 87.5 | 87.5 | 25 | 62.5 | 75 | 87.5 | 75 | 62.5 | 100 | 87.5 | 100 | 100 | |
| 49 | 25 | 62.5 | 12.5 | 100 | 75 | 87.5 | 87.5 | 87.5 | 25 | 50 | 75 | 87.5 | 75 | 62.5 | 100 | 87.5 | 100 | 100 | |
| 50 | 12.5 | 62.5 | 12.5 | 100 | 75 | 87.5 | 87.5 | 87.5 | 25 | 37.5 | 75 | 87.5 | 75 | 62.5 | 100 | 87.5 | 100 | 100 | |
| 51 | 0 | 62.5 | 12.5 | 100 | 75 | 87.5 | 87.5 | 87.5 | 25 | 37.5 | 75 | 87.5 | 75 | 62.5 | 100 | 87.5 | 100 | 100 | |
| 55 | 0 | 62.5 | 12.5 | 100 | 75 | 87.5 | 87.5 | 87.5 | 25 | 25 | 75 | 87.5 | 75 | 62.5 | 100 | 87.5 | 100 | 100 | |
| 56 | 0 | 62.5 | 12.5 | 100 | 75 | 87.5 | 87.5 | 87.5 | 25 | 25 | 75 | 75 | 50 | 50 | 87.5 | 75 | 100 | 100 | |
| 57 | 0 | 62.5 | 12.5 | 100 | 75 | 87.5 | 87.5 | 87.5 | 12.5 | 25 | 75 | 75 | 37.5 | 50 | 87.5 | 75 | 100 | 100 | |
| 58 | 0 | 62.5 | 12.5 | 87.5 | 75 | 87.5 | 75 | 87.5 | 0 | 0 | 75 | 75 | 37.5 | 50 | 87.5 | 75 | 100 | 100 | |
| 63 | 0 | 62.5 | 12.5 | 87.5 | 75 | 87.5 | 62.5 | 87.5 | 0 | 0 | 75 | 75 | 37.5 | 50 | 87.5 | 75 | 100 | 100 | |

Untreated control mice showed a 1200% increase in tumor volume by day 35 (week 5). Mice in groups 5-8 treated with GLV-1h68 and PSA exhibited a peak in tumor volume on day 28 or day 35 (approximately 600% relative change) with a decrease in tumor volume from days 35 to 63. Mice treated only lx/week with PSA (group 7) exhibited the smallest increase in tumor volume (approximately 500% relative change), and after 63 days, the tumor size shrunk to only approximately 100% relative change in tumor volume. In addition, GLV-1h68 was more effective in the presence of antibiotics (groups 5-7) than in treatment with GLV-1h68 alone (group 8). GLV-1h74, GLV-1h96, GLV-1h99, GLV-1h108, and GLV-1h163 (groups 9-18) were all effective in reducing tumor volume in the presence and absence of PSA. In general, virus-infected animals, with or without antibiotics exhibited less net body weight gain than the untreated controls animals. Animal death contributed to fluctuation in median tumor volume and median net body weight values. These results show that vaccinia virus treatment of tumors is more effective in the presence of antibiotics.

### Example 5

### Effect of Gut Bacteria Depletion on Viral Colonization

The effect of depletion of gut bacteria on viral colonization was determined in A549 tumor bearing mice.

### A. Study Design

The A549 tumor bearing mice were split into 7 groups, with 4 or 8 mice in each group, as shown in Table 13 below. Groups 1, 2, and 3 were control groups receiving no treatment, treatment with antibiotics only or treatment with virus GLV-1h68 only, respectively. Treatment with antibiotics began after the tumors grew to about 200 mm³ in size (3 weeks). A combination of 4 antibiotics was administered to deplete gut bacteria. Ampicillin, neomycin, metronidazole and vancomycin were administered via oral gavage in an amount of 10 mg/antibiotic once a day from days 1-5, and again on days 14 and 15. All four antibiotics were also administered via drinking water containing 1 g/L ampicillin, neomycin and metronidazole and 500 mg/L vancomycin. Group 4 received antibiotics via drinking water on days 5-16 and Group 5 received antibiotics via drinking water on days 5-10, and Groups 6 and 7 received antibiotics via drinking water starting on day 5 and continuing through the end of the study (71 days post virus injection). Group 5 mice were recolonized with bacteria from feces of untreated mice by orally administering 100 μL mouse feces solution (2 feces in 1 mL deionized water) to each mouse on day 10. 5×10⁶ plaque forming units (pfu) GLV-1h68 in 100 μl phosphate buffered saline (PBS) was administered via the retro-orbital (r.o.) sinus vein on day 16 to mice in groups 3, 4, 5 and 6. Mice were observed weekly to assess tumor volume, weight and any signs of toxicity.

TABLE 13
Study Design

| | | A549 | Antibiotics | | Bacteria | |
|---|---|---|---|---|---|---|
| | | Implan- | Oral | | Recoloni- | Virus |
| Group | n | tation | Gavage | Water | zation | GLV-1h68 |
| 1 | 4 | day −20 | | | | |
| 2 | 4 | day −20 | days 1-5, | days 5-16 | | |
| | | | 14, 15 | | | |
| 3 | 8 | day −20 | | | | day 16 |
| 4 | 8 | day −20 | days 1-5, | days 5-16 | | day 16 |
| | | | 14, 15 | | | |
| 5 | 8 | day −20 | days 1-5 | days 5-10 | day 10 | day 16 |
| 6 | 8 | day −20 | days 1-5, | days 5-71 | | day 16 |
| | | | 14, 15 | | | |
| 7 | 4 | day −20 | days 1-5, | days 5-71 | | |
| | | | 14, 15 | | | |

### B. Bacterial Depletion and Recolonization

Feces were cultured to monitor intestinal bacteria after antibiotic treatment. Two (2) feces per mouse were collected on days 8, 12, and 16 after antibiotic treatment (4-5 mice per group). Serial dilutions of feces in PBS were spread on HBI blood agar plates and incubated at 37° C. under anaerobic conditions. Bacterial colonies were counted 2 days after incubation and reported as colony forming units/grams feces (CFU/g feces).

The results are shown in Table 14 below. The results show that, on day 8, mice in Group 3 which were not treated with antibiotics had a 3.46×10⁸ times reduction in colony forming units of bacteria compared to the antibiotic treated group (Group 5). After recolonization on day 10, bacteria were reestablished to normal levels by day 12.

TABLE 14
Bacterial Load (CFU/g)

| Group | Day 8 | Day 12 |
|---|---|---|
| Untreated (Group 3) | 8.5E+09 | 6.0E+09 |
| Ab.T + Recolonization (Group 5) | 2E+01 | 1E+09 |

C. Treatment with Antibiotics Increases Virus Intensity in Tumors

Mice in Groups 3-6 were analyzed for the presence of GLV-1h68 virus-dependent luciferase activity 6 days post virus injection. In short, mice were injected intraperitoneally with a mixture of 5 μL coelenterazine (Sigma; 0.5 mg/mL diluted ethanol solution) and 95 μL luciferase assay buffer (0.5 M NaCl, 1 mM EDTA and 0.1 M potassium phosphate, pH 7.4). Bioluminescence was measured from dorsal views of the animals using the Carestream Imaging System and reported as relative luminescence units (RLU).

The results are shown in Table 15 below. The results show a reduction in gut bacteria resulted in increased viral replication in tumors. Treatment with antibiotics and GLV-1h68 (Group 4 and Group 6) resulted in mice with increased tumor luminescence intensity as compared to mice treated with GLV-1h68 alone (Group 3) or mice recolonized with bacteria prior to viral treatment (Group 4).

TABLE 15
Luminescent intensity in Tumors (RLU)

| Treatment Group | Median | Average |
|---|---|---|
| GLV-1h68 | 3.04E+04 | 6.13E+04 ± 6.44E+04 |
| (Group 3) | | |
| AbT + GLV-1h68 | 1.36E+05 | 1.98E+05 ± 2.23E+05 |
| (Group 4) | | |
| AbT + Recolonization + GLV-1h68 | 4.27E+04 | 8.02E+04 ± 7.44E+04 |
| (Group 5) | | |
| AbT + GLV-1h68 + AbT-W | 2.71E+05 | 2.11E+05 ± 1.49E+05 |
| (Group 6) | | |

### D. Viral Distribution in Tumor and Organ Tissue

Three mice each from groups 3, 4 and 5 were sacrificed 7 days post virus injection and internal organs were removed. Viral distribution in tumor and organ tissue was determined by standard plaque assays. In short, tumor tissue, and tissue from organs, including lung, kidney, heart, brain and liver, were suspended in 500 mL PBS containing protease inhibitor and homogenized for 30 seconds at a speed of 6500 rpm. After homogenization, samples were subjected to 3 freeze-thaw cycles. Samples were then centrifuged for 5 minutes at 3000 g at 4° C., supernatants were collected, and serial dilutions made. Standard plaque assays were performed on CV-1 cell monolayers and recorded as plaque forming units/grams (pfu/g).

The results are shown in Table 16 below. The results show that treatment with antibiotics increased viral titers in tumors, but not in healthy organs. Mice from Group 4, which were treated with antibiotics and GLV-1h68, had a 2.5-fold increase in viral titer compared to mice in Groups 3 and 5 which were either not treated with antibiotics, or were treated with antibiotics but recolonized with bacteria prior to virus injection. GLV-1h68 was not detected in the healthy organs of 8 of 9 mice sampled from groups 3, 4, and 5. One mice from Group 4, treated with antibiotics and GLV-1h68, contained trace amounts of GLV-1h68 in brain tissue, with no observation of GLV-1h68 in the liver, kidney, lung or heart of the same mice.

TABLE 16
Virus distribution in tumors and healthy organs (pfu/g)

| Virus (Group) | Tumor | Liver | Kidney | Lung | Heart | Brain |
|---|---|---|---|---|---|---|
| GLV-1h68 | 2.96E+05 | nd | nd | nd | nd | nd |
| (Group 3) | 5.55E+05 | nd | nd | nd | nd | nd |
| | 3.67E+04 | nd | nd | nd | nd | nd |
| AbT + GLV-1h68 | 2.54E+05 | nd | nd | nd | nd | 58 |
| (Group 4) | 1.05E+06 | nd | nd | nd | nd | nd |
| | 9.38E+05 | nd | nd | nd | nd | nd |
| AbT + | 8.71E+04 | nd | nd | nd | nd | nd |
| Recolonization + | 5.64E+05 | nd | nd | nd | nd | nd |
| GLV-1h68 | 5.12E+04 | nd | nd | nd | nd | nd |
| (Group 5) | | | | | | |

nd = not detected

### E. Effect on Tumor Growth

Tumor growth was measured, as described in Example 2B above, on the day antibiotic treatment commenced and weekly thereafter for 10 weeks, i.e., on days 0, 7, 14, 21, 28, 35, 42, 49, 56, 63, and 70 days post antibiotic treatment. The average tumor volume and corresponding standard deviation for each group at each time point is set forth in Table 17.

TABLE 17
Tumor Volume (mm³)

| days post anti- | Group 1 | | Group 2 | | Group 3 | | Group 4 | |
|---|---|---|---|---|---|---|---|---|
| biotic treatment | Average | SD | Average | SD | Average | SD | Average | SD |
| 0 | 175.80 | 41.00 | 199.46 | 59.47 | 159.88 | 28.75 | 161.18 | 50.05 |
| 7 | 224.17 | 86.87 | 379.75 | 93.21 | 353.74 | 58.89 | 285.11 | 102.33 |
| 14 | 314.99 | 101.03 | 561.38 | 193.59 | 406.10 | 86.40 | 355.70 | 162.89 |
| 21 | 468.93 | 151.97 | 791.75 | 143.07 | 623.76 | 131.01 | 577.18 | 281.59 |
| 28 | 491.39 | 180.54 | 975.44 | 247.05 | 736.75 | 236.25 | 614.33 | 380.24 |
| 35 | 619.04 | 257.86 | 1246.36 | 412.22 | 845.94 | 406.94 | 635.94 | 376.31 |
| 42 | 719.98 | 337.48 | 1465.88 | 491.51 | 809.41 | 439.85 | 510.50 | 338.53 |
| 49 | 969.82 | 433.21 | 1803.79 | 514.70 | 672.39 | 466.30 | 460.84 | 364.86 |
| 56 | 1042.12 | 530.81 | 2302.55 | 841.56 | 528.51 | 408.83 | 267.91 | 207.47 |
| 63 | 1228.29 | 643.95 | 2813.46 | 816.79 | 429.06 | 395.21 | 219.93 | 144.79 |
| 70 | 1526.78 | 762.54 | 3106.10 | 793.44 | 393.13 | 332.74 | 153.91 | 124.16 |

| days post anti- | Group 5 | | Group 6 | | Group 7 | |
|---|---|---|---|---|---|---|
| biotic treatment | Average | SD | Average | SD | Average | SD |
| 0 | 219.21 | 41.85 | 193.29 | 55.54 | 209.26 | 46.82 |
| 7 | 316.22 | 46.96 | 350.24 | 102.36 | 354.39 | 92.24 |
| 14 | 443.50 | 71.74 | 458.61 | 169.34 | 549.08 | 169.67 |
| 21 | 562.80 | 110.45 | 685.60 | 251.50 | 799.82 | 182.18 |
| 28 | 821.00 | 105.03 | 865.41 | 385.21 | 961.67 | 288.85 |
| 35 | 968.85 | 210.36 | 922.88 | 338.80 | 1159.41 | 372.14 |
| 42 | 942.40 | 379.27 | 689.40 | 307.01 | 1532.33 | 433.03 |
| 49 | 814.06 | 453.80 | 509.63 | 320.21 | 1806.61 | 595.91 |
| 56 | 732.58 | 674.74 | 355.59 | 205.03 | 2051.64 | 595.23 |
| 63 | 713.85 | 742.48 | 293.91 | 142.88 | 2585.24 | 778.44 |
| 70 | 497.35 | 497.01 | 239.88 | 118.72 | 2730.33 | 732.32 |

Untreated animals (Group 1) exhibited progressive tumor growth, resulting in a tumor volume that was approximately 9 times the starting tumor volume by day 70 (1527 mm³ vs. 176 mm³). Animals treated with antibiotic only, whether receiving antibiotics through day 15 (Group 2) or through day 71 (Group 7), exhibited accelerated tumor growth compared to untreated, Group 1 animals, that continued through the course of the study. For example, by day 70, the average tumor size of Group 2 animals, which began the study with an average tumor volume of 160 mm³, was 3106 mm³, almost a 20-fold increase. Group 7 animals exhibited a similar increase in tumor volume, increasing from 209 mm³ to 2730 mm³. The tumor volume of animals administered GLV-1h68 only (Group 3), GLV-1h68 plus antibiotics (Group 4), GLV-h68 plus antibiotics and recolonization (Group 5), and GLV-1h68 plus antibiotics for the duration of the study, peaked at day 35 with a tumor volume that was 4 to 5 times the size of the average starting tumor volume, which was similar to the average tumor volume observed for untreated animals (Group 1). After day 35, the tumors of the animals in Groups 3-6 decreased in volume. The average tumor volume in animals treated with GLV-1h68 and antibiotics reduced to a volume that was approximately the same (Group 6) or less than (Group 4) the starting tumor volume. By the end of the study, the tumors animals receiving virus only (Group 3) were still approximately 2.5 times the volume of the average tumors at the start of the study. These results demonstrate that treatment of tumor bearing animals with antibiotics alone feeds tumor progression, treatment with GLV-1h68 is effective in reducing and reversing tumor growth, and GLV-1h68 in combination with antibiotic treatment is more effective at reversing tumor growth than virus treatment alone.

### F. Effect on Immune Cell Populations

Immune cell populations were determined by measuring changes in immune cells and interferon gamma production in mice sacrificed 7 days post virus injection. Flow cytometry was used to determine the change in immune cells, including NK cells, dendritic cells (DCs), Macrophages and B cells, in blood and spleen samples.

1. Blood Samples

For blood samples, whole blood from the tumor bearing mouse was collected by cardiac puncture in anti-coagulated tubes. Red blood cells were lysed with 1× red blood cell lysis buffer (BD Biosciences). About 400,000 peripheral blood mononuclear cells (PBMC) were labeled with the antibody mixes set forth in Table 18 to detect 1) Macrophages, 2) dendritic cells (DCs), and 3) NK and B cells. Labeled cells were analyzed using a Beckman Coulter Cell Lab Quanta SC flow cytometer. The results are set forth in Table 19 below.

TABLE 18
Antibody (Ab) mixes

| Fluorescent | | | | PerCp- | APC or | |
|---|---|---|---|---|---|---|
| Conjugate | V450 | FITC | PE | Cy5.5 | Alexa Fluor 647 | APC-Cy7 |
| Macrophage Ab Mix | CD14 | | IA-IE | F4/80 | CXCR4 | CD11b |
| | | | | | (CD184-APC) | |
| DC Ab Mix | | CD282 | IA-IE | CD80 | CD205 | CD11c |
| | | (TLR2) | | | | |
| NK & B cell Ab Mix | | CD45R | DX5 | NKp46 | CD19 | Gr-1 |
| | | (B220) | (CD49b) | (CD335) | | (Ly-6C/Ly-6G) |

TABLE 19
Number of Immune Cells per mL of Blood

| | Mac | | Monocytes | | NK cells | | B cells | |
|---|---|---|---|---|---|---|---|---|
| | Average | SD | Average | SD | Average | SD | Average | SD |
| GLV-1h68 | 507168 | 481257 | 750906 | 546651 | 518148 | 193191 | 673002 | 261116 |
| (Group 3) | | | | | | | | |
| AbT-GLV-1h68 | 1456920 | 1012808 | 999378 | 214826 | 751743 | 67921 | 698643 | 427974 |
| (Group 4) | | | | | | | | |
| AbT-Rec-GLV-1h68 | 1415952 | 746160 | 874359 | 200426 | 835668 | 192494 | 670365 | 259781 |
| (Group 5) | | | | | | | | |

The number of circulating macrophages was increased approximately 3-fold in antibiotic-treated cells (Groups 4 and 5) compared to GLV-1h68 infected cells alone (Group 3). Recolonization did not affect the number of circulating macrophages. The other immune cells, monocytes, NK cells and B cells were present in the blood at similar levels for all three treatment Groups.

2. Spleen Samples

For spleen samples, spleens were freshly excised and used to generate single cell suspensions by gently crushing with a frosted microscope slide into a Petri dish. Splenocyte suspensions were passed 3 times through a 20-G1″ needle and then through a 70 gm nylon mesh Cell Strainer (BD Bioscience). Cells were harvested by centrifugation at 1200 rpm for 10 min. The red blood cells were lysed with 1× red blood cell lysis buffer (BD Biosciences). RPMI, supplemented with 5% FBS was added to stop the lysis reaction. The resulting cell suspension was passed through a 40 μm nylon mesh Cell Strainer (BD Biosciences) to remove remaining cell aggregates. Single cell suspensions containing about 900,000 cells were labeled using the antibody mixes described in Table 18 above to detect Macrophages, NK and B cells. Labeled cells were analyzed using a Beckman Coulter Cell Lab Quanta SC flow cytometer. The results are set forth in Table 20 below.

TABLE 20
Number of Immune Cells Detected per Spleen

| | Macrophages | | NK cells | | B cells | |
|---|---|---|---|---|---|---|
| | Average | SD | Average | SD | Average | SD |
| GLV-1h68 | 1712900 | 718132 | 4419833 | 872629 | 28235467 | 5843562 |
| (Group 3) | | | | | | |
| AbT-GLV-1h68 | 1569500 | 483063 | 4351200 | 378159 | 25071167 | 5013345 |
| (Group 4) | | | | | | |
| AbT-Rec-GLV-1h68 | 1712333 | 95438 | 3786767 | 378159 | 22596933 | 11705837 |
| (Group 5) | | | | | | |

Animals infected with GLV-1h68 and treated with antibiotics, with or without bacterial Recolonization (Groups 4 and 5) alone (Group 3), contained similar numbers of macrophages, NK cells and B cells as animals infected with virus alone. Taken together with the results from the blood samples above, these results indicate the elevated macrophage levels in the blood are not a result of general upregulation of macrophage production, but likely represent increased differentiation and/or infiltration of inflammatory macrophages.

### Example 6

### In vivo Treatment with GLV-1h68 and Antibiotics

In this example, the effect of vaccinia virus treatment in conjugation with the administration of antibiotics was determined in vivo in cancer patients treated with GLV-1h68.

### A. Methods

1. Administration of GLV-1h68 and Antibiotics

A cancer patient was administered 1×10⁷ pfu GLV-1h68 intraperitoneally (i.p.) (10 minute infusion, 500 mL volume) on day on 1 of the treatment cycle. The patient was subsequently administered tazobactam, meropenem and vancomycin intravenously between days 9 and 22 as set forth in Table 21. A second cancer patient was administered 1×10⁷ pfu GLV-1h68 intraperitoneally (10 minute infusion, 500 mL volume) on day 1 of the treatment cycle but was not administered antibiotics. Efficacy of viral therapy was determined by measuring viral replication, e.g., shedding, using a viral plaque assay and detection of virus encoded reporter protein β-glucuronidase, inflammatory responses and oncolytic efficacy.

TABLE 21
Dosage regimen for treatment with vaccinia virus and antibiotics

| | Days of | | | | |
|---|---|---|---|---|---|
| | Admin- | | | Frequency of | Route of |
| Treatment | istration | Dosage | | Administration | Administration |
| GLV-1h68 | 1 | 1 × 10⁷ | pfu | 1 dose | i.p. |
| Tazobactam | 7-9 | 4.5 | g | 3x/day | i.v. |
| Meropenem | 9-22 | 1 | g | 3x/day | i.v. |
| Vancomycin | 9-21 | 1 | g | 1x/day | i.v. |

2. Viral Replication

Viral replication was assessed by viral plaque assay (VPA) and β-glucuronidase production as described below.

a. Viral Particle Assay

The number of infectious virus particles in body fluids and samples was assessed using a standard viral plaque assay, using serial dilutions on CV-1 cells, and expressed as pfu/mL (Yu et al., (2004) Nat Biotechnol. 22:313-320). Viral particles were stained with a specific anti-A27L antibody, which was custom made against a VACV synthetic peptide (GenScript Corporation). Body fluids and samples tested included peritoneal fluid, full blood, blood cells, blood lysates, urine, sputum and anal swab. For sampling of peritoneal fluid from the patient receiving viral therapy and antibiotics, ascites were sampled on days 4-14 and peritoneal lavage was sampled on days 16-59. Ascites were tested in the patient receiving only viral therapy. VPA was assessed 2 hours post viral treatment, and daily for 59 days. Viral particle counts were reported as pfu/mL.

b. β-Glucuronidase Assay

The GLV-1h68 virus contains a gene encoding β-glucuronidase which can be used as a marker protein for viral replication and cell lysis. β-glucuronidase release was measured in peritoneal fluid and EDTA plasma. Samples (20 μL) were incubated with 3.75 μg 4-MUG for one hour at 37° C. Fluorescence was then determined using a SpectraMax M5 fluorometer and was reported as relative fluorescence units per mL (RFU/mL).

### B. Results

The results show the patient receiving viral therapy and antibiotics had significant prolonged inherent (in situ) intraperitoneal production of GLV-1h68 progeny viral particles compared to the patient that only received viral therapy. Virus particles were observed in the antibiotic treated patient on days 4 through day 22, whereas viral particles tapered off after day 8, with only small amounts detected on days 10-12 for the patient that did not receive antibiotics. On day 8, virus yield in the patient treated with antibiotics was 1.54×10⁸, 15 times higher than the input virus dosage. Viral shedding was not observed in other fluids or samples that were tested.

β-Glucuronidase activity was detected in the peritoneal fluid and EDTA plasma of the patient treated with GLV-1h68 and antibiotics between days 4 and 59, with a peak in activity on days 10-11. In contrast, β-glucuronidase activity was only detected through day 12, with a peak in activity in peritoneal fluid at day 9 in the patient that only received viral therapy.

Overall, the results show that treatment with GLV-1h68 and antibiotics resulted in increased and prolonged viral efficacy as compared to treatment with viral therapy alone.

Since modifications will be apparent to those of skill in this art, it is intended that this invention be limited only by the scope of the appended claims.

1. A method for enhancing the effectiveness of a therapeutic virus, comprising administering an antibiotic with, before, after or during treatment with the therapeutic virus, to inhibit the growth of or kill commensal gut bacteria to thereby reduce the number of gut bacteria, wherein:
the antibiotic is an antibiotic that inhibits the growth of or kills commensal gut bacteria and is not an anti-cancer antibiotic; and
the antibiotic is administered in an amount that reduces or eliminates commensal gut bacteria.

2. The method of claim 1, wherein the therapeutic virus is administered to provide gene therapy and/or to treat cancers and tumors.

3. The method of claim 1, wherein the therapeutic virus is an oncolytic virus.

4. The method of claim 1, wherein the therapeutic virus is selected from among a retrovirus, adenovirus, lentivirus, herpes simplex virus, poxvirus and adeno-associated virus (AAV).

5. The method of claim 4, wherein the therapeutic virus is an oncolytic virus selected from among Newcastle Disease virus, parvovirus, vaccinia virus, measles virus, reovirus, oncolytic adenoviruses and vesicular stomatitis virus (VSV).

6. The method of claim 1, wherein the therapeutic virus is a vaccinia virus.

7. A method for treating cancers or tumors, comprising:
administering a therapeutic virus for treatment of cancers, tumors or metastases, wherein the therapeutic virus is effective for treating one or more of cancers, tumors or metastases; and
administering an antibiotic that is effective against commensal gut bacteria, wherein:
the antibiotic is administered before, after or with the therapeutic virus; and
the antibiotic is administered in an amount that reduces or eliminates commensal gut bacteria.

8. The method of claim 7, wherein the therapeutic virus is an oncolytic virus.

9. The method of claim 7, wherein the therapeutic virus is selected from among a retrovirus, adenovirus, lentivirus, herpes simplex virus, poxvirus and adeno-associated virus (AAV).

10. The method of claim 8, wherein the therapeutic virus is an oncolytic virus selected from among Newcastle Disease virus, parvovirus, a pox virus, measles virus, reovirus, vesicular stomatitis virus (VSV), oncolytic adenovirus, poliovirus and herpes simplex virus.

11. The method of claim 7, wherein the therapeutic virus is a vaccinia virus.

12. The method of claim 10, wherein the therapeutic virus is a pox virus that is a strain selected from among Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP and WR 65-16 strains and modified forms of the strains.

13. The method of claim 6, wherein the therapeutic virus is a Wyeth strain derived virus designated JX-294 or JX-594 or is an LIVP virus that is the virus designated GLV-1h68 and derivatives and modified forms thereof.

14. The method of claim 11, wherein the vaccinia virus a Lister strain virus.

15. The method of claim 14, wherein the virus is an LIVP virus, a clonal strain of an LIVP virus, or a modified form thereof containing nucleic acid encoding a heterologous gene product.

16. The method of claim 15, wherein the nucleic acid encoding the heterologous gene product is inserted into or in place of a non-essential gene or region in the genome of the virus.

17. The method of claim 16, wherein the nucleic acid encoding the heterologous gene product is inserted at the hemagglutinin (HA), thymidine kinase TK), F14.5L, vaccinia growth factor (VGF), A35R, N1L, E2L/E3L, K1L/K2L, superoxide dismutase locus, 7.5K, C7-K1L, B13R+B14R, A26L or 14L gene loci in the genome of the virus.

18. The method of claim 15, wherein the virus is an LIVP virus or modified form thereof comprising a sequence of nucleotides set forth in SEQ ID NO:2, or a sequence of nucleotides that has at least 95% sequence identity to SEQ ID NO:2.

19. The method of claim 15, wherein the virus is a clonal strain of LIVP or a modified form thereof comprising a sequence of nucleotides selected from:
a) nucleotides 2,256-180,095 of SEQ ID NO:3, nucleotides 11,243-182,721 of SEQ ID NO:4, nucleotides 6,264-181,390 of SEQ ID NO:5, nucleotides 7,044-181,820 of SEQ ID NO:6, nucleotides 6,674-181,409 of SEQ ID NO:7, nucleotides 6,716-181,367 of SEQ ID NO:8 or nucleotides 6,899-181,870 of SEQ ID NO:9;
b) a sequence of nucleotides that has at least 97% sequence identity to a sequence of nucleotides 2,256-180,095 of SEQ ID NO:3, nucleotides 11,243-182,721 of SEQ ID NO:4, nucleotides 6,264-181,390 of SEQ ID NO:5, nucleotides 7,044-181,820 of SEQ ID NO:6, nucleotides 6,674-181,409 of SEQ ID NO:7, nucleotides 6,716-181,367 of SEQ ID NO:8 or nucleotides 6,899-181,870 of SEQ ID NO:9.

20. The method of claim 19, wherein the virus comprises a sequence of nucleotides set forth in any of SEQ ID NOS: 3-9, or a sequence of nucleotides that has at least 97% sequence identity to a sequence of nucleotides set forth in any of SEQ ID NOS: 3-9.

21. The method of claim 15, wherein the virus comprises heterologous nucleic acid that comprises a reporter gene or encodes a detectable gene product or a product that produces a detectable signal.

22. The method of claim 21, wherein the reporter gene encodes a fluorescent protein, a bioluminescent protein, a receptor or an enzyme.

23. The method of claim 22, wherein the encoded gene product is a fluorescent protein selected from among a green fluorescent protein, an enhanced green fluorescent protein, a blue fluorescent protein, a cyan fluorescent protein, a yellow fluorescent protein, a red fluorescent protein, or a far-red fluorescent protein.

24. The method of claim 15, wherein the virus encodes a product that is detectable or that induces a detectable signal.

25. The method of any of claim 15, wherein the virus comprises nucleic acid encoding a heterologous gene product that is a therapeutic agent a diagnostic agent or comprises a plurality thereof.

26. The method of claim 25, wherein the heterologous gene product is selected from among an anticancer agent, an antimetastatic agent, an antiangiogenic agent, an immunomodulatory molecule, an antigen, a cell matrix degradative gene, genes for tissue regeneration and reprogramming human somatic cells to pluripotency, enzymes that modify a substrate to produce a detectable product or signal or are detectable by antibodies, proteins that can bind a contrasting agent, genes for optical imaging or detection, genes for PET imaging and genes for MRI imaging.

27. The method of claim 15, wherein the virus comprises a sequence of nucleotides selected from among any of SEQ ID NOS:1 and 10-19, or a sequence of nucleotides that exhibits at least 99% sequence identity to any of SEQ ID NOS: 1 and 10-19.

28. The method of claim 7, wherein the antibiotic is administered in an amount between about 1 mg and about 1000 mg.

29. The method of claim 7, wherein the antibiotic is administered prior to the administration of the virus.

30. The method of claim 29, wherein the antibiotic is administered at least, at about or at 1, 2, 3, 4, 5, 6, 7, 8, 9, 10, 11, 12, 13, 14, 15, 16, 17, 18, 19, 20, 21, 22, 23, 24, 36 or 48 or more hours prior to the administration of the virus.

31. The method of claim 7, wherein the antibiotic is administered at the same time as the administration of the virus.

32. The method of claim 7, wherein the antibiotic is administered after the administration of the virus.

33. The method of claim 32, wherein the antibiotic is administered at least, at about or at 1, 2, 3, 4, 5, 6, 7, 8, 9, 10, 11, 12, 13, 14, 15, 16, 17, 18, 19, 20, 21, 22, 23, 24 or more hours, or 2, 3, 4, 5, 6, 7, 8, 9, 10, 11, 12, 13, 14 or more days after the administration of the virus.

34. The method of claim 7, wherein the antibiotic is administered a plurality of times.

35. The method of claim 7, wherein the antibiotic is selected from among penicillins, penicillin combinations, tetracyclines, β-lactam antibiotics, carbacephems, glycopeptides, aminoglycosides, ansamycins, macrolides, monobactams, nitrofurans, sulfonamides, lincosamides, lipopeptides, polypeptides, quinolones, drugs against mycobacteria, oxazolidinones, arsphenamine, chloramphenicol, fosfomycin, fusidic acid, metronidazole, tazobactam, mupirocin, platensimycin, quinupristin/dalfopristin, thiamphenicol, tigecycline, tinidazole or trimethoprim and mixtures thereof.

36. The method of claim 7, wherein the antibiotic is selected from among penicillin, streptomycin, ampicillin, neomycin, metronidazole, vancomycin, tazobactam, meropenem, a mixture of penicillin and streptomycin, a mixture of ampicillin, neomycin, metronidazole and vancomycin, and a mixture of tazobactam, meropenem and vancomycin.

37. The method of claim 7, further comprising administering an antimycotic with the antibiotic or before or after the administration of the antibiotic or with the administration of the virus or before or after the administration of the virus, wherein the antimycotic is administered in an amount effective for treatment of any fungal infections.

38. A combination, comprising:
a first composition, comprising a therapeutic virus in a pharmaceutically acceptable vehicle, and
a second composition, comprising an antibiotic in a pharmaceutically acceptable vehicle, wherein the antibiotic inhibits the growth of or kills commensal gut bacteria to thereby reduce the number of gut bacteria and is not an anti-cancer antibiotic.

39. The combination of claim 38, wherein the therapeutic virus provides gene therapy and/or treats cancers and tumors.

40. The combination of claim 38, wherein the therapeutic virus is an oncolytic virus.

41. The combination of claim 38, wherein the therapeutic virus is a pox virus.

42. The combination of claim 41, wherein the therapeutic virus is a vaccinia virus.

43. The combination of claim 41, wherein the therapeutic virus is a pox virus that is a strain selected from among Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP and WR 65-16 strains and modified forms of the strains.

44. The combination of claim 43, wherein the therapeutic virus is an LIVP strain virus.

45. The combination of claim 44, wherein the LIVP strain virus is the virus designated GLV-1h68 and derivatives and modified forms thereof.

46. The combination of claim 43, wherein the therapeutic virus is a Lister strain virus.

47. The combination of claim 38, wherein the antibiotic is selected from among penicillins, penicillin combinations, tetracyclines, β-lactam antibiotics, carbacephems, glycopeptides, aminoglycosides, ansamycins, macrolides, monobactams, nitrofurans, sulfonamides, lincosamides, lipopeptides, polypeptides, quinolones, drugs against mycobacteria, oxazolidinones, arsphenamine, chloramphenicol, fosfomycin, fusidic acid, metronidazole, tazobactam, mupirocin, platensimycin, quinupristin/dalfopristin, thiamphenicol, tigecycline, tinidazole or trimethoprim and mixtures thereof.
